(19) United States
(12) Patent Application Publication
(10) Pub. No.: US 2025/0361298 A1
(43) Pub. Date: Nov. 27, 2025

(54) LY6G6D BINDING PROTEINS, NUCLEIC ACIDS ENCODING SUCH PROTEINS, AND METHODS FOR THE PREPARATION AND USE THEREOF

(71) Applicant: CARTOGRAPHY BIOSCIENCES, INC., SOUTH SAN FRANCISCO, CA (US)

(72) Inventors: Alexander John MARTINKO, SAN FRANCISCO, CA (US); David Geoffrey KUGLER, SEATTLE, WA (US); Burcu YIGIT, PALO ALTO, CA (US); Robert CHEN, BELMONT, CA (US); Prasuna PALURU, SAN FRANCISCO, CA (US); Janine SCHUURMAN, DIEMEN (NL)

(73) Assignee: CARTOGRAPHY BIOSCIENCES, INC., SOUTH SAN FRANCISCO, CA (US)

(21) Appl. No.: 19/188,935

(22) Filed: Apr. 24, 2025

Related U.S. Application Data
(60) Provisional application No. 63/638,332, filed on Apr. 24, 2024.

(51) Int. Cl. C07K 16/28

(52) U.S. Cl. CPC C07K 16/2803; C07K 2317/33; C07K 2317/52; C07K 2317/92

(57) ABSTRACT

Immunoglobulin complementarity determining regions (“CDRs”), and immunoglobulin binding domains comprising those CDRs, that bind human lymphocyte antigen 6 family member G6D (LY6G6D), and their use for the preparation of LY6G6D-binding proteins finding use as immunotherapeutics.

### CROSS-REFERENCE TO RELATED APPLICATIONS

The present application claims the benefit of U.S. Provisional Application No. 63/638,332 filed on Apr. 24, 2024, from which priority is claimed and which is hereby incorporated by reference in its entirety.

### SEQUENCE LISTING

The application contains a Sequence Listing which has been submitted electronically in .XML format and is hereby incorporated by reference in its entirety. Said. XML copy, created on Jul. 3, 2025, is named “CART-002-US1_SeqListing.xml” and is 2,335 kilobytes in size. The sequence listing contained in this .XML file is part of the specification and is hereby incorporated by reference herein in its entirety.

### FIELD OF THE INVENTION

The present invention relates to the preparation of immunoglobulin complementarity determining regions (“CDRs”), and immunoglobulin binding domains comprising those CDRs, that bind human lymphocyte antigen 6 family member G6D (LY6G6D), and their use for the preparation of LY6G6D-binding proteins in various formats.

### BACKGROUND OF THE INVENTION

LY6G6D belongs to a cluster of leukocyte antigen-6 (LY6) genes located in the major histocompatibility complex (MHC) class III region on chromosome 6. Members of the LY6 superfamily typically contain 70 to 80 amino acids, including 8 to 10 cysteines. Most LY6 proteins are attached to the cell surface by a glycosylphosphatidylinositol (GPI) anchor that is directly involved in signal transduction. Human LY6G6D (UniProt entry 095868) is expressed as a 133-residue polypeptide that is processed to a mature form comprising 85 amino acids.

LY6G6D is reportedly expressed in colorectal cancer, and particularly in microsatellite stable (MSS) forms of the disease. MSS CRC represents greater than 90% of patients with metastatic CRC. MSS colorectal tumors are considered to be immunologically “cold” due to a relatively low tumor mutational burden and a tumor microenvironment that lacks substantial T cell infiltration.

A T cell engager known as LY6G6D-TDB (also known as BLYG8824A) has been shown to have single agent efficacy in murine xenograph CRC models, and that efficacy reportedly improves in combination with checkpoint blockade. Wang et al., Mol. Cancer Ther. 2022 Jun. 1; 21 (6): 974-985 (doi: 10.1158/1535-7163.MCT-21-0599). BLYG8824A is in phase I clinical trials in MSS CRC.

### SUMMARY OF THE INVENTION

The present invention relates to human LY6G6D-binding proteins and related compositions and methods.

Mature human LY6G6D (hLY6G6D, residues 20-104 of Swiss-Prot Accession 095868) has the following sequence (SEQ ID NO: 1):

NRMRCYNCGG SPSSSCKEAV TTCGEGRPQP GLEQIKLPGN
PPVTLIHQHP ACVAAHHCNQ VETESVGDVT YPAHRDCYLG DLCNS

In a first aspect, the present invention provides hLY6G6D binding proteins comprising an immunoglobulin variable region that binds hLY6G6D, wherein the immunoglobulin variable region comprises heavy chain CDRs H1, H2, and H3 and light chain CDRs L1, L2, and L3 as recited for one of the Identifiers in Table 1.

TABLE 1
(SEQ ID NOs: 2-1993, with numbering across rows (so row 1 is SEQ
ID NOs: 2-7, row 2 is SEQ ID NOs: 8-13, etc.):

| Identifier | CDR H1 | CDR H2 | CDR H3 | CDR L1 | CDR L2 | CDR L3 |
|---|---|---|---|---|---|---|
| CB21-469 | SYAMN | GISGNGGRIYYA | DLDV | RSSQSLVHS | KISNRFS | MQATQFPLT |
| | | DSVKG | | DGNTYLN | | |
| CB21-470 | SYGMH | VLWYDGINKNY | VQGDY | RASQGIRN | DTSSLQS | LQHNSYPFT |
| | | ADSVKG | | DLT | | |
| CB21-471 | SYGMH | VIWYDGSNKDY | PGEGDY | RASQGIRN | GASSLQS | LQQNSYPWT |
| | | VDSVKG | | DFG | | |
| CB21-477 | SYGMH | VIWYDGSNKDY | PGEGDY | RASQGIRN | AASSLQS | LQQNSYPWT |
| | | ADSVKG | | DLG | | |
| CB21-500 | SYAMN | GMSGSGGRIYY | DLDV | RSSQNLVHS | KISNRFS | MQATQFPLT |
| | | ADSVKG | | DGNTYLN | | |
| CB21-502 | SYGMH | VLWYDGINKNY | VQGDY | RASQGIRN | DTSSLQS | LQHNIYPFT |
| | | VDSVKG | | DLT | | |
| CB21-512 | SYGMH | VLWYDGINKNY | VQGDY | RASQGIRN | DTSSLQS | LQHNIYPFT |
| | | ADSVKG | | DLT | | |
| CB21-513 | SYAMT | GISGSGGVTDYA | DLGV | RSSQSLVHS | KISNRFS | MQATQFPLT |
| | | DSVKG | | DGNTYLN | | |
| CB21-522 | GYFIH | WINPNSGGTYY | DISLVGD | RSSQSLVYS | KVSNRDS | MQGSHWPLT |
| | | AQEFQG | Y | DGNTYLN | | |
| CB21-498 | SYAMN | GISGNGGRIYYA | DLDV | RSSQSLVHS | KISNRFS | MQATQFPLT |
| | | ESVKG | | DGNTYLN | | |
| CB21-498- | SYAMN | GISGNGGRIYYA | DLDV | RSSQSLVHS | KISNRFS | MQATQFPLT |
| 001 | | ESVKG | | DGNTYLN | | |
| CB21-498- | SYAMN | GISGNGGRIYYA | DLDV | RSSQSLVHS | KISNRFS | MQATQFPLT |
| 002 | | ESVKG | | DGNTYLN | | |
| CB21-498- | SYAMN | GISGNAGRIYYA | DLDV | RSSQSLVHS | KISNRFS | MQATQFPLT |
| 003 | | ESVKG | | DGNTYLN | | |
| CB21-498- | SYAMN | GISGNPGRIYYA | DLDV | RSSQSLVHS | KISNRFS | MQATQFPLT |
| 004 | | ESVKG | | DGNTYLN | | |
| CB21-498- | SYAMN | GISGQGGRIYYA | DLDV | RSSQSLVHS | KISNRFS | MQATQFPLT |
| 005 | | ESVKG | | DGNTYLN | | |
| CB21-498- | SYAMN | GISGSGGRIYYA | DLDV | RSSQSLVHS | KISNRFS | MQATQFPLT |
| 006 | | ESVKG | | DGNTYLN | | |
| CB21-498- | SYAMN | GISGNGGRIYYA | DLDV | RSSQSLVHS | KISNRFS | MQATQFPLT |
| 007 | | ESVKG | | DGNTYLN | | |
| CB21-498- | SYAMN | GISGNGGRIYYA | DLDV | RSSQSLVHS | KISNRFS | MQATQFPLT |
| 008 | | ESVKG | | DGNTYLN | | |
| CB21-498- | SYAMN | GISGNGGSIYYA | DLDV | RSSQSLVHS | KISNRFS | MQATQFPLT |
| 009 | | ESVKG | | DGNTYLN | | |
| CB21-498- | SYAMN | GISGNGGRIYYA | DLDV | RSSQSLVHS | KISNRFS | MQATQFPLT |
| 010 | | ESVKG | | DGNTYLN | | |
| CB21-498- | SYAMN | GISGNGGRIYYA | DLDV | RSSQSLVHS | KISNRFS | MQATQFPLT |
| 011 | | ESVKG | | DANTYLN | | |
| CB21-498- | SYAMN | GISGNGGRIYYA | DLDV | RSSQSLVHS | KISNRFS | MQATQFPLT |
| 012 | | ESVKG | | DINTYLN | | |
| CB21-498- | SYAMN | GISGNGGRIYYA | DLDV | RSSQSLVHS | KISNRFS | MQATQFPLT |
| 013 | | ESVKG | | IGNTYLN | | |
| CB21-498- | SYAMN | GISGNGGRIYYA | DLDV | RSSQSLVHS | KISNRFS | MQATQFPLT |
| 014 | | ESVKG | | EGNTYLN | | |
| CB21-498- | SYAMN | GISGNGGRIYYA | DLDV | RSSQSLVHS | KISNRFS | MQATQFPLT |
| 015 | | ESVKG | | DGNTYLA | | |
| CB21-498- | SYAMN | GISGNGGRIYYA | DLDV | RSSQSLVHS | KISNRFS | MQATQFPLT |
| 016 | | ESVKG | | DGNTYLN | | |
| CB21-498- | SYAMN | GISGNGGRIYYA | DLDV | RSSQSLVHS | KISNRFS | MQATQFPLT |
| 017 | | ESVKG | | DGNTYLN | | |
| CB21-498- | SYAMN | GISGNGGRIYYA | DLDV | RSSQSLVHS | KISNRFS | MQATQFPLT |
| 018 | | ESVKG | | DGNTYLN | | |
| CB21-498- | SYAMN | GISGNGGRIYYA | DLDV | RSSQSLVHS | KASNRFS | MQATQFPLT |
| 019 | | ESVKG | | DGNTYLN | | |
| CB21-498- | SYAMN | GISGNGGRIYYA | DLDV | RSSQSLVHS | KISNRFS | MQATQFPLT |
| 020 | | ESVKG | | DGNTYLN | | |
| CB21-498- | SYAMN | GISGNGGRIYYA | DLDV | RSSQSLVHS | KISNRFS | MQATQFPLT |
| 021 | | ESVKG | | DGNTYLN | | |
| CB21-498- | SYAMN | GISGNGGRIYYA | DLDV | RSSQSLVHS | KISNRFS | MQATQFPLT |
| 022 | | ESVKG | | DGNTYLN | | |
| CB21-498- | SYAMN | GISGNGGRIYYA | DLDV | RSSQSLVHS | KISNRFS | MQATQFPLT |
| 023 | | ESVKG | | DGNTYLN | | |
| CB21-498- | SYAMN | GISGNGGRIYYA | DLDV | RSSQSLVHS | KISNRFS | MQATQFPLT |
| 024 | | ESVKG | | DGNTYLN | | |
| CB21-498- | SYAMN | GISGNGGRIYYA | DLDV | RSSQSLVHS | KISNRFS | MQATQFPLT |
| 025 | | ESVKG | | DGNTYLN | | |
| CB21-498- | SYAMN | GISGNGGRIYYA | DLDV | RSSQSLVHS | KISNRFS | MQATQFPLT |
| 026 | | ESVKG | | DGNTYLN | | |
| CB21-498- | SYAMN | GISGNGGRIYYA | DLDV | RSSQSLVHS | KISNRFS | MQATQFPLT |
| 027 | | ESVKG | | DGNTYLN | | |
| CB21-498- | SYAMN | GISGNAGRIYYA | DLDV | RSSQSLVHS | KISNRFS | MQATQFPLT |
| 028 | | ESVKG | | DGNTYLN | | |
| CB21-498- | SYAMN | GISGNGGRIYYA | DLDV | RSSQSLVHS | RISNRFS | MQATQFPLT |
| 029 | | ESVKG | | DGNTYLN | | |
| CB21-498- | SYAMN | GISGNGGRIYYA | DLDV | RSSQSLVHS | KASNRFS | MQATQFPLT |
| 030 | | ESVKG | | DGNTYLN | | |
| CB21-498- | SYAMN | GISGNGGRIYYA | DLDV | RSSQSLVHS | KNSNRFS | MQATQFPLT |
| 031 | | ESVKG | | DGNTYLN | | |
| CB21-498- | SYAMN | GISGNGGRIYYA | DLDV | RSSQSLVHS | KTSNRFS | MQATQFPLT |
| 032 | | ESVKG | | DGNTYLN | | |
| CB21-498- | SYAMN | GISGNGGRIYYA | DLDV | RSSQSLVHS | KVSNRFS | MQATQFPLT |
| 033 | | ESVKG | | DGNTYLN | | |
| CB21-498- | SYAMN | GISGNGGRIYYA | DLDV | RSSQSLVHS | KISNLFS | MQATQFPLT |
| 034 | | ESVKG | | DGNTYLN | | |
| CB21-498- | SYAMN | GISGNGGRIYYA | DLDV | RSSQSLVHS | KISNDFS | MQATQFPLT |
| 035 | | ESVKG | | DGNTYLN | | |
| CB21-498- | SYAMN | GISGNGGRIYYA | DLDV | RSSQSLVHS | KISNEFS | MQATQFPLT |
| 036 | | ESVKG | | DGNTYLN | | |
| CB21-498- | SYAMN | GISGNGGRIYYA | DLDV | RSSQSLVHS | KISNSFS | MQATQFPLT |
| 037 | | ESVKG | | DGNTYLN | | |
| CB21-498- | SYAMN | GISGNGGRIYYA | DLDV | RSSQSLVHS | KISNPFS | MQATQFPLT |
| 038 | | ESVKG | | DGNTYLN | | |
| CB21-498- | SYAMN | GISGNGGRIYYA | DLDV | RSSQSLVHS | KISNRPS | MQATQFPLT |
| 039 | | ESVKG | | DGNTYLN | | |
| CB21-498- | SYAMN | GISGNGGRIYYA | DLDV | RSSQSLVHS | KISNRAS | MQATQFPLT |
| 040 | | ESVKG | | DGNTYLN | | |
| CB21-498- | SYAMN | GISGNGGRIYYA | DLDV | RSSQSLVHS | KISNRGS | MQATQFPLT |
| 041 | | ESVKG | | DGNTYLN | | |
| CB21-498- | SYAMN | GISGNGGRIYYA | DLDV | RSSQSLVHS | KISNRSS | MQATQFPLT |
| 042 | | ESVKG | | DGNTYLN | | |
| CB21-498- | SYAMN | GISGNGGRIYYA | DLDV | RSSQSLVHS | KISNRNS | MQATQFPLT |
| 043 | | ESVKG | | DGNTYLN | | |
| CB21-498- | SYAMN | GISGNGGRIYYA | DLDV | RSSQSLVHS | KISNRQS | MQATQFPLT |
| 044 | | ESVKG | | DGNTYLN | | |
| CB21-498- | SYAMN | GISGNGGRIYYA | DLDV | RSSQSLVHS | KISNRES | MQATQFPLT |
| 045 | | ESVKG | | DGNTYLN | | |
| CB21-498- | SYAMN | GISGNGGRIYYA | DLDV | RSSQSLVHS | KISNRDS | MQATQFPLT |
| 046 | | ESVKG | | DGNTYLN | | |
| CB21-498- | SYAMN | GISGNGGRIYYA | DLDV | RSSQSLVHS | KISNRFP | MQATQFPLT |
| 047 | | ESVKG | | DGNTYLN | | |
| CB21-498- | SYAMN | GISGNGGRIYYA | DLDV | RSSQSLVHS | KISNRFS | MQATQFPLT |
| 048 | | ESVKG | | DGNTYLN | | |
| CB21-498- | SYAMN | GISGNGGRIYYA | DLDV | RSSQSLVHS | KISNRFS | NQATQFPLT |
| 049 | | ESVKG | | DGNTYLN | | |
| CB21-498- | SYAMN | GISGNGGRIYYA | DLDV | RSSQSLVHS | KISNRFS | WQATQFPLT |
| 050 | | ESVKG | | DGNTYLN | | |
| CB21-498- | SYAMN | GISGNGGRIYYA | DLDV | RSSQSLVHS | KISNRFS | MQGTQFPLT |
| 051 | | ESVKG | | DGNTYLN | | |
| CB21-498- | SYAMN | GISGNGGRIYYA | DLDV | RSSQSLVHS | KISNRFS | MQNTQFPLT |
| 052 | | ESVKG | | DGNTYLN | | |
| CB21-498- | SYAMN | GISGNGGRIYYA | DLDV | RSSQSLVHS | KISNRFS | MQATQSPLT |
| 053 | | ESVKG | | DGNTYLN | | |
| CB21-498- | SYAMN | GISGNGGRIYYA | DLDV | RSSQSLVHS | KISNRFS | MQATQTPLT |
| 054 | | ESVKG | | DGNTYLN | | |
| CB21-498- | SYAMN | GISGNGGRIYYA | DLDV | RSSQSLVHS | KISNRFS | MQATQYPLT |
| 055 | | ESVKG | | DGNTYLN | | |
| CB21-498- | SYAMN | GISGNGGRIYYA | DLDV | RSSQSLVHS | KISNRFS | MQATQNPLT |
| 056 | | ESVKG | | DGNTYLN | | |
| CB21-498- | SYAMN | GISGNGGRIYYA | DLDV | RSSQSLVHS | KISNRFS | MQATQLPLT |
| 057 | | ESVKG | | DGNTYLN | | |
| CB21-498- | SYAMN | GISGNGGRIYYA | DLDV | RSSQSLVHS | KISNRFS | MQATQAPLT |
| 058 | | ESVKG | | DGNTYLN | | |
| CB21-498- | SYAMN | GISGNGGRIYYA | DLDV | RSSQSLVHS | KISNRFS | MQATQFNLT |
| 059 | | ESVKG | | DGNTYLN | | |
| CB21-498- | SYAMN | GISGNGGRIYYA | DLDV | RSSQSLVHS | KISNRFS | MQATQFQLT |
| 060 | | ESVKG | | DGNTYLN | | |
| CB21-498- | SYAMN | GISGNGGRIYYA | DLDV | RSSQSLVHS | KISNRFS | MQATQFDLT |
| 061 | | ESVKG | | DGNTYLN | | |
| CB21-498- | SYAMN | GISGNGGRIYYA | DLDV | RSSQSLVHS | KISNRFS | MQATQFSLT |
| 062 | | ESVKG | | DGNTYLN | | |
| CB21-498- | SYAMN | GISGNGGRIYYA | DLDV | RSSQSLVHS | KISNRFS | MQATQFPST |
| 063 | | ESVKG | | DGNTYLN | | |
| CB21-498- | SYAMN | GISGNGGRIYYA | DLDV | RSSQSLVHS | KISNRFS | MQATQFPGT |
| 064 | | ESVKG | | DGNTYLN | | |
| CB21-498- | SYAMN | GISGNGGRIYYA | DLDV | RSSQSLVHS | KISNRFS | MQATQFPYT |
| 065 | | ESVKG | | DGNTYLN | | |
| CB21-498- | SYAMN | GISGNGGRIYYA | DLDV | RSSQSLVHS | KISNRFS | MQATQFPWT |
| 066 | | ESVKG | | DGNTYLN | | |
| CB21-498- | SYAMN | GISGNGGRIYYA | DLDV | RSSQSLVHS | KISNRFS | MQATQFSLP |
| 067 | | ESVKG | | DGNTYLN | | |
| CB21-498- | SYAMN | GISGSGGRIYYA | DLDV | RSSQSLVHS | KISNRFS | MQATQFPLT |
| 068 | | ESVKG | | DGNTYLN | | |
| CB21-498- | SYAMN | GISGNGGRIYYA | DLDV | RSSQSLVHS | KISNRFS | MQATQFPLT |
| 069 | | ESVKG | | DGNTYLN | | |
| CB21-498- | SYAMN | GISGSGGRIYYA | DLDV | RSSQSLVHS | KISNRFS | MQATQFPLT |
| 070 | | ESVKG | | DGNTYLN | | |
| CB21-498- | SYAMN | GISGNGGRIYYA | DLDV | RSSQSLVHS | KISNRFS | MQATQFPLT |
| 071 | | ESVKG | | DANTYLN | | |
| CB21-498- | SYAMN | GISGSGGRIYYA | DLDV | RSSQSLVHS | KISNRFS | MQATQFPLT |
| 072 | | ESVKG | | DANTYLN | | |
| CB21-498- | SYAMN | GISGNGGRIYYA | DLDV | RSSQSLVHS | KISNRFS | MQATQFPLT |
| 073 | | ESVKG | | DANTYLN | | |
| CB21-498- | SYAMN | GISGSGGRIYYA | DLDV | RSSQSLVHS | KISNRFS | MQATQFPLT |
| 074 | | ESVKG | | DANTYLN | | |
| CB21-498- | SYAMN | GISGNGGRIYYA | DLDV | RSSQSLVHS | KISNRFS | MQATQFPLT |
| 075 | | ESVKG | | DANTYLN | | |
| CB21-498- | SYAMN | GISGSGGRIYYA | DLDV | RSSQSLVHS | KISNRFS | MQATQFPLT |
| 076 | | ESVKG | | DANTYLN | | |
| CB21-498- | SYAMN | GISGNGGRIYYA | DLDV | RSSQSLVHS | KISNRFS | MQATQFPLT |
| 077 | | ESVKG | | DANTYLN | | |
| CB21-498- | SYAMN | GISGNGGRIYYA | DLDV | RSSQSLVHS | KISNRFS | MQATQFPLT |
| 078 | | ESVKG | | DANTYLN | | |
| CB21-498- | SYAMN | GISGSGGRIYYA | DLDV | RSSQSLVHS | KISNRFS | MQATQFPLT |
| 079 | | ESVKG | | DANTYLN | | |
| CB21-498- | SYAMN | GISGNGGRIYYA | DLDV | RSSQSLVHS | KISNRFS | MQATQFPLT |
| 080 | | ESVKG | | DANTYLN | | |
| CB21-498- | SYAMN | GISGSGGRIYYA | DLDV | RSSQSLVHS | KISNRFS | MQATQFPLT |
| 081 | | ESVKG | | DANTYLN | | |
| CB21-498- | SYAMN | GISGNGGRIYYA | DLDV | RSSQSLVHS | KISNRFS | MQATQFPLT |
| 082 | | ESVKG | | DANTYLN | | |
| CB21-498- | SYAMN | GISGSGGRIYYA | DLDV | RSSQSLVHS | KISNRFS | MQATQFPLT |
| 083 | | ESVKG | | DANTYLN | | |
| CB21-498- | SYAMN | GISGNGGRIYYA | DLDV | RSSQSLVHS | KISNRFS | MQATQFPLT |
| 084 | | ESVKG | | DANTYLN | | |
| CB21-498- | SYAMN | GISGNGGRIYYA | DLDV | RSSQSLVHS | KISNRFS | MQATQFPLT |
| 085 | | ESVKG | | DANTYLN | | |
| CB21-498- | SYAMN | GISGSGGRIYYA | DLDV | RSSQSLVHS | KISNRFS | MQATQFPLT |
| 086 | | ESVKG | | DANTYLN | | |
| CB21-498- | SYAMN | GISGNGGRIYYA | DLDV | RSSQSLVHS | KISNRFS | MQATQFPLT |
| 087 | | ESVKG | | DANTYLN | | |
| CB21-498- | SYAMN | GISGSGGRIYYA | DLDV | RSSQSLVHS | KISNRFS | MQATQFPLT |
| 088 | | ESVKG | | DANTYLN | | |
| CB21-498- | SYAMN | GISGNGGRIYYA | DLDV | RSSQSLVHS | KISNRFS | MQATQFPLT |
| 089 | | ESVKG | | DANTYLN | | |
| CB21-498- | SYAMN | GISGSGGRIYYA | DLDV | RSSQSLVHS | KISNRFS | MQATQFPLT |
| 090 | | ESVKG | | DANTYLN | | |
| CB21-498- | SYAMS | GISGSGGRIYYA | DLDV | RSSQSLVHS | KISNRFS | MQATQFPLT |
| 091 | | ESVKG | | DGNTYLN | | |
| CB21-498- | SYAMH | GISGSGGRIYYA | DLDV | RSSQSLVHS | KISNRFS | MQATQFPLT |
| 092 | | ESVKG | | DGNTYLN | | |
| CB21-498- | SYAMT | GISGSGGRIYYA | DLDV | RSSQSLVHS | KISNRFS | MQATQFPLT |
| 093 | | ESVKG | | DGNTYLN | | |
| CB21-498- | SYAMN | GISGSGGRIYYA | DLDV | RSSQSLVHS | KISNRFS | MQATQFPLT |
| 094 | | ESVKG | | DGNTYLN | | |
| CB21-498- | SYAMN | GISGSGGRIYYA | DLDV | RSSQSLVHS | KISNRFS | MQATQFPLT |
| 095 | | ESVKG | | DGNTYLN | | |
| CB21-498- | SYAMN | GISGSGGRIYYA | DLDV | RSSQSLVHS | KISNRFS | MQATQFPLT |
| 096 | | ESVKG | | DGNTYLN | | |
| CB21-498- | SYAMN | GISGSGGRIYKA | DLDV | RSSQSLVHS | KISNRFS | MQATQFPLT |
| 097 | | ESVKG | | DGNTYLN | | |
| CB21-498- | SYAMN | GISGSGGRIYYA | DLDV | RSSQSLVHS | KISNRFS | MQATQFPLT |
| 098 | | ESVKG | | DGNTYLN | | |
| CB21-498- | SYAMN | GISGSGGRIYYA | DLDV | RSSQSLVHS | KISNRFS | MQATQFPLT |
| 099 | | ESVKG | | DGNTYLN | | |
| CB21-498- | SYAMN | GISGSGGRIYYA | DLDV | RSSQSLVHS | KISNRFS | MQATQFPLT |
| 100 | | ESVKG | | DGNTYLN | | |
| CB21-498- | SYAMN | GISGSGGRIYYA | DLDV | RSSQSLVHS | KISNRFS | MQATQFPLT |
| 101 | | ESVKG | | DGNTYLN | | |
| CB21-498- | SYAMN | GISGSGGRIYYA | DLDV | RSSQSLVHS | KISNRFS | MQATQFPLT |
| 102 | | ESVKG | | DGNTYLN | | |
| CB21-498- | SYAMN | GISGSGGRIYYA | DLDV | RSSQSLVHS | KISNRFS | MQATQFPLT |
| 103 | | ESVKG | | DGNTYLN | | |
| CB21-498- | SYAMN | GISGSGGRIYYA | DLDV | RSSQSLVHS | KISNRFS | MQATQFPLT |
| 104 | | ESVKG | | DGNTYLN | | |
| CB21-498- | SYAMN | GISGSGGRIYYA | DLDV | RSSQSLVHS | KISNRFS | MQATQFPLT |
| 105 | | ESVKG | | DGNTYLN | | |
| CB21-498- | DYAMN | GISGSGGRIYYA | DLDV | RSSQSLVHS | KISNRFS | MQATQFPLT |
| 106 | | ESVKG | | DGNTYLN | | |
| CB21-498- | SYAMN | GISGSGGAIYYA | DLDV | RSSQSLVHS | KISNRFS | MQATQFPLT |
| 107 | | ESVKG | | DGNTYLN | | |
| CB21-498- | SYAMN | GISGSGGRAYYA | DLDV | RSSQSLVHS | KISNRFS | MQATQFPLT |
| 108 | | ESVKG | | DGNTYLN | | |
| CB21-498- | SYAMN | GISGSGGSTYYA | DLDV | RSSQSLVHS | KISNRFS | MQATQFPLT |
| 109 | | ESVKG | | DGNTYLN | | |
| CB21-498- | SYAMN | GISGSGGRIFYA | DLDV | RSSQSLVHS | KISNRFS | MQATQFPLT |
| 110 | | ESVKG | | DGNTYLN | | |
| CB21-498- | SYAMN | GISGSGGRIHYA | DLDV | RSSQSLVHS | KISNRFS | MQATQFPLT |
| 111 | | ESVKG | | DGNTYLN | | |
| CB21-498- | SYAMN | GISGSGGRINYA | DLDV | RSSQSLVHS | KISNRFS | MQATQFPLT |
| 112 | | ESVKG | | DGNTYLN | | |
| CB21-498- | SYAMN | GISGSGGRIK YA | DLDV | RSSQSLVHS | KISNRFS | MQATQFPLT |
| 113 | | ESVKG | | DGNTYLN | | |
| CB21-498- | SYAMN | GISGSGGRIDYA | DLDV | RSSQSLVHS | KISNRFS | MQATQFPLT |
| 114 | | ESVKG | | DGNTYLN | | |
| CB21-498- | SYAMN | GISGSGGRIAYA | DLDV | RSSQSLVHS | KISNRFS | MQATQFPLT |
| 115 | | ESVKG | | DGNTYLN | | |
| CB21-498- | SYAMN | GISGSGGRIYFA | DLDV | RSSQSLVHS | KISNRFS | MQATQFPLT |
| 116 | | ESVKG | | DGNTYLN | | |
| CB21-498- | SYAMN | GISGSGGRIYHA | DLDV | RSSQSLVHS | KISNRFS | MQATQFPLT |
| 117 | | ESVKG | | DGNTYLN | | |
| CB21-498- | SYAMN | GISGSGGRIYPA | DLDV | RSSQSLVHS | KISNRFS | MQATQFPLT |
| 118 | | ESVKG | | DGNTYLN | | |
| CB21-498- | SYAMN | GISGSGGRIYAA | DLDV | RSSQSLVHS | KISNRFS | MQATQFPLT |
| 119 | | ESVKG | | DGNTYLN | | |
| CB21-498- | SYAMN | GISGSGGRIYYA | DLDV | RSSQSLVHS | KISNRFS | MQATQFPLT |
| 120 | | ESVKG | | DGNTYLN | | |
| CB21-498- | SYAMN | GISGSGGRIYYA | DLDV | RSSQSLVHS | KISNRFS | MQATQFPLT |
| 121 | | ESVKG | | DGNTYLN | | |
| CB21-498- | SYAMN | GISGSGGRIYSA | DLDV | RSSQSLVHS | KISNRFS | MQATQFPLT |
| 122 | | ESVKG | | DGNTYLN | | |
| CB21-498- | SYAMN | GISGNGGRIYYA | DLDV | RSSQSLVHS | KISNRFD | MQATQFPLT |
| 123 | | ESVKG | | DGNTYLN | | |
| CB21-498- | SYAMN | GISGNGGRIYYA | DLDV | RSSQSLVHS | KISNRFE | MQATQFPLT |
| 124 | | ESVKG | | DGNTYLN | | |
| CB21-498- | SYAMN | GISGNGGRIYYA | DLDV | RSSQSLVHS | KISNRFQ | MQATQFPLT |
| 125 | | ESVKG | | DGNTYLN | | |
| CB21-498- | SYAMN | GISGNGGRIYYA | DLDV | RSSQSLVHS | KISNRFS | MQATQFPLT |
| 126 | | ESVKG | | DGNTYLN | | |
| CB21-498- | SYAMN | GISGNGGRIYYA | DLDV | RSSQSLVHS | KISNRFS | MQATQFPLT |
| 127 | | ESVKG | | DGNTYLN | | |
| CB21-498- | SYAMN | GISGNGGRIYYA | DLDV | RSSQSLVHS | KISNRFS | MQATQFPLT |
| 128 | | ESVKG | | DGNTYLN | | |
| CB21-498- | SYAMN | GISGNGGRIYYA | DLDV | RSSQSLVHS | KISNRFS | MQATQFPLT |
| 129 | | ESVKG | | DGNTYLN | | |
| CB21-498- | SYAMN | GISGNGGRIYYA | DLDV | RSSQSLVHS | KISNRFS | MQATQFPLT |
| 130 | | ESVKG | | DGNTYLN | | |
| CB21-498- | SYAMN | GISGNGGRIYYA | DLDV | RSSQSLVHS | KISNRFS | MQATQFPLT |
| 131 | | ESVKG | | DGNTYLN | | |
| CB21-498- | SYAMN | GISGNGGRIYYA | DLDV | RSSQSLVHS | KIDNRFS | MQATQFPLT |
| 132 | | ESVKG | | DGNTYLN | | |
| CB21-498- | SYAMN | GISGNGGRIYYA | DLDV | RSSQSLVHS | KIDNRFS | MQATQFPLT |
| 133 | | ESVKG | | DGNTYLN | | |
| CB21-498- | SYAMN | GISGSGGRAYYA | DLDV | RSSQSLVHS | KISNRFS | MQATQFPLT |
| 134 | | ESVKG | | DANTYLN | | |
| CB21-498- | SYAMN | GISGSGGRIYYA | DLDV | RSSQSLVHS | KISNRFS | MQATQFPLT |
| 135 | | ESVKG | | DANTYLN | | |
| CB21-498- | DYAMN | GISGSGGRIYYA | DLDV | RSSQSLVHS | KISNRFS | MQATQFPLT |
| 136 | | ESVKG | | DANTYLN | | |
| CB21-498- | SYAMN | GISGSGGRAYYA | DLDV | RSSQSLVHS | KISNRFS | MQATQFPLT |
| 137 | | ESVKG | | DANTYLN | | |
| CB21-498- | DYAMN | GISGSGGRAYYA | DLDV | RSSQSLVHS | KISNRFS | MQATQFPLT |
| 138 | | ESVKG | | DANTYLN | | |
| CB21-498- | DYAMN | GISGSGGRIYYA | DLDV | RSSQSLVHS | KISNRFS | MQATQFPLT |
| 139 | | ESVKG | | DANTYLN | | |
| CB21-498- | DYAMN | GISGSGGRAYYA | DLDV | RSSQSLVHS | KISNRFS | MQATQFPLT |
| 140 | | ESVKG | | DANTYLN | | |
| CB21-498 | SYAMN | GISGSGGRIYYA | DLDV | RSSQSLVHS | KISNRFS | MQATQFPLT |
| 141 | | ESVKG | | DANTYLN | | |
| CB21-498- | SYAMN | GISGSGGRIYYA | DLDV | RSSQSLVHS | KISNRFS | MQATQFPLT |
| 142 | | ESVKG | | DANTYLN | | |
| CB21-498- | SYAMN | GISGSGGRIYYA | DLDV | RSSQSLVHS | KISNRFS | MQATQFPLT |
| 143 | | ESVKG | | DGNTYLN | | |
| CB21-499 | SYGMH | VIWYDGSNKDY | PGEGDY | RASQGVRN | GASSLQS | LQQNSYPWT |
| | | ADSVKG | | DFG | | |
| CB21-499- | SYGMH | VIWYDASNKDY | PGEGDY | RASQGVRN | GASSLQS | LQQNSYPWT |
| 001 | | ADSVKG | | DFG | | |
| CB21-499- | SYGMH | VIWYDPSNKDY | PGEGDY | RASQGVRN | GASSLQS | LQQNSYPWT |
| 002 | | ADSVKG | | DFG | | |
| CB21-499- | SYGMH | VIWYEGSNKDY | PGEGDY | RASQGVRN | GASSLQS | LQQNSYPWT |
| 003 | | ADSVKG | | DFG | | |
| CB21-499- | SYGMH | VIWYSGSNKDY | PGEGDY | RASQGVRN | GASSLQS | LQQNSYPWT |
| 004 | | ADSVKG | | DFG | | |
| CB21-499- | SYGMH | VIWYDGSNKDY | PGEGDY | RASQGVRN | GASSLQS | LQQNSYPWT |
| 005 | | APSVKG | | DFG | | |
| CB21-499- | SYGMH | VIWYDGSNKDY | PGEGDY | RASQGVRN | GASSLQS | LQQNSYPWT |
| 006 | | AQSVKG | | DFG | | |
| CB21-499- | SYGMH | VIWYDGSNKDY | PGEGDY | RASQGVRN | GASSLQS | LQQNSYPWT |
| 007 | | AESVKG | | DFG | | |
| CB21-499- | SYGMH | VIWYDGSNKDY | PGEGDY | RASQGVRN | GASSLQS | LQQNSYPWT |
| 008 | | ADAVKG | | DFG | | |
| CB21-499- | SYGMH | VIWYDGSNKDY | DGEGDY | RASQGVRN | GASSLQS | LQQNSYPWT |
| 009 | | ADSVKG | | DFG | | |
| CB21-499- | SYGMH | VIWYDGSNKDY | PGEGDY | RASQGVRN | GASSLQS | LQQNSYPWT |
| 010 | | ADSVKG | | DFG | | |
| CB21-499- | SYGMH | VIWYDGSNKYY | PGEGDY | RASQGVRN | GASSLQS | LQQNSYPWT |
| 011 | | ADSVKG | | DFG | | |
| CB21-499- | SYGMH | VIWYDGSSKDY | PGEGDY | RASQGVRN | GASSLQS | LQQNSYPWT |
| 012 | | ADSVKG | | DFG | | |
| CB21-499- | SYGMH | VIWYDGSNKDY | PGYGDY | RASQGVRN | GASSLQS | LQQNSYPWT |
| 013 | | ADSVKG | | DFG | | |
| CB21-499- | SYGMH | VIWYDGSNKDY | PGEGDY | RASQGVRN | GASSLQS | LQQNSYPWT |
| 014 | | ADSVKG | | DFG | | |
| CB21-499- | SYGMH | VIWYDGSNKDY | PGEGDY | RASQGVRN | GASSLQS | LQQNSYPWT |
| 015 | | ADSVKG | | DFG | | |
| CB21-499- | SYGMH | VIWYDGSNKDY | PGEFDY | RASQGVRN | GASSLQS | LQQNSYPWT |
| 016 | | ADSVKG | | DFG | | |
| CB21-499- | SYGMH | VIWYDGSNKDY | PGEGDY | RASQGVRN | GASSLQS | LQQNSYPWT |
| 017 | | ADSVKG | | DFG | | |
| CB21-499- | SYGMH | VIWYDGSNKDY | PGEGDY | RASQGVRN | GASSLQS | LQQNSYPWT |
| 018 | | ADSVKG | | DLG | | |
| CB21-499- | SYGMH | VIWYDGSNKDY | PGEGDY | RASQGVRN | GASSLQS | QQQNSYPWT |
| 019 | | ADSVKG | | DFG | | |
| CB21-499- | SYGMH | VIWYDGSNKDY | PGEGDY | RASQGVSN | GASSLQS | LQQNSYPWT |
| 020 | | ADSVKG | | DFG | | |
| CB21-499- | SYGMH | VIWYDGSNKDY | PGEGDY | RASQGVRN | GASSLQS | LQQNSYPWT |
| 021 | | ADSVKG | | DFG | | |
| CB21-499- | SYGMH | VIWYDGSNKDY | PGEGDY | RASQGVRN | GASSLQS | LQYNSYPWT |
| 022 | | ADSVKG | | DFG | | |
| CB21-499- | SYGMH | VIWYDGSNKDY | PGEGDY | RASQGVRN | GASSLQS | LQQNSYPFT |
| 023 | | ADSVKG | | DFG | | |
| CB21-499- | SYGMH | VIWYDASNKDY | PGEGDY | RASQGVRN | GASSLQS | LQQNSYPWT |
| 024 | | AESVKG | | DFG | | |
| CB21-499- | SYGMH | VIWYDASNKDY | PGEGDY | RASQGVRN | GASSLQS | LQQNSYPWT |
| 025 | | APSVKG | | DFG | | |
| CB21-499- | SYGMH | VIWYDGSNKDY | PGEGDY | RASQGVRN | GASSLQS | LQQNSYPWT |
| 026 | | ADSVKG | | DFG | | |
| CB21-499- | SYGMH | VIWYDGSNKDY | PGEGDY | RASQGVRN | GASSLQS | LQQNSYPWT |
| 027 | | ADSVKG | | DFG | | |
| CB21-499- | SYGMH | VIWYDGSNKDY | PGEGDY | RASQGVRN | GASSLQS | LQQNSYPWT |
| 028 | | ADSVKG | | DFG | | |
| CB21-499- | SYGMH | VIWYDGSNKDY | PGEGDY | RASQGVRN | GASSLQS | LQQNSYPWT |
| 029 | | ADSVKG | | DFG | | |
| CB21-499- | SYGMH | VIWYDGSNKDY | PGEGDY | RASQGVRN | GASSLQS | LQQNSYPWT |
| 030 | | ADSVKG | | DFG | | |
| CB21-499- | SYGMH | VIWYDGSNKDY | PGEGDY | RASQGVRN | GASSLQS | LQQNSYPWT |
| 031 | | ADSVKG | | DFG | | |
| CB21-499- | SYGMH | VIWYDGSNKDY | PGEGDY | RASQGVRN | GASSLQS | LQQNSYPWT |
| 032 | | ADSVKG | | DFG | | |
| CB21-499- | SYGMH | VIWYDGSNKDY | PGEGDY | RASQGVRN | GASSLQS | LQQNSYPWT |
| 033 | | ADSVKG | | DFG | | |
| CB21-499- | SYGMH | VIWYDGSNKDY | PGEGDY | RASQGVRN | GASSLQS | LQQNSYPWT |
| 034 | | ADSVKG | | DFG | | |
| CB21-499- | SYAMH | VIWYDGSNKDY | PGEGDY | RASQGVRN | GASSLQS | LQQNSYPWT |
| 035 | | ADSVKG | | DFG | | |
| CB21-499- | SYTMH | VIWYDGSNKDY | PGEGDY | RASQGVRN | GASSLQS | LQQNSYPWT |
| 036 | | ADSVKG | | DFG | | |
| CB21-499- | SYNMH | VIWYDGSNKDY | PGEGDY | RASQGVRN | GASSLQS | LQQNSYPWT |
| 037 | | ADSVKG | | DFG | | |
| CB21-499- | SYQMH | VIWYDGSNKDY | PGEGDY | RASQGVRN | GASSLQS | LQQNSYPWT |
| 038 | | ADSVKG | | DFG | | |
| CB21-499- | SYEMH | VIWYDGSNKDY | PGEGDY | RASQGVRN | GASSLQS | LQQNSYPWT |
| 039 | | ADSVKG | | DFG | | |
| CB21-499- | SYRMH | VIWYDGSNKDY | PGEGDY | RASQGVRN | GASSLQS | LQQNSYPWT |
| 040 | | ADSVKG | | DFG | | |
| CB21-499- | SYWMH | VIWYDGSNKDY | PGEGDY | RASQGVRN | GASSLQS | LQQNSYPWT |
| 041 | | ADSVKG | | DFG | | |
| CB21-499- | SYPMH | VIWYDGSNKDY | PGEGDY | RASQGVRN | GASSLQS | LQQNSYPWT |
| 042 | | ADSVKG | | DFG | | |
| CB21-499- | SYGMH | VIWYDGSNKDY | PGEGDY | RASQGVRN | GASSLQS | LQQNSYPWT |
| 043 | | ADSVKG | | DFG | | |
| CB21-499- | SYGMH | VIWYDGSNKDY | PGEGDY | RASQGVRN | GASSLQS | LQQNSYPWT |
| 044 | | ADSVKG | | DFG | | |
| CB21-499- | SYGMH | VIWYDGSNKDY | PGEGDY | RASQGVRN | GASSLQS | LQQNSYPWT |
| 045 | | ADSVKG | | DFG | | |
| CB21-499- | SYGMH | VIWYDGSNKDY | PGEGDY | RASQGVRN | GASSLQS | LQQNSYPWT |
| 046 | | ADSVKG | | DFG | | |
| CB21-499- | SYGMH | VIWYDGSNKDY | PGEGDY | RASQGVRN | GASSLQS | LQQNSYPWT |
| 047 | | ADSVKG | | DFG | | |
| CB21-499- | SYGMH | VIWYDGSNKDY | PGEGDY | RASQGVRN | GASSLQS | LQQNSYPWT |
| 048 | | ADSVKG | | DFG | | |
| CB21-499- | SYGMH | VIWYDGSNKDY | PGEGDY | RASQGVRN | GASSLQS | LQQNSYPWT |
| 049 | | ADSVKG | | DFG | | |
| CB21-499- | SYGMH | VIWYDGSNTDY | PGEGDY | RASQGVRN | GASSLQS | LQQNSYPWT |
| 050 | | ADSVKG | | DFG | | |
| CB21-499- | SYGMH | VIWYDGSNIDY | PGEGDY | RASQGVRN | GASSLQS | LQQNSYPWT |
| 051 | | ADSVKG | | DFG | | |
| CB21-499- | SYGMH | VIWYDGSNNDY | PGEGDY | RASQGVRN | GASSLQS | LQQNSYPWT |
| 052 | | ADSVKG | | DFG | | |
| CB21-499- | SYGMH | VIWYDGSNRDY | PGEGDY | RASQGVRN | GASSLQS | LQQNSYPWT |
| 053 | | ADSVKG | | DFG | | |
| CB21-499- | SYGMH | VIWYDGSNKFY | PGEGDY | RASQGVRN | GASSLQS | LQQNSYPWT |
| 054 | | ADSVKG | | DFG | | |
| CB21-499- | SYGMH | VIWYDGSNKNY | PGEGDY | RASQGVRN | GASSLQS | LQQNSYPWT |
| 055 | | ADSVKG | | DFG | | |
| CB21-499- | SYGMH | VIWYDGSNKEY | PGEGDY | RASQGVRN | GASSLQS | LQQNSYPWT |
| 056 | | ADSVKG | | DFG | | |
| CB21-499- | SYGMH | VIWYDGSNKDY | PGEGDY | RASQGVRN | GASSLQS | LQQNSYPWT |
| 057 | | ADSVKG | | DFG | | |
| CB21-499- | SYGMH | VIWYDGSNKDY | PGEGDY | RASQGVRN | GASSLQS | LQQNSYPWT |
| 058 | | ADSVKG | | DFG | | |
| CB21-499- | SYGMH | VIWYDGSNKDY | PGEGDY | RASQGVRN | GASSLQS | LQQNSYPWT |
| 059 | | ADSVKG | | DFG | | |
| CB21-499- | SYGMH | VIWYDGSNKDY | PGEGDY | RASQGVRN | GASSLQS | LQQNSYPWT |
| 060 | | ADSVKG | | DFG | | |
| CB21-499- | SYGMH | VIWYDGSNKDY | PGEGDY | RASQGVRN | GASSLQS | LQQNSYPWT |
| 061 | | ADSVKG | | DFG | | |
| CB21-499- | SYGMH | VIWYDGSNKDY | PGEGDY | RASQGVRN | GASSLQS | LQQNSYPWT |
| 062 | | ADSVKG | | DFG | | |
| CB21-499- | SYGMH | VIWYDGSNKDY | PGEGDY | RASQIVRN | GASSLQS | LQQNSYPWT |
| 063 | | ADSVKG | | DFG | | |
| CB21-499- | SYGMH | VIWYDGSNKDY | PGEGDY | RASQSVRN | GASSLQS | LQQNSYPWT |
| 064 | | ADSVKG | | DFG | | |
| CB21-499- | SYGMH | VIWYDGSNKDY | PGEGDY | RASQGIRN | GASSLQS | LQQNSYPWT |
| 065 | | ADSVKG | | DFG | | |
| CB21-499- | SYGMH | VIWYDGSNKDY | PGEGDY | RASQGVRN | GASSLQS | LQQNSYPWT |
| 066 | | ADSVKG | | EFG | | |
| CB21-499- | SYGMH | VIWYDGSNKDY | PGEGDY | RASQGVRN | GASSLQS | LQQNSYPWT |
| 067 | | ADSVKG | | LFG | | |
| CB21-499- | SYGMH | VIWYDGSNKDY | PGEGDY | RASQGVRN | GASSLQS | LQQNSYPWT |
| 068 | | ADSVKG | | YFG | | |
| CB21-499- | SYGMH | VIWYDGSNKDY | PGEGDY | RASQGVRN | GASSLQS | LQQNSYPWT |
| 069 | | ADSVKG | | DFG | | |
| CB21-499- | SYGMH | VIWYDGSNKDY | PGEGDY | RASQGVRN | GASSLQS | LQQNSYPWT |
| 070 | | ADSVKG | | DFG | | |
| CB21-499- | SYGMH | VIWYDGSNKDY | PGEGDY | RASQGVRN | GASSLQS | LQQNSYPWT |
| 071 | | ADSVKG | | DFG | | |
| CB21-499- | SYGMH | VIWYDGSNKDY | PGEGDY | RASQGVRN | GASQLQS | LQQNSYPWT |
| 072 | | ADSVKG | | DFG | | |
| CB21-499- | SYGMH | VIWYDGSNKDY | PGEGDY | RASQGVRN | GASNLQS | LQQNSYPWT |
| 073 | | ADSVKG | | DFG | | |
| CB21-499- | SYGMH | VIWYDGSNKDY | PGEGDY | RASQGVRN | GASTLQS | LQQNSYPWT |
| 074 | | ADSVKG | | DFG | | |
| CB21-499- | SYGMH | VIWYDGSNKDY | PGEGDY | RASQGVRN | GASSLQS | LQQNSYPWT |
| 075 | | ADSVKG | | DFG | | |
| CB21-499- | SYGMH | VIWYDGSNKDY | PGEGDY | RASQGVRN | GASSLQS | LQQNSYPWT |
| 076 | | ADSVKG | | DFG | | |
| CB21-499- | SYGMH | VIWYDGSNKDY | PGEGDY | RASQGVRN | GASSLQS | LQQNSYPWT |
| 077 | | ADSVKG | | DFG | | |
| CB21-499- | SYGMH | VIWYDGSNKDY | PGEGDY | RASQGVRN | GASSLQS | LQQNSYPWT |
| 078 | | ADSVKG | | DFG | | |
| CB21-499- | SYGMH | VIWYDGSNKDY | PGEGDY | RASQGVRN | GASSLQS | LQQNSYPWT |
| 079 | | ADSVKG | | DFG | | |
| CB21-499- | SYGMH | VIWYDGSNKDY | PGEGDY | RASQGVRN | GASSLQS | LQQNSYPWT |
| 080 | | ADSVKG | | DFG | | |
| CB21-499- | SYGMH | VIWYDGSNKDY | PGEGDY | RASQGVRN | GASSLQS | LQQNSYPWT |
| 081 | | ADSVKG | | DFG | | |
| CB21-499- | SYAMH | VISYDGSNKYY | PGEGDY | RASQGVRN | GASSLQS | LQQNSYPWT |
| 082 | | ADSVKG | | DFG | | |
| CB21-499- | SYGMH | VIWYDASNKDY | PGEGDY | RASQGVRN | GASSLQS | LQQNSYPWT |
| 083 | | APSVKG | | DFG | | |
| CB21-499- | SYGMH | VIWYDASNKDY | PGEGDY | RASQGVRN | GASSLQS | LQQNSYPWT |
| 084 | | VPSVKG | | DFG | | |
| CB21-499- | SYGMH | VIWYDASNKDY | PGEGDY | RASQGVRN | GASSLQS | LQQNSYPWT |
| 085 | | APSVKG | | DFG | | |
| CB21-499- | SYGMH | VIWYDASNKDY | PGEGDY | RASQGVRN | GASSLQS | LQQNSYPWT |
| 086 | | VPSVKG | | DFG | | |
| CB21-499- | SYGMH | VIWYDASNKDY | PGEGDY | RASQGVRN | GASSLQS | LQQNSYPWT |
| 087 | | APSVKG | | DFG | | |
| CB21-499- | SYGMH | VIWYDASNKDY | PGEGDY | RASQGVRN | GASSLQS | LQQNSYPWT |
| 088 | | VPSVKG | | DFG | | |
| CB21-499- | SYGMH | VIWYDASNKDY | PGEGDY | RASQGVRN | GASSLQS | LQQNSYPWT |
| 089 | | VPSVKG | | DFG | | |
| CB21-499- | VYGMH | VIWYDASNKDY | PGEGDY | RASQGVRN | GASSLQS | LQQNSYPWT |
| 090 | | APSVKG | | DFG | | |
| CB21-499- | TYGMH | VIWYDASNKDY | PGEGDY | RASQGVRN | GASSLQS | LQQNSYPWT |
| 091 | | APSVKG | | DFG | | |
| CB21-499- | SYGMN | VIWYDASNKDY | PGEGDY | RASQGVRN | GASSLQS | LQQNSYPWT |
| 092 | | APSVKG | | DFG | | |
| CB21-499- | SYGMH | IIWYDASNKDY | PGEGDY | RASQGVRN | GASSLQS | LQQNSYPWT |
| 093 | | APSVKG | | DFG | | |
| CB21-499- | SYGMH | MIWYDASNKDY | PGEGDY | RASQGVRN | GASSLQS | LQQNSYPWT |
| 094 | | APSVKG | | DFG | | |
| CB21-499- | SYGMH | RIWYDASNKDY | PGEGDY | RASQGVRN | GASSLQS | LQQNSYPWT |
| 095 | | APSVKG | | DFG | | |
| CB21-499- | SYGMH | DIWYDASNKDY | PGEGDY | RASQGVRN | GASSLQS | LQQNSYPWT |
| 096 | | APSVKG | | DFG | | |
| CB21-499- | SYGMH | EIWYDASNKDY | PGEGDY | RASQGVRN | GASSLQS | LQQNSYPWT |
| 097 | | APSVKG | | DFG | | |
| CB21-499- | SYGMH | AIWYDASNKDY | PGEGDY | RASQGVRN | GASSLQS | LQQNSYPWT |
| 098 | | APSVKG | | DFG | | |
| CB21-499- | SYGMH | VIWYDASNKDY | PGEGDY | RASQGVRN | GASSLQS | LQQNSYPWT |
| 099 | | APSVKG | | DFG | | |
| CB21-499- | SYGMH | VIWYDASNKDY | PGEGDY | RASQGVRN | GASSLQS | LQQNSYPWT |
| 100 | | APSVKG | | DFG | | |
| CB21-499- | SYGMH | VIWYDASNKDY | PGEGDY | RASQGVRN | GASSLQS | LQQNSYPWT |
| 101 | | APSVKG | | DFG | | |
| CB21-499- | SYGMH | VIWYDASNKDY | PGEGDY | RASQGVRN | GASSLQS | LQQNSYPWT |
| 102 | | APSVKG | | DFG | | |
| CB21-499- | SYGMH | VIWYDASNKDY | PGEGDY | RASQGVRN | GASSLQS | LQQNSYPWT |
| 103 | | APSVKG | | DFG | | |
| CB21-499- | SYGMH | VIWYDASNKDY | PGEGDY | RASQGVRN | GASSLQS | LQQNSYPWT |
| 104 | | APSVKG | | DFG | | |
| CB21-499- | SYGMH | VIWYDASNKDY | PGEGDY | RASQGVRN | GASSLQS | LQQNSYPWT |
| 105 | | APSVKG | | DFG | | |
| CB21-499- | SYGMH | VIWYDASNKDY | PGEGDY | RASQGVRN | GASSLQS | LQQNSYPWT |
| 106 | | APSVKG | | DFG | | |
| CB21-499- | SYGMH | VIWYDASNKDY | PGEGDY | RASQGVRN | GASSLQS | LQQNSYPWT |
| 107 | | APSVKG | | DFG | | |
| CB21-499- | SYGMH | VIWYDASNKDY | PGEGDY | RASQGVRN | GASSLQS | LQQNSYPWT |
| 108 | | APSVKG | | DFG | | |
| CB21-499- | SYGMH | VIWYDASNKDY | PGEGDY | RASQGVRN | GASSLQS | LQQNSYPWT |
| 109 | | APSVKG | | DFG | | |
| CB21-499- | SYGMH | VIWYDASNKDY | PGEGDY | RASQGVRN | GASSLQS | LQKNSYPWT |
| 110 | | APSVKG | | DFG | | |
| CB21-499- | SYGMH | VIWYDASNKDY | PGEGCY | RASQGVRN | GASSLQS | LQCNSYPWT |
| 111 | | APSVKG | | DFG | | |
| CB21-499- | SYGMH | VIWYDASNKDY | PGEGDY | RASQGVRN | GASSLQS | LQQNSYPWT |
| 112 | | APSVKG | | DFG | | |
| CB21-499- | SYGMH | VIWYDASNKDY | PGEGDY | RASQGVRN | GASSLQS | LQQNSYPWT |
| 113 | | APSVKG | | DFG | | |
| CB21-499- | DYGMH | VIWYDASNKDY | PGEGDY | RASQGVRN | GASSLQS | LQQNSYPWT |
| 114 | | APSVKG | | DFG | | |
| CB21-499- | SDGMH | VIWYDASNKDY | PGEGDY | RASQGVRN | GASSLQS | LQQNSYPWT |
| 115 | | APSVKG | | DFG | | |
| CB21-499- | SYGMH | VIWYDASNKDY | PGEGDY | RASQGVRN | GASSLQS | LQQNSYPWT |
| 116 | | APSVKG | | DFG | | |
| CB21-499- | SYGMH | VIWYDASNKDY | PGEGDY | RASQGVRN | GASSLQS | LQQNSYPWT |
| 117 | | APSVKG | | DFG | | |
| CB21-499- | SYGMH | VIWYDASNKDY | PGEGDY | RASQGVRN | GASSLQS | LQQNSYPWT |
| 118 | | APSVKG | | DFG | | |
| CB21-499- | SYGMH | VIWYDASNKDY | PGEGDY | RASQGVRN | GASSLQS | LQQNSYPWT |
| 119 | | APSVKG | | DFG | | |
| CB21-499- | SYGMH | VIWYDASNKDY | PGEGDY | RASQGVRN | GASSLQS | LQQNSYPWT |
| 120 | | APSVKG | | DFG | | |
| CB21-499- | SYGMH | VIWYDASNKDY | PGEGDY | RASQGVRN | GASSLQS | LQQNSYPWT |
| 121 | | APSVKG | | DFG | | |
| CB21-499- | SYGMH | VIWYDASNKDY | PGECDY | RASQGVRN | GASSLQS | LQQNSYPWT |
| 122 | | APSVKG | | DFG | | |
| CB21-499- | SYGMH | VIWYDASNKDY | PGEGDY | RASQGVRN | GASSLQS | LQQNSYPWT |
| 123 | | APSVKG | | DFG | | |
| CB21-499- | SYGMH | VIWYDASNKDY | PGEGDY | RASQGVRN | GASSLQS | LQQNSYPWT |
| 124 | | APSVKG | | DFG | | |
| CB21-499- | PYGMH | VIWYDASNKDY | PGEGDY | RASQGVRN | GASSLQS | LQQNSYPWT |
| 125 | | APSVKG | | DFG | | |
| CB21-499- | SYPMH | VIWYDASNKDY | PGEGDY | RASQGVRN | GASSLQS | LQQNSYPWT |
| 126 | | APSVKG | | DFG | | |
| CB21-499- | SYGMH | VIWYDAPNKDY | PGEGDY | RASQGVRN | GASSLQS | LQQNSYPWT |
| 127 | | APSVKG | | DFG | | |
| CB21-499- | SYGMH | VISYDASNKDY | PGEGDY | RASQGVRN | GASSLQS | LQQNSYPWT |
| 128 | | APSVKG | | DFG | | |
| CB21-499- | SYGMH | VINYDASNKDY | PGEGDY | RASQGVRN | GASSLQS | LQQNSYPWT |
| 129 | | APSVKG | | DFG | | |
| CB21-499- | SYGMH | VIDYDASNKDY | PGEGDY | RASQGVRN | GASSLQS | LQQNSYPWT |
| 130 | | APSVKG | | DFG | | |
| CB21-499- | SYGMH | VIRYDASNKDY | PGEGDY | RASQGVRN | GASSLQS | LQQNSYPWT |
| 131 | | APSVKG | | DFG | | |
| CB21-499- | SYGMH | VIKYDASNKDY | PGEGDY | RASQGVRN | GASSLQS | LQQNSYPWT |
| 132 | | APSVKG | | DFG | | |
| CB21-499- | SYGMH | VIAYDASNKDY | PGEGDY | RASQGVRN | GASSLQS | LQQNSYPWT |
| 133 | | APSVKG | | DFG | | |
| CB21-499- | SYGMH | VIWDDASNKDY | PGEGDY | RASQGVRN | GASSLQS | LQQNSYPWT |
| 134 | | APSVKG | | DFG | | |
| CB21-499- | SYGMH | VIWPDASNKDY | PGEGDY | RASQGVRN | GASSLQS | LQQNSYPWT |
| 135 | | APSVKG | | DFG | | |
| CB21-499- | SYGMH | VIWNDASNKDY | PGEGDY | RASQGVRN | GASSLQS | LQQNSYPWT |
| 136 | | APSVKG | | DFG | | |
| CB21-499- | SYGMH | VIWKDASNKDY | PGEGDY | RASQGVRN | GASSLQS | LQQNSYPWT |
| 137 | | APSVKG | | DFG | | |
| CB21-499- | SYGMH | VIWGDASNKDY | PGEGDY | RASQGVRN | GASSLQS | LQQNSYPWT |
| 138 | | APSVKG | | DFG | | |
| CB21-499- | SYGMH | VIWSDASNKDY | PGEGDY | RASQGVRN | GASSLQS | LQQNSYPWT |
| 139 | | APSVKG | | DFG | | |
| CB21-499- | SYGMH | VIWYDASNKDY | PGEGDV | RASQGVRN | GASSLQS | LQQNSYPWT |
| 140 | | APSVKG | | DFG | | |
| CB21-499- | SYGMH | VIWYDASNKDY | PGEGDR | RASQGVRN | GASSLQS | LQQNSYPWT |
| 141 | | APSVKG | | DFG | | |
| CB21-499- | SYGMH | VIWYDASNKDY | PGEGDP | RASQGVRN | GASSLQS | LQQNSYPWT |
| 142 | | APSVKG | | DFG | | |
| CB21-499- | SYGMH | VIWYDASNKDY | PGEGDA | RASQGVRN | GASSLQS | LQQNSYPWT |
| 143 | | APSVKG | | DFG | | |
| CB21-499- | SYGMH | VIWYDASNKDY | PGEGDY | RASQGVRN | GDSSLQS | LQQNSYPWT |
| 144 | | APSVKG | | DFG | | |
| CB21-499- | SYGMH | VIWYDASNKDY | PGEGDY | RASQGVRN | GADSLQS | LQQNSYPWT |
| 145 | | APSVKG | | DFG | | |
| CB21-499- | SYGMH | VIWYDASNKDY | PGEGDY | RASQGVRN | GDDSLQS | LQQNSYPWT |
| 146 | | APSVKG | | DFG | | |
| CB21-499- | SYGMH | VIWYDASNKDY | PGEGDY | RASQGVRN | GASSLQS | LQQNSYPWT |
| 147 | | APSVKG | | DFG | | |
| CB21-499- | SYGMH | VIWYDASNKDY | PGEGDY | RASQGVRN | AASSLQS | LQQNSYPWT |
| 148 | | APSVKG | | DFG | | |
| CB21-499- | SYGMH | VIWYDASNKDY | PGEGDY | RASQGVRN | GASSLQS | LQQNSYPWT |
| 149 | | APSVKG | | DFG | | |
| CB21-499- | SYGMH | VIWYDASNKDY | PGEGDY | RASQGVRN | GASSLQS | LQQNSYPWT |
| 150 | | APSVKG | | DFG | | |
| CB21-499- | SYGMH | VIWYDASNKDY | PGEGDY | RASQGVRN | GASSLQS | LQQNSYPWT |
| 151 | | APSVKG | | DFG | | |
| CB21-499- | SYGMH | VIWYDASNKDY | PGEGDY | RASQGVRN | GASSLCS | LQQNSYPWT |
| 152 | | APSVKG | | DFG | | |
| CB21-499- | SYGMH | VIWYDASNKDY | PGEGDY | RASQGVRN | DASSLQS | LQQNSYPWT |
| 153 | | APSVKG | | DFG | | |
| CB21-499- | SYGMH | VIWYDASNKDY | PGEGDY | RASQGVRN | EASSLQS | LQQNSYPWT |
| 154 | | APSVKG | | DFG | | |
| CB21-499- | SYGMH | VIWYDASNKDY | PGEGDY | RASQGVRN | KASSLQS | LQQNSYPWT |
| 155 | | APSVKG | | DFG | | |
| CB21-499- | SYGMH | VIWYDASNKDY | PGEGDY | RASQGVRN | LASSLQS | LQQNSYPWT |
| 156 | | APSVKG | | DFG | | |
| CB21-499- | SYGMH | VIWYDASNKDY | PGEGDY | RASQGVRN | SASSLQS | LQQNSYPWT |
| 157 | | APSVKG | | DFG | | |
| CB21-499- | SYGMH | VIWYDASNKDY | PGEGDY | RASQGVRN | GVSSLQS | LQQNSYPWT |
| 158 | | APSVKG | | DFG | | |
| CB21-499- | SYGMH | VIWYDASNKDY | PGEGDY | RASQGVRN | GESSLQS | LQQNSYPWT |
| 159 | | APSVKG | | DFG | | |
| CB21-499- | SYGMH | VIWYDASNKDY | PGEGDY | RASQGVRN | GPSSLQS | LQQNSYPWT |
| 160 | | APSVKG | | DFG | | |
| CB21-499- | SYGMH | VIWYDASNKDY | PGEGDY | RASQGVRN | GATSLQS | LQQNSYPWT |
| 161 | | APSVKG | | DFG | | |
| CB21-499- | SYGMH | VIWYDASNKDY | PGEGDY | RASQGVRN | GAPSLQS | LQQNSYPWT |
| 162 | | APSVKG | | DFG | | |
| CB21-499- | SYGMH | VIWYDASNKDY | PGEGDY | RASQGVRN | GASRLQS | LQQNSYPWT |
| 163 | | APSVKG | | DFG | | |
| CB21-499- | SYGMH | VIWYDASNKDY | PGEGDY | RASQGVRN | GASKLQS | LQQNSYPWT |
| 164 | | APSVKG | | DFG | | |
| CB21-499- | SYGMH | VIWYDASNKDY | PGEGDY | RASQGVRN | GASDLQS | LQQNSYPWT |
| 165 | | APSVKG | | DFG | | |
| CB21-499- | SYGMH | VIWYDASNKDY | PGEGDY | RASQGVRN | GASILQS | LQQNSYPWT |
| 166 | | APSVKG | | DFG | | |
| CB21-499- | SYGMH | VIWYDASNKDY | PGEGDY | RASQGVRN | GASLLQS | LQQNSYPWT |
| 167 | | APSVKG | | DFG | | |
| CB21-499- | SYGMH | VIWYDASNKDY | PGEGDY | RASQGVRN | GASSRQS | LQQNSYPWT |
| 168 | | APSVKG | | DFG | | |
| CB21-499- | SYGMH | VIWYDASNKDY | PGEGDY | RASQGVRN | GASSPQS | LQQNSYPWT |
| 169 | | APSVKG | | DFG | | |
| CB21-499- | SYGMH | VIWYDASNKDY | PGEGDY | RASQGVRN | GASSLPS | LQQNSYPWT |
| 170 | | APSVKG | | DFG | | |
| CB21-499- | SYGMH | VIWYDASNKDY | PGEGDY | RASQGVRN | GASSLAS | LQQNSYPWT |
| 171 | | APSVKG | | DFG | | |
| CB21-499- | SYGMH | VIWYDASNKDY | PGEGDY | RASQGVRN | GASSLES | LQQNSYPWT |
| 172 | | APSVKG | | DFG | | |
| CB21-499- | SYGMH | VIWYDASNKDY | PGEGDY | RASQGIRN | AASSLQS | LQHNSYPWT |
| 174 | | APSVKG | | DLG | | |
| CB21-499- | SYGMH | VIWYDASNKDY | PGEGDY | RASQSVSS | DASNRAT | QQRSNWPYT |
| 175 | | APSVKG | | YLA | | |
| CB21-499- | SYGMH | VIWYDASNKDY | PGEGDY | RASQSISSY | AASSLQS | QQSYSTPFT |
| 176 | | APSVKG | | LN | | |
| CB21-499- | SYGMH | VIWYDASNKEY | PGEGDY | RASQGVRN | GASSLQS | LQQNSYPWT |
| 177 | | APSVKG | | DFG | | |
| CB21-499- | SYGMH | VIWYDASNKEY | PGEGDY | RASQSVSS | GASSLQS | LQQNSYPWT |
| 178 | | APSVKG | | YLA | | |
| CB21-499- | SYGMH | VIWYDASNKDY | PGEGDY | RASQSVSS | GASSLQS | LQQNSYPWT |
| 179 | | APSVKG | | YLA | | |

In a related aspect, the present invention provides hLY6G6D binding proteins comprising an immunoglobulin variable region that binds hLY6G6D, wherein the immunoglobulin variable region comprises a heavy chain variable region and a light chain variable region as recited for one of the Identifiers in Table 2.

TABLE 2
(SEQ ID NOs: 1994-2657, with numbering across rows (so row 1 is
SEQ ID NOs: 1994-1995, row 2 is SEQ ID NOs: 1996-1997, etc.):

| Identifier | VH | VL |
|---|---|---|
| CB21-469 | EVQLVESGGGLVQPGGSLRLSCAASG | DVVMTQTPLSSPVTLGQSASISCRSSQ |
| | FTFSSYAMNWVRQAPGKGLEWVSGI | SLVHSDGNTYLNWLHQRPGQPPRLLI |
| | SGNGGRIYYADSVKGRFTISRDNSKN | YKISNRFSGVPDRFSGSGAGTDFTLKI |
| | TLYLQMNSLRVEDTAVYYCAKDLDV | SRVEAEDVGVYYCMQATQFPLTFGG |
| | WGKGTTVTVSS | GTKLEIK |
| CB21-470 | EVQLVESGGGVVQPGRSLRLSCAASG | DIQMTQSPSSLSASVGDRVTITCRASQ |
| | FTFNSYGMHWVRQAPGKGLEWVAVL | GIRNDLTWYQQKPGKAPKRLIYDTSS |
| | WYDGINKNYADSVKGRFAISRDNSKN | LQSGVPSRFSGSRSGTEFTLTISSLQPE |
| | TLYLQMNSLRVEDTAVYYCVRVQGD | DFASYFCLQHNSYPFTFGQGTKLEIK |
| | YWGQGTLVTVSS | |
| CB21-471 | EVQLVESGGGVVQPGRSLRLSCAASG | DIQMTQSPSSLSASVGDRVTITCRASQ |
| | FTFSSYGMHWVRQVPGKGLEWVAVI | GIRNDFGWYQQKPGKAPKRLIYGASS |
| | WYDGSNKDYVDSVKGRFTISRDNSK | LQSGVPSRFSGSGSGTEFTLTISSLQPE |
| | NTLYLQMNSLRAEDTAVYYCARPGE | DFATYYCLQQNSYPWTFGQGTKVEIK |
| | GDYWGQGTLVTVSS | |
| CB21-477 | EVQLVESGGGVVQPGRSLRLSCAASG | DIQMTQSPSSLSASVGDRVTITCRASQ |
| | FTFSSYGMHWVRQAPGKGLEWVAVI | GIRNDLGWYQQKPGRAPKRLIYAASS |
| | WYDGSNKDYADSVKGRFTISRDNSK | LQSGVPSRFSGSGSGTEFTLTISSLQPE |
| | NTLYLQMNSLRAEDTAVYYCARPGE | DFATYYCLQQNSYPWTFGQGTKVEIK |
| | GDYWGQGTLVTVSS | |
| CB21-500 | EVQLVESGGGLVQPGGSLRLSCAASG | DVVMTQTPLSSPVTLGQPASISCRSSQ |
| | FTFSSYAMNWVRQVPGKGLEGVSGM | NLVHSDGNTYLNWLQQRPGQPPRLLI |
| | SGSGGRIYYADSVKGRFTMSRDNSKN | YKISNRFSGVPDRFSGSGAGTDFTLKI |
| | MVYLQMNSLRAEDTALYYCAKDLDV | SRVEAEDVGVYYCMQATQFPLTFGG |
| | WGKGTTVTVSS | GTKLEIK |
| CB21-502 | EVQLVESGGGVVQPGRSLRLSCAASG | DIQMTQSPSSLSASVGDRVTITCRASQ |
| | FTFSSYGMHWVRQAPGKGLEWVAVL | GIRNDLTWYQQKPGKAPKRLIYDTSS |
| | WYDGINKNYVDSVKGRFAISRDNSK | LQSGVPSRFSGSGSGTDFTLTISSLQPE |
| | NTLYLQMNSLRVEDTAVYFCVRVQG | DFASYFCLQHNIYPFTFGQGTKLEIK |
| | DYWGQGTLVTVSS | |
| CB21-512 | EVQLVESGGGVVQPGRSLRLSCAASG | DIQMTQSPSSLSASVGDRVTITCRASQ |
| | FTFSSYGMHWVRQAPGKGLEWVAVL | GIRNDLTWYQQKPGKAPKRLIYDTSS |
| | WYDGINKNYADSVKGRFAISRDNSKN | LQSGVPSRFSGSRSGTEFTLTISSLQPE |
| | TLYLQMNSLRVEDTAVYFCVRVQGD | DFASYFCLQHNIYPFTFGQGTKLEIK |
| | YWGQGTLVTVSS | |
| CB21-513 | EVQLVDSGGGLVEPGGSLRLSCAASG | DIVMTQTPLSSPVTLGQPASISCRSSQS |
| | FTFSSYAMTWVRQAPGKGLEWVSGIS | LVHSDGNTYLNWLQQRPGQPPRLLIY |
| | GSGGVTDYADSVKGRFTISRDNSKNT | KISNRFSGVPDRFSGSGAGTDFTLKIS |
| | LYMQMNSLRAEDTAVYYCAKDLGV | RVEAEDVGVYYCMQATQFPLTFGGG |
| | WGKGTTVTVSS | TKVEIK |
| CB21-522 | EVQLVQSGAEVKKPGASVKVSCKAS | DVVMTQSPLSLPVTLGQPASISCRSSQ |
| | GYTFTGYFIHWVRQAPGQGLEWMG | SLVYSDGNTYLNWFQQRPGQSPRRLI |
| | WINPNSGGTYYAQEFQGRVTMTRDTS | YKVSNRDSGVPERFSGIGSGTDFTLEI |
| | ISTAYMELIRLRSDDSAVYYCARDISL | SRVEAEDVGVYYCMQGSHWPLTFGG |
| | VGDYWGQGTLVTVSS | GTKVEIK |
| CB21-498 | EMQLVESGGGLVQPGGSLRLSCAASG | DVVMTQTPLSSPVTLGQPASISCRSSQ |
| | FTFSSYAMNWVRQAPGKGLECVSGIS | SLVHSDGNTYLNWLQQRPGQPPRLLI |
| | GNGGRIYYAESVKGRFTISRDNSKSTF | YKISNRFSGVPDRFSGSGAGTDFTLKI |
| | YLQMNSLRAEDTAVYYCAKDLDVW | SRVEAEDVGVYYCMQATQFPLTFGG |
| | GKGTTVTVSS | GTKVEIK |
| CB21-498-001 | EVQLVESGGGLVQPGGSLRLSCAASG | DVVMTQTPLSSPVTLGQPASISCRSSQ |
| | FTFSSYAMNWVRQAPGKGLEWVSGI | SLVHSDGNTYLNWLQQRPGQPPRLLI |
| | SGNGGRIYYAESVKGRFTISRDNSKST | YKISNRFSGVPDRFSGSGAGTDFTLKI |
| | FYLQMNSLRAEDTAVYYCAKDLDVW | SRVEAEDVGVYYCMQATQFPLTFGG |
| | GKGTTVTVSS | GTKVEIK |
| CB21-498-002 | EVQLVESGGGLVQPGGSLRLSCAASG | DVVMTQTPLSSPVTLGQPASISCRSSQ |
| | FTFSSYAMNWVRQAPGKGLESVSGIS | SLVHSDGNTYLNWLQQRPGQPPRLLI |
| | GNGGRIYYAESVKGRFTISRDNSKSTF | YKISNRFSGVPDRFSGSGAGTDFTLKI |
| | YLQMNSLRAEDTAVYYCAKDLDVW | SRVEAEDVGVYYCMQATQFPLTFGG |
| | GKGTTVTVSS | GTKVEIK |
| CB21-498-003 | EVQLVESGGGLVQPGGSLRLSCAASG | DVVMTQTPLSSPVTLGQPASISCRSSQ |
| | FTFSSYAMNWVRQAPGKGLECVSGIS | SLVHSDGNTYLNWLQQRPGQPPRLLI |
| | GNAGRIYYAESVKGRFTISRDNSKSTF | YKISNRFSGVPDRFSGSGAGTDFTLKI |
| | YLQMNSLRAEDTAVYYCAKDLDVW | SRVEAEDVGVYYCMQATQFPLTFGG |
| | GKGTTVTVSS | GTKVEIK |
| CB21-498-004 | EVQLVESGGGLVQPGGSLRLSCAASG | DVVMTQTPLSSPVTLGQPASISCRSSQ |
| | FTFSSYAMNWVRQAPGKGLECVSGIS | SLVHSDGNTYLNWLQQRPGQPPRLLI |
| | GNPGRIYYAESVKGRFTISRDNSKSTF | YKISNRFSGVPDRFSGSGAGTDFTLKI |
| | YLQMNSLRAEDTAVYYCAKDLDVW | SRVEAEDVGVYYCMQATQFPLTFGG |
| | GKGTTVTVSS | GTKVEIK |
| CB21-498-005 | EVQLVESGGGLVQPGGSLRLSCAASG | DVVMTQTPLSSPVTLGQPASISCRSSQ |
| | FTFSSYAMNWVRQAPGKGLECVSGIS | SLVHSDGNTYLNWLQQRPGQPPRLLI |
| | GQGGRIYYAESVKGRFTISRDNSKSTF | YKISNRFSGVPDRFSGSGAGTDFTLKI |
| | YLQMNSLRAEDTAVYYCAKDLDVW | SRVEAEDVGVYYCMQATQFPLTFGG |
| | GKGTTVTVSS | GTKVEIK |
| CB21-498-006 | EVQLVESGGGLVQPGGSLRLSCAASG | DVVMTQTPLSSPVTLGQPASISCRSSQ |
| | FTFSSYAMNWVRQAPGKGLECVSGIS | SLVHSDGNTYLNWLQQRPGQPPRLLI |
| | GSGGRIYYAESVKGRFTISRDNSKSTF | YKISNRFSGVPDRFSGSGAGTDFTLKI |
| | YLQMNSLRAEDTAVYYCAKDLDVW | SRVEAEDVGVYYCMQATQFPLTFGG |
| | GKGTTVTVSS | GTKVEIK |
| CB21-498-007 | EVQLVESGGGLVQPGGSLRLSCAASG | DVVMTQTPLSSPVTLGQPASISCRSSQ |
| | FTFSSYAMNWVRQAPGKGLECVSGIS | SLVHSDGNTYLNWLQQRPGQPPRLLI |
| | GNGGRIYYAESVKGRFTISRDNSKSTL | YKISNRFSGVPDRFSGSGAGTDFTLKI |
| | YLQMNSLRAEDTAVYYCAKDLDVW | SRVEAEDVGVYYCMQATQFPLTFGG |
| | GQGTTVTVSS | GTKVEIK |
| CB21-498-008 | EVQLVESGGGLVQPGGSLRLSCAASG | DVVMTQTPLSSPVTLGQPASISCRSSQ |
| | FTFSSYAMNWVRQAPGKGLECVSGIS | SLVHSDGNTYLNWLQQRPGQPPRLLI |
| | GNGGRIYYAESVKGRFTISRDNSKSTF | YKISNRFSGVPDRFSGSGAGTDFTLKI |
| | YLQMNSLRAEDTAVYYCAKDLDVW | SRVEAEDVGVYYCMQATQFPLTFGG |
| | GKGTTVTVSS | GTKVEIK |
| CB21-498-009 | EVQLVESGGGLVQPGGSLRLSCAASG | DVVMTQTPLSSPVTLGQPASISCRSSQ |
| | FTFSSYAMNWVRQAPGKGLECVSGIS | SLVHSDGNTYLNWLQQRPGQPPRLLI |
| | GNGGSIYYAESVKGRFTISRDNSKSTF | YKISNRFSGVPDRFSGSGAGTDFTLKI |
| | YLQMNSLRAEDTAVYYCAKDLDVW | SRVEAEDVGVYYCMQATQFPLTFGG |
| | GKGTTVTVSS | GTKVEIK |
| CB21-498-010 | EVQLVESGGGLVQPGGSLRLSCAASG | DVVMTQTPLSSPVTLGQPASISCRSSQ |
| | FTFSSYAMNWVRQAPGKGLECVSGIS | SLVHSDGNTYLNWLQQRPGQPPRLLI |
| | GNGGRIYYAESVKGRFTISRDNSKNTF | YKISNRFSGVPDRFSGSGAGTDFTLKI |
| | YLQMNSLRAEDTAVYYCAKDLDVW | SRVEAEDVGVYYCMQATQFPLTFGG |
| | GKGTTVTVSS | GTKVEIK |
| CB21-498-011 | EMQLVESGGGLVQPGGSLRLSCAASG | DVVMTQTPLSSPVTLGQPASISCRSSQ |
| | FTFSSYAMNWVRQAPGKGLECVSGIS | SLVHSDANTYLNWLQQRPGQPPRLLI |
| | GNGGRIYYAESVKGRFTISRDNSKSTF | YKISNRFSGVPDRFSGSGAGTDFTLKI |
| | YLQMNSLRAEDTAVYYCAKDLDVW | SRVEAEDVGVYYCMQATQFPLTFGG |
| | GKGTTVTVSS | GTKVEIK |
| CB21-498-012 | EMQLVESGGGLVQPGGSLRLSCAASG | DVVMTQTPLSSPVTLGQPASISCRSSQ |
| | FTFSSYAMNWVRQAPGKGLECVSGIS | SLVHSDINTYLNWLQQRPGQPPRLLIY |
| | GNGGRIYYAESVKGRFTISRDNSKSTF | KISNRFSGVPDRFSGSGAGTDFTLKIS |
| | YLQMNSLRAEDTAVYYCAKDLDVW | RVEAEDVGVYYCMQATQFPLTFGGG |
| | GKGTTVTVSS | TKVEIK |
| CB21-498-013 | EMQLVESGGGLVQPGGSLRLSCAASG | DVVMTQTPLSSPVTLGQPASISCRSSQ |
| | FTFSSYAMNWVRQAPGKGLECVSGIS | SLVHSIGNTYLNWLQQRPGQPPRLLIY |
| | GNGGRIYYAESVKGRFTISRDNSKSTF | KISNRFSGVPDRFSGSGAGTDFTLKIS |
| | YLQMNSLRAEDTAVYYCAKDLDVW | RVEAEDVGVYYCMQATQFPLTFGGG |
| | GKGTTVTVSS | TKVEIK |
| CB21-498-014 | EMQLVESGGGLVQPGGSLRLSCAASG | DVVMTQTPLSSPVTLGQPASISCRSSQ |
| | FTFSSYAMNWVRQAPGKGLECVSGIS | SLVHSEGNTYLNWLQQRPGQPPRLLI |
| | GNGGRIYYAESVKGRFTISRDNSKSTF | YKISNRFSGVPDRFSGSGAGTDFTLKI |
| | YLQMNSLRAEDTAVYYCAKDLDVW | SRVEAEDVGVYYCMQATQFPLTFGG |
| | GKGTTVTVSS | GTKVEIK |
| CB21-498-015 | EMQLVESGGGLVQPGGSLRLSCAASG | DVVMTQTPLSSPVTLGQPASISCRSSQ |
| | FTFSSYAMNWVRQAPGKGLECVSGIS | SLVHSDGNTYLAWLQQRPGQPPRLLI |
| | GNGGRIYYAESVKGRFTISRDNSKSTF | YKISNRFSGVPDRFSGSGAGTDFTLKI |
| | YLQMNSLRAEDTAVYYCAKDLDVW | SRVEAEDVGVYYCMQATQFPLTFGG |
| | GKGTTVTVSS | GTKVEIK |
| CB21-498-016 | EMQLVESGGGLVQPGGSLRLSCAASG | DVVMTQTPLSSPVTLGQPASISCRSSQ |
| | FTFSSYAMNWVRQAPGKGLECVSGIS | SLVHSDGNTYLNWLQQRPGQPPRLLI |
| | GNGGRIYYAESVKGRFTISRDNSKSTF | YKISNRFSGVPDRFSGSGSGTDFTLKI |
| | YLQMNSLRAEDTAVYYCAKDLDVW | SRVEAEDVGVYYCMQATQFPLTFGG |
| | GKGTTVTVSS | GTKVEIK |
| CB21-498-017 | EMQLVESGGGLVQPGGSLRLSCAASG | DVVMTQTPLSLPVTLGQPASISCRSSQ |
| | FTFSSYAMNWVRQAPGKGLECVSGIS | SLVHSDGNTYLNWLQQRPGQPPRLLI |
| | GNGGRIYYAESVKGRFTISRDNSKSTF | YKISNRFSGVPDRFSGSGAGTDFTLKI |
| | YLQMNSLRAEDTAVYYCAKDLDVW | SRVEAEDVGVYYCMQATQFPLTFGG |
| | GKGTTVTVSS | GTKVEIK |
| CB21-498-018 | EMQLVESGGGLVQPGGSLRLSCAASG | DVVMTQTPLSSPVTLGQPASISCRSSQ |
| | FTFSSYAMNWVRQAPGKGLECVSGIS | SLVHSDGNTYLNWYQQRPGQPPRLLI |
| | GNGGRIYYAESVKGRFTISRDNSKSTF | YKISNRFSGVPDRFSGSGAGTDFTLKI |
| | YLQMNSLRAEDTAVYYCAKDLDVW | SRVEAEDVGVYYCMQATQFPLTFGG |
| | GKGTTVTVSS | GTKVEIK |
| CB21-498-019 | EMQLVESGGGLVQPGGSLRLSCAASG | DVVMTQTPLSSPVTLGQPASISCRSSQ |
| | FTFSSYAMNWVRQAPGKGLECVSGIS | SLVHSDGNTYLNWLQQRPGQPPRLLI |
| | GNGGRIYYAESVKGRFTISRDNSKSTF | YKASNRFSGVPDRFSGSGAGTDFTLK |
| | YLQMNSLRAEDTAVYYCAKDLDVW | ISRVEAEDVGVYYCMQATQFPLTFGG |
| | GKGTTVTVSS | GTKVEIK |
| CB21-498-020 | EMQLVESGGGLVQPGGSLRLSCAASG | DVVMTQTPLSSPVTLGQPASISCRSSQ |
| | FTFSSYAMNWVRQAPGKGLESVSGIS | SLVHSDGNTYLNWLQQRPGQPPRLLI |
| | GNGGRIYYAESVKGRFTISRDNSKSTF | YKISNRFSGVPDRFSGSGAGTDFTLKI |
| | YLQMNSLRAEDTAVYYCAKDLDVW | SRVEAEDVGVYYCMQATQFPLTFGG |
| | GKGTTVTVSS | GTKVEIK |
| CB21-498-021 | EMQLVESGGGLVQPGGSLRLSCAASG | DVVMTQTPLSSPVTLGQPASISCRSSQ |
| | FTFSSYAMNWVRQAPGKGLEAVSGIS | SLVHSDGNTYLNWLQQRPGQPPRLLI |
| | GNGGRIYYAESVKGRFTISRDNSKSTF | YKISNRFSGVPDRFSGSGAGTDFTLKI |
| | YLQMNSLRAEDTAVYYCAKDLDVW | SRVEAEDVGVYYCMQATQFPLTFGG |
| | GKGTTVTVSS | GTKVEIK |
| CB21-498-022 | EMQLVESGGGLVQPGGSLRLSCAASG | DVVMTQTPLSSPVTLGQPASISCRSSQ |
| | FTFSSYAMNWVRQAPGKGLEGVSGIS | SLVHSDGNTYLNWLQQRPGQPPRLLI |
| | GNGGRIYYAESVKGRFTISRDNSKSTF | YKISNRFSGVPDRFSGSGAGTDFTLKI |
| | YLQMNSLRAEDTAVYYCAKDLDVW | SRVEAEDVGVYYCMQATQFPLTFGG |
| | GKGTTVTVSS | GTKVEIK |
| CB21-498-023 | EMQLVESGGGLVQPGGSLRLSCAASG | DVVMTQTPLSSPVTLGQPASISCRSSQ |
| | FTFSSYAMNWVRQAPGKGLEIVSGIS | SLVHSDGNTYLNWLQQRPGQPPRLLI |
| | GNGGRIYYAESVKGRFTISRDNSKSTF | YKISNRFSGVPDRFSGSGAGTDFTLKI |
| | YLQMNSLRAEDTAVYYCAKDLDVW | SRVEAEDVGVYYCMQATQFPLTFGG |
| | GKGTTVTVSS | GTKVEIK |
| CB21-498-024 | EMQLVESGGGLVQPGGSLRLSCAASG | DVVMTQTPLSSPVTLGQPASISCRSSQ |
| | FTFSSYAMNWVRQAPGKGLEPVSGIS | SLVHSDGNTYLNWLQQRPGQPPRLLI |
| | GNGGRIYYAESVKGRFTISRDNSKSTF | YKISNRFSGVPDRFSGSGAGTDFTLKI |
| | YLQMNSLRAEDTAVYYCAKDLDVW | SRVEAEDVGVYYCMQATQFPLTFGG |
| | GKGTTVTVSS | GTKVEIK |
| CB21-498-025 | EMQLVESGGGLVQPGGSLRLSCAASG | DVVMTQTPLSSPVTLGQPASISCRSSQ |
| | FTFSSYAMNWVRQAPGKGLETVSGIS | SLVHSDGNTYLNWLQQRPGQPPRLLI |
| | GNGGRIYYAESVKGRFTISRDNSKSTF | YKISNRFSGVPDRFSGSGAGTDFTLKI |
| | YLQMNSLRAEDTAVYYCAKDLDVW | SRVEAEDVGVYYCMQATQFPLTFGG |
| | GKGTTVTVSS | GTKVEIK |
| CB21-498-026 | EIQLVESGGGLVQPGGSLRLSCAASGF | DVVMTQTPLSSPVTLGQPASISCRSSQ |
| | TFSSYAMNWVRQAPGKGLECVSGISG | SLVHSDGNTYLNWLQQRPGQPPRLLI |
| | NGGRIYYAESVKGRFTISRDNSKSTFY | YKISNRFSGVPDRFSGSGAGTDFTLKI |
| | LQMNSLRAEDTAVYYCAKDLDVWG | SRVEAEDVGVYYCMQATQFPLTFGG |
| | KGTTVTVSS | GTKVEIK |
| CB21-498-027 | EAQLVESGGGLVQPGGSLRLSCAASG | DVVMTQTPLSSPVTLGQPASISCRSSQ |
| | FTFSSYAMNWVRQAPGKGLECVSGIS | SLVHSDGNTYLNWLQQRPGQPPRLLI |
| | GNGGRIYYAESVKGRFTISRDNSKSTF | YKISNRFSGVPDRFSGSGAGTDFTLKI |
| | YLQMNSLRAEDTAVYYCAKDLDVW | SRVEAEDVGVYYCMQATQFPLTFGG |
| | GKGTTVTVSS | GTKVEIK |
| CB21-498-028 | EMQLVESGGGLVQPGGSLRLSCAASG | DVVMTQTPLSSPVTLGQPASISCRSSQ |
| | FTFSSYAMNWVRQAPGKGLESVSGIS | SLVHSDGNTYLNWLQQRPGQPPRLLI |
| | GNAGRIYYAESVKGRFTISRDNSKSTF | YKISNRFSGVPDRFSGSGAGTDFTLKI |
| | YLQMNSLRAEDTAVYYCAKDLDVW | SRVEAEDVGVYYCMQATQFPLTFGG |
| | GKGTTVTVSS | GTKVEIK |
| CB21-498-029 | EMQLVESGGGLVQPGGSLRLSCAASG | DVVMTQTPLSSPVTLGQSASISCRSSQ |
| | FTFSSYAMNWVRQAPGKGLECVSGIS | SLVHSDGNTYLNWLHQRPGQPPRLLI |
| | GNGGRIYYAESVKGRFTISRDNSKSTF | YRISNRFSGVPDRFSGSGAGTDFTLKI |
| | YLQMNSLRAEDTAVYYCAKDLDVW | SRVEAEDVGVYYCMQATQFPLTFGG |
| | GKGTTVTVSS | GTKLEIK |
| CB21-498-030 | EMQLVESGGGLVQPGGSLRLSCAASG | DVVMTQTPLSSPVTLGQSASISCRSSQ |
| | FTFSSYAMNWVRQAPGKGLECVSGIS | SLVHSDGNTYLNWLHQRPGQPPRLLI |
| | GNGGRIYYAESVKGRFTISRDNSKSTF | YKASNRFSGVPDRFSGSGAGTDFTLK |
| | YLQMNSLRAEDTAVYYCAKDLDVW | ISRVEAEDVGVYYCMQATQFPLTFGG |
| | GKGTTVTVSS | GTKLEIK |
| CB21-498-031 | EMQLVESGGGLVQPGGSLRLSCAASG | DVVMTQTPLSSPVTLGQSASISCRSSQ |
| | FTFSSYAMNWVRQAPGKGLECVSGIS | SLVHSDGNTYLNWLHQRPGQPPRLLI |
| | GNGGRIYYAESVKGRFTISRDNSKSTF | YKNSNRFSGVPDRFSGSGAGTDFTLK |
| | YLQMNSLRAEDTAVYYCAKDLDVW | ISRVEAEDVGVYYCMQATQFPLTFGG |
| | GKGTTVTVSS | GTKLEIK |
| CB21-498-032 | EMQLVESGGGLVQPGGSLRLSCAASG | DVVMTQTPLSSPVTLGQSASISCRSSQ |
| | FTFSSYAMNWVRQAPGKGLECVSGIS | SLVHSDGNTYLNWLHQRPGQPPRLLI |
| | GNGGRIYYAESVKGRFTISRDNSKSTF | YKTSNRFSGVPDRFSGSGAGTDFTLKI |
| | YLQMNSLRAEDTAVYYCAKDLDVW | SRVEAEDVGVYYCMQATQFPLTFGG |
| | GKGTTVTVSS | GTKLEIK |
| CB21-498-033 | EMQLVESGGGLVQPGGSLRLSCAASG | DVVMTQTPLSSPVTLGQSASISCRSSQ |
| | FTFSSYAMNWVRQAPGKGLECVSGIS | SLVHSDGNTYLNWLHQRPGQPPRLLI |
| | GNGGRIYYAESVKGRFTISRDNSKSTF | YKVSNRFSGVPDRFSGSGAGTDFTLK |
| | YLQMNSLRAEDTAVYYCAKDLDVW | ISRVEAEDVGVYYCMQATQFPLTFGG |
| | GKGTTVTVSS | GTKLEIK |
| CB21-498-034 | EMQLVESGGGLVQPGGSLRLSCAASG | DVVMTQTPLSSPVTLGQSASISCRSSQ |
| | FTFSSYAMNWVRQAPGKGLECVSGIS | SLVHSDGNTYLNWLHQRPGQPPRLLI |
| | GNGGRIYYAESVKGRFTISRDNSKSTF | YKISNLFSGVPDRFSGSGAGTDFTLKI |
| | YLQMNSLRAEDTAVYYCAKDLDVW | SRVEAEDVGVYYCMQATQFPLTFGG |
| | GKGTTVTVSS | GTKLEIK |
| CB21-498-035 | EMQLVESGGGLVQPGGSLRLSCAASG | DVVMTQTPLSSPVTLGQSASISCRSSQ |
| | FTFSSYAMNWVRQAPGKGLECVSGIS | SLVHSDGNTYLNWLHQRPGQPPRLLI |
| | GNGGRIYYAESVKGRFTISRDNSKSTF | YKISNDFSGVPDRFSGSGAGTDFTLKI |
| | YLQMNSLRAEDTAVYYCAKDLDVW | SRVEAEDVGVYYCMQATQFPLTFGG |
| | GKGTTVTVSS | GTKLEIK |
| CB21-498-036 | EMQLVESGGGLVQPGGSLRLSCAASG | DVVMTQTPLSSPVTLGQSASISCRSSQ |
| | FTFSSYAMNWVRQAPGKGLECVSGIS | SLVHSDGNTYLNWLHQRPGQPPRLLI |
| | GNGGRIYYAESVKGRFTISRDNSKSTF | YKISNEFSGVPDRFSGSGAGTDFTLKI |
| | YLQMNSLRAEDTAVYYCAKDLDVW | SRVEAEDVGVYYCMQATQFPLTFGG |
| | GKGTTVTVSS | GTKLEIK |
| CB21-498-037 | EMQLVESGGGLVQPGGSLRLSCAASG | DVVMTQTPLSSPVTLGQSASISCRSSQ |
| | FTFSSYAMNWVRQAPGKGLECVSGIS | SLVHSDGNTYLNWLHQRPGQPPRLLI |
| | GNGGRIYYAESVKGRFTISRDNSKSTF | YKISNSFSGVPDRFSGSGAGTDFTLKI |
| | YLQMNSLRAEDTAVYYCAKDLDVW | SRVEAEDVGVYYCMQATQFPLTFGG |
| | GKGTTVTVSS | GTKLEIK |
| CB21-498-038 | EMQLVESGGGLVQPGGSLRLSCAASG | DVVMTQTPLSSPVTLGQSASISCRSSQ |
| | FTFSSYAMNWVRQAPGKGLECVSGIS | SLVHSDGNTYLNWLHQRPGQPPRLLI |
| | GNGGRIYYAESVKGRFTISRDNSKSTF | YKISNPFSGVPDRFSGSGAGTDFTLKI |
| | YLQMNSLRAEDTAVYYCAKDLDVW | SRVEAEDVGVYYCMQATQFPLTFGG |
| | GKGTTVTVSS | GTKLEIK |
| CB21-498-039 | EMQLVESGGGLVQPGGSLRLSCAASG | DVVMTQTPLSSPVTLGQSASISCRSSQ |
| | FTFSSYAMNWVRQAPGKGLECVSGIS | SLVHSDGNTYLNWLHQRPGQPPRLLI |
| | GNGGRIYYAESVKGRFTISRDNSKSTF | YKISNRPSGVPDRFSGSGAGTDFTLKI |
| | YLQMNSLRAEDTAVYYCAKDLDVW | SRVEAEDVGVYYCMQATQFPLTFGG |
| | GKGTTVTVSS | GTKLEIK |
| CB21-498-040 | EMQLVESGGGLVQPGGSLRLSCAASG | DVVMTQTPLSSPVTLGQSASISCRSSQ |
| | FTFSSYAMNWVRQAPGKGLECVSGIS | SLVHSDGNTYLNWLHQRPGQPPRLLI |
| | GNGGRIYYAESVKGRFTISRDNSKSTF | YKISNRASGVPDRFSGSGAGTDFTLKI |
| | YLQMNSLRAEDTAVYYCAKDLDVW | SRVEAEDVGVYYCMQATQFPLTFGG |
| | GKGTTVTVSS | GTKLEIK |
| CB21-498-041 | EMQLVESGGGLVQPGGSLRLSCAASG | DVVMTQTPLSSPVTLGQSASISCRSSQ |
| | FTFSSYAMNWVRQAPGKGLECVSGIS | SLVHSDGNTYLNWLHQRPGQPPRLLI |
| | GNGGRIYYAESVKGRFTISRDNSKSTF | YKISNRGSGVPDRFSGSGAGTDFTLKI |
| | YLQMNSLRAEDTAVYYCAKDLDVW | SRVEAEDVGVYYCMQATQFPLTFGG |
| | GKGTTVTVSS | GTKLEIK |
| CB21-498-042 | EMQLVESGGGLVQPGGSLRLSCAASG | DVVMTQTPLSSPVTLGQSASISCRSSQ |
| | FTFSSYAMNWVRQAPGKGLECVSGIS | SLVHSDGNTYLNWLHQRPGQPPRLLI |
| | GNGGRIYYAESVKGRFTISRDNSKSTF | YKISNRSSGVPDRFSGSGAGTDFTLKI |
| | YLQMNSLRAEDTAVYYCAKDLDVW | SRVEAEDVGVYYCMQATQFPLTFGG |
| | GKGTTVTVSS | GTKLEIK |
| CB21-498-043 | EMQLVESGGGLVQPGGSLRLSCAASG | DVVMTQTPLSSPVTLGQSASISCRSSQ |
| | FTFSSYAMNWVRQAPGKGLECVSGIS | SLVHSDGNTYLNWLHQRPGQPPRLLI |
| | GNGGRIYYAESVKGRFTISRDNSKSTF | YKISNRNSGVPDRFSGSGAGTDFTLKI |
| | YLQMNSLRAEDTAVYYCAKDLDVW | SRVEAEDVGVYYCMQATQFPLTFGG |
| | GKGTTVTVSS | GTKLEIK |
| CB21-498-044 | EMQLVESGGGLVQPGGSLRLSCAASG | DVVMTQTPLSSPVTLGQSASISCRSSQ |
| | FTFSSYAMNWVRQAPGKGLECVSGIS | SLVHSDGNTYLNWLHQRPGQPPRLLI |
| | GNGGRIYYAESVKGRFTISRDNSKSTF | YKISNRQSGVPDRFSGSGAGTDFTLKI |
| | YLQMNSLRAEDTAVYYCAKDLDVW | SRVEAEDVGVYYCMQATQFPLTFGG |
| | GKGTTVTVSS | GTKLEIK |
| CB21-498-045 | EMQLVESGGGLVQPGGSLRLSCAASG | DVVMTQTPLSSPVTLGQSASISCRSSQ |
| | FTFSSYAMNWVRQAPGKGLECVSGIS | SLVHSDGNTYLNWLHQRPGQPPRLLI |
| | GNGGRIYYAESVKGRFTISRDNSKSTF | YKISNRESGVPDRFSGSGAGTDFTLKI |
| | YLQMNSLRAEDTAVYYCAKDLDVW | SRVEAEDVGVYYCMQATQFPLTFGG |
| | GKGTTVTVSS | GTKLEIK |
| CB21-498-046 | EMQLVESGGGLVQPGGSLRLSCAASG | DVVMTQTPLSSPVTLGQSASISCRSSQ |
| | FTFSSYAMNWVRQAPGKGLECVSGIS | SLVHSDGNTYLNWLHQRPGQPPRLLI |
| | GNGGRIYYAESVKGRFTISRDNSKSTF | YKISNRDSGVPDRFSGSGAGTDFTLKI |
| | YLQMNSLRAEDTAVYYCAKDLDVW | SRVEAEDVGVYYCMQATQFPLTFGG |
| | GKGTTVTVSS | GTKLEIK |
| CB21-498-047 | EMQLVESGGGLVQPGGSLRLSCAASG | DVVMTQTPLSSPVTLGQSASISCRSSQ |
| | FTFSSYAMNWVRQAPGKGLECVSGIS | SLVHSDGNTYLNWLHQRPGQPPRLLI |
| | GNGGRIYYAESVKGRFTISRDNSKSTF | YKISNRFPGVPDRFSGSGAGTDFTLKI |
| | YLQMNSLRAEDTAVYYCAKDLDVW | SRVEAEDVGVYYCMQATQFPLTFGG |
| | GKGTTVTVSS | GTKLEIK |
| CB21-498-048 | EMQLVESGGGLVQPGGSLRLSCAASG | DVVMTQTPLSSPVTLGQSASISCRSSQ |
| | FTFSSYAMNWVRQAPGKGLECVSGIS | SLVHSDGNTYLNWLHQRPGQPPRLLI |
| | GNGGRIYYAESVKGRFTISRDNSKSTF | YKISNRFSPVPDRFSGSGAGTDFTLKI |
| | YLQMNSLRAEDTAVYYCAKDLDVW | SRVEAEDVGVYYCMQATQFPLTFGG |
| | GKGTTVTVSS | GTKLEIK |
| CB21-498-049 | EMQLVESGGGLVQPGGSLRLSCAASG | DVVMTQTPLSSPVTLGQSASISCRSSQ |
| | FTFSSYAMNWVRQAPGKGLECVSGIS | SLVHSDGNTYLNWLHQRPGQPPRLLI |
| | GNGGRIYYAESVKGRFTISRDNSKSTF | YKISNRFSGVPDRFSGSGAGTDFTLKI |
| | YLQMNSLRAEDTAVYYCAKDLDVW | SRVEAEDVGVYYCNQATQFPLTFGGG |
| | GKGTTVTVSS | TKLEIK |
| CB21-498-050 | EMQLVESGGGLVQPGGSLRLSCAASG | DVVMTQTPLSSPVTLGQSASISCRSSQ |
| | FTFSSYAMNWVRQAPGKGLECVSGIS | SLVHSDGNTYLNWLHQRPGQPPRLLI |
| | GNGGRIYYAESVKGRFTISRDNSKSTF | YKISNRFSGVPDRFSGSGAGTDFTLKI |
| | YLQMNSLRAEDTAVYYCAKDLDVW | SRVEAEDVGVYYCWQATQFPLTFGG |
| | GKGTTVTVSS | GTKLEIK |
| CB21-498-051 | EMQLVESGGGLVQPGGSLRLSCAASG | DVVMTQTPLSSPVTLGQSASISCRSSQ |
| | FTFSSYAMNWVRQAPGKGLECVSGIS | SLVHSDGNTYLNWLHQRPGQPPRLLI |
| | GNGGRIYYAESVKGRFTISRDNSKSTF | YKISNRFSGVPDRFSGSGAGTDFTLKI |
| | YLQMNSLRAEDTAVYYCAKDLDVW | SRVEAEDVGVYYCMQGTQFPLTFGG |
| | GKGTTVTVSS | GTKLEIK |
| CB21-498-052 | EMQLVESGGGLVQPGGSLRLSCAASG | DVVMTQTPLSSPVTLGQSASISCRSSQ |
| | FTFSSYAMNWVRQAPGKGLECVSGIS | SLVHSDGNTYLNWLHQRPGQPPRLLI |
| | GNGGRIYYAESVKGRFTISRDNSKSTF | YKISNRFSGVPDRFSGSGAGTDFTLKI |
| | YLQMNSLRAEDTAVYYCAKDLDVW | SRVEAEDVGVYYCMQNTQFPLTFGG |
| | GKGTTVTVSS | GTKLEIK |
| CB21-498-053 | EMQLVESGGGLVQPGGSLRLSCAASG | DVVMTQTPLSSPVTLGQSASISCRSSQ |
| | FTFSSYAMNWVRQAPGKGLECVSGIS | SLVHSDGNTYLNWLHQRPGQPPRLLI |
| | GNGGRIYYAESVKGRFTISRDNSKSTF | YKISNRFSGVPDRFSGSGAGTDFTLKI |
| | YLQMNSLRAEDTAVYYCAKDLDVW | SRVEAEDVGVYYCMQATQSPLTFGG |
| | GKGTTVTVSS | GTKLEIK |
| CB21-498-054 | EMQLVESGGGLVQPGGSLRLSCAASG | DVVMTQTPLSSPVTLGQSASISCRSSQ |
| | FTFSSYAMNWVRQAPGKGLECVSGIS | SLVHSDGNTYLNWLHQRPGQPPRLLI |
| | GNGGRIYYAESVKGRFTISRDNSKSTF | YKISNRFSGVPDRFSGSGAGTDFTLKI |
| | YLQMNSLRAEDTAVYYCAKDLDVW | SRVEAEDVGVYYCMQATQTPLTFGG |
| | GKGTTVTVSS | GTKLEIK |
| CB21-498-055 | EMQLVESGGGLVQPGGSLRLSCAASG | DVVMTQTPLSSPVTLGQSASISCRSSQ |
| | FTFSSYAMNWVRQAPGKGLECVSGIS | SLVHSDGNTYLNWLHQRPGQPPRLLI |
| | GNGGRIYYAESVKGRFTISRDNSKSTF | YKISNRFSGVPDRFSGSGAGTDFTLKI |
| | YLQMNSLRAEDTAVYYCAKDLDVW | SRVEAEDVGVYYCMQATQYPLTFGG |
| | GKGTTVTVSS | GTKLEIK |
| CB21-498-056 | EMQLVESGGGLVQPGGSLRLSCAASG | DVVMTQTPLSSPVTLGQSASISCRSSQ |
| | FTFSSYAMNWVRQAPGKGLECVSGIS | SLVHSDGNTYLNWLHQRPGQPPRLLI |
| | GNGGRIYYAESVKGRFTISRDNSKSTF | YKISNRFSGVPDRFSGSGAGTDFTLKI |
| | YLQMNSLRAEDTAVYYCAKDLDVW | SRVEAEDVGVYYCMQATQNPLTFGG |
| | GKGTTVTVSS | GTKLEIK |
| CB21-498-057 | EMQLVESGGGLVQPGGSLRLSCAASG | DVVMTQTPLSSPVTLGQSASISCRSSQ |
| | FTFSSYAMNWVRQAPGKGLECVSGIS | SLVHSDGNTYLNWLHQRPGQPPRLLI |
| | GNGGRIYYAESVKGRFTISRDNSKSTF | YKISNRFSGVPDRFSGSGAGTDFTLKI |
| | YLQMNSLRAEDTAVYYCAKDLDVW | SRVEAEDVGVYYCMQATQLPLTFGG |
| | GKGTTVTVSS | GTKLEIK |
| CB21-498-058 | EMQLVESGGGLVQPGGSLRLSCAASG | DVVMTQTPLSSPVTLGQSASISCRSSQ |
| | FTFSSYAMNWVRQAPGKGLECVSGIS | SLVHSDGNTYLNWLHQRPGQPPRLLI |
| | GNGGRIYYAESVKGRFTISRDNSKSTF | YKISNRFSGVPDRFSGSGAGTDFTLKI |
| | YLQMNSLRAEDTAVYYCAKDLDVW | SRVEAEDVGVYYCMQATQAPLTFGG |
| | GKGTTVTVSS | GTKLEIK |
| CB21-498-059 | EMQLVESGGGLVQPGGSLRLSCAASG | DVVMTQTPLSSPVTLGQSASISCRSSQ |
| | FTFSSYAMNWVRQAPGKGLECVSGIS | SLVHSDGNTYLNWLHQRPGQPPRLLI |
| | GNGGRIYYAESVKGRFTISRDNSKSTF | YKISNRFSGVPDRFSGSGAGTDFTLKI |
| | YLQMNSLRAEDTAVYYCAKDLDVW | SRVEAEDVGVYYCMQATQFNLTFGG |
| | GKGTTVTVSS | GTKLEIK |
| CB21-498-060 | EMQLVESGGGLVQPGGSLRLSCAASG | DVVMTQTPLSSPVTLGQSASISCRSSQ |
| | FTFSSYAMNWVRQAPGKGLECVSGIS | SLVHSDGNTYLNWLHQRPGQPPRLLI |
| | GNGGRIYYAESVKGRFTISRDNSKSTF | YKISNRFSGVPDRFSGSGAGTDFTLKI |
| | YLQMNSLRAEDTAVYYCAKDLDVW | SRVEAEDVGVYYCMQATQFQLTFGG |
| | GKGTTVTVSS | GTKLEIK |
| CB21-498-061 | EMQLVESGGGLVQPGGSLRLSCAASG | DVVMTQTPLSSPVTLGQSASISCRSSQ |
| | FTFSSYAMNWVRQAPGKGLECVSGIS | SLVHSDGNTYLNWLHQRPGQPPRLLI |
| | GNGGRIYYAESVKGRFTISRDNSKSTF | YKISNRFSGVPDRFSGSGAGTDFTLKI |
| | YLQMNSLRAEDTAVYYCAKDLDVW | SRVEAEDVGVYYCMQATQFDLTFGG |
| | GKGTTVTVSS | GTKLEIK |
| CB21-498-062 | EMQLVESGGGLVQPGGSLRLSCAASG | DVVMTQTPLSSPVTLGQSASISCRSSQ |
| | FTFSSYAMNWVRQAPGKGLECVSGIS | SLVHSDGNTYLNWLHQRPGQPPRLLI |
| | GNGGRIYYAESVKGRFTISRDNSKSTF | YKISNRFSGVPDRFSGSGAGTDFTLKI |
| | YLQMNSLRAEDTAVYYCAKDLDVW | SRVEAEDVGVYYCMQATQFSLTFGG |
| | GKGTTVTVSS | GTKLEIK |
| CB21-498-063 | EMQLVESGGGLVQPGGSLRLSCAASG | DVVMTQTPLSSPVTLGQSASISCRSSQ |
| | FTFSSYAMNWVRQAPGKGLECVSGIS | SLVHSDGNTYLNWLHQRPGQPPRLLI |
| | GNGGRIYYAESVKGRFTISRDNSKSTF | YKISNRFSGVPDRFSGSGAGTDFTLKI |
| | YLQMNSLRAEDTAVYYCAKDLDVW | SRVEAEDVGVYYCMQATQFPSTFGG |
| | GKGTTVTVSS | GTKLEIK |
| CB21-498-064 | EMQLVESGGGLVQPGGSLRLSCAASG | DVVMTQTPLSSPVTLGQSASISCRSSQ |
| | FTFSSYAMNWVRQAPGKGLECVSGIS | SLVHSDGNTYLNWLHQRPGQPPRLLI |
| | GNGGRIYYAESVKGRFTISRDNSKSTF | YKISNRFSGVPDRFSGSGAGTDFTLKI |
| | YLQMNSLRAEDTAVYYCAKDLDVW | SRVEAEDVGVYYCMQATQFPGTFGG |
| | GKGTTVTVSS | GTKLEIK |
| CB21-498-065 | EMQLVESGGGLVQPGGSLRLSCAASG | DVVMTQTPLSSPVTLGQSASISCRSSQ |
| | FTFSSYAMNWVRQAPGKGLECVSGIS | SLVHSDGNTYLNWLHQRPGQPPRLLI |
| | GNGGRIYYAESVKGRFTISRDNSKSTF | YKISNRFSGVPDRFSGSGAGTDFTLKI |
| | YLQMNSLRAEDTAVYYCAKDLDVW | SRVEAEDVGVYYCMQATQFPYTFGG |
| | GKGTTVTVSS | GTKLEIK |
| CB21-498-066 | EMQLVESGGGLVQPGGSLRLSCAASG | DVVMTQTPLSSPVTLGQSASISCRSSQ |
| | FTFSSYAMNWVRQAPGKGLECVSGIS | SLVHSDGNTYLNWLHQRPGQPPRLLI |
| | GNGGRIYYAESVKGRFTISRDNSKSTF | YKISNRFSGVPDRFSGSGAGTDFTLKI |
| | YLQMNSLRAEDTAVYYCAKDLDVW | SRVEAEDVGVYYCMQATQFPWTFGG |
| | GKGTTVTVSS | GTKLEIK |
| CB21-498-067 | EMQLVESGGGLVQPGGSLRLSCAASG | DVVMTQTPLSSPVTLGQSASISCRSSQ |
| | FTFSSYAMNWVRQAPGKGLECVSGIS | SLVHSDGNTYLNWLHQRPGQPPRLLI |
| | GNGGRIYYAESVKGRFTISRDNSKSTF | YKISNRFSGVPDRFSGSGAGTDFTLKI |
| | YLQMNSLRAEDTAVYYCAKDLDVW | SRVEAEDVGVYYCMQATQFSLPFGG |
| | GKGTTVTVSS | GTKLEIK |
| CB21-498-068 | EMQLVESGGGLVQPGGSLRLSCAASG | DVVMTQTPLSSPVTLGQPASISCRSSQ |
| | FTFSSYAMNWVRQAPGKGLEGVSGIS | SLVHSDGNTYLNWLQQRPGQPPRLLI |
| | GSGGRIYYAESVKGRFTISRDNSKSTF | YKISNRFSGVPDRFSGSGAGTDFTLKI |
| | YLQMNSLRAEDTAVYYCAKDLDVW | SRVEAEDVGVYYCMQATQFPLTFGG |
| | GKGTTVTVSS | GTKVEIK |
| CB21-498-069 | EVQLVESGGGLVQPGGSLRLSCAASG | DVVMTQTPLSSPVTLGQPASISCRSSQ |
| | FTFSSYAMNWVRQAPGKGLEGVSGIS | SLVHSDGNTYLNWLQQRPGQPPRLLI |
| | GNGGRIYYAESVKGRFTISRDNSKSTF | YKISNRFSGVPDRFSGSGAGTDFTLKI |
| | YLQMNSLRAEDTAVYYCAKDLDVW | SRVEAEDVGVYYCMQATQFPLTFGG |
| | GKGTTVTVSS | GTKVEIK |
| CB21-498-070 | EVQLVESGGGLVQPGGSLRLSCAASG | DVVMTQTPLSSPVTLGQPASISCRSSQ |
| | FTFSSYAMNWVRQAPGKGLEGVSGIS | SLVHSDGNTYLNWLQQRPGQPPRLLI |
| | GSGGRIYYAESVKGRFTISRDNSKSTF | YKISNRFSGVPDRFSGSGAGTDFTLKI |
| | YLQMNSLRAEDTAVYYCAKDLDVW | SRVEAEDVGVYYCMQATQFPLTFGG |
| | GKGTTVTVSS | GTKVEIK |
| CB21-498-071 | EMQLVESGGGLVQPGGSLRLSCAASG | DVVMTQTPLSSPVTLGQPASISCRSSQ |
| | FTFSSYAMNWVRQAPGKGLEGVSGIS | SLVHSDANTYLNWLQQRPGQPPRLLI |
| | GNGGRIYYAESVKGRFTISRDNSKSTF | YKISNRFSGVPDRFSGSGAGTDFTLKI |
| | YLQMNSLRAEDTAVYYCAKDLDVW | SRVEAEDVGVYYCMQATQFPLTFGG |
| | GKGTTVTVSS | GTKVEIK |
| CB21-498-072 | EMQLVESGGGLVQPGGSLRLSCAASG | DVVMTQTPLSSPVTLGQPASISCRSSQ |
| | FTFSSYAMNWVRQAPGKGLEGVSGIS | SLVHSDANTYLNWLQQRPGQPPRLLI |
| | GSGGRIYYAESVKGRFTISRDNSKSTF | YKISNRFSGVPDRFSGSGAGTDFTLKI |
| | YLQMNSLRAEDTAVYYCAKDLDVW | SRVEAEDVGVYYCMQATQFPLTFGG |
| | GKGTTVTVSS | GTKVEIK |
| CB21-498-073 | EVQLVESGGGLVQPGGSLRLSCAASG | DVVMTQTPLSSPVTLGQPASISCRSSQ |
| | FTFSSYAMNWVRQAPGKGLECVSGIS | SLVHSDANTYLNWLQQRPGQPPRLLI |
| | GNGGRIYYAESVKGRFTISRDNSKSTF | YKISNRFSGVPDRFSGSGAGTDFTLKI |
| | YLQMNSLRAEDTAVYYCAKDLDVW | SRVEAEDVGVYYCMQATQFPLTFGG |
| | GKGTTVTVSS | GTKVEIK |
| CB21-498-074 | EVQLVESGGGLVQPGGSLRLSCAASG | DVVMTQTPLSSPVTLGQPASISCRSSQ |
| | FTFSSYAMNWVRQAPGKGLECVSGIS | SLVHSDANTYLNWLQQRPGQPPRLLI |
| | GSGGRIYYAESVKGRFTISRDNSKSTF | YKISNRFSGVPDRFSGSGAGTDFTLKI |
| | YLQMNSLRAEDTAVYYCAKDLDVW | SRVEAEDVGVYYCMQATQFPLTFGG |
| | GKGTTVTVSS | GTKVEIK |
| CB21-498-075 | EVQLVESGGGLVQPGGSLRLSCAASG | DVVMTQTPLSSPVTLGQPASISCRSSQ |
| | FTFSSYAMNWVRQAPGKGLEGVSGIS | SLVHSDANTYLNWLQQRPGQPPRLLI |
| | GNGGRIYYAESVKGRFTISRDNSKSTF | YKISNRFSGVPDRFSGSGAGTDFTLKI |
| | YLQMNSLRAEDTAVYYCAKDLDVW | SRVEAEDVGVYYCMQATQFPLTFGG |
| | GKGTTVTVSS | GTKVEIK |
| CB21-498-076 | EVQLVESGGGLVQPGGSLRLSCAASG | DVVMTQTPLSSPVTLGQPASISCRSSQ |
| | FTFSSYAMNWVRQAPGKGLEGVSGIS | SLVHSDANTYLNWLQQRPGQPPRLLI |
| | GSGGRIYYAESVKGRFTISRDNSKSTF | YKISNRFSGVPDRFSGSGAGTDFTLKI |
| | YLQMNSLRAEDTAVYYCAKDLDVW | SRVEAEDVGVYYCMQATQFPLTFGG |
| | GKGTTVTVSS | GTKVEIK |
| CB21-498-077 | EMQLVESGGGLVQPGGSLRLSCAASG | DVVMTQTPLSSPVTLGQPASISCRSSQ |
| | FTFSSYAMNWVRQAPGKGLECVSGIS | SLVHSDANTYLNWLQQRPGQPPRLLI |
| | GNGGRIYYAESVKGRFTISRDNSKSTF | YKISNRFSGVPDRFSGSGSGTDFTLKI |
| | YLQMNSLRAEDTAVYYCAKDLDVW | SRVEAEDVGVYYCMQATQFPLTFGG |
| | GKGTTVTVSS | GTKVEIK |
| CB21-498-078 | EMQLVESGGGLVQPGGSLRLSCAASG | DVVMTQTPLSSPVTLGQPASISCRSSQ |
| | FTFSSYAMNWVRQAPGKGLEGVSGIS | SLVHSDANTYLNWLQQRPGQPPRLLI |
| | GNGGRIYYAESVKGRFTISRDNSKSTF | YKISNRFSGVPDRFSGSGSGTDFTLKI |
| | YLQMNSLRAEDTAVYYCAKDLDVW | SRVEAEDVGVYYCMQATQFPLTFGG |
| | GKGTTVTVSS | GTKVEIK |
| CB21-498-079 | EMQLVESGGGLVQPGGSLRLSCAASG | DVVMTQTPLSSPVTLGQPASISCRSSQ |
| | FTFSSYAMNWVRQAPGKGLEGVSGIS | SLVHSDANTYLNWLQQRPGQPPRLLI |
| | GSGGRIYYAESVKGRFTISRDNSKSTF | YKISNRFSGVPDRFSGSGSGTDFTLKI |
| | YLQMNSLRAEDTAVYYCAKDLDVW | SRVEAEDVGVYYCMQATQFPLTFGG |
| | GKGTTVTVSS | GTKVEIK |
| CB21-498-080 | EVQLVESGGGLVQPGGSLRLSCAASG | DVVMTQTPLSSPVTLGQPASISCRSSQ |
| | FTFSSYAMNWVRQAPGKGLECVSGIS | SLVHSDANTYLNWLQQRPGQPPRLLI |
| | GNGGRIYYAESVKGRFTISRDNSKSTF | YKISNRFSGVPDRFSGSGSGTDFTLKI |
| | YLQMNSLRAEDTAVYYCAKDLDVW | SRVEAEDVGVYYCMQATQFPLTFGG |
| | GKGTTVTVSS | GTKVEIK |
| CB21-498-081 | EVQLVESGGGLVQPGGSLRLSCAASG | DVVMTQTPLSSPVTLGQPASISCRSSQ |
| | FTFSSYAMNWVRQAPGKGLECVSGIS | SLVHSDANTYLNWLQQRPGQPPRLLI |
| | GSGGRIYYAESVKGRFTISRDNSKSTF | YKISNRFSGVPDRFSGSGSGTDFTLKI |
| | YLQMNSLRAEDTAVYYCAKDLDVW | SRVEAEDVGVYYCMQATQFPLTFGG |
| | GKGTTVTVSS | GTKVEIK |
| CB21-498-082 | EVQLVESGGGLVQPGGSLRLSCAASG | DVVMTQTPLSSPVTLGQPASISCRSSQ |
| | FTFSSYAMNWVRQAPGKGLEGVSGIS | SLVHSDANTYLNWLQQRPGQPPRLLI |
| | GNGGRIYYAESVKGRFTISRDNSKSTF | YKISNRFSGVPDRFSGSGSGTDFTLKI |
| | YLQMNSLRAEDTAVYYCAKDLDVW | SRVEAEDVGVYYCMQATQFPLTFGG |
| | GKGTTVTVSS | GTKVEIK |
| CB21-498-083 | EVQLVESGGGLVQPGGSLRLSCAASG | DVVMTQTPLSSPVTLGQPASISCRSSQ |
| | FTFSSYAMNWVRQAPGKGLEGVSGIS | SLVHSDANTYLNWLQQRPGQPPRLLI |
| | GSGGRIYYAESVKGRFTISRDNSKSTF | YKISNRFSGVPDRFSGSGSGTDFTLKI |
| | YLQMNSLRAEDTAVYYCAKDLDVW | SRVEAEDVGVYYCMQATQFPLTFGG |
| | GKGTTVTVSS | GTKVEIK |
| CB21-498-084 | EMQLVESGGGLVQPGGSLRLSCAASG | DVVMTQTPLSSPVTLGQSASISCRSSQ |
| | FTFSSYAMNWVRQAPGKGLECVSGIS | SLVHSDANTYLNWLHQRPGQPPRLLI |
| | GNGGRIYYAESVKGRFTISRDNSKSTF | YKISNRFSGVPDRFSGSGSGTDFTLKI |
| | YLQMNSLRAEDTAVYYCAKDLDVW | SRVEAEDVGVYYCMQATQFPLTFGG |
| | GKGTTVTVSS | GTKLEIK |
| CB21-498-085 | EMQLVESGGGLVQPGGSLRLSCAASG | DVVMTQTPLSSPVTLGQSASISCRSSQ |
| | FTFSSYAMNWVRQAPGKGLEGVSGIS | SLVHSDANTYLNWLHQRPGQPPRLLI |
| | GNGGRIYYAESVKGRFTISRDNSKSTF | YKISNRFSGVPDRFSGSGSGTDFTLKI |
| | YLQMNSLRAEDTAVYYCAKDLDVW | SRVEAEDVGVYYCMQATQFPLTFGG |
| | GKGTTVTVSS | GTKLEIK |
| CB21-498-086 | EMQLVESGGGLVQPGGSLRLSCAASG | DVVMTQTPLSSPVTLGQSASISCRSSQ |
| | FTFSSYAMNWVRQAPGKGLEGVSGIS | SLVHSDANTYLNWLHQRPGQPPRLLI |
| | GSGGRIYYAESVKGRFTISRDNSKSTF | YKISNRFSGVPDRFSGSGSGTDFTLKI |
| | YLQMNSLRAEDTAVYYCAKDLDVW | SRVEAEDVGVYYCMQATQFPLTFGG |
| | GKGTTVTVSS | GTKLEIK |
| CB21-498-087 | EVQLVESGGGLVQPGGSLRLSCAASG | DVVMTQTPLSSPVTLGQSASISCRSSQ |
| | FTFSSYAMNWVRQAPGKGLECVSGIS | SLVHSDANTYLNWLHQRPGQPPRLLI |
| | GNGGRIYYAESVKGRFTISRDNSKSTF | YKISNRFSGVPDRFSGSGSGTDFTLKI |
| | YLQMNSLRAEDTAVYYCAKDLDVW | SRVEAEDVGVYYCMQATQFPLTFGG |
| | GKGTTVTVSS | GTKLEIK |
| CB21-498-088 | EVQLVESGGGLVQPGGSLRLSCAASG | DVVMTQTPLSSPVTLGQSASISCRSSQ |
| | FTFSSYAMNWVRQAPGKGLECVSGIS | SLVHSDANTYLNWLHQRPGQPPRLLI |
| | GSGGRIYYAESVKGRFTISRDNSKSTF | YKISNRFSGVPDRFSGSGSGTDFTLKI |
| | YLQMNSLRAEDTAVYYCAKDLDVW | SRVEAEDVGVYYCMQATQFPLTFGG |
| | GKGTTVTVSS | GTKLEIK |
| CB21-498-089 | EVQLVESGGGLVQPGGSLRLSCAASG | DVVMTQTPLSSPVTLGQSASISCRSSQ |
| | FTFSSYAMNWVRQAPGKGLEGVSGIS | SLVHSDANTYLNWLHQRPGQPPRLLI |
| | GNGGRIYYAESVKGRFTISRDNSKSTF | YKISNRFSGVPDRFSGSGSGTDFTLKI |
| | YLQMNSLRAEDTAVYYCAKDLDVW | SRVEAEDVGVYYCMQATQFPLTFGG |
| | GKGTTVTVSS | GTKLEIK |
| CB21-498-090 | EVQLVESGGGLVQPGGSLRLSCAASG | DVVMTQTPLSSPVTLGQSASISCRSSQ |
| | FTFSSYAMNWVRQAPGKGLEGVSGIS | SLVHSDANTYLNWLHQRPGQPPRLLI |
| | GSGGRIYYAESVKGRFTISRDNSKSTF | YKISNRFSGVPDRFSGSGSGTDFTLKI |
| | YLQMNSLRAEDTAVYYCAKDLDVW | SRVEAEDVGVYYCMQATQFPLTFGG |
| | GKGTTVTVSS | GTKLEIK |
| CB21-498-091 | EVQLVESGGGLVQPGGSLRLSCAASG | DVVMTQTPLSSPVTLGQPASISCRSSQ |
| | FTFSSYAMSWVRQAPGKGLEGVSGIS | SLVHSDGNTYLNWLQQRPGQPPRLLI |
| | GSGGRIYYAESVKGRFTISRDNSKSTF | YKISNRFSGVPDRFSGSGAGTDFTLKI |
| | YLQMNSLRAEDTAVYYCAKDLDVW | SRVEAEDVGVYYCMQATQFPLTFGG |
| | GKGTTVTVSS | GTKVEIK |
| CB21-498-092 | EVQLVESGGGLVQPGGSLRLSCAASG | DVVMTQTPLSSPVTLGQPASISCRSSQ |
| | FTFSSYAMHWVRQAPGKGLEGVSGIS | SLVHSDGNTYLNWLQQRPGQPPRLLI |
| | GSGGRIYYAESVKGRFTISRDNSKSTF | YKISNRFSGVPDRFSGSGAGTDFTLKI |
| | YLQMNSLRAEDTAVYYCAKDLDVW | SRVEAEDVGVYYCMQATQFPLTFGG |
| | GKGTTVTVSS | GTKVEIK |
| CB21-498-093 | EVQLVESGGGLVQPGGSLRLSCAASG | DVVMTQTPLSSPVTLGQPASISCRSSQ |
| | FTFSSYAMTWVRQAPGKGLEGVSGIS | SLVHSDGNTYLNWLQQRPGQPPRLLI |
| | GSGGRIYYAESVKGRFTISRDNSKSTF | YKISNRFSGVPDRFSGSGAGTDFTLKI |
| | YLQMNSLRAEDTAVYYCAKDLDVW | SRVEAEDVGVYYCMQATQFPLTFGG |
| | GKGTTVTVSS | GTKVEIK |
| CB21-498-094 | EVQLVESGGGLVQPGGSLRLSCAASG | DVVMTQTPLSSPVTLGQPASISCRSSQ |
| | FTFSSYAMNWVRQAPGRGLEGVSGIS | SLVHSDGNTYLNWLQQRPGQPPRLLI |
| | GSGGRIYYAESVKGRFTISRDNSKSTF | YKISNRFSGVPDRFSGSGAGTDFTLKI |
| | YLQMNSLRAEDTAVYYCAKDLDVW | SRVEAEDVGVYYCMQATQFPLTFGG |
| | GKGTTVTVSS | GTKVEIK |
| CB21-498-095 | EVQLVESGGGLVQPGGSLRLSCAASG | DVVMTQTPLSSPVTLGQPASISCRSSQ |
| | FTFSSYAMNWVRQAPGQGLEGVSGIS | SLVHSDGNTYLNWLQQRPGQPPRLLI |
| | GSGGRIYYAESVKGRFTISRDNSKSTF | YKISNRFSGVPDRFSGSGAGTDFTLKI |
| | YLQMNSLRAEDTAVYYCAKDLDVW | SRVEAEDVGVYYCMQATQFPLTFGG |
| | GKGTTVTVSS | GTKVEIK |
| CB21-498-096 | EVQLVESGGGLVQPGGSLRLSCAASG | DVVMTQTPLSSPVTLGQPASISCRSSQ |
| | FTFSSYAMNWVRQAPGEGLEGVSGIS | SLVHSDGNTYLNWLQQRPGQPPRLLI |
| | GSGGRIYYAESVKGRFTISRDNSKSTF | YKISNRFSGVPDRFSGSGAGTDFTLKI |
| | YLQMNSLRAEDTAVYYCAKDLDVW | SRVEAEDVGVYYCMQATQFPLTFGG |
| | GKGTTVTVSS | GTKVEIK |
| CB21-498-097 | EVQLVESGGGLVQPGGSLRLSCAASG | DVVMTQTPLSSPVTLGQPASISCRSSQ |
| | FTFSSYAMNWVRQAPGKGLEGVSGIS | SLVHSDGNTYLNWLQQRPGQPPRLLI |
| | GSGGRIYKAESVKGRFDISRDNSKSTF | YKISNRFSGVPDRFSGSGAGTDFTLKI |
| | YLQMNSLRAEDTAVYYCAKDLDVW | SRVEAEDVGVYYCMQATQFPLTFGG |
| | GKGTTVTVSS | GTKVEIK |
| CB21-498-098 | EVQLVESGGGLVQPGGSLRLSCAASG | DVVMTQTPLSSPVTLGQPASISCRSSQ |
| | FTFSSYAMNWVRQAPGKGLEGVSGIS | SLVHSDGNTYLNWLQQRPGQPPRLLI |
| | GSGGRIYYAESVKGRFMISRDNSKSTF | YKISNRFSGVPDRFSGSGAGTDFTLKI |
| | YLQMNSLRAEDTAVYYCAKDLDVW | SRVEAEDVGVYYCMQATQFPLTFGG |
| | GKGTTVTVSS | GTKVEIK |
| CB21-498-099 | EVQLVESGGGLVQPGGSLRLSCAASG | DVVMTQTPLSSPVTLGQPASISCRSSQ |
| | FTFSSYAMNWVRQAPGKGLEGVSGIS | SLVHSDGNTYLNWLQQRPGQPPRLLI |
| | GSGGRIYYAESVKGRFQISRDNSKSTF | YKISNRFSGVPDRFSGSGAGTDFTLKI |
| | YLQMNSLRAEDTAVYYCAKDLDVW | SRVEAEDVGVYYCMQATQFPLTFGG |
| | GKGTTVTVSS | GTKVEIK |
| CB21-498-100 | EVQLVESGGGLVQPGGSLRLSCAASG | DVVMTQTPLSSPVTLGQPASISCRSSQ |
| | FTFSSYAMNWVRQAPGKGLEGVSGIS | SLVHSDGNTYLNWLQQRPGQPPRLLI |
| | GSGGRIYYAESVKGRFTILRDNSKSTF | YKISNRFSGVPDRFSGSGAGTDFTLKI |
| | YLQMNSLRAEDTAVYYCAKDLDVW | SRVEAEDVGVYYCMQATQFPLTFGG |
| | GKGTTVTVSS | GTKVEIK |
| CB21-498-101 | EVQLVESGGGLVQPGGSLRLSCAASG | DVVMTQTPLSSPVTLGQPASISCRSSQ |
| | FTFSSYAMNWVRQAPGKGLEGVSGIS | SLVHSDGNTYLNWLQQRPGQPPRLLI |
| | GSGGRIYYAESVKGRFTIQRDNSKSTF | YKISNRFSGVPDRFSGSGAGTDFTLKI |
| | YLQMNSLRAEDTAVYYCAKDLDVW | SRVEAEDVGVYYCMQATQFPLTFGG |
| | GKGTTVTVSS | GTKVEIK |
| CB21-498-102 | EVQLVESGGGLVQPGGSLRLSCAASG | DVVMTQTPLSSPVTLGQPASISCRSSQ |
| | FTFSSYAMNWVRQAPGKGLEGVSGIS | SLVHSDGNTYLNWLQQRPGQPPRLLI |
| | GSGGRIYYAESVKGRFTINRDNSKSTF | YKISNRFSGVPDRFSGSGAGTDFTLKI |
| | YLQMNSLRAEDTAVYYCAKDLDVW | SRVEAEDVGVYYCMQATQFPLTFGG |
| | GKGTTVTVSS | GTKVEIK |
| CB21-498-103 | EVQLVESGGGLVQPGGSLRLSCAASG | DVVMTQTPLSSPVTLGQPASISCRSSQ |
| | FTFSSYAMNWVRQAPGKGLEGVSGIS | SLVHSDGNTYLNWLQQRPGQPPRLLI |
| | GSGGRIYYAESVKGRFTISRDNSKSTF | YKISNRFSGVPDRFSGSGAGTDFTLKI |
| | YLQMNELRAEDTAVYYCAKDLDVW | SRVEAEDVGVYYCMQATQFPLTFGG |
| | GKGTTVTVSS | GTKVEIK |
| CB21-498-104 | EVQLVESGGGLVQPGGSLRLSCAASG | DVVMTQTPLSSPVTLGQPASISCRSSQ |
| | FTFSSYAMNWVRQAPGKGLEGVSGIS | SLVHSDGNTYLNWLQQRPGQPPRLLI |
| | GSGGRIYYAESVKGRFTISRDNSKSTF | YKISNRFSGVPDRFSGSGAGTDFTLKI |
| | YLQMNRLRAEDTAVYYCAKDLDVW | SRVEAEDVGVYYCMQATQFPLTFGG |
| | GKGTTVTVSS | GTKVEIK |
| CB21-498-105 | EVQLVESGGGLVQPGGSLRLSCAASG | DVVMTQTPLSSPVTLGQPASISCRSSQ |
| | FTFSSYAMNWVRQAPGKGLEGVSGIS | SLVHSDGNTYLNWLQQRPGQPPRLLI |
| | GSGGRIYYAESVKGRFTISRDNSKSTF | YKISNRFSGVPDRFSGSGAGTDFTLKI |
| | YLQMNTLRAEDTAVYYCAKDLDVW | SRVEAEDVGVYYCMQATQFPLTFGG |
| | GKGTTVTVSS | GTKVEIK |
| CB21-498-106 | EVQLVESGGGLVQPGGSLRLSCAASG | DVVMTQTPLSSPVTLGQPASISCRSSQ |
| | FTFSDYAMNWVRQAPGKGLEGVSGIS | SLVHSDGNTYLNWLQQRPGQPPRLLI |
| | GSGGRIYYAESVKGRFTISRDNSKSTF | YKISNRFSGVPDRFSGSGAGTDFTLKI |
| | YLQMNSLRAEDTAVYYCAKDLDVW | SRVEAEDVGVYYCMQATQFPLTFGG |
| | GKGTTVTVSS | GTKVEIK |
| CB21-498-107 | EVQLVESGGGLVQPGGSLRLSCAASG | DVVMTQTPLSSPVTLGQPASISCRSSQ |
| | FTFSSYAMNWVRQAPGKGLEGVSGIS | SLVHSDGNTYLNWLQQRPGQPPRLLI |
| | GSGGAIYYAESVKGRFTISRDNSKSTF | YKISNRFSGVPDRFSGSGAGTDFTLKI |
| | YLQMNSLRAEDTAVYYCAKDLDVW | SRVEAEDVGVYYCMQATQFPLTFGG |
| | GKGTTVTVSS | GTKVEIK |
| CB21-498-108 | EVQLVESGGGLVQPGGSLRLSCAASG | DVVMTQTPLSSPVTLGQPASISCRSSQ |
| | FTFSSYAMNWVRQAPGKGLEGVSGIS | SLVHSDGNTYLNWLQQRPGQPPRLLI |
| | GSGGRAYYAESVKGRFTISRDNSKSTF | YKISNRFSGVPDRFSGSGAGTDFTLKI |
| | YLQMNSLRAEDTAVYYCAKDLDVW | SRVEAEDVGVYYCMQATQFPLTFGG |
| | GKGTTVTVSS | GTKVEIK |
| CB21-498-109 | EVQLVESGGGLVQPGGSLRLSCAASG | DVVMTQTPLSSPVTLGQPASISCRSSQ |
| | FTFSSYAMNWVRQAPGKGLEGVSGIS | SLVHSDGNTYLNWLQQRPGQPPRLLI |
| | GSGGSTYYAESVKGRFTISRDNSKSTF | YKISNRFSGVPDRFSGSGAGTDFTLKI |
| | YLQMNSLRAEDTAVYYCAKDLDVW | SRVEAEDVGVYYCMQATQFPLTFGG |
| | GKGTTVTVSS | GTKVEIK |
| CB21-498-110 | EVQLVESGGGLVQPGGSLRLSCAASG | DVVMTQTPLSSPVTLGQPASISCRSSQ |
| | FTFSSYAMNWVRQAPGKGLEGVSGIS | SLVHSDGNTYLNWLQQRPGQPPRLLI |
| | GSGGRIFYAESVKGRFTISRDNSKSTF | YKISNRFSGVPDRFSGSGAGTDFTLKI |
| | YLQMNSLRAEDTAVYYCAKDLDVW | SRVEAEDVGVYYCMQATQFPLTFGG |
| | GKGTTVTVSS | GTKVEIK |
| CB21-498-111 | EVQLVESGGGLVQPGGSLRLSCAASG | DVVMTQTPLSSPVTLGQPASISCRSSQ |
| | FTFSSYAMNWVRQAPGKGLEGVSGIS | SLVHSDGNTYLNWLQQRPGQPPRLLI |
| | GSGGRIHYAESVKGRFTISRDNSKSTF | YKISNRFSGVPDRFSGSGAGTDFTLKI |
| | YLQMNSLRAEDTAVYYCAKDLDVW | SRVEAEDVGVYYCMQATQFPLTFGG |
| | GKGTTVTVSS | GTKVEIK |
| CB21-498-112 | EVQLVESGGGLVQPGGSLRLSCAASG | DVVMTQTPLSSPVTLGQPASISCRSSQ |
| | FTFSSYAMNWVRQAPGKGLEGVSGIS | SLVHSDGNTYLNWLQQRPGQPPRLLI |
| | GSGGRINYAESVKGRFTISRDNSKSTF | YKISNRFSGVPDRFSGSGAGTDFTLKI |
| | YLQMNSLRAEDTAVYYCAKDLDVW | SRVEAEDVGVYYCMQATQFPLTFGG |
| | GKGTTVTVSS | GTKVEIK |
| CB21-498-113 | EVQLVESGGGLVQPGGSLRLSCAASG | DVVMTQTPLSSPVTLGQPASISCRSSQ |
| | FTFSSYAMNWVRQAPGKGLEGVSGIS | SLVHSDGNTYLNWLQQRPGQPPRLLI |
| | GSGGRIKYAESVKGRFTISRDNSKSTF | YKISNRFSGVPDRFSGSGAGTDFTLKI |
| | YLQMNSLRAEDTAVYYCAKDLDVW | SRVEAEDVGVYYCMQATQFPLTFGG |
| | GKGTTVTVSS | GTKVEIK |
| CB21-498-114 | EVQLVESGGGLVQPGGSLRLSCAASG | DVVMTQTPLSSPVTLGQPASISCRSSQ |
| | FTFSSYAMNWVRQAPGKGLEGVSGIS | SLVHSDGNTYLNWLQQRPGQPPRLLI |
| | GSGGRIDYAESVKGRFTISRDNSKSTF | YKISNRFSGVPDRFSGSGAGTDFTLKI |
| | YLQMNSLRAEDTAVYYCAKDLDVW | SRVEAEDVGVYYCMQATQFPLTFGG |
| | GKGTTVTVSS | GTKVEIK |
| CB21-498-115 | EVQLVESGGGLVQPGGSLRLSCAASG | DVVMTQTPLSSPVTLGQPASISCRSSQ |
| | FTFSSYAMNWVRQAPGKGLEGVSGIS | SLVHSDGNTYLNWLQQRPGQPPRLLI |
| | GSGGRIAYAESVKGRFTISRDNSKSTF | YKISNRFSGVPDRFSGSGAGTDFTLKI |
| | YLQMNSLRAEDTAVYYCAKDLDVW | SRVEAEDVGVYYCMQATQFPLTFGG |
| | GKGTTVTVSS | GTKVEIK |
| CB21-498-116 | EVQLVESGGGLVQPGGSLRLSCAASG | DVVMTQTPLSSPVTLGQPASISCRSSQ |
| | FTFSSYAMNWVRQAPGKGLEGVSGIS | SLVHSDGNTYLNWLQQRPGQPPRLLI |
| | GSGGRIYFAESVKGRFTISRDNSKSTF | YKISNRFSGVPDRFSGSGAGTDFTLKI |
| | YLQMNSLRAEDTAVYYCAKDLDVW | SRVEAEDVGVYYCMQATQFPLTFGG |
| | GKGTTVTVSS | GTKVEIK |
| CB21-498-117 | EVQLVESGGGLVQPGGSLRLSCAASG | DVVMTQTPLSSPVTLGQPASISCRSSQ |
| | FTFSSYAMNWVRQAPGKGLEGVSGIS | SLVHSDGNTYLNWLQQRPGQPPRLLI |
| | GSGGRIYHAESVKGRFTISRDNSKSTF | YKISNRFSGVPDRFSGSGAGTDFTLKI |
| | YLQMNSLRAEDTAVYYCAKDLDVW | SRVEAEDVGVYYCMQATQFPLTFGG |
| | GKGTTVTVSS | GTKVEIK |
| CB21-498-118 | EVQLVESGGGLVQPGGSLRLSCAASG | DVVMTQTPLSSPVTLGQPASISCRSSQ |
| | FTFSSYAMNWVRQAPGKGLEGVSGIS | SLVHSDGNTYLNWLQQRPGQPPRLLI |
| | GSGGRIYPAESVKGRFTISRDNSKSTF | YKISNRFSGVPDRFSGSGAGTDFTLKI |
| | YLQMNSLRAEDTAVYYCAKDLDVW | SRVEAEDVGVYYCMQATQFPLTFGG |
| | GKGTTVTVSS | GTKVEIK |
| CB21-498-119 | EVQLVESGGGLVQPGGSLRLSCAASG | DVVMTQTPLSSPVTLGQPASISCRSSQ |
| | FTFSSYAMNWVRQAPGKGLEGVSGIS | SLVHSDGNTYLNWLQQRPGQPPRLLI |
| | GSGGRIYAAESVKGRFTISRDNSKSTF | YKISNRFSGVPDRFSGSGAGTDFTLKI |
| | YLQMNSLRAEDTAVYYCAKDLDVW | SRVEAEDVGVYYCMQATQFPLTFGG |
| | GKGTTVTVSS | GTKVEIK |
| CB21-498-120 | EVQLVESGGGLVQPGGSLKLSCVASG | DVVMTQTPLSSPVTLGQPASISCRSSQ |
| | FTFSSYAMNWVRQAPGKGLEGVSGIS | SLVHSDGNTYLNWLQQRPGQPPRLLI |
| | GSGGRIYYAESVKGRFTISRDNSKSTF | YKISNRFSGVPDRFSGSGAGTDFTLKI |
| | YLQMNSLRAEDTAVYYCAKDLDVW | SRVEAEDVGVYYCMQATQFPLTFGG |
| | GKGTTVTVSS | GTKVEIK |
| CB21-498-121 | EVQLVESGGGLVQPGGSLKLSCVASG | DVVMTQTPLSSPVTLGQPASISCRSSQ |
| | FTFSSYAMNWVRQAPGKGLEGVCGIS | SLVHSDGNTYLNWLQQRPGQPPRLLI |
| | GSGGRIYYAESVKGRFTCSRDNSKSTF | YKISNRFSGVPDRFSGSGAGTDFTLKI |
| | YLQMNSLRAEDTAVYYCAKDLDVW | SRVEAEDVGVYYCMQATQFPLTFGG |
| | GKGTTVTVSS | GTKVEIK |
| CB21-498-122 | EVQLVESGGGLVQPGGSLKLSCVASG | DVVMTQTPLSSPVTLGQPASISCRSSQ |
| | FTFSSYAMNWVRQAPGKGLEGVCGIS | SLVHSDGNTYLNWLQQRPGQPPRLLI |
| | GSGGRIYSAESVKGRFTCSRDNSKSTF | YKISNRFSGVPDRFSGSGAGTDFTLKI |
| | YLQMNSLRAEDTAVYYCAKDLDVW | SRVEAEDVGVYYCMQATQFPLTFGG |
| | GKGTTVTVSS | GTKVEIK |
| CB21-498-123 | EMQLVESGGGLVQPGGSLRLSCAASG | DVVMTQTPLSSPVTLGQSASISCRSSQ |
| | FTFSSYAMNWVRQAPGKGLEGVSGIS | SLVHSDGNTYLNWLHQRPGQPPRLLI |
| | GNGGRIYYAESVKGRFTISRDNSKSTF | YKISNRFDGVPDRFSGSGAGTDFTLKI |
| | YLQMNSLRAEDTAVYYCAKDLDVW | SRVEAEDVGVYYCMQATQFPLTFGG |
| | GKGTTVTVSS | GTKLEIK |
| CB21-498-124 | EMQLVESGGGLVQPGGSLRLSCAASG | DVVMTQTPLSSPVTLGQSASISCRSSQ |
| | FTFSSYAMNWVRQAPGKGLEGVSGIS | SLVHSDGNTYLNWLHQRPGQPPRLLI |
| | GNGGRIYYAESVKGRFTISRDNSKSTF | YKISNRFEGVPDRFSGSGAGTDFTLKI |
| | YLQMNSLRAEDTAVYYCAKDLDVW | SRVEAEDVGVYYCMQATQFPLTFGG |
| | GKGTTVTVSS | GTKLEIK |
| CB21-498-125 | EMQLVESGGGLVQPGGSLRLSCAASG | DVVMTQTPLSSPVTLGQSASISCRSSQ |
| | FTFSSYAMNWVRQAPGKGLEGVSGIS | SLVHSDGNTYLNWLHQRPGQPPRLLI |
| | GNGGRIYYAESVKGRFTISRDNSKSTF | YKISNRFQGVPDRFSGSGAGTDFTLKI |
| | YLQMNSLRAEDTAVYYCAKDLDVW | SRVEAEDVGVYYCMQATQFPLTFGG |
| | GKGTTVTVSS | GTKLEIK |
| CB21-498-126 | EMQLVESGGGLVQPGGSLRLSCAASG | DVVMTQTPLSSPVTLGQSASISCRSSQ |
| | FTFSSYAMNWVRQAPGKGLEGVSGIS | SLVHSDGNTYLNWLHQRPGQPPRLLI |
| | GNGGRIYYAESVKGRFTISRDNSKSTF | YKISNRFSGVPDRFTGSGAGTDFTLKI |
| | YLQMNSLRAEDTAVYYCAKDLDVW | SRVEAEDVGVYYCMQATQFPLTFGG |
| | GKGTTVTVSS | GTKLEIK |
| CB21-498-127 | EMQLVESGGGLVQPGGSLRLSCAASG | DVVMTQTPLSSPVTLGQSASISCRSSQ |
| | FTFSSYAMNWVRQAPGKGLEGVSGIS | SLVHSDGNTYLNWLHQRPGQPPRLLI |
| | GNGGRIYYAESVKGRFTISRDNSKSTF | YKISNRFSGVPDRFQGSGAGTDFTLKI |
| | YLQMNSLRAEDTAVYYCAKDLDVW | SRVEAEDVGVYYCMQATQFPLTFGG |
| | GKGTTVTVSS | GTKLEIK |
| CB21-498-128 | EMQLVESGGGLVQPGGSLRLSCAASG | DVVMTQTPLSSPVTLGQSASISCRSSQ |
| | FTFSSYAMNWVRQAPGKGLEGVSGIS | SLVHSDGNTYLNWLHQRPGQPPRLLI |
| | GNGGRIYYAESVKGRFTISRDNSKSTF | YKISNRFSGVPDRFGGSGAGTDFTLKI |
| | YLQMNSLRAEDTAVYYCAKDLDVW | SRVEAEDVGVYYCMQATQFPLTFGG |
| | GKGTTVTVSS | GTKLEIK |
| CB21-498-129 | EMQLVESGGGLVQPGGSLRLSCAASG | DVVMTQTPLSSPVTLGQSASISCRSSQ |
| | FTFSSYAMNWVRQAPGKGLEGVSGIS | SLVHSDGNTYLNWLHQRPGQPPRLLI |
| | GNGGRIYYAESVKGRFTISRDNSKSTF | YKISNRFSGVPDRFSGSGAGTDFTLTI |
| | YLQMNSLRAEDTAVYYCAKDLDVW | SRVEAEDVGVYYCMQATQFPLTFGG |
| | GKGTTVTVSS | GTKLEIK |
| CB21-498-130 | EMQLVESGGGLVQPGGSLRLSCAASG | DVVMTQTPLSSPVTLGQSASISCRSSQ |
| | FTFSSYAMNWVRQAPGKGLEGVSGIS | SLVHSDGNTYLNWLHQRPGQPPRLLI |
| | GNGGRIYYAESVKGRFTISRDNSKSTF | YKISNRFSGVPDRFSGSGAGTDFTLRI |
| | YLQMNSLRAEDTAVYYCAKDLDVW | SRVEAEDVGVYYCMQATQFPLTFGG |
| | GKGTTVTVSS | GTKLEIK |
| CB21-498-131 | EMQLVESGGGLVQPGGSLRLSCAASG | DVVMTQTPLSSPVTLGQSASISCRSSQ |
| | FTFSSYAMNWVRQAPGKGLEGVSGIS | SLVHSDGNTYLNWLHQRPGQPPRLLI |
| | GNGGRIYYAESVKGRFTISRDNSKSTF | YKISNRFSGVPDRFSGSGAGTDFTLEI |
| | YLQMNSLRAEDTAVYYCAKDLDVW | SRVEAEDVGVYYCMQATQFPLTFGG |
| | GKGTTVTVSS | GTKLEIK |
| CB21-498-132 | EMQLVESGGGLVQPGGSLRLSCAASG | DVVMTQTPLSSPVTLGQSASISCRSSQ |
| | FTFSSYAMNWVRQAPGKGLEGVSGIS | SLVHSDGNTYLNWLHQRPGQPPRLLI |
| | GNGGRIYYAESVKGRFTISRDNSKSTF | YKIDNRFSGVPDRFSGSGAGTDFTLKI |
| | YLQMNSLRAEDTAVYYCAKDLDVW | SRVEAEDVGVYYCMQATQFPLTFGG |
| | GKGTTVTVSS | GTKLEIK |
| CB21-498-133 | EMQLVESGGGLVQPGGSLRLSCAASG | DVVMTQTPLSSAVLLGQSASISCRSSQ |
| | FTFSSYAMNWVRQAPGKGLEGVSGIS | SLVHSDGNTYLNWLHQRPGQPPRLLI |
| | GNGGRIYYAESVKGRFTISRDNSKSTF | YKIDNRFSGVPDRFSGSGAGTDFTLKI |
| | YLQMNSLRAEDTAVYYCAKDLDVW | SRVEAEDVGVYYCMQATQFPLTFGG |
| | GKGTTVTVSS | GTKLEIK |
| CB21-498-134 | EVQLVESGGGLVQPGGSLRLSCAASG | DVVMTQTPLSSPVTLGQSASISCRSSQ |
| | FTFSSYAMNWVRQAPGKGLEGVSGIS | SLVHSDANTYLNWLHQRPGQPPRLLI |
| | GSGGRAYYAESVKGRFQISRDNSKST | YKISNRFSGVPDRFSGSGSGTDFTLKI |
| | FYLQMNSLRAEDTAVYYCAKDLDVW | SRVEAEDVGVYYCMQATQFPLTFGG |
| | GKGTTVTVSS | GTKLEIK |
| CB21-498-135 | EVQLVESGGGLVQPGGSLRLSCAASG | DVVMTQTPLSSPVTLGQSASISCRSSQ |
| | FTFSSYAMNWVRQAPGRGLEGVSGIS | SLVHSDANTYLNWLHQRPGQPPRLLI |
| | GSGGRIYYAESVKGRFQISRDNSKSTF | YKISNRFSGVPDRFSGSGSGTDFTLKI |
| | YLQMNSLRAEDTAVYYCAKDLDVW | SRVEAEDVGVYYCMQATQFPLTFGG |
| | GKGTTVTVSS | GTKLEIK |
| CB21-498-136 | EVQLVESGGGLVQPGGSLRLSCAASG | DVVMTQTPLSSPVTLGQSASISCRSSQ |
| | FTFSDYAMNWVRQAPGKGLEGVSGIS | SLVHSDANTYLNWLHQRPGQPPRLLI |
| | GSGGRIYYAESVKGRFQISRDNSKSTF | YKISNRFSGVPDRFSGSGSGTDFTLKI |
| | YLQMNSLRAEDTAVYYCAKDLDVW | SRVEAEDVGVYYCMQATQFPLTFGG |
| | GKGTTVTVSS | GTKLEIK |
| CB21-498-137 | EVQLVESGGGLVQPGGSLRLSCAASG | DVVMTQTPLSSPVTLGQSASISCRSSQ |
| | FTFSSYAMNWVRQAPGRGLEGVSGIS | SLVHSDANTYLNWLHQRPGQPPRLLI |
| | GSGGRAYYAESVKGRFTISRDNSKSTF | YKISNRFSGVPDRFSGSGSGTDFTLKI |
| | YLQMNSLRAEDTAVYYCAKDLDVW | SRVEAEDVGVYYCMQATQFPLTFGG |
| | GKGTTVTVSS | GTKLEIK |
| CB21-498-138 | EVQLVESGGGLVQPGGSLRLSCAASG | DVVMTQTPLSSPVTLGQSASISCRSSQ |
| | FTFSDYAMNWVRQAPGKGLEGVSGIS | SLVHSDANTYLNWLHQRPGQPPRLLI |
| | GSGGRAYYAESVKGRFTISRDNSKSTF | YKISNRFSGVPDRFSGSGSGTDFTLKI |
| | YLQMNSLRAEDTAVYYCAKDLDVW | SRVEAEDVGVYYCMQATQFPLTFGG |
| | GKGTTVTVSS | GTKLEIK |
| CB21-498-139 | EVQLVESGGGLVQPGGSLRLSCAASG | DVVMTQTPLSSPVTLGQSASISCRSSQ |
| | FTFSDYAMNWVRQAPGRGLEGVSGIS | SLVHSDANTYLNWLHQRPGQPPRLLI |
| | GSGGRIYYAESVKGRFTISRDNSKSTF | YKISNRFSGVPDRFSGSGSGTDFTLKI |
| | YLQMNSLRAEDTAVYYCAKDLDVW | SRVEAEDVGVYYCMQATQFPLTFGG |
| | GKGTTVTVSS | GTKLEIK |
| CB21-498-140 | EVQLVESGGGLVQPGGSLRLSCAASG | DVVMTQTPLSSPVTLGQSASISCRSSQ |
| | FTFSDYAMNWVRQAPGRGLEGVSGIS | SLVHSDANTYLNWLHQRPGQPPRLLI |
| | GSGGRAYYAESVKGRFTISRDNSKSTF | YKISNRFSGVPDRFSGSGSGTDFTLKI |
| | YLQMNSLRAEDTAVYYCAKDLDVW | SRVEAEDVGVYYCMQATQFPLTFGG |
| | GKGTTVTVSS | GTKLEIK |
| CB21-498-141 | EVQLVESGGGLVQPGGSLRLSCAASG | DVVMTQTPLSSPVTLGQSASISCRSSQ |
| | FTFSSYAMNWVRQAPGRGLEGVSGIS | SLVHSDANTYLNWLHQRPGQPPRLLI |
| | GSGGRIYYAESVKGRFTISRDNSKSTF | YKISNRFSGVPDRFSGSGSGTDFTLKI |
| | YLQMNSLRAEDTAVYYCAKDLDVW | SRVEAEDVGVYYCMQATQFPLTFGG |
| | GKGTTVTVSS | GTKLEIK |
| CB21-498-142 | EVQLVESGGGLVQPGGSLRLSCAASG | DVVMTQTPLSSPVTLGQSASISCRSSQ |
| | FTFSSYAMNWVRQAPGKGLEGVSGIS | SLVHSDANTYLNWLHQRPGQPPRLLI |
| | GSGGRIYYAESVKGRFQISRDNSKSTF | YKISNRFSGVPDRFSGSGSGTDFTLKI |
| | YLQMNSLRAEDTAVYYCAKDLDVW | SRVEAEDVGVYYCMQATQFPLTFGG |
| | GKGTTVTVSS | GTKLEIK |
| CB21-498-143 | EVQLVESGGGLVQPGGSLRLSCAASG | DVVMTQTPLSSPVTLGQPASISCRSSQ |
| | FTFSSYAMNWVRQAPGRGLEGVSGIS | SLVHSDGNTYLNWLQQRPGQPPRLLI |
| | GSGGRIYYAESVKGRFQISRDNSKSTF | YKISNRFSGVPDRFSGSGAGTDFTLKI |
| | YLQMNSLRAEDTAVYYCAKDLDVW | SRVEAEDVGVYYCMQATQFPLTFGG |
| | GKGTTVTVSS | GTKVEIK |
| CB21-499 | EVQLVESGGGVVQPGRSLRLSCAASG | DIQMTQSPSSLSASVGDRVTITCRASQ |
| | FTFSSYGMHWVRQAPGKGLEWVAVI | GVRNDFGWFQQKPGKAPKRLIYGAS |
| | WYDGSNKDYADSVKGRFTISRDNSK | SLQSGVPSRFSGSGSGTEFTLTISSLQP |
| | NTLYLQMNSLRAEDTAIYYCARPGEG | EDFATYYCLQQNSYPWTFGQGTKLEI |
| | DYWGQGTLVTVSS | K |
| CB21-499-001 | EVQLVESGGGVVQPGRSLRLSCAASG | DIQMTQSPSSLSASVGDRVTITCRASQ |
| | FTFSSYGMHWVRQAPGKGLEWVAVI | GVRNDFGWFQQKPGKAPKRLIYGAS |
| | WYDASNKDYADSVKGRFTISRDNSK | SLQSGVPSRFSGSGSGTEFTLTISSLQP |
| | NTLYLQMNSLRAEDTAIYYCARPGEG | EDFATYYCLQQNSYPWTFGQGTKLEI |
| | DYWGQGTLVTVSS | K |
| CB21-499-002 | EVQLVESGGGVVQPGRSLRLSCAASG | DIQMTQSPSSLSASVGDRVTITCRASQ |
| | FTFSSYGMHWVRQAPGKGLEWVAVI | GVRNDFGWFQQKPGKAPKRLIYGAS |
| | WYDPSNKDYADSVKGRFTISRDNSKN | SLQSGVPSRFSGSGSGTEFTLTISSLQP |
| | TLYLQMNSLRAEDTAIYYCARPGEGD | EDFATYYCLQQNSYPWTFGQGTKLEI |
| | YWGQGTLVTVSS | K |
| CB21-499-003 | EVQLVESGGGVVQPGRSLRLSCAASG | DIQMTQSPSSLSASVGDRVTITCRASQ |
| | FTFSSYGMHWVRQAPGKGLEWVAVI | GVRNDFGWFQQKPGKAPKRLIYGAS |
| | WYEGSNKDYADSVKGRFTISRDNSK | SLQSGVPSRFSGSGSGTEFTLTISSLQP |
| | NTLYLQMNSLRAEDTAIYYCARPGEG | EDFATYYCLQQNSYPWTFGQGTKLEI |
| | DYWGQGTLVTVSS | K |
| CB21-499-004 | EVQLVESGGGVVQPGRSLRLSCAASG | DIQMTQSPSSLSASVGDRVTITCRASQ |
| | FTFSSYGMHWVRQAPGKGLEWVAVI | GVRNDFGWFQQKPGKAPKRLIYGAS |
| | WYSGSNKDYADSVKGRFTISRDNSKN | SLQSGVPSRFSGSGSGTEFTLTISSLQP |
| | TLYLQMNSLRAEDTAIYYCARPGEGD | EDFATYYCLQQNSYPWTFGQGTKLEI |
| | YWGQGTLVTVSS | K |
| CB21-499-005 | EVQLVESGGGVVQPGRSLRLSCAASG | DIQMTQSPSSLSASVGDRVTITCRASQ |
| | FTFSSYGMHWVRQAPGKGLEWVAVI | GVRNDFGWFQQKPGKAPKRLIYGAS |
| | WYDGSNKDYAPSVKGRFTISRDNSKN | SLQSGVPSRFSGSGSGTEFTLTISSLQP |
| | TLYLQMNSLRAEDTAIYYCARPGEGD | EDFATYYCLQQNSYPWTFGQGTKLEI |
| | YWGQGTLVTVSS | K |
| CB21-499-006 | EVQLVESGGGVVQPGRSLRLSCAASG | DIQMTQSPSSLSASVGDRVTITCRASQ |
| | FTFSSYGMHWVRQAPGKGLEWVAVI | GVRNDFGWFQQKPGKAPKRLIYGAS |
| | WYDGSNKDYAQSVKGRFTISRDNSK | SLQSGVPSRFSGSGSGTEFTLTISSLQP |
| | NTLYLQMNSLRAEDTAIYYCARPGEG | EDFATYYCLQQNSYPWTFGQGTKLEI |
| | DYWGQGTLVTVSS | K |
| CB21-499-007 | EVQLVESGGGVVQPGRSLRLSCAASG | DIQMTQSPSSLSASVGDRVTITCRASQ |
| | FTFSSYGMHWVRQAPGKGLEWVAVI | GVRNDFGWFQQKPGKAPKRLIYGAS |
| | WYDGSNKDYAESVKGRFTISRDNSK | SLQSGVPSRFSGSGSGTEFTLTISSLQP |
| | NTLYLQMNSLRAEDTAIYYCARPGEG | EDFATYYCLQQNSYPWTFGQGTKLEI |
| | DYWGQGTLVTVSS | K |
| CB21-499-008 | EVQLVESGGGVVQPGRSLRLSCAASG | DIQMTQSPSSLSASVGDRVTITCRASQ |
| | FTFSSYGMHWVRQAPGKGLEWVAVI | GVRNDFGWFQQKPGKAPKRLIYGAS |
| | WYDGSNKDYADAVKGRFTISRDNSK | SLQSGVPSRFSGSGSGTEFTLTISSLQP |
| | NTLYLQMNSLRAEDTAIYYCARPGEG | EDFATYYCLQQNSYPWTFGQGTKLEI |
| | DYWGQGTLVTVSS | K |
| CB21-499-009 | EVQLVESGGGVVQPGRSLRLSCAASG | DIQMTQSPSSLSASVGDRVTITCRASQ |
| | FTFSSYGMHWVRQAPGKGLEWVAVI | GVRNDFGWFQQKPGKAPKRLIYGAS |
| | WYDGSNKDYADSVKGRFTISRDNSK | SLQSGVPSRFSGSGSGTEFTLTISSLQP |
| | NTLYLQMNSLRAEDTAIYYCARDGEG | EDFATYYCLQQNSYPWTFGQGTKLEI |
| | DYWGQGTLVTVSS | K |
| CB21-499-010 | EVQLVESGGGVVQPGGSLRLSCAASG | DIQMTQSPSSLSASVGDRVTITCRASQ |
| | FTFSSYGMHWVRQAPGKGLEWVAVI | GVRNDFGWFQQKPGKAPKRLIYGAS |
| | WYDGSNKDYADSVKGRFTISRDNSK | SLQSGVPSRFSGSGSGTEFTLTISSLQP |
| | NTLYLQMNSLRAEDTAIYYCARPGEG | EDFATYYCLQQNSYPWTFGQGTKLEI |
| | DYWGQGTLVTVSS | K |
| CB21-499-011 | EVQLVESGGGVVQPGRSLRLSCAASG | DIQMTQSPSSLSASVGDRVTITCRASQ |
| | FTFSSYGMHWVRQAPGKGLEWVAVI | GVRNDFGWFQQKPGKAPKRLIYGAS |
| | WYDGSNKYYADSVKGRFTISRDNSK | SLQSGVPSRFSGSGSGTEFTLTISSLQP |
| | NTLYLQMNSLRAEDTAIYYCARPGEG | EDFATYYCLQQNSYPWTFGQGTKLEI |
| | DYWGQGTLVTVSS | K |
| CB21-499-012 | EVQLVESGGGVVQPGRSLRLSCAASG | DIQMTQSPSSLSASVGDRVTITCRASQ |
| | FTFSSYGMHWVRQAPGKGLEWVAVI | GVRNDFGWFQQKPGKAPKRLIYGAS |
| | WYDGSSKDYADSVKGRFTISRDNSKN | SLQSGVPSRFSGSGSGTEFTLTISSLQP |
| | TLYLQMNSLRAEDTAIYYCARPGEGD | EDFATYYCLQQNSYPWTFGQGTKLEI |
| | YWGQGTLVTVSS | K |
| CB21-499-013 | EVQLVESGGGVVQPGRSLRLSCAASG | DIQMTQSPSSLSASVGDRVTITCRASQ |
| | FTFSSYGMHWVRQAPGKGLEWVAVI | GVRNDFGWFQQKPGKAPKRLIYGAS |
| | WYDGSNKDYADSVKGRFTISRDNSK | SLQSGVPSRFSGSGSGTEFTLTISSLQP |
| | NTLYLQMNSLRAEDTAIYYCARPGYG | EDFATYYCLQQNSYPWTFGQGTKLEI |
| | DYWGQGTLVTVSS | K |
| CB21-499-014 | EVQLVESGGGVVQPGRSLRLSCAASG | DIQMTQSPSSLSASVGDRVTITCRASQ |
| | FTFSSYGMHWVRQAPGKGLEWVAVI | GVRNDFGWFQQKPGKAPKRLIYGAS |
| | WYDGSNKDYADSVKGRFTISRDNSK | SLQSGVPSRFSGSGSGTEFTLTISSLQP |
| | NTLYLQMNSLRAEDTAIYYCARPGEG | EDFATYYCLQQNSYPWTFGQGTKLEI |
| | DYWGQGTKVTVSS | K |
| CB21-499-015 | EVQLVESGGGVVQPGRSLRLSCAASG | DIQMTQSPSSLSASVGDRVTITCRASQ |
| | FTFSSYGMHWVRQAPGKGLEWVAVI | GVRNDFGWFQQKPGKAPKRLIYGAS |
| | WYDGSNKDYADSVKGRFTISRDNSK | SLQSGVPSRFSGSGSGTEFTLTISSLQP |
| | NTLYLQMNSLRAEDTAVYYCARPGE | EDFATYYCLQQNSYPWTFGQGTKLEI |
| | GDYWGQGTLVTVSS | K |
| CB21-499-016 | EVQLVESGGGVVQPGRSLRLSCAASG | DIQMTQSPSSLSASVGDRVTITCRASQ |
| | FTFSSYGMHWVRQAPGKGLEWVAVI | GVRNDFGWFQQKPGKAPKRLIYGAS |
| | WYDGSNKDYADSVKGRFTISRDNSK | SLQSGVPSRFSGSGSGTEFTLTISSLQP |
| | NTLYLQMNSLRAEDTAIYYCARPGEF | EDFATYYCLQQNSYPWTFGQGTKLEI |
| | DYWGQGTLVTVSS | K |
| CB21-499-017 | EVQLVESGGGVVQPGRSLRLSCAASG | DIQMTQSPSSLSASVGDRVTITCRASQ |
| | FTFSSYGMHWVRQAPGKGLEWVAVI | GVRNDFGWFQQKPGKAPKLLIYGAS |
| | WYDGSNKDYADSVKGRFTISRDNSK | SLQSGVPSRFSGSGSGTEFTLTISSLQP |
| | NTLYLQMNSLRAEDTAIYYCARPGEG | EDFATYYCLQQNSYPWTFGQGTKLEI |
| | DYWGQGTLVTVSS | K |
| CB21-499-018 | EVQLVESGGGVVQPGRSLRLSCAASG | DIQMTQSPSSLSASVGDRVTITCRASQ |
| | FTFSSYGMHWVRQAPGKGLEWVAVI | GVRNDLGWFQQKPGKAPKRLIYGAS |
| | WYDGSNKDYADSVKGRFTISRDNSK | SLQSGVPSRFSGSGSGTEFTLTISSLQP |
| | NTLYLQMNSLRAEDTAIYYCARPGEG | EDFATYYCLQQNSYPWTFGQGTKLEI |
| | DYWGQGTLVTVSS | K |
| CB21-499-019 | EVQLVESGGGVVQPGRSLRLSCAASG | DIQMTQSPSSLSASVGDRVTITCRASQ |
| | FTFSSYGMHWVRQAPGKGLEWVAVI | GVRNDFGWFQQKPGKAPKRLIYGAS |
| | WYDGSNKDYADSVKGRFTISRDNSK | SLQSGVPSRFSGSGSGTEFTLTISSLQP |
| | NTLYLQMNSLRAEDTAIYYCARPGEG | EDFATYYCQQQNSYPWTFGQGTKLEI |
| | DYWGQGTLVTVSS | K |
| CB21-499-020 | EVQLVESGGGVVQPGRSLRLSCAASG | DIQMTQSPSSLSASVGDRVTITCRASQ |
| | FTFSSYGMHWVRQAPGKGLEWVAVI | GVSNDFGWFQQKPGKAPKRLIYGASS |
| | WYDGSNKDYADSVKGRFTISRDNSK | LQSGVPSRFSGSGSGTEFTLTISSLQPE |
| | NTLYLQMNSLRAEDTAIYYCARPGEG | DFATYYCLQQNSYPWTFGQGTKLEIK |
| | DYWGQGTLVTVSS | |
| CB21-499-021 | EVQLVESGGGVVQPGRSLRLSCAASG | DIQMTQSPSSLSASVGDRVTITCRASQ |
| | FTFSSYGMHWVRQAPGKGLEWVAVI | GVRNDFGWFQQKPGKAPKRLIYGAS |
| | WYDGSNKDYADSVKGRFTISRDNSK | SLQSGVPSRFSGSGSGTDFTLTISSLQP |
| | NTLYLQMNSLRAEDTAIYYCARPGEG | EDFATYYCLQQNSYPWTFGQGTKLEI |
| | DYWGQGTLVTVSS | K |
| CB21-499-022 | EVQLVESGGGVVQPGRSLRLSCAASG | DIQMTQSPSSLSASVGDRVTITCRASQ |
| | FTFSSYGMHWVRQAPGKGLEWVAVI | GVRNDFGWFQQKPGKAPKRLIYGAS |
| | WYDGSNKDYADSVKGRFTISRDNSK | SLQSGVPSRFSGSGSGTEFTLTISSLQP |
| | NTLYLQMNSLRAEDTAIYYCARPGEG | EDFATYYCLQYNSYPWTFGQGTKLEI |
| | DYWGQGTLVTVSS | K |
| CB21-499-023 | EVQLVESGGGVVQPGRSLRLSCAASG | DIQMTQSPSSLSASVGDRVTITCRASQ |
| | FTFSSYGMHWVRQAPGKGLEWVAVI | GVRNDFGWFQQKPGKAPKRLIYGAS |
| | WYDGSNKDYADSVKGRFTISRDNSK | SLQSGVPSRFSGSGSGTEFTLTISSLQP |
| | NTLYLQMNSLRAEDTAIYYCARPGEG | EDFATYYCLQQNSYPFTFGQGTKLEIK |
| | DYWGQGTLVTVSS | |
| CB21-499-024 | EVQLVESGGGVVQPGRSLRLSCAASG | DIQMTQSPSSLSASVGDRVTITCRASQ |
| | FTFSSYGMHWVRQAPGKGLEWVAVI | GVRNDFGWFQQKPGKAPKRLIYGAS |
| | WYDASNKDYAESVKGRFTISRDNSK | SLQSGVPSRFSGSGSGTEFTLTISSLQP |
| | NTLYLQMNSLRAEDTAIYYCARPGEG | EDFATYYCLQQNSYPWTFGQGTKLEI |
| | DYWGQGTLVTVSS | K |
| CB21-499-025 | EVQLVESGGGVVQPGRSLRLSCAASG | DIQMTQSPSSLSASVGDRVTITCRASQ |
| | FTFSSYGMHWVRQAPGKGLEWVAVI | GVRNDFGWFQQKPGKAPKRLIYGAS |
| | WYDASNKDYAPSVKGRFTISRDNSKN | SLQSGVPSRFSGSGSGTEFTLTISSLQP |
| | TLYLQMNSLRAEDTAIYYCARPGEGD | EDFATYYCLQQNSYPWTFGQGTKLEI |
| | YWGQGTLVTVSS | K |
| CB21-499-026 | EVQLVESGGGVVQPGRSLRLSCKASG | DIQMTQSPSSLSASVGDRVTITCRASQ |
| | FTFSSYGMHWVRQAPGKGLEWVAVI | GVRNDFGWFQQKPGKAPKRLIYGAS |
| | WYDGSNKDYADSVKGRFTISRDNSK | SLQSGVPSRFSGSGSGTEFTLTISSLQP |
| | NTLYLQMNSLRAEDTAIYYCARPGEG | EDFATYYCLQQNSYPWTFGQGTKLEI |
| | DYWGQGTLVTVSS | K |
| CB21-499-027 | EVQLVESGGGVVQPGRSLRLSCNASG | DIQMTQSPSSLSASVGDRVTITCRASQ |
| | FTFSSYGMHWVRQAPGKGLEWVAVI | GVRNDFGWFQQKPGKAPKRLIYGAS |
| | WYDGSNKDYADSVKGRFTISRDNSK | SLQSGVPSRFSGSGSGTEFTLTISSLQP |
| | NTLYLQMNSLRAEDTAIYYCARPGEG | EDFATYYCLQQNSYPWTFGQGTKLEI |
| | DYWGQGTLVTVSS | K |
| CB21-499-028 | EVQLVESGGGVVQPGRSLRLSCQASG | DIQMTQSPSSLSASVGDRVTITCRASQ |
| | FTFSSYGMHWVRQAPGKGLEWVAVI | GVRNDFGWFQQKPGKAPKRLIYGAS |
| | WYDGSNKDYADSVKGRFTISRDNSK | SLQSGVPSRFSGSGSGTEFTLTISSLQP |
| | NTLYLQMNSLRAEDTAIYYCARPGEG | EDFATYYCLQQNSYPWTFGQGTKLEI |
| | DYWGQGTLVTVSS | K |
| CB21-499-029 | EVQLVESGGGVVQPGRSLRLSCATSG | DIQMTQSPSSLSASVGDRVTITCRASQ |
| | FTFSSYGMHWVRQAPGKGLEWVAVI | GVRNDFGWFQQKPGKAPKRLIYGAS |
| | WYDGSNKDYADSVKGRFTISRDNSK | SLQSGVPSRFSGSGSGTEFTLTISSLQP |
| | NTLYLQMNSLRAEDTAIYYCARPGEG | EDFATYYCLQQNSYPWTFGQGTKLEI |
| | DYWGQGTLVTVSS | K |
| CB21-499-030 | EVQLVESGGGVVQPGRSLRLSCAVSG | DIQMTQSPSSLSASVGDRVTITCRASQ |
| | FTFSSYGMHWVRQAPGKGLEWVAVI | GVRNDFGWFQQKPGKAPKRLIYGAS |
| | WYDGSNKDYADSVKGRFTISRDNSK | SLQSGVPSRFSGSGSGTEFTLTISSLQP |
| | NTLYLQMNSLRAEDTAIYYCARPGEG | EDFATYYCLQQNSYPWTFGQGTKLEI |
| | DYWGQGTLVTVSS | K |
| CB21-499-031 | EVQLVESGGGVVQPGRSLRLSCAASG | DIQMTQSPSSLSASVGDRVTITCRASQ |
| | FTFTSYGMHWVRQAPGKGLEWVAVI | GVRNDFGWFQQKPGKAPKRLIYGAS |
| | WYDGSNKDYADSVKGRFTISRDNSK | SLQSGVPSRFSGSGSGTEFTLTISSLQP |
| | NTLYLQMNSLRAEDTAIYYCARPGEG | EDFATYYCLQQNSYPWTFGQGTKLEI |
| | DYWGQGTLVTVSS | K |
| CB21-499-032 | EVQLVESGGGVVQPGRSLRLSCAASG | DIQMTQSPSSLSASVGDRVTITCRASQ |
| | FTFNSYGMHWVRQAPGKGLEWVAVI | GVRNDFGWFQQKPGKAPKRLIYGAS |
| | WYDGSNKDYADSVKGRFTISRDNSK | SLQSGVPSRFSGSGSGTEFTLTISSLQP |
| | NTLYLQMNSLRAEDTAIYYCARPGEG | EDFATYYCLQQNSYPWTFGQGTKLEI |
| | DYWGQGTLVTVSS | K |
| CB21-499-033 | EVQLVESGGGVVQPGRSLRLSCAASG | DIQMTQSPSSLSASVGDRVTITCRASQ |
| | FTFQSYGMHWVRQAPGKGLEWVAVI | GVRNDFGWFQQKPGKAPKRLIYGAS |
| | WYDGSNKDYADSVKGRFTISRDNSK | SLQSGVPSRFSGSGSGTEFTLTISSLQP |
| | NTLYLQMNSLRAEDTAIYYCARPGEG | EDFATYYCLQQNSYPWTFGQGTKLEI |
| | DYWGQGTLVTVSS | K |
| CB21-499-034 | EVQLVESGGGVVQPGRSLRLSCAASG | DIQMTQSPSSLSASVGDRVTITCRASQ |
| | FTFWSYGMHWVRQAPGKGLEWVAVI | GVRNDFGWFQQKPGKAPKRLIYGAS |
| | WYDGSNKDYADSVKGRFTISRDNSK | SLQSGVPSRFSGSGSGTEFTLTISSLQP |
| | NTLYLQMNSLRAEDTAIYYCARPGEG | EDFATYYCLQQNSYPWTFGQGTKLEI |
| | DYWGQGTLVTVSS | K |
| CB21-499-035 | EVQLVESGGGVVQPGRSLRLSCAASG | DIQMTQSPSSLSASVGDRVTITCRASQ |
| | FTFSSYAMHWVRQAPGKGLEWVAVI | GVRNDFGWFQQKPGKAPKRLIYGAS |
| | WYDGSNKDYADSVKGRFTISRDNSK | SLQSGVPSRFSGSGSGTEFTLTISSLQP |
| | NTLYLQMNSLRAEDTAIYYCARPGEG | EDFATYYCLQQNSYPWTFGQGTKLEI |
| | DYWGQGTLVTVSS | K |
| CB21-499-036 | EVQLVESGGGVVQPGRSLRLSCAASG | DIQMTQSPSSLSASVGDRVTITCRASQ |
| | FTFSSYTMHWVRQAPGKGLEWVAVI | GVRNDFGWFQQKPGKAPKRLIYGAS |
| | WYDGSNKDYADSVKGRFTISRDNSK | SLQSGVPSRFSGSGSGTEFTLTISSLQP |
| | NTLYLQMNSLRAEDTAIYYCARPGEG | EDFATYYCLQQNSYPWTFGQGTKLEI |
| | DYWGQGTLVTVSS | K |
| CB21-499-037 | EVQLVESGGGVVQPGRSLRLSCAASG | DIQMTQSPSSLSASVGDRVTITCRASQ |
| | FTFSSYNMHWVRQAPGKGLEWVAVI | GVRNDFGWFQQKPGKAPKRLIYGAS |
| | WYDGSNKDYADSVKGRFTISRDNSK | SLQSGVPSRFSGSGSGTEFTLTISSLQP |
| | NTLYLQMNSLRAEDTAIYYCARPGEG | EDFATYYCLQQNSYPWTFGQGTKLEI |
| | DYWGQGTLVTVSS | K |
| CB21-499-038 | EVQLVESGGGVVQPGRSLRLSCAASG | DIQMTQSPSSLSASVGDRVTITCRASQ |
| | FTFSSYQMHWVRQAPGKGLEWVAVI | GVRNDFGWFQQKPGKAPKRLIYGAS |
| | WYDGSNKDYADSVKGRFTISRDNSK | SLQSGVPSRFSGSGSGTEFTLTISSLQP |
| | NTLYLQMNSLRAEDTAIYYCARPGEG | EDFATYYCLQQNSYPWTFGQGTKLEI |
| | DYWGQGTLVTVSS | K |
| CB21-499-039 | EVQLVESGGGVVQPGRSLRLSCAASG | DIQMTQSPSSLSASVGDRVTITCRASQ |
| | FTFSSYEMHWVRQAPGKGLEWVAVI | GVRNDFGWFQQKPGKAPKRLIYGAS |
| | WYDGSNKDYADSVKGRFTISRDNSK | SLQSGVPSRFSGSGSGTEFTLTISSLQP |
| | NTLYLQMNSLRAEDTAIYYCARPGEG | EDFATYYCLQQNSYPWTFGQGTKLEI |
| | DYWGQGTLVTVSS | K |
| CB21-499-040 | EVQLVESGGGVVQPGRSLRLSCAASG | DIQMTQSPSSLSASVGDRVTITCRASQ |
| | FTFSSYRMHWVRQAPGKGLEWVAVI | GVRNDFGWFQQKPGKAPKRLIYGAS |
| | WYDGSNKDYADSVKGRFTISRDNSK | SLQSGVPSRFSGSGSGTEFTLTISSLQP |
| | NTLYLQMNSLRAEDTAIYYCARPGEG | EDFATYYCLQQNSYPWTFGQGTKLEI |
| | DYWGQGTLVTVSS | K |
| CB21-499-041 | EVQLVESGGGVVQPGRSLRLSCAASG | DIQMTQSPSSLSASVGDRVTITCRASQ |
| | FTFSSYWMHWVRQAPGKGLEWVAVI | GVRNDFGWFQQKPGKAPKRLIYGAS |
| | WYDGSNKDYADSVKGRFTISRDNSK | SLQSGVPSRFSGSGSGTEFTLTISSLQP |
| | NTLYLQMNSLRAEDTAIYYCARPGEG | EDFATYYCLQQNSYPWTFGQGTKLEI |
| | DYWGQGTLVTVSS | K |
| CB21-499-042 | EVQLVESGGGVVQPGRSLRLSCAASG | DIQMTQSPSSLSASVGDRVTITCRASQ |
| | FTFSSYPMHWVRQAPGKGLEWVAVI | GVRNDFGWFQQKPGKAPKRLIYGAS |
| | WYDGSNKDYADSVKGRFTISRDNSK | SLQSGVPSRFSGSGSGTEFTLTISSLQP |
| | NTLYLQMNSLRAEDTAIYYCARPGEG | EDFATYYCLQQNSYPWTFGQGTKLEI |
| | DYWGQGTLVTVSS | K |
| CB21-499-043 | EVQLVESGGGVVQPGRSLRLSCAASG | DIQMTQSPSSLSASVGDRVTITCRASQ |
| | FTFSSYGMHWVRQAPGRGLEWVAVI | GVRNDFGWFQQKPGKAPKRLIYGAS |
| | WYDGSNKDYADSVKGRFTISRDNSK | SLQSGVPSRFSGSGSGTEFTLTISSLQP |
| | NTLYLQMNSLRAEDTAIYYCARPGEG | EDFATYYCLQQNSYPWTFGQGTKLEI |
| | DYWGQGTLVTVSS | K |
| CB21-499-044 | EVQLVESGGGVVQPGRSLRLSCAASG | DIQMTQSPSSLSASVGDRVTITCRASQ |
| | FTFSSYGMHWVRQAPGQGLEWVAVI | GVRNDFGWFQQKPGKAPKRLIYGAS |
| | WYDGSNKDYADSVKGRFTISRDNSK | SLQSGVPSRFSGSGSGTEFTLTISSLQP |
| | NTLYLQMNSLRAEDTAIYYCARPGEG | EDFATYYCLQQNSYPWTFGQGTKLEI |
| | DYWGQGTLVTVSS | K |
| CB21-499-045 | EVQLVESGGGVVQPGRSLRLSCAASG | DIQMTQSPSSLSASVGDRVTITCRASQ |
| | FTFSSYGMHWVRQAPGLGLEWVAVI | GVRNDFGWFQQKPGKAPKRLIYGAS |
| | WYDGSNKDYADSVKGRFTISRDNSK | SLQSGVPSRFSGSGSGTEFTLTISSLQP |
| | NTLYLQMNSLRAEDTAIYYCARPGEG | EDFATYYCLQQNSYPWTFGQGTKLEI |
| | DYWGQGTLVTVSS | K |
| CB21-499-046 | EVQLVESGGGVVQPGRSLRLSCAASG | DIQMTQSPSSLSASVGDRVTITCRASQ |
| | FTFSSYGMHWVRQAPGEGLEWVAVI | GVRNDFGWFQQKPGKAPKRLIYGAS |
| | WYDGSNKDYADSVKGRFTISRDNSK | SLQSGVPSRFSGSGSGTEFTLTISSLQP |
| | NTLYLQMNSLRAEDTAIYYCARPGEG | EDFATYYCLQQNSYPWTFGQGTKLEI |
| | DYWGQGTLVTVSS | K |
| CB21-499-047 | EVQLVESGGGVVQPGRSLRLSCAASG | DIQMTQSPSSLSASVGDRVTITCRASQ |
| | FTFSSYGMHWVRQAPGKRLEWVAVI | GVRNDFGWFQQKPGKAPKRLIYGAS |
| | WYDGSNKDYADSVKGRFTISRDNSK | SLQSGVPSRFSGSGSGTEFTLTISSLQP |
| | NTLYLQMNSLRAEDTAIYYCARPGEG | EDFATYYCLQQNSYPWTFGQGTKLEI |
| | DYWGQGTLVTVSS | K |
| CB21-499-048 | EVQLVESGGGVVQPGRSLRLSCAASG | DIQMTQSPSSLSASVGDRVTITCRASQ |
| | FTFSSYGMHWVRQAPGKALEWVAVI | GVRNDFGWFQQKPGKAPKRLIYGAS |
| | WYDGSNKDYADSVKGRFTISRDNSK | SLQSGVPSRFSGSGSGTEFTLTISSLQP |
| | NTLYLQMNSLRAEDTAIYYCARPGEG | EDFATYYCLQQNSYPWTFGQGTKLEI |
| | DYWGQGTLVTVSS | K |
| CB21-499-049 | EVQLVESGGGVVQPGRSLRLSCAASG | DIQMTQSPSSLSASVGDRVTITCRASQ |
| | FTFSSYGMHWVRQAPGKNLEWVAVI | GVRNDFGWFQQKPGKAPKRLIYGAS |
| | WYDGSNKDYADSVKGRFTISRDNSK | SLQSGVPSRFSGSGSGTEFTLTISSLQP |
| | NTLYLQMNSLRAEDTAIYYCARPGEG | EDFATYYCLQQNSYPWTFGQGTKLEI |
| | DYWGQGTLVTVSS | K |
| CB21-499-050 | EVQLVESGGGVVQPGRSLRLSCAASG | DIQMTQSPSSLSASVGDRVTITCRASQ |
| | FTFSSYGMHWVRQAPGKGLEWVAVI | GVRNDFGWFQQKPGKAPKRLIYGAS |
| | WYDGSNTDYADSVKGRFTISRDNSK | SLQSGVPSRFSGSGSGTEFTLTISSLQP |
| | NTLYLQMNSLRAEDTAIYYCARPGEG | EDFATYYCLQQNSYPWTFGQGTKLEI |
| | DYWGQGTLVTVSS | K |
| CB21-499-051 | EVQLVESGGGVVQPGRSLRLSCAASG | DIQMTQSPSSLSASVGDRVTITCRASQ |
| | FTFSSYGMHWVRQAPGKGLEWVAVI | GVRNDFGWFQQKPGKAPKRLIYGAS |
| | WYDGSNIDYADSVKGRFTISRDNSKN | SLQSGVPSRFSGSGSGTEFTLTISSLQP |
| | TLYLQMNSLRAEDTAIYYCARPGEGD | EDFATYYCLQQNSYPWTFGQGTKLEI |
| | YWGQGTLVTVSS | K |
| CB21-499-052 | EVQLVESGGGVVQPGRSLRLSCAASG | DIQMTQSPSSLSASVGDRVTITCRASQ |
| | FTFSSYGMHWVRQAPGKGLEWVAVI | GVRNDFGWFQQKPGKAPKRLIYGAS |
| | WYDGSNNDYADSVKGRFTISRDNSK | SLQSGVPSRFSGSGSGTEFTLTISSLQP |
| | NTLYLQMNSLRAEDTAIYYCARPGEG | EDFATYYCLQQNSYPWTFGQGTKLEI |
| | DYWGQGTLVTVSS | K |
| CB21-499-053 | EVQLVESGGGVVQPGRSLRLSCAASG | DIQMTQSPSSLSASVGDRVTITCRASQ |
| | FTFSSYGMHWVRQAPGKGLEWVAVI | GVRNDFGWFQQKPGKAPKRLIYGAS |
| | WYDGSNRDYADSVKGRFTISRDNSK | SLQSGVPSRFSGSGSGTEFTLTISSLQP |
| | NTLYLQMNSLRAEDTAIYYCARPGEG | EDFATYYCLQQNSYPWTFGQGTKLEI |
| | DYWGQGTLVTVSS | K |
| CB21-499-054 | EVQLVESGGGVVQPGRSLRLSCAASG | DIQMTQSPSSLSASVGDRVTITCRASQ |
| | FTFSSYGMHWVRQAPGKGLEWVAVI | GVRNDFGWFQQKPGKAPKRLIYGAS |
| | WYDGSNKFYADSVKGRFTISRDNSKN | SLQSGVPSRFSGSGSGTEFTLTISSLQP |
| | TLYLQMNSLRAEDTAIYYCARPGEGD | EDFATYYCLQQNSYPWTFGQGTKLEI |
| | YWGQGTLVTVSS | K |
| CB21-499-055 | EVQLVESGGGVVQPGRSLRLSCAASG | DIQMTQSPSSLSASVGDRVTITCRASQ |
| | FTFSSYGMHWVRQAPGKGLEWVAVI | GVRNDFGWFQQKPGKAPKRLIYGAS |
| | WYDGSNKNYADSVKGRFTISRDNSK | SLQSGVPSRFSGSGSGTEFTLTISSLQP |
| | NTLYLQMNSLRAEDTAIYYCARPGEG | EDFATYYCLQQNSYPWTFGQGTKLEI |
| | DYWGQGTLVTVSS | K |
| CB21-499-056 | EVQLVESGGGVVQPGRSLRLSCAASG | DIQMTQSPSSLSASVGDRVTITCRASQ |
| | FTFSSYGMHWVRQAPGKGLEWVAVI | GVRNDFGWFQQKPGKAPKRLIYGAS |
| | WYDGSNKEYADSVKGRFTISRDNSK | SLQSGVPSRFSGSGSGTEFTLTISSLQP |
| | NTLYLQMNSLRAEDTAIYYCARPGEG | EDFATYYCLQQNSYPWTFGQGTKLEI |
| | DYWGQGTLVTVSS | K |
| CB21-499-057 | EVQLVESGGGVVQPGRSLRLSCAASG | DIQMTQSPSSLSASVGDRVTITCRASQ |
| | FTFSSYGMHWVRQAPGKGLEWVAVI | GVRNDFGWFQQKPGKAPKRLIYGAS |
| | WYDGSNKDYADSVKGRFTITRDNSK | SLQSGVPSRFSGSGSGTEFTLTISSLQP |
| | NTLYLQMNSLRAEDTAIYYCARPGEG | EDFATYYCLQQNSYPWTFGQGTKLEI |
| | DYWGQGTLVTVSS | K |
| CB21-499-058 | EVQLVESGGGVVQPGRSLRLSCAASG | DIQMTQSPSSLSASVGDRVTITCRASQ |
| | FTFSSYGMHWVRQAPGKGLEWVAVI | GVRNDFGWFQQKPGKAPKRLIYGAS |
| | WYDGSNKDYADSVKGRFTILRDNSK | SLQSGVPSRFSGSGSGTEFTLTISSLQP |
| | NTLYLQMNSLRAEDTAIYYCARPGEG | EDFATYYCLQQNSYPWTFGQGTKLEI |
| | DYWGQGTLVTVSS | K |
| CB21-499-059 | EVQLVESGGGVVQPGRSLRLSCAASG | DIQMTQSPSSLSASVGDRVTITCRASQ |
| | FTFSSYGMHWVRQAPGKGLEWVAVI | GVRNDFGWFQQKPGKAPKRLIYGAS |
| | WYDGSNKDYADSVKGRFTINRDNSK | SLQSGVPSRFSGSGSGTEFTLTISSLQP |
| | NTLYLQMNSLRAEDTAIYYCARPGEG | EDFATYYCLQQNSYPWTFGQGTKLEI |
| | DYWGQGTLVTVSS | K |
| CB21-499-060 | EVQLVESGGGVVQPGRSLRLSCAASG | DIQMTQSPSSLSASVGDRVTITCRASQ |
| | FTFSSYGMHWVRQAPGKGLEWVAVI | GVRNDFGWFQQKPGKAPKRLIYGAS |
| | WYDGSNKDYADSVKGRFTISRDNSK | SLQSGVPSRFSGSGSGTEFTLTISSLQP |
| | NTLYLQMNNLRAEDTAIYYCARPGEG | EDFATYYCLQQNSYPWTFGQGTKLEI |
| | DYWGQGTLVTVSS | K |
| CB21-499-061 | EVQLVESGGGVVQPGRSLRLSCAASG | DIQMTQSPSSLSASVGDRVTITCRASQ |
| | FTFSSYGMHWVRQAPGKGLEWVAVI | GVRNDFGWFQQKPGKAPKRLIYGAS |
| | WYDGSNKDYADSVKGRFTISRDNSK | SLQSGVPSRFSGSGSGTEFTLTISSLQP |
| | NTLYLQMNILRAEDTAIYYCARPGEG | EDFATYYCLQQNSYPWTFGQGTKLEI |
| | DYWGQGTLVTVSS | K |
| CB21-499-062 | EVQLVESGGGVVQPGRSLRLSCAASG | DIQMTQSPSSLSASVGDRVTITCRASQ |
| | FTFSSYGMHWVRQAPGKGLEWVAVI | GVRNDFGWFQQKPGKAPKRLIYGAS |
| | WYDGSNKDYADSVKGRFTISRDNSK | SLQSGVPSRFSGSGSGTEFTLTISSLQP |
| | NTLYLQMNELRAEDTAIYYCARPGEG | EDFATYYCLQQNSYPWTFGQGTKLEI |
| | DYWGQGTLVTVSS | K |
| CB21-499-063 | EVQLVESGGGVVQPGRSLRLSCAASG | DIQMTQSPSSLSASVGDRVTITCRASQ |
| | FTFSSYGMHWVRQAPGKGLEWVAVI | IVRNDFGWFQQKPGKAPKRLIYGASS |
| | WYDGSNKDYADSVKGRFTISRDNSK | LQSGVPSRFSGSGSGTEFTLTISSLQPE |
| | NTLYLQMNSLRAEDTAIYYCARPGEG | DFATYYCLQQNSYPWTFGQGTKLEIK |
| | DYWGQGTLVTVSS | |
| CB21-499-064 | EVQLVESGGGVVQPGRSLRLSCAASG | DIQMTQSPSSLSASVGDRVTITCRASQ |
| | FTFSSYGMHWVRQAPGKGLEWVAVI | SVRNDFGWFQQKPGKAPKRLIYGASS |
| | WYDGSNKDYADSVKGRFTISRDNSK | LQSGVPSRFSGSGSGTEFTLTISSLQPE |
| | NTLYLQMNSLRAEDTAIYYCARPGEG | DFATYYCLQQNSYPWTFGQGTKLEIK |
| | DYWGQGTLVTVSS | |
| CB21-499-065 | EVQLVESGGGVVQPGRSLRLSCAASG | DIQMTQSPSSLSASVGDRVTITCRASQ |
| | FTFSSYGMHWVRQAPGKGLEWVAVI | GIRNDFGWFQQKPGKAPKRLIYGASS |
| | WYDGSNKDYADSVKGRFTISRDNSK | LQSGVPSRFSGSGSGTEFTLTISSLQPE |
| | NTLYLQMNSLRAEDTAIYYCARPGEG | DFATYYCLQQNSYPWTFGQGTKLEIK |
| | DYWGQGTLVTVSS | |
| CB21-499-066 | EVQLVESGGGVVQPGRSLRLSCAASG | DIQMTQSPSSLSASVGDRVTITCRASQ |
| | FTFSSYGMHWVRQAPGKGLEWVAVI | GVRNEFGWFQQKPGKAPKRLIYGASS |
| | WYDGSNKDYADSVKGRFTISRDNSK | LQSGVPSRFSGSGSGTEFTLTISSLQPE |
| | NTLYLQMNSLRAEDTAIYYCARPGEG | DFATYYCLQQNSYPWTFGQGTKLEIK |
| | DYWGQGTLVTVSS | |
| CB21-499-067 | EVQLVESGGGVVQPGRSLRLSCAASG | DIQMTQSPSSLSASVGDRVTITCRASQ |
| | FTFSSYGMHWVRQAPGKGLEWVAVI | GVRNLFGWFQQKPGKAPKRLIYGASS |
| | WYDGSNKDYADSVKGRFTISRDNSK | LQSGVPSRFSGSGSGTEFTLTISSLQPE |
| | NTLYLQMNSLRAEDTAIYYCARPGEG | DFATYYCLQQNSYPWTFGQGTKLEIK |
| | DYWGQGTLVTVSS | |
| CB21-499-068 | EVQLVESGGGVVQPGRSLRLSCAASG | DIQMTQSPSSLSASVGDRVTITCRASQ |
| | FTFSSYGMHWVRQAPGKGLEWVAVI | GVRNYFGWFQQKPGKAPKRLIYGAS |
| | WYDGSNKDYADSVKGRFTISRDNSK | SLQSGVPSRFSGSGSGTEFTLTISSLQP |
| | NTLYLQMNSLRAEDTAIYYCARPGEG | EDFATYYCLQQNSYPWTFGQGTKLEI |
| | DYWGQGTLVTVSS | K |
| CB21-499-069 | EVQLVESGGGVVQPGRSLRLSCAASG | DIQMTQSPSSLSASVGDRVTITCRASQ |
| | FTFSSYGMHWVRQAPGKGLEWVAVI | GVRNDFGWFQQKPGQAPKRLIYGAS |
| | WYDGSNKDYADSVKGRFTISRDNSK | SLQSGVPSRFSGSGSGTEFTLTISSLQP |
| | NTLYLQMNSLRAEDTAIYYCARPGEG | EDFATYYCLQQNSYPWTFGQGTKLEI |
| | DYWGQGTLVTVSS | K |
| CB21-499-070 | EVQLVESGGGVVQPGRSLRLSCAASG | DIQMTQSPSSLSASVGDRVTITCRASQ |
| | FTFSSYGMHWVRQAPGKGLEWVAVI | GVRNDFGWFQQKPGTAPKRLIYGASS |
| | WYDGSNKDYADSVKGRFTISRDNSK | LQSGVPSRFSGSGSGTEFTLTISSLQPE |
| | NTLYLQMNSLRAEDTAIYYCARPGEG | DFATYYCLQQNSYPWTFGQGTKLEIK |
| | DYWGQGTLVTVSS | |
| CB21-499-071 | EVQLVESGGGVVQPGRSLRLSCAASG | DIQMTQSPSSLSASVGDRVTITCRASQ |
| | FTFSSYGMHWVRQAPGKGLEWVAVI | GVRNDFGWFQQKPGRAPKRLIYGAS |
| | WYDGSNKDYADSVKGRFTISRDNSK | SLQSGVPSRFSGSGSGTEFTLTISSLQP |
| | NTLYLQMNSLRAEDTAIYYCARPGEG | EDFATYYCLQQNSYPWTFGQGTKLEI |
| | DYWGQGTLVTVSS | K |
| CB21-499-072 | EVQLVESGGGVVQPGRSLRLSCAASG | DIQMTQSPSSLSASVGDRVTITCRASQ |
| | FTFSSYGMHWVRQAPGKGLEWVAVI | GVRNDFGWFQQKPGKAPKRLIYGAS |
| | WYDGSNKDYADSVKGRFTISRDNSK | QLQSGVPSRFSGSGSGTEFTLTISSLQP |
| | NTLYLQMNSLRAEDTAIYYCARPGEG | EDFATYYCLQQNSYPWTFGQGTKLEI |
| | DYWGQGTLVTVSS | K |
| CB21-499-073 | EVQLVESGGGVVQPGRSLRLSCAASG | DIQMTQSPSSLSASVGDRVTITCRASQ |
| | FTFSSYGMHWVRQAPGKGLEWVAVI | GVRNDFGWFQQKPGKAPKRLIYGAS |
| | WYDGSNKDYADSVKGRFTISRDNSK | NLQSGVPSRFSGSGSGTEFTLTISSLQP |
| | NTLYLQMNSLRAEDTAIYYCARPGEG | EDFATYYCLQQNSYPWTFGQGTKLEI |
| | DYWGQGTLVTVSS | K |
| CB21-499-074 | EVQLVESGGGVVQPGRSLRLSCAASG | DIQMTQSPSSLSASVGDRVTITCRASQ |
| | FTFSSYGMHWVRQAPGKGLEWVAVI | GVRNDFGWFQQKPGKAPKRLIYGAS |
| | WYDGSNKDYADSVKGRFTISRDNSK | TLQSGVPSRFSGSGSGTEFTLTISSLQP |
| | NTLYLQMNSLRAEDTAIYYCARPGEG | EDFATYYCLQQNSYPWTFGQGTKLEI |
| | DYWGQGTLVTVSS | K |
| CB21-499-075 | EVQLVESGGGVVQPGRSLRLSCAASG | DIQMTQSPSSLSASVGDRVTITCRASQ |
| | FTFSSYGMHWVRQAPGKGLEWVAVI | GVRNDFGWFQQKPGKAPKRLIYGAS |
| | WYDGSNKDYADSVKGRFTISRDNSK | SLQSGVPSRFSGSKSGTEFTLTISSLQP |
| | NTLYLQMNSLRAEDTAIYYCARPGEG | EDFATYYCLQQNSYPWTFGQGTKLEI |
| | DYWGQGTLVTVSS | K |
| CB21-499-076 | EVQLVESGGGVVQPGRSLRLSCAASG | DIQMTQSPSSLSASVGDRVTITCRASQ |
| | FTFSSYGMHWVRQAPGKGLEWVAVI | GVRNDFGWFQQKPGKAPKRLIYGAS |
| | WYDGSNKDYADSVKGRFTISRDNSK | SLQSGVPSRFSGSGSGTEFTLTISRLQP |
| | NTLYLQMNSLRAEDTAIYYCARPGEG | EDFATYYCLQQNSYPWTFGQGTKLEI |
| | DYWGQGTLVTVSS | K |
| CB21-499-077 | EVQLVESGGGVVQPGRSLRLSCAASG | DIQMTQSPSSLSASVGDRVTITCRASQ |
| | FTFSSYGMHWVRQAPGKGLEWVAVI | GVRNDFGWFQQKPGKAPKRLIYGAS |
| | WYDGSNKDYADSVKGRFTISRDNSK | SLQSGVPSRFSGSGSGTEFTLTISLLQP |
| | NTLYLQMNSLRAEDTAIYYCARPGEG | EDFATYYCLQQNSYPWTFGQGTKLEI |
| | DYWGQGTLVTVSS | K |
| CB21-499-078 | EVQLVESGGGVVQPGRSLRLSCAASG | DIQMTQSPSSLSASVGDRVTITCRASQ |
| | FTFSSYGMHWVRQAPGKGLEWVAVI | GVRNDFGWFQQKPGKAPKRLIYGAS |
| | WYDGSNKDYADSVKGRFTISRDNSK | SLQSGVPSRFSGSGSGTEFTLTISTLQP |
| | NTLYLQMNSLRAEDTAIYYCARPGEG | EDFATYYCLQQNSYPWTFGQGTKLEI |
| | DYWGQGTLVTVSS | K |
| CB21-499-079 | EVQLVESGGGVVQPGRSLRLSCAASG | DIQMTQSPSSLSASVGDRVTITCRASQ |
| | FTFSSYGMHWVRQAPGKGLEWVAVI | GVRNDFGWFQQKPGKAPKRLIYGAS |
| | WYDGSNKDYADSVKGRFTISRDNSK | SLQSGVPSRFSGSGSGTEFTLTISSLQP |
| | NTLYLQMNSLRAEDTAIYYCARPGEG | EDFADYYCLQQNSYPWTFGQGTKLEI |
| | DYWGQGTLVTVSS | K |
| CB21-499-080 | EVQLVESGGGVVQPGRSLRLSCAASG | DIQMTQSPSSLSASVGDRVTITCRASQ |
| | FTFSSYGMHWVRQAPGKGLEWVAVI | GVRNDFGWFQQKPGKAPKRLIYGAS |
| | WYDGSNKDYADSVKGRFTISRDNSK | SLQSGVPSRFSGSGSGTEFTLTISSLQP |
| | NTLYLQMNSLRAEDTAIYYCARPGEG | EDFAVYYCLQQNSYPWTFGQGTKLEI |
| | DYWGQGTLVTVSS | K |
| CB21-499-081 | EVQLVESGGGVVQPGRSLRLSCAASG | DIQMTQSPSSLSASVGDRVTITCRASQ |
| | FTFSSYGMHWVRQAPGKGLEWVAVI | GVRNDFGWFQQKPGKAPKRLIYGAS |
| | WYDGSNKDYADSVKGRFTISRDNSK | SLQSGVPSRFSGSGSGTEFTLTISSLQP |
| | NTLYLQMNSLRAEDTAIYYCARPGEG | EDFATYYCLQQNSYPWTFGQGTKVEI |
| | DYWGQGTLVTVSS | K |
| CB21-499-082 | EVQLVESGGGVVQPGRSLRLSCAASG | DIQMTQSPSSLSASVGDRVTITCRASQ |
| | FTFSSYAMHWVRQAPGKGLEWVAVIS | GVRNDFGWFQQKPGKAPKRLIYGAS |
| | YDGSNKYYADSVKGRFTISRDNSKNT | SLQSGVPSRFSGSGSGTEFTLTISSLQP |
| | LYLQMNSLRAEDTAVYYCARPGEGD | EDFATYYCLQQNSYPWTFGQGTKLEI |
| | YWGQGTLVTVSS | K |
| CB21-499-083 | EVQLVESGGGVVQPGRSLRLSCAASG | DIQMTQSPSSLSASVGDRVTITCRASQ |
| | FTFSSYGMHWVRQVPGKGLEWVAVI | GVRNDFGWFQQKPGKAPKRLIYGAS |
| | WYDASNKDYAPSVKGRFTISRDNSKN | SLQSGVPSRFSGSGSGTEFTLTISSLQP |
| | TLYLQMNSLRAEDTAIYYCARPGEGD | EDFATYYCLQQNSYPWTFGQGTKLEI |
| | YWGQGTLVTVSS | K |
| CB21-499-084 | EVQLVESGGGVVQPGRSLRLSCAASG | DIQMTQSPSSLSASVGDRVTITCRASQ |
| | FTFSSYGMHWVRQAPGKGLEWVAVI | GVRNDFGWFQQKPGKAPKRLIYGAS |
| | WYDASNKDYVPSVKGRFTISRDNSK | SLQSGVPSRFSGSGSGTEFTLTISSLQP |
| | NTLYLQMNSLRAEDTAIYYCARPGEG | EDFATYYCLQQNSYPWTFGQGTKLEI |
| | DYWGQGTLVTVSS | K |
| CB21-499-085 | EVQLVESGGGVVQPGRSLRLSCAASG | DIQMTQSPSSLSASVGDRVTITCRASQ |
| | FTFSSYGMHWVRQAPGKGLEWVAVI | GVRNDFGWFQQKPGKAPKRLIYGAS |
| | WYDASNKDYAPSVKGRFTISRDNSKN | SLQSGVPSRFSGSGSGTEFTLTISSLQP |
| | TLYLQMNSLRAEDTAVYYCARPGEG | EDFATYYCLQQNSYPWTFGQGTKLEI |
| | DYWGQGTLVTVSS | K |
| CB21-499-086 | EVQLVESGGGVVQPGRSLRLSCAASG | DIQMTQSPSSLSASVGDRVTITCRASQ |
| | FTFSSYGMHWVRQVPGKGLEWVAVI | GVRNDFGWFQQKPGKAPKRLIYGAS |
| | WYDASNKDYVPSVKGRFTISRDNSK | SLQSGVPSRFSGSGSGTEFTLTISSLQP |
| | NTLYLQMNSLRAEDTAIYYCARPGEG | EDFATYYCLQQNSYPWTFGQGTKLEI |
| | DYWGQGTLVTVSS | K |
| CB21-499-087 | EVQLVESGGGVVQPGRSLRLSCAASG | DIQMTQSPSSLSASVGDRVTITCRASQ |
| | FTFSSYGMHWVRQVPGKGLEWVAVI | GVRNDFGWFQQKPGKAPKRLIYGAS |
| | WYDASNKDYAPSVKGRFTISRDNSKN | SLQSGVPSRFSGSGSGTEFTLTISSLQP |
| | TLYLQMNSLRAEDTAVYYCARPGEG | EDFATYYCLQQNSYPWTFGQGTKLEI |
| | DYWGQGTLVTVSS | K |
| CB21-499-088 | EVQLVESGGGVVQPGRSLRLSCAASG | DIQMTQSPSSLSASVGDRVTITCRASQ |
| | FTFSSYGMHWVRQAPGKGLEWVAVI | GVRNDFGWFQQKPGKAPKRLIYGAS |
| | WYDASNKDYVPSVKGRFTISRDNSK | SLQSGVPSRFSGSGSGTEFTLTISSLQP |
| | NTLYLQMNSLRAEDTAVYYCARPGE | EDFATYYCLQQNSYPWTFGQGTKLEI |
| | GDYWGQGTLVTVSS | K |
| CB21-499-089 | EVQLVESGGGVVQPGRSLRLSCAASG | DIQMTQSPSSLSASVGDRVTITCRASQ |
| | FTFSSYGMHWVRQVPGKGLEWVAVI | GVRNDFGWFQQKPGKAPKRLIYGAS |
| | WYDASNKDYVPSVKGRFTISRDNSK | SLQSGVPSRFSGSGSGTEFTLTISSLQP |
| | NTLYLQMNSLRAEDTAVYYCARPGE | EDFATYYCLQQNSYPWTFGQGTKLEI |
| | GDYWGQGTLVTVSS | K |
| CB21-499-090 | EVQLVESGGGVVQPGRSLRLSCAASG | DIQMTQSPSSLSASVGDRVTITCRASQ |
| | FTFSVYGMHWVRQAPGKGLEWVAVI | GVRNDFGWFQQKPGKAPKRLIYGAS |
| | WYDASNKDYAPSVKGRFTISRDNSKN | SLQSGVPSRFSGSGSGTEFTLTISSLQP |
| | TLYLQMNSLRAEDTAIYYCARPGEGD | EDFATYYCLQQNSYPWTFGQGTKLEI |
| | YWGQGTLVTVSS | K |
| CB21-499-091 | EVQLVESGGGVVQPGRSLRLSCAASG | DIQMTQSPSSLSASVGDRVTITCRASQ |
| | FTFSTYGMHWVRQAPGKGLEWVAVI | GVRNDFGWFQQKPGKAPKRLIYGAS |
| | WYDASNKDYAPSVKGRFTISRDNSKN | SLQSGVPSRFSGSGSGTEFTLTISSLQP |
| | TLYLQMNSLRAEDTAIYYCARPGEGD | EDFATYYCLQQNSYPWTFGQGTKLEI |
| | YWGQGTLVTVSS | K |
| CB21-499-092 | EVQLVESGGGVVQPGRSLRLSCAASG | DIQMTQSPSSLSASVGDRVTITCRASQ |
| | FTFSSYGMNWVRQAPGKGLEWVAVI | GVRNDFGWFQQKPGKAPKRLIYGAS |
| | WYDASNKDYAPSVKGRFTISRDNSKN | SLQSGVPSRFSGSGSGTEFTLTISSLQP |
| | TLYLQMNSLRAEDTAIYYCARPGEGD | EDFATYYCLQQNSYPWTFGQGTKLEI |
| | YWGQGTLVTVSS | K |
| CB21-499-093 | EVQLVESGGGVVQPGRSLRLSCAASG | DIQMTQSPSSLSASVGDRVTITCRASQ |
| | FTFSSYGMHWVRQAPGKGLEWVAII | GVRNDFGWFQQKPGKAPKRLIYGAS |
| | WYDASNKDYAPSVKGRFTISRDNSKN | SLQSGVPSRFSGSGSGTEFTLTISSLQP |
| | TLYLQMNSLRAEDTAIYYCARPGEGD | EDFATYYCLQQNSYPWTFGQGTKLEI |
| | YWGQGTLVTVSS | K |
| CB21-499-094 | EVQLVESGGGVVQPGRSLRLSCAASG | DIQMTQSPSSLSASVGDRVTITCRASQ |
| | FTFSSYGMHWVRQAPGKGLEWVAMI | GVRNDFGWFQQKPGKAPKRLIYGAS |
| | WYDASNKDYAPSVKGRFTISRDNSKN | SLQSGVPSRFSGSGSGTEFTLTISSLQP |
| | TLYLQMNSLRAEDTAIYYCARPGEGD | EDFATYYCLQQNSYPWTFGQGTKLEI |
| | YWGQGTLVTVSS | K |
| CB21-499-095 | EVQLVESGGGVVQPGRSLRLSCAASG | DIQMTQSPSSLSASVGDRVTITCRASQ |
| | FTFSSYGMHWVRQAPGKGLEWVARI | GVRNDFGWFQQKPGKAPKRLIYGAS |
| | WYDASNKDYAPSVKGRFTISRDNSKN | SLQSGVPSRFSGSGSGTEFTLTISSLQP |
| | TLYLQMNSLRAEDTAIYYCARPGEGD | EDFATYYCLQQNSYPWTFGQGTKLEI |
| | YWGQGTLVTVSS | K |
| CB21-499-096 | EVQLVESGGGVVQPGRSLRLSCAASG | DIQMTQSPSSLSASVGDRVTITCRASQ |
| | FTFSSYGMHWVRQAPGKGLEWVADI | GVRNDFGWFQQKPGKAPKRLIYGAS |
| | WYDASNKDYAPSVKGRFTISRDNSKN | SLQSGVPSRFSGSGSGTEFTLTISSLQP |
| | TLYLQMNSLRAEDTAIYYCARPGEGD | EDFATYYCLQQNSYPWTFGQGTKLEI |
| | YWGQGTLVTVSS | K |
| CB21-499-097 | EVQLVESGGGVVQPGRSLRLSCAASG | DIQMTQSPSSLSASVGDRVTITCRASQ |
| | FTFSSYGMHWVRQAPGKGLEWVAEI | GVRNDFGWFQQKPGKAPKRLIYGAS |
| | WYDASNKDYAPSVKGRFTISRDNSKN | SLQSGVPSRFSGSGSGTEFTLTISSLQP |
| | TLYLQMNSLRAEDTAIYYCARPGEGD | EDFATYYCLQQNSYPWTFGQGTKLEI |
| | YWGQGTLVTVSS | K |
| CB21-499-098 | EVQLVESGGGVVQPGRSLRLSCAASG | DIQMTQSPSSLSASVGDRVTITCRASQ |
| | FTFSSYGMHWVRQAPGKGLEWVAAI | GVRNDFGWFQQKPGKAPKRLIYGAS |
| | WYDASNKDYAPSVKGRFTISRDNSKN | SLQSGVPSRFSGSGSGTEFTLTISSLQP |
| | TLYLQMNSLRAEDTAIYYCARPGEGD | EDFATYYCLQQNSYPWTFGQGTKLEI |
| | YWGQGTLVTVSS | K |
| CB21-499-099 | EVQLVESGGGVVQPGRSLRLSCAASG | DIQMTQSPSSLSASVGDRVTITCRASQ |
| | FTFSSYGMHWVRQAPGKGLEWVAVI | GVRNDFGWFQQKPGKAPKRLIYGAS |
| | WYDASNKDYAPSVKGRFTISRDNSKN | SLQSGVPSRFSGSGSGTEFTLTISSLQP |
| | TLYLQMNGLRAEDTAIYYCARPGEGD | EDFATYYCLQQNSYPWTFGQGTKLEI |
| | YWGQGTLVTVSS | K |
| CB21-499-100 | EVQLVESGGGVVQPGRSLRLSCAASG | DIQMTQSPSSLSASVGDRVTITCRASQ |
| | FTFSSYGMHWVRQAPGKGLEWVAVI | GVRNDFGWFQQKPGKAPKRLIYGAS |
| | WYDASNKDYAPSVKGRFTISRDNSKN | SLQSGVPSRFSGSGSGTEFTLTISSLQP |
| | TLYLQMNRLRAEDTAIYYCARPGEGD | EDFATYYCLQQNSYPWTFGQGTKLEI |
| | YWGQGTLVTVSS | K |
| CB21-499-101 | EVQLVESGGGVVQPGRSLRLSCAASG | DIQMTQSPSSLSASVGDRVTITCRASQ |
| | FTFSSYGMHWVRQAPGKGLEWVAVI | GVRNDFGWFQQKPGKAPKRLIYGAS |
| | WYDASNKDYAPSVKGRFTISRDNSKN | SLQSGVPSRFSGSGSGTEFTLTISSLQP |
| | TLYLQMNSLRAEDTAIYYCARPGEGD | EDFATYYCLQQNSYPWTFGQGTKLEI |
| | YWGRGTLVTVSS | K |
| CB21-499-102 | EVQLVESGGGVVQPGRSLRLSCAASG | DIQMTQSPSSLSASVGDRVTITCRASQ |
| | FTFSSYGMHWVRQAPGKGLEWVAVI | GVRNDFGWFQQKPGKAPKRLIYGAS |
| | WYDASNKDYAPSVKGRFTISRDNSKN | SLQSGVPSRFSGSGSGTEFTLTISSLQP |
| | TLYLQMNSLRAEDTAIYYCARPGEGD | EDFATYYCLQQNSYPWTFGQGTKLEI |
| | YWGQGTTVTVSS | K |
| CB21-499-103 | EVQLVESGGGVVQPGRSLRLSCAASG | DIQMTQSPSSLSASVGDRVTITCRASQ |
| | FTFSSYGMHWVRQAPGKGLEWVAVI | GVRNDFGWFQQKPGKAPKRLIYGAS |
| | WYDASNKDYAPSVKGRFTISRDNSKN | SLQSGVPSRFSGSGSGTEFTLTISSLQP |
| | TLYLQMNSLRAEDTAIYYCARPGEGD | EDFATYYCLQQNSYPWTFGQGTKLEI |
| | YWGQGTMVTVSS | K |
| CB21-499-104 | EVQLVESGGGVVQPGRSLRLSCAASG | DIQMTQSPSSLSASVGDRVTITCRASQ |
| | FTFSSYGMHWVRDAPGKGLEWVAVI | GVRNDFGWFQKKPGKAPKRLIYGAS |
| | WYDASNKDYAPSVKGRFTISRDNSKN | SLQSGVPSRFSGSGSGTEFTLTISSLQP |
| | TLYLQMNSLRAEDTAIYYCARPGEGD | EDFATYYCLQQNSYPWTFGQGTKLEI |
| | YWGQGTLVTVSS | K |
| CB21-499-105 | EVQLVESGGGVVQPGRSLRLSCAASG | DIQMTQSPSSLSASVGDRVTITCRASQ |
| | FTFSSYGMHWVRKAPGKGLEWVAVI | GVRNDFGWFQDKPGKAPKRLIYGAS |
| | WYDASNKDYAPSVKGRFTISRDNSKN | SLQSGVPSRFSGSGSGTEFTLTISSLQP |
| | TLYLQMNSLRAEDTAIYYCARPGEGD | EDFATYYCLQQNSYPWTFGQGTKLEI |
| | YWGQGTLVTVSS | K |
| CB21-499-106 | EVQLVESGGGVVQPGRSLRLSCAASG | DIQMTQSPSSLSASVGDRVTITCRASQ |
| | FTFSSYGMHWVRCAPGKGLEWVAVI | GVRNDFGWFQCKPGKAPKRLIYGAS |
| | WYDASNKDYAPSVKGRFTISRDNSKN | SLQSGVPSRFSGSGSGTEFTLTISSLQP |
| | TLYLQMNSLRAEDTAIYYCARPGEGD | EDFATYYCLQQNSYPWTFGQGTKLEI |
| | YWGQGTLVTVSS | K |
| CB21-499-107 | EVQLVESGGGVVQPGRSLRLSCAASG | DIQMTQSPSSLSASVGDRVTITCRASQ |
| | FTFSSYGMHWVRQAPGKGLEWVAVI | GVRNDFGWFQQKPGKKPKRLIYGAS |
| | WYDASNKDYAPSVKGRFTISRDNSKN | SLQSGVPSRFSGSGSGTEFTLTISSLQP |
| | TLYLQMNSLRAEDTAIYYCARPGEGD | EDFATYYCLQQNSYPWTFGQGTKLEI |
| | YWDQGTLVTVSS | K |
| CB21-499-108 | EVQLVESGGGVVQPGRSLRLSCAASG | DIQMTQSPSSLSASVGDRVTITCRASQ |
| | FTFSSYGMHWVRQAPGKGLEWVAVI | GVRNDFGWFQQKPGKDPKRLIYGAS |
| | WYDASNKDYAPSVKGRFTISRDNSKN | SLQSGVPSRFSGSGSGTEFTLTISSLQP |
| | TLYLQMNSLRAEDTAIYYCARPGEGD | EDFATYYCLQQNSYPWTFGQGTKLEI |
| | YWKQGTLVTVSS | K |
| CB21-499-109 | EVQLVESGGGVVQPGRSLRLSCAASG | DIQMTQSPSSLSASVGDRVTITCRASQ |
| | FTFSSYGMHWVRQAPGKGLEWVAVI | GVRNDFGWFQQKPGKCPKRLIYGAS |
| | WYDASNKDYAPSVKGRFTISRDNSKN | SLQSGVPSRFSGSGSGTEFTLTISSLQP |
| | TLYLQMNSLRAEDTAIYYCARPGEGD | EDFATYYCLQQNSYPWTFGQGTKLEI |
| | YWCQGTLVTVSS | K |
| CB21-499-110 | EVQLVESGGGVVQPGRSLRLSCAASG | DIQMTQSPSSLSASVGDRVTITCRASQ |
| | FTFSSYGMHWVRQAPGKGLEWVAVI | GVRNDFGWFQQKPGKAPKRLIYGAS |
| | WYDASNKDYAPSVKGRFTISRDNSKN | SLQSGVPSRFSGSGSGTEFTLTISSLQP |
| | TLYLQMNSLRAEDTAIYYCARPGEGD | EDFATYYCLQKNSYPWTFGQGTKLEI |
| | YWGQGTLVTVSS | K |
| CB21-499-111 | EVQLVESGGGVVQPGRSLRLSCAASG | DIQMTQSPSSLSASVGDRVTITCRASQ |
| | FTFSSYGMHWVRQAPGKGLEWVAVI | GVRNDFGWFQQKPGKAPKRLIYGAS |
| | WYDASNKDYAPSVKGRFTISRDNSKN | SLQSGVPSRFSGSGSGTEFTLTISSLQP |
| | TLYLQMNSLRAEDTAIYYCARPGEGC | EDFATYYCLQCNSYPWTFGQGTKLEI |
| | YWGQGTLVTVSS | K |
| CB21-499-112 | EVQLVESGGGVVQPGRSLRLSCAASG | DIQMTQSPSSLSASVGDRVTITCRASQ |
| | FTFSSYGMHWVRQAPGKGLEWVDVI | GVRNDFGWFQQKPGKAPKRLIYGAS |
| | WYDASNKDYAPSVKGRFTKSRDNSK | SLQSGVPSRFSGSGSGTEFTLTISSLQP |
| | NTLYLQMNSLRAEDTAIYYCARPGEG | EDFATYYCLQQNSYPWTFGQGTKLEI |
| | DYWGQGTLVTVSS | K |
| CB21-499-113 | EVQLVESGGGVVQPGRSLRLSCAASG | DIQMTQSPSSLSASVGDRVTITCRASQ |
| | FTFSSYGMHWVRQAPGKGLEWVKVI | GVRNDFGWFQQKPGKAPKRLIYGAS |
| | WYDASNKDYAPSVKGRFTDSRDNSK | SLQSGVPSRFSGSGSGTEFTLTISSLQP |
| | NTLYLQMNSLRAEDTAIYYCARPGEG | EDFATYYCLQQNSYPWTFGQGTKLEI |
| | DYWGQGTLVTVSS | K |
| CB21-499-114 | EVQLVESGGGVVQPGRSLRLSCAASG | DIQMTQSPSSLSASVGDRVTITCRASQ |
| | FTFSDYGMHWVRQAPGKGLEWVAVI | GVRNDFGWFQQKPGKAPKRLIYGAS |
| | WYDASNKDYAPSVKGRFTISRDNSKN | SLQSGVPSRFSGSGSGTEFTLTISSLQP |
| | TLYLQMNSLRAEDTAIYYCARPGEGD | EDFATYYCLQQNSYPWTFGQGTKLEI |
| | YWGQGTLVTVSS | K |
| CB21-499-115 | EVQLVESGGGVVQPGRSLRLSCAASG | DIQMTQSPSSLSASVGDRVTITCRASQ |
| | FTFSSDGMHWVRQAPGKGLEWVAVI | GVRNDFGWFQQKPGKAPKRLIYGAS |
| | WYDASNKDYAPSVKGRFTISRDNSKN | SLQSGVPSRFSGSGSGTEFTLTISSLQP |
| | TLYLQMNSLRAEDTAIYYCARPGEGD | EDFATYYCLQQNSYPWTFGQGTKLEI |
| | YWGQGTLVTVSS | K |
| CB21-499-116 | EVQLVESGGGVVQPGRSLKLSCAASG | DIQMTQSPSSLSASVGDRVTITCRASQ |
| | FTFSSYGMHWVRQAPGKGLEWVAVI | GVRNDFGWFQQKPGKAPKRLIYGAS |
| | WYDASNKDYAPSVKGRFTISRDNSKN | SLQSGVPSRFSGSGSGTEFTLTISSLQP |
| | TLYLQMNSLRAEDTAIYYCARPGEGD | EDFATYYCLQQNSYPWTFGQGTKLEI |
| | YWGQGTLVTVSS | K |
| CB21-499-117 | EVQLVESGGGVVQPGRSLRLSCVASG | DIQMTQSPSSLSASVGDRVTITCRASQ |
| | FTFSSYGMHWVRQAPGKGLEWVAVI | GVRNDFGWFQQKPGKAPKRLIYGAS |
| | WYDASNKDYAPSVKGRFTISRDNSKN | SLQSGVPSRFSGSGSGTEFTLTISSLQP |
| | TLYLQMNSLRAEDTAIYYCARPGEGD | EDFATYYCLQQNSYPWTFGQGTKLEI |
| | YWGQGTLVTVSS | K |
| CB21-499-118 | EVQLVESGGGVVQPGRSLKLSCVASG | DIQMTQSPSSLSASVGDRVTITCRASQ |
| | FTFSSYGMHWVRQAPGKGLEWVAVI | GVRNDFGWFQQKPGKAPKRLIYGAS |
| | WYDASNKDYAPSVKGRFTISRDNSKN | SLQSGVPSRFSGSGSGTEFTLTISSLQP |
| | TLYLQMNSLRAEDTAIYYCARPGEGD | EDFATYYCLQQNSYPWTFGQGTKLEI |
| | YWGQGTLVTVSS | K |
| CB21-499-119 | EVQLVESGGGVVQPGRSLRLSCAASG | DIQMTQSPSSLSASVGDRVTITCRASQ |
| | FTFSSYGMHWVRQAPGKGLEWVCVI | GVRNDFGWFQQKPGKAPKRLIYGAS |
| | WYDASNKDYAPSVKGRFTCSRDNSK | SLQSGVPSRFSGSGSGTEFTLTISSLQP |
| | NTLYLQMNSLRAEDTAIYYCARPGEG | EDFATYYCLQQNSYPWTFGQGTKLEI |
| | DYWGQGTLVTVSS | K |
| CB21-499-120 | EVQLVESGGGVVQPGRSLKLSCVASG | DIQMTQSPSSLSASVGDRVTITCRASQ |
| | FTFSSYGMHWVRQAPGKGLEWVCVI | GVRNDFGWFQQKPGKAPKRLIYGAS |
| | WYDASNKDYAPSVKGRFTCSRDNSK | SLQSGVPSRFSGSGSGTEFTLTISSLQP |
| | NTLYLQMNSLRAEDTAIYYCARPGEG | EDFATYYCLQQNSYPWTFGQGTKLEI |
| | DYWGQGTLVTVSS | K |
| CB21-499-121 | EVQLVESGGGVVQPGRSLRLSCAASG | DIQMTQSPSSLSASVGDRVTITCRASQ |
| | FTFSSYGMHWVRQAPGKGLEWVAVI | GVRNDFGWFQQKPGKAPKRLIYGAS |
| | WYDASNKDYAPSVKGCFTISRDNSKN | SLQSGVPSRFSGSGSGTEFTLTISSLQP |
| | TLYLQMNSLRAECTAIYYCARPGEGD | EDFATYYCLQQNSYPWTFGQGTKLEI |
| | YWGQGTLVTVSS | K |
| CB21-499-122 | EVQLVESGGGVVQPGRSLRLSCAASG | DIQMTQSPSSLSASVGDRVTITCRASQ |
| | FTFSSYGMHWVRQAPGKGLEWVAVI | GVRNDFGWFQQKPGKAPKRLIYGAS |
| | WYDASNKDYAPSVKGRFTISRDNSKN | SLQSGVPSRFSGSGSGTEFTLTISSLQP |
| | TLYLQMNSLRAEDTAIYYCARPGECD | EDFATYYCLQQNSYPWTFGQGTKLEI |
| | YWGQGTLVTVSS | K |
| CB21-499-123 | EVQLVESGGGVVQPGRSLRLSCAASG | DIQMTQSPSSLSASVGDRVTITCRASQ |
| | FPFSSYGMHWVRQAPGKGLEWVAVI | GVRNDFGWFQQKPGKAPKRLIYGAS |
| | WYDASNKDYAPSVKGRFTISRDNSKN | SLQSGVPSRFSGSGSGTEFTLTISSLQP |
| | TLYLQMNSLRAEDTAIYYCARPGEGD | EDFATYYCLQQNSYPWTFGQGTKLEI |
| | YWGQGTLVTVSS | K |
| CB21-499-124 | EVQLVESGGGVVQPGRSLRLSCAASG | DIQMTQSPSSLSASVGDRVTITCRASQ |
| | FTFPSYGMHWVRQAPGKGLEWVAVI | GVRNDFGWFQQKPGKAPKRLIYGAS |
| | WYDASNKDYAPSVKGRFTISRDNSKN | SLQSGVPSRFSGSGSGTEFTLTISSLQP |
| | TLYLQMNSLRAEDTAIYYCARPGEGD | EDFATYYCLQQNSYPWTFGQGTKLEI |
| | YWGQGTLVTVSS | K |
| CB21-499-125 | EVQLVESGGGVVQPGRSLRLSCAASG | DIQMTQSPSSLSASVGDRVTITCRASQ |
| | FTFSPYGMHWVRQAPGKGLEWVAVI | GVRNDFGWFQQKPGKAPKRLIYGAS |
| | WYDASNKDYAPSVKGRFTISRDNSKN | SLQSGVPSRFSGSGSGTEFTLTISSLQP |
| | TLYLQMNSLRAEDTAIYYCARPGEGD | EDFATYYCLQQNSYPWTFGQGTKLEI |
| | YWGQGTLVTVSS | K |
| CB21-499-126 | EVQLVESGGGVVQPGRSLRLSCAASG | DIQMTQSPSSLSASVGDRVTITCRASQ |
| | FTFSSYPMHWVRQAPGKGLEWVAVI | GVRNDFGWFQQKPGKAPKRLIYGAS |
| | WYDASNKDYAPSVKGRFTISRDNSKN | SLQSGVPSRFSGSGSGTEFTLTISSLQP |
| | TLYLQMNSLRAEDTAIYYCARPGEGD | EDFATYYCLQQNSYPWTFGQGTKLEI |
| | YWGQGTLVTVSS | K |
| CB21-499-127 | EVQLVESGGGVVQPGRSLRLSCAASG | DIQMTQSPSSLSASVGDRVTITCRASQ |
| | FTFSSYGMHWVRQAPGKGLEWVAVI | GVRNDFGWFQQKPGKAPKRLIYGAS |
| | WYDAPNKDYAPSVKGRFTISRDNSKN | SLQSGVPSRFSGSGSGTEFTLTISSLQP |
| | TLYLQMNSLRAEDTAIYYCARPGEGD | EDFATYYCLQQNSYPWTFGQGTKLEI |
| | YWGQGTLVTVSS | K |
| CB21-499-128 | EVQLVESGGGVVQPGRSLRLSCAASG | DIQMTQSPSSLSASVGDRVTITCRASQ |
| | FTFSSYGMHWVRQAPGKGLEWVAVI | GVRNDFGWFQQKPGKAPKRLIYGAS |
| | SYDASNKDYAPSVKGRFTISRDNSKN | SLQSGVPSRFSGSGSGTEFTLTISSLQP |
| | TLYLQMNSLRAEDTAIYYCARPGEGD | EDFATYYCLQQNSYPWTFGQGTKLEI |
| | YWGQGTLVTVSS | K |
| CB21-499-129 | EVQLVESGGGVVQPGRSLRLSCAASG | DIQMTQSPSSLSASVGDRVTITCRASQ |
| | FTFSSYGMHWVRQAPGKGLEWVAVI | GVRNDFGWFQQKPGKAPKRLIYGAS |
| | NYDASNKDYAPSVKGRFTISRDNSKN | SLQSGVPSRFSGSGSGTEFTLTISSLQP |
| | TLYLQMNSLRAEDTAIYYCARPGEGD | EDFATYYCLQQNSYPWTFGQGTKLEI |
| | YWGQGTLVTVSS | K |
| CB21-499-130 | EVQLVESGGGVVQPGRSLRLSCAASG | DIQMTQSPSSLSASVGDRVTITCRASQ |
| | FTFSSYGMHWVRQAPGKGLEWVAVI | GVRNDFGWFQQKPGKAPKRLIYGAS |
| | DYDASNKDYAPSVKGRFTISRDNSKN | SLQSGVPSRFSGSGSGTEFTLTISSLQP |
| | TLYLQMNSLRAEDTAIYYCARPGEGD | EDFATYYCLQQNSYPWTFGQGTKLEI |
| | YWGQGTLVTVSS | K |
| CB21-499-131 | EVQLVESGGGVVQPGRSLRLSCAASG | DIQMTQSPSSLSASVGDRVTITCRASQ |
| | FTFSSYGMHWVRQAPGKGLEWVAVI | GVRNDFGWFQQKPGKAPKRLIYGAS |
| | RYDASNKDYAPSVKGRFTISRDNSKN | SLQSGVPSRFSGSGSGTEFTLTISSLQP |
| | TLYLQMNSLRAEDTAIYYCARPGEGD | EDFATYYCLQQNSYPWTFGQGTKLEI |
| | YWGQGTLVTVSS | K |
| CB21-499-132 | EVQLVESGGGVVQPGRSLRLSCAASG | DIQMTQSPSSLSASVGDRVTITCRASQ |
| | FTFSSYGMHWVRQAPGKGLEWVAVI | GVRNDFGWFQQKPGKAPKRLIYGAS |
| | KYDASNKDYAPSVKGRFTISRDNSKN | SLQSGVPSRFSGSGSGTEFTLTISSLQP |
| | TLYLQMNSLRAEDTAIYYCARPGEGD | EDFATYYCLQQNSYPWTFGQGTKLEI |
| | YWGQGTLVTVSS | K |
| CB21-499-133 | EVQLVESGGGVVQPGRSLRLSCAASG | DIQMTQSPSSLSASVGDRVTITCRASQ |
| | FTFSSYGMHWVRQAPGKGLEWVAVI | GVRNDFGWFQQKPGKAPKRLIYGAS |
| | AYDASNKDYAPSVKGRFTISRDNSKN | SLQSGVPSRFSGSGSGTEFTLTISSLQP |
| | TLYLQMNSLRAEDTAIYYCARPGEGD | EDFATYYCLQQNSYPWTFGQGTKLEI |
| | YWGQGTLVTVSS | K |
| CB21-499-134 | EVQLVESGGGVVQPGRSLRLSCAASG | DIQMTQSPSSLSASVGDRVTITCRASQ |
| | FTFSSYGMHWVRQAPGKGLEWVAVI | GVRNDFGWFQQKPGKAPKRLIYGAS |
| | WDDASNKDYAPSVKGRFTISRDNSKN | SLQSGVPSRFSGSGSGTEFTLTISSLQP |
| | TLYLQMNSLRAEDTAIYYCARPGEGD | EDFATYYCLQQNSYPWTFGQGTKLEI |
| | YWGQGTLVTVSS | K |
| CB21-499-135 | EVQLVESGGGVVQPGRSLRLSCAASG | DIQMTQSPSSLSASVGDRVTITCRASQ |
| | FTFSSYGMHWVRQAPGKGLEWVAVI | GVRNDFGWFQQKPGKAPKRLIYGAS |
| | WPDASNKDYAPSVKGRFTISRDNSKN | SLQSGVPSRFSGSGSGTEFTLTISSLQP |
| | TLYLQMNSLRAEDTAIYYCARPGEGD | EDFATYYCLQQNSYPWTFGQGTKLEI |
| | YWGQGTLVTVSS | K |
| CB21-499-136 | EVQLVESGGGVVQPGRSLRLSCAASG | DIQMTQSPSSLSASVGDRVTITCRASQ |
| | FTFSSYGMHWVRQAPGKGLEWVAVI | GVRNDFGWFQQKPGKAPKRLIYGAS |
| | WNDASNKDYAPSVKGRFTISRDNSKN | SLQSGVPSRFSGSGSGTEFTLTISSLQP |
| | TLYLQMNSLRAEDTAIYYCARPGEGD | EDFATYYCLQQNSYPWTFGQGTKLEI |
| | YWGQGTLVTVSS | K |
| CB21-499-137 | EVQLVESGGGVVQPGRSLRLSCAASG | DIQMTQSPSSLSASVGDRVTITCRASQ |
| | FTFSSYGMHWVRQAPGKGLEWVAVI | GVRNDFGWFQQKPGKAPKRLIYGAS |
| | WKDASNKDYAPSVKGRFTISRDNSKN | SLQSGVPSRFSGSGSGTEFTLTISSLQP |
| | TLYLQMNSLRAEDTAIYYCARPGEGD | EDFATYYCLQQNSYPWTFGQGTKLEI |
| | YWGQGTLVTVSS | K |
| CB21-499-138 | EVQLVESGGGVVQPGRSLRLSCAASG | DIQMTQSPSSLSASVGDRVTITCRASQ |
| | FTFSSYGMHWVRQAPGKGLEWVAVI | GVRNDFGWFQQKPGKAPKRLIYGAS |
| | WGDASNKDYAPSVKGRFTISRDNSKN | SLQSGVPSRFSGSGSGTEFTLTISSLQP |
| | TLYLQMNSLRAEDTAIYYCARPGEGD | EDFATYYCLQQNSYPWTFGQGTKLEI |
| | YWGQGTLVTVSS | K |
| CB21-499-139 | EVQLVESGGGVVQPGRSLRLSCAASG | DIQMTQSPSSLSASVGDRVTITCRASQ |
| | FTFSSYGMHWVRQAPGKGLEWVAVI | GVRNDFGWFQQKPGKAPKRLIYGAS |
| | WSDASNKDYAPSVKGRFTISRDNSKN | SLQSGVPSRFSGSGSGTEFTLTISSLQP |
| | TLYLQMNSLRAEDTAIYYCARPGEGD | EDFATYYCLQQNSYPWTFGQGTKLEI |
| | YWGQGTLVTVSS | K |
| CB21-499-140 | EVQLVESGGGVVQPGRSLRLSCAASG | DIQMTQSPSSLSASVGDRVTITCRASQ |
| | FTFSSYGMHWVRQAPGKGLEWVAVI | GVRNDFGWFQQKPGKAPKRLIYGAS |
| | WYDASNKDYAPSVKGRFTISRDNSKN | SLQSGVPSRFSGSGSGTEFTLTISSLQP |
| | TLYLQMNSLRAEDTAIYYCARPGEGD | EDFATYYCLQQNSYPWTFGQGTKLEI |
| | VWGQGTLVTVSS | K |
| CB21-499-141 | EVQLVESGGGVVQPGRSLRLSCAASG | DIQMTQSPSSLSASVGDRVTITCRASQ |
| | FTFSSYGMHWVRQAPGKGLEWVAVI | GVRNDFGWFQQKPGKAPKRLIYGAS |
| | WYDASNKDYAPSVKGRFTISRDNSKN | SLQSGVPSRFSGSGSGTEFTLTISSLQP |
| | TLYLQMNSLRAEDTAIYYCARPGEGD | EDFATYYCLQQNSYPWTFGQGTKLEI |
| | RWGQGTLVTVSS | K |
| CB21-499-142 | EVQLVESGGGVVQPGRSLRLSCAASG | DIQMTQSPSSLSASVGDRVTITCRASQ |
| | FTFSSYGMHWVRQAPGKGLEWVAVI | GVRNDFGWFQQKPGKAPKRLIYGAS |
| | WYDASNKDYAPSVKGRFTISRDNSKN | SLQSGVPSRFSGSGSGTEFTLTISSLQP |
| | TLYLQMNSLRAEDTAIYYCARPGEGD | EDFATYYCLQQNSYPWTFGQGTKLEI |
| | PWGQGTLVTVSS | K |
| CB21-499-143 | EVQLVESGGGVVQPGRSLRLSCAASG | DIQMTQSPSSLSASVGDRVTITCRASQ |
| | FTFSSYGMHWVRQAPGKGLEWVAVI | GVRNDFGWFQQKPGKAPKRLIYGAS |
| | WYDASNKDYAPSVKGRFTISRDNSKN | SLQSGVPSRFSGSGSGTEFTLTISSLQP |
| | TLYLQMNSLRAEDTAIYYCARPGEGD | EDFATYYCLQQNSYPWTFGQGTKLEI |
| | AWGQGTLVTVSS | K |
| CB21-499-144 | EVQLVESGGGVVQPGRSLRLSCAASG | DIQMTQSPSSLSASVGDRVTITCRASQ |
| | FTFSSYGMHWVRQAPGKGLEWVAVI | GVRNDFGWFQQKPGKAPKRLIYGDS |
| | WYDASNKDYAPSVKGRFTISRDNSKN | SLQSGVPSRFSGSGSGTEFTLTISSLQP |
| | TLYLQMNSLRAEDTAIYYCARPGEGD | EDFATYYCLQQNSYPWTFGQGTKLEI |
| | YWGQGTLVTVSS | K |
| CB21-499-145 | EVQLVESGGGVVQPGRSLRLSCAASG | DIQMTQSPSSLSASVGDRVTITCRASQ |
| | FTFSSYGMHWVRQAPGKGLEWVAVI | GVRNDFGWFQQKPGKAPKRLIYGAD |
| | WYDASNKDYAPSVKGRFTISRDNSKN | SLQSGVPSRFSGSGSGTEFTLTISSLQP |
| | TLYLQMNSLRAEDTAIYYCARPGEGD | EDFATYYCLQQNSYPWTFGQGTKLEI |
| | YWGQGTLVTVSS | K |
| CB21-499-146 | EVQLVESGGGVVQPGRSLRLSCAASG | DIQMTQSPSSLSASVGDRVTITCRASQ |
| | FTFSSYGMHWVRQAPGKGLEWVAVI | GVRNDFGWFQQKPGKAPKRLIYGDD |
| | WYDASNKDYAPSVKGRFTISRDNSKN | SLQSGVPSRFSGSGSGTEFTLTISSLQP |
| | TLYLQMNSLRAEDTAIYYCARPGEGD | EDFATYYCLQQNSYPWTFGQGTKLEI |
| | YWGQGTLVTVSS | K |
| CB21-499-147 | EVQLVESGGGVVQPGRSLRLSCAASG | DIQMTQSPSSLSASVGDRVTITCRASQ |
| | FTFSSYGMHWVRQAPGKGLEWVAVI | GVRNDFGWYQQKPGKAPKRLIYGAS |
| | WYDASNKDYAPSVKGRFTISRDNSKN | SLQSGVPSRFSGSGSGTEFTLTISSLQP |
| | TLYLQMNSLRAEDTAIYYCARPGEGD | EDFATYYCLQQNSYPWTFGQGTKLEI |
| | YWGQGTLVTVSS | K |
| CB21-499-148 | EVQLVESGGGVVQPGRSLRLSCAASG | DIQMTQSPSSLSASVGDRVTITCRASQ |
| | FTFSSYGMHWVRQAPGKGLEWVAVI | GVRNDFGWFQQKPGKAPKRLIYAASS |
| | WYDASNKDYAPSVKGRFTISRDNSKN | LQSGVPSRFSGSGSGTEFTLTISSLQPE |
| | TLYLQMNSLRAEDTAIYYCARPGEGD | DFATYYCLQQNSYPWTFGQGTKLEIK |
| | YWGQGTLVTVSS | |
| CB21-499-149 | EVQLVESGGGVVQPGRSLRLSCAASG | DIQMTQSPSSLSASVGDRVTITCRASQ |
| | FTFSSYGMHWVRQAPGKGLEWVAVI | GVRNDFGWFQQKPGKAPKRLCYGAS |
| | WYDASNKDYAPSVKGRFTISRDNSKN | SLQSGVPSRFSCSGSGTEFTLTISSLQP |
| | TLYLQMNSLRAEDTAIYYCARPGEGD | EDFATYYCLQQNSYPWTFGQGTKLEI |
| | YWGQGTLVTVSS | K |
| CB21-499-150 | EVQLVESGGGVVQPGRSLRLSCAASG | DIQMTQSPSSLSASVGDRVTITCRASQ |
| | FTFSSYGMHWVRQAPGKGLEWVAVI | GVRNDFGWFQQKPGKAPKRLDYGAS |
| | WYDASNKDYAPSVKGRFTISRDNSKN | SLQSGVPSRFSKSGSGTEFTLTISSLQP |
| | TLYLQMNSLRAEDTAIYYCARPGEGD | EDFATYYCLQQNSYPWTFGQGTKLEI |
| | YWGQGTLVTVSS | K |
| CB21-499-151 | EVQLVESGGGVVQPGRSLRLSCAASG | DIQMTQSPSSLSASVGDRVTITCRASQ |
| | FTFSSYGMHWVRQAPGKGLEWVAVI | GVRNDFGWFQQKPGKAPKRLKYGAS |
| | WYDASNKDYAPSVKGRFTISRDNSKN | SLQSGVPSRFSDSGSGTEFTLTISSLQP |
| | TLYLQMNSLRAEDTAIYYCARPGEGD | EDFATYYCLQQNSYPWTFGQGTKLEI |
| | YWGQGTLVTVSS | K |
| CB21-499-152 | EVQLVESGGGVVQPGRSLRLSCAASG | DIQMTQSPSSLSASVGDRVTITCRASQ |
| | FTFSSYGMHWVRQAPGKGLEWVAVI | GVRNDFGWFQQKPGKAPKRLIYGAS |
| | WYDASNKDYAPSVKGRFTISRDNSKN | SLCSGVPSRFSGSGSGTEFTLTISSLQP |
| | TLYLQMNSLRAEDTAIYYCARPGEGD | EDFATYYCLQQNSYPWTFGQGTKLEI |
| | YWGQGTLVTVSS | K |
| CB21-499-153 | EVQLVESGGGVVQPGRSLRLSCAASG | DIQMTQSPSSLSASVGDRVTITCRASQ |
| | FTFSSYGMHWVRQAPGKGLEWVAVI | GVRNDFGWFQQKPGKAPKRLIYDAS |
| | WYDASNKDYAPSVKGRFTISRDNSKN | SLQSGVPSRFSGSGSGTEFTLTISSLQP |
| | TLYLQMNSLRAEDTAIYYCARPGEGD | EDFATYYCLQQNSYPWTFGQGTKLEI |
| | YWGQGTLVTVSS | K |
| CB21-499-154 | EVQLVESGGGVVQPGRSLRLSCAASG | DIQMTQSPSSLSASVGDRVTITCRASQ |
| | FTFSSYGMHWVRQAPGKGLEWVAVI | GVRNDFGWFQQKPGKAPKRLIYEASS |
| | WYDASNKDYAPSVKGRFTISRDNSKN | LQSGVPSRFSGSGSGTEFTLTISSLQPE |
| | TLYLQMNSLRAEDTAIYYCARPGEGD | DFATYYCLQQNSYPWTFGQGTKLEIK |
| | YWGQGTLVTVSS | |
| CB21-499-155 | EVQLVESGGGVVQPGRSLRLSCAASG | DIQMTQSPSSLSASVGDRVTITCRASQ |
| | FTFSSYGMHWVRQAPGKGLEWVAVI | GVRNDFGWFQQKPGKAPKRLIYKAS |
| | WYDASNKDYAPSVKGRFTISRDNSKN | SLQSGVPSRFSGSGSGTEFTLTISSLQP |
| | TLYLQMNSLRAEDTAIYYCARPGEGD | EDFATYYCLQQNSYPWTFGQGTKLEI |
| | YWGQGTLVTVSS | K |
| CB21-499-156 | EVQLVESGGGVVQPGRSLRLSCAASG | DIQMTQSPSSLSASVGDRVTITCRASQ |
| | FTFSSYGMHWVRQAPGKGLEWVAVI | GVRNDFGWFQQKPGKAPKRLIYLASS |
| | WYDASNKDYAPSVKGRFTISRDNSKN | LQSGVPSRFSGSGSGTEFTLTISSLQPE |
| | TLYLQMNSLRAEDTAIYYCARPGEGD | DFATYYCLQQNSYPWTFGQGTKLEIK |
| | YWGQGTLVTVSS | |
| CB21-499-157 | EVQLVESGGGVVQPGRSLRLSCAASG | DIQMTQSPSSLSASVGDRVTITCRASQ |
| | FTFSSYGMHWVRQAPGKGLEWVAVI | GVRNDFGWFQQKPGKAPKRLIYSASS |
| | WYDASNKDYAPSVKGRFTISRDNSKN | LQSGVPSRFSGSGSGTEFTLTISSLQPE |
| | TLYLQMNSLRAEDTAIYYCARPGEGD | DFATYYCLQQNSYPWTFGQGTKLEIK |
| | YWGQGTLVTVSS | |
| CB21-499-158 | EVQLVESGGGVVQPGRSLRLSCAASG | DIQMTQSPSSLSASVGDRVTITCRASQ |
| | FTFSSYGMHWVRQAPGKGLEWVAVI | GVRNDFGWFQQKPGKAPKRLIYGVS |
| | WYDASNKDYAPSVKGRFTISRDNSKN | SLQSGVPSRFSGSGSGTEFTLTISSLQP |
| | TLYLQMNSLRAEDTAIYYCARPGEGD | EDFATYYCLQQNSYPWTFGQGTKLEI |
| | YWGQGTLVTVSS | K |
| CB21-499-159 | EVQLVESGGGVVQPGRSLRLSCAASG | DIQMTQSPSSLSASVGDRVTITCRASQ |
| | FTFSSYGMHWVRQAPGKGLEWVAVI | GVRNDFGWFQQKPGKAPKRLIYGESS |
| | WYDASNKDYAPSVKGRFTISRDNSKN | LQSGVPSRFSGSGSGTEFTLTISSLQPE |
| | TLYLQMNSLRAEDTAIYYCARPGEGD | DFATYYCLQQNSYPWTFGQGTKLEIK |
| | YWGQGTLVTVSS | |
| CB21-499-160 | EVQLVESGGGVVQPGRSLRLSCAASG | DIQMTQSPSSLSASVGDRVTITCRASQ |
| | FTFSSYGMHWVRQAPGKGLEWVAVI | GVRNDFGWFQQKPGKAPKRLIYGPSS |
| | WYDASNKDYAPSVKGRFTISRDNSKN | LQSGVPSRFSGSGSGTEFTLTISSLQPE |
| | TLYLQMNSLRAEDTAIYYCARPGEGD | DFATYYCLQQNSYPWTFGQGTKLEIK |
| | YWGQGTLVTVSS | |
| CB21-499-161 | EVQLVESGGGVVQPGRSLRLSCAASG | DIQMTQSPSSLSASVGDRVTITCRASQ |
| | FTFSSYGMHWVRQAPGKGLEWVAVI | GVRNDFGWFQQKPGKAPKRLIYGAT |
| | WYDASNKDYAPSVKGRFTISRDNSKN | SLQSGVPSRFSGSGSGTEFTLTISSLQP |
| | TLYLQMNSLRAEDTAIYYCARPGEGD | EDFATYYCLQQNSYPWTFGQGTKLEI |
| | YWGQGTLVTVSS | K |
| CB21-499-162 | EVQLVESGGGVVQPGRSLRLSCAASG | DIQMTQSPSSLSASVGDRVTITCRASQ |
| | FTFSSYGMHWVRQAPGKGLEWVAVI | GVRNDFGWFQQKPGKAPKRLIYGAP |
| | WYDASNKDYAPSVKGRFTISRDNSKN | SLQSGVPSRFSGSGSGTEFTLTISSLQP |
| | TLYLQMNSLRAEDTAIYYCARPGEGD | EDFATYYCLQQNSYPWTFGQGTKLEI |
| | YWGQGTLVTVSS | K |
| CB21-499-163 | EVQLVESGGGVVQPGRSLRLSCAASG | DIQMTQSPSSLSASVGDRVTITCRASQ |
| | FTFSSYGMHWVRQAPGKGLEWVAVI | GVRNDFGWFQQKPGKAPKRLIYGAS |
| | WYDASNKDYAPSVKGRFTISRDNSKN | RLQSGVPSRFSGSGSGTEFTLTISSLQP |
| | TLYLQMNSLRAEDTAIYYCARPGEGD | EDFATYYCLQQNSYPWTFGQGTKLEI |
| | YWGQGTLVTVSS | K |
| CB21-499-164 | EVQLVESGGGVVQPGRSLRLSCAASG | DIQMTQSPSSLSASVGDRVTITCRASQ |
| | FTFSSYGMHWVRQAPGKGLEWVAVI | GVRNDFGWFQQKPGKAPKRLIYGAS |
| | WYDASNKDYAPSVKGRFTISRDNSKN | KLQSGVPSRFSGSGSGTEFTLTISSLQP |
| | TLYLQMNSLRAEDTAIYYCARPGEGD | EDFATYYCLQQNSYPWTFGQGTKLEI |
| | YWGQGTLVTVSS | K |
| CB21-499-165 | EVQLVESGGGVVQPGRSLRLSCAASG | DIQMTQSPSSLSASVGDRVTITCRASQ |
| | FTFSSYGMHWVRQAPGKGLEWVAVI | GVRNDFGWFQQKPGKAPKRLIYGAS |
| | WYDASNKDYAPSVKGRFTISRDNSKN | DLQSGVPSRFSGSGSGTEFTLTISSLQP |
| | TLYLQMNSLRAEDTAIYYCARPGEGD | EDFATYYCLQQNSYPWTFGQGTKLEI |
| | YWGQGTLVTVSS | K |
| CB21-499-166 | EVQLVESGGGVVQPGRSLRLSCAASG | DIQMTQSPSSLSASVGDRVTITCRASQ |
| | FTFSSYGMHWVRQAPGKGLEWVAVI | GVRNDFGWFQQKPGKAPKRLIYGASI |
| | WYDASNKDYAPSVKGRFTISRDNSKN | LQSGVPSRFSGSGSGTEFTLTISSLQPE |
| | TLYLQMNSLRAEDTAIYYCARPGEGD | DFATYYCLQQNSYPWTFGQGTKLEIK |
| | YWGQGTLVTVSS | |
| CB21-499-167 | EVQLVESGGGVVQPGRSLRLSCAASG | DIQMTQSPSSLSASVGDRVTITCRASQ |
| | FTFSSYGMHWVRQAPGKGLEWVAVI | GVRNDFGWFQQKPGKAPKRLIYGAS |
| | WYDASNKDYAPSVKGRFTISRDNSKN | LLQSGVPSRFSGSGSGTEFTLTISSLQP |
| | TLYLQMNSLRAEDTAIYYCARPGEGD | EDFATYYCLQQNSYPWTFGQGTKLEI |
| | YWGQGTLVTVSS | K |
| CB21-499-168 | EVQLVESGGGVVQPGRSLRLSCAASG | DIQMTQSPSSLSASVGDRVTITCRASQ |
| | FTFSSYGMHWVRQAPGKGLEWVAVI | GVRNDFGWFQQKPGKAPKRLIYGAS |
| | WYDASNKDYAPSVKGRFTISRDNSKN | SRQSGVPSRFSGSGSGTEFTLTISSLQP |
| | TLYLQMNSLRAEDTAIYYCARPGEGD | EDFATYYCLQQNSYPWTFGQGTKLEI |
| | YWGQGTLVTVSS | K |
| CB21-499-169 | EVQLVESGGGVVQPGRSLRLSCAASG | DIQMTQSPSSLSASVGDRVTITCRASQ |
| | FTFSSYGMHWVRQAPGKGLEWVAVI | GVRNDFGWFQQKPGKAPKRLIYGAS |
| | WYDASNKDYAPSVKGRFTISRDNSKN | SPQSGVPSRFSGSGSGTEFTLTISSLQP |
| | TLYLQMNSLRAEDTAIYYCARPGEGD | EDFATYYCLQQNSYPWTFGQGTKLEI |
| | YWGQGTLVTVSS | K |
| CB21-499-170 | EVQLVESGGGVVQPGRSLRLSCAASG | DIQMTQSPSSLSASVGDRVTITCRASQ |
| | FTFSSYGMHWVRQAPGKGLEWVAVI | GVRNDFGWFQQKPGKAPKRLIYGAS |
| | WYDASNKDYAPSVKGRFTISRDNSKN | SLPSGVPSRFSGSGSGTEFTLTISSLQP |
| | TLYLQMNSLRAEDTAIYYCARPGEGD | EDFATYYCLQQNSYPWTFGQGTKLEI |
| | YWGQGTLVTVSS | K |
| CB21-499-171 | EVQLVESGGGVVQPGRSLRLSCAASG | DIQMTQSPSSLSASVGDRVTITCRASQ |
| | FTFSSYGMHWVRQAPGKGLEWVAVI | GVRNDFGWFQQKPGKAPKRLIYGAS |
| | WYDASNKDYAPSVKGRFTISRDNSKN | SLASGVPSRFSGSGSGTEFTLTISSLQP |
| | TLYLQMNSLRAEDTAIYYCARPGEGD | EDFATYYCLQQNSYPWTFGQGTKLEI |
| | YWGQGTLVTVSS | K |
| CB21-499-172 | EVQLVESGGGVVQPGRSLRLSCAASG | DIQMTQSPSSLSASVGDRVTITCRASQ |
| | FTFSSYGMHWVRQAPGKGLEWVAVI | GVRNDFGWFQQKPGKAPKRLIYGAS |
| | WYDASNKDYAPSVKGRFTISRDNSKN | SLESGVPSRFSGSGSGTEFTLTISSLQP |
| | TLYLQMNSLRAEDTAIYYCARPGEGD | EDFATYYCLQQNSYPWTFGQGTKLEI |
| | YWGQGTLVTVSS | K |
| CB21-499-174 | EVQLVESGGGVVQPGRSLRLSCAASG | DIQMTQSPSSLSASVGDRVTITCRASQ |
| | FTFSSYGMHWVRQAPGKGLEWVAVI | GIRNDLGWYQQKPGKAPKRLIYAASS |
| | WYDASNKDYAPSVKGRFTISRDNSKN | LQSGVPSRFSGSGSGTEFTLTISSLQPE |
| | TLYLQMNSLRAEDTAIYYCARPGEGD | DFATYYCLQHNSYPWTFGQGTKVEIK |
| | YWGQGTLVTVSS | |
| CB21-499-175 | EVQLVESGGGVVQPGRSLRLSCAASG | EIVLTQSPATLSLSPGERATLSCRASQS |
| | FTFSSYGMHWVRQAPGKGLEWVAVI | VSSYLAWYQQKPGQAPRLLIYDASNR |
| | WYDASNKDYAPSVKGRFTISRDNSKN | ATGIPARFSGSGSGTDFTLTISSLEPEDF |
| | TLYLQMNSLRAEDTAIYYCARPGEGD | AVYYCQQRSNWPYTFGQGTKLEIK |
| | YWGQGTLVTVSS | |
| CB21-499-176 | EVQLVESGGGVVQPGRSLRLSCAASG | DIQMTQSPSSLSASVGDRVTITCRASQ |
| | FTFSSYGMHWVRQAPGKGLEWVAVI | SISSYLNWYQQKPGKAPKLLIYAASSL |
| | WYDASNKDYAPSVKGRFTISRDNSKN | QSGVPSRFSGSGSGTDFTLTISSLQPED |
| | TLYLQMNSLRAEDTAIYYCARPGEGD | FATYYCQQSYSTPFTFGPGTKVDIK |
| | YWGQGTLVTVSS | |
| CB21-499-177 | EVQLVESGGGVVQPGRSLRLSCAASG | DIQMTQSPSSLSASVGDRVTITCRASQ |
| | FTFSSYGMHWVRQAPGKGLEWVAVI | GVRNDFGWFQQKPGKAPKRLIYGAS |
| | WYDASNKEYAPSVKGRFTISRDNSKN | SLQSGVPSRFSGSGSGTEFTLTISSLQP |
| | TLYLQMNSLRAEDTAIYYCARPGEGD | EDFATYYCLQQNSYPWTFGQGTKLEI |
| | YWGQGTLVTVSS | K |
| CB21-499-178 | EVQLVESGGGVVQPGRSLRLSCAASG | DIQMTQSPSSLSASVGDRVTITCRASQ |
| | FTFSSYGMHWVRQAPGKGLEWVAVI | GIRNDFGWYQQKPGKAPKRLIYGASS |
| | WYDASNKEYAPSVKGRFTISRDNSKN | LQSGVPSRFSGSGSGTEFTLTISSLQPE |
| | TLYLQMNSLRAEDTAIYYCARPGEGD | DFATYYCLQQNSYPWTFGQGTKVEIK |
| | YWGQGTLVTVSS | |
| CB21-499-179 | EVQLVESGGGVVQPGRSLRLSCAASG | DIQMTQSPSSLSASVGDRVTITCRASQ |
| | FTFSSYGMHWVRQAPGKGLEWVAVI | GIRNDFGWYQQKPGKAPKRLIYGASS |
| | WYDASNKDYAPSVKGRFTISRDNSKN | LQSGVPSRFSGSGSGTEFTLTISSLQPE |
| | TLYLQMNSLRAEDTAIYYCARPGEGD | DFATYYCLQQNSYPWTFGQGTKVEIK |
| | YWGQGTLVTVSS | |

In certain embodiments, the hLY6G6D binding protein is an antibody. The term “antibody” as used herein is used in the broadest sense and specifically covers, but is not limited to, monoclonal antibodies (including full length monoclonal antibodies) and multispecific antibodies (e.g., bispecific antibodies) such as based on the Duobody® technology (Genmab) or Hexabody® technology (Genmab), antibody fragments, and artificial constructs such as single-chain variable fragments (scFvs) that comprise immunoglobulin variable regions.

“Antibody fragment” and “antibody binding fragment” mean antigen-binding fragments and analogues of an antibody, typically including at least a portion of the antigen binding or variable regions (e.g. one or more CDRs) of the parental antibody. An antibody fragment retains at least some of the binding specificity of the parental antibody. Typically, an antibody fragment retains at least 10% of the parental binding activity when that activity is expressed on a molar basis. Preferably, an antibody fragment retains at least 20%, 50%, 70%, 80%, 90%, 95% or 100% or more of the parental antibody's binding affinity for the target. Examples of antibody fragments include, but are not limited to, Fab, Fab′, F(ab′)2, and Fv fragments; diabodies; linear antibodies; single-chain antibody molecules, e.g., sc-Fv, unibodies (technology from Genmab); nanobodies (technology from Ablynx); domain antibodies (technology from Domantis); and multispecific antibodies formed from antibody fragments. Engineered antibody variants are reviewed in Holliger and Hudson, 2005, Nat. Biotechnol. 23:1126-1136.

In certain embodiments, an hLY6G6D binding protein of the invention is a T cell engaging antibody (e.g., a bispecific T cell engaging antibody, or BiTE® (Micromet)), a pro-Bispecific T Cell Engager (pro-BiTE) molecule, pro-Chimeric Antigen Receptor (pro-CAR) modified T cell, or other engineered receptor or other immune effector cell, such as a CAR modified NK cell, at least one arm of which binds hLY6G6D. T cell engagers are multivalent molecules that typically bind to both a tumor associated antigen and to CD3 on the surface of CD3+ T cells, thereby bridging the two cell types. Alternative multispecifics directed to tumor antigens can bridge to cell surface receptors such as CD16, γδ TCR, CTLA-4, PD-1, PD-L1, and 4-1BB, etc., or can bridge to cytokines such as IL-13, IL-4, VEGF, etc. Preferably, an antibody of the invention is a multispecific antibody, and most preferably a BiTE, which acts through the simultaneous engagement of hLY6G6D and CD3, resulting in the activation of T-cells irrespective of MHC.

An “Fab fragment” is comprised of one light chain and the C_{H}1 and variable regions of one heavy chain. The heavy chain of a Fab molecule cannot form a disulfide bond with another heavy chain molecule.

An “Fc” region contains two heavy chain fragments comprising the C_{H}1 and C_{H}2 domains of an antibody. The two heavy chain fragments are held together by two or more disulfide bonds and by hydrophobic interactions of the C_{H}3 domains.

An “Fab′ fragment” contains one light chain and a portion of one heavy chain that contains the V_{H} domain and the C_{H}1 domain and also the region between the C_{H}1 and C_{H}2 domains, such that an interchain disulfide bond can be formed between the two heavy chains of two Fab′ fragments to form a F(ab′)₂ molecule.

An “F(ab′)₂ fragment” contains two light chains and two heavy chains containing a portion of the constant region between the C_{H}1 and C_{H}2 domains, such that an interchain disulfide bond is formed between the two heavy chains. A F(ab′)₂ fragment thus is composed of two Fab′ fragments that are held together by a disulfide bond between the two heavy chains.

The “Fv region” comprises the variable regions from both the heavy and light chains, but lacks the constant regions.

A “single-chain Fv antibody” (or “scFv antibody”) refers to antibody fragments comprising the V_{H} and V_{L} domains of an antibody, wherein these domains are present in a single polypeptide chain. Generally, the Fv polypeptide further comprises a polypeptide linker between the V_{H} and V_{L} domains which enables the scFv to form the desired structure for antigen binding. For a review of scFv, see Pluckthun, 1994, The Pharmacology of Monoclonal Antibodies, vol. 113, Rosenburg and Moore eds. Springer-Verlag, New York, pp. 269-315. See also, International Patent Application Publication No. WO 88/01649 and U.S. Pat. Nos. 4,946,778 and 5,260,203.

A “diabody” is a small antibody fragment with two antigen-binding sites. The fragments comprise a heavy chain variable domain (V_{H}) connected to a light chain variable domain (V_{L}) in the same polypeptide chain (V_{H}-V_{L} or V_{L}-V_{H}). By using a linker that is too short to allow pairing between the two domains on the same chain, the domains are forced to pair with the complementary domains of another chain and create two antigen-binding sites. Diabodies are described more fully in, e.g., EP 404,097; WO 93/11161; and Holliger et al., 1993, Proc. Natl. Acad. Sci. USA 90:6444-6448.

A “Duobody®” (Genmab) is a bispecific antibody with normal IgG structures (Labrijn et al., 2013, Proc. Natl. Acad. Sci. USA 110 (13): 5145-5150).

“Hexabodies” are antibodies that, while retaining regular structure and specificity, have an increased killing ability (Diebolder et al., 2014, Science 343 (6176): 1260-3).

A “domain antibody fragment” is an immunologically functional immunoglobulin fragment containing only the variable region of a heavy chain or the variable region of a light chain. In some instances, two or more VH regions are covalently joined with a peptide linker to create a bivalent domain antibody fragment. The two VH regions of a bivalent domain antibody fragment may target the same or different antigens.

In certain embodiments, the hLY6G6D binding protein is a single chain variable fragment (scFv), a BiTE®, a (SCFV) 2, a NANOBODY®, a nanobody-HSA VHH-scAb, a VHH-Fab, a Dual scFab, a F(ab′)2, a diabody, a CROSSMAB®, a DAF (two-in-one), a DAE (four-in-one), a DUTAMAB®, a DT-TgG, a knobs-in-holes common light chain, a knobs-in-holes assembly, a charge pair, a Fab-arm exchange, a SEEDbody, a LUZ-Y, a FcAb, a kl-body, an orthogonal Fab, a DVD-IgG, a IgG (H)-scFv, a scFv-(H) IgG, IgG (L)-scFv, scFv-(L) IgG, IgG (L,H)-Fv, IgG (H)-V, V(H)-IgG, IgG (L)-V, V(L)-IgG, KIH IgG-scFab, 2scFv-IgG, IgG-2scFv, scFv4-Ig, ZYBODY™, DVI-IgG, Diabody-CH3, a triple body, a miniantibody, a minibody, a TriBi minibody, scFv-CH3 KIH, Fab-scFv, a F(ab′)2-scFv2, a scFv-KIH, a Fab-scFv-Fc, a tetravalent HCAb, a scDiabody-Fc, a Diabody-Fc, a tandem scFv-Fc, a VHH-Fc, a tandem VHH-Fc, a LTIH-Fc KiH, a Fab-VHH-Fc, an Intrabody, a dock and lock, an ImmTAC® (immune-mobilizing monoclonal TCRs (T cell receptors) against cancer), an IgG-IgG conjugate, a Cov-X-Body, a scFv1-PEG-scFv2, an Adnectin, a DARPin, or a fibronectin, an IgG, an IgM, an IgA, an IgE, an IgD, or a DEP conjugate, TMEAbody™, SAFEbody®, TRITAC®, a dual affinity retargeting (DART®) bispecific antibody, a simultaneous multiple interaction T-cell engagers (SMITE), or a SHIELD.

In some embodiments, the hLY6G6D binding protein is or comprises an IgG, IgM, IgA, IgE, or IgD antibody or fragment thereof. In some embodiments, the target binding protein is an IgG1, IgG2, IgG3, or IgG4 antibody. In some embodiments, the target binding protein is humanized.

An antibody fragment of the invention may comprise a sufficient portion of the constant region to permit dimerization (or multimerization) of heavy chains that have reduced disulfide linkage capability, for example where at least one of the hinge cysteines normally involved in inter-heavy chain disulfide linkage is altered as described herein. In another embodiment, an antibody fragment, for example one that comprises the Fc region, retains at least one of the biological functions normally associated with the Fc region when present in an intact antibody, such as FcRn binding, antibody half life modulation, ADCC (antibody dependent cellular cytotoxicity) function, and/or complement binding (for example, where the antibody has a glycosylation profile necessary for ADCC function or complement binding).

The hLY6G6D binding protein of the present invention also includes antibodies with modified (or blocked) Fc regions to provide altered effector functions. See, e.g. U.S. Pat. No. 5,624,821; WO2003/086310; WO2005/120571; WO2006/0057702; Presta, 2006, Adv. Drug Delivery Rev. 58:640-656. Such modification can be used to enhance or suppress various reactions of the immune system, with possible beneficial effects in diagnosis and therapy. Alterations of the Fc region include amino acid changes (substitutions, deletions and insertions), glycosylation or deglycosylation, and adding multiple Fc. Changes to the Fc can also alter the half-life of antibodies in therapeutic antibodies, and a longer half-life would result in less frequent dosing, with the concomitant increased convenience and decreased use of material. See Presta, 2005, J. Allergy Clin. Immunol. 116:731 at 734-35. In certain embodiments, the target binding protein of the present invention is a hLY6G6D/CD3 BiTE in an IgG1 format with reduced or absent effector functions relative to an unmodified IgG1.

The hLY6G6D binding proteins of the present invention also include antibodies with intact Fc regions that provide full effector functions, e.g. antibodies of isotype IgG1, which induce complement-dependent cytotoxicity (CDC) or antibody dependent cellular cytotoxicity (ADCC) in a targeted cell.

In some embodiments, the hLY6G6D binding protein is a hLY6G6D/CD3 BiTE that further comprises a masking moiety that inhibits binding of the target binding protein to CD3 in an inactive state. In some embodiments, the masking moiety is coupled to the target binding protein via a cleavable moiety (either directly or indirectly, e.g., via one or more linkers), and the cleavable moiety is a substrate for a protease. In some embodiments, the protease is ADAMS, AD AMTS, ADAM8, ADAM9, ADAM10, ADAM12, ADAM15, ADAM17/TACE, ADAMDEC1, ADAMTS1, ADAMTS4, ADAMTS5, Aspartate proteases, BACE, Renin, Aspartic cathepsins, Cathepsin D, Cathepsin E, Caspases, Caspase 1, Caspase 2, Caspase 3, Caspase 4, Caspase 5, Caspase 6, Caspase 7, Caspase 8, Caspase 9, Caspase 10, Caspase 14, Cysteine cathepsins, Cathepsin B, Cathepsin C, Cathepsin K, Cathepsin L, Cathepsin S, Cathepsin V/L2, Cathepsin X/Z/P, Cysteine proteinases, Cruzipain, Legumain, Otubain-2, KLKs, KLK4, KLK5, KLK6, KLK7, KLK8, KLK10, KLK11, KLK13, KLK14, Metallo proteinases, Meprin, Neprilysin, PSMA, BMP-1, MMPs, MMP1, MMP2, MMP3, MMP7, MMP8, MMP9, MMP10, MMP11, MMP12, MMP13, MMP14, MMP15, MMP16, MMP17, MMP19, MMP20, MMP23, MMP24, MMP26, MMP27, Serine proteases, activated protein C, Cathepsin A, Cathepsin G, Chymase, coagulation factor proteases, FVIIa, FIXa, FXa, FXIa, FXIIa, Elastase, Granzyme B, Guanidinobenzoatase, HtrAl, Human Neutrophil Elastase, Lactoferrin, Marapsin, NS3/4A, PACE4, Plasmin, PSA, tPA, Thrombin, Tryptase, uPA, Type II Transmembrane, Serine Proteases, TTSPs, DESCI, DPP-4, FAP, Hepsin, Matriptase-2, MT-SPl/Matriptase, TMPRSS2, TMPRSS3, or TMPRSS4.

In some embodiments, the hLY6G6D binding protein further comprises a second immunoglobulin variable region that specifically binds to a second target antigen.

In some embodiments, the hLY6G6D binding protein is an antibody-drug conjugate. In certain embodiments, the antibody is conjugated to a toxin, radioisotope, small molecule, diagnostic agent, therapeutic macromolecule, targeting moiety, or detectable moiety, via a conjugating moiety. In some embodiments, the conjugating moiety is cleavable by a protease. In some embodiments, the conjugating moiety is non-cleavable by a protease.

In another aspect, the present disclosure provides a pharmaceutical composition comprising a hLY6G6D binding protein of the invention and a pharmaceutically acceptable carrier.

In another aspect, the present disclosure provides a nucleic acid encoding hLY6G6D binding proteins comprising an immunoglobulin variable region that binds hLY6G6D, wherein the immunoglobulin variable region comprises heavy chain CDRs H1, H2, and H3 and light chain CDRs L1, L2, and L3 as recited for one of the Identifiers in Table 1.

The invention also provides isolated nucleic acids encoding anyone of the anti-hLY6G6D antibodies or antigen binding fragments of the invention.

The invention also provides expression vectors comprising one or more nucleic acids of the present invention. An expression vector is a DNA molecule comprising the regulatory elements necessary for transcription of a target nucleic acid in a host cell. Typically, the target nucleic acid is placed under the control of certain regulatory elements including constitutive or inducible promoters, tissue-specific regulatory elements, and enhancer elements. Such a target nucleic acid is said to be “operably linked to” the regulatory elements when the regulating element controls the expression of the gene.

These isolated nucleic acids and the expression vectors comprising them may be used to express the antibodies of the invention or antigen binding fragments thereof in recombinant host cells. Thus, the invention also provides host cells comprising an expression vector of the present invention.

The invention also provides a vessel or injection device comprising anyone of the anti-hLY6G6D antibodies or antigen binding fragments of the invention.

The invention also provides a method of producing an anti-hLY6G6D antibody or antigen binding fragment of the invention comprising: culturing a host cell comprising a polynucleotide encoding a heavy chain and/or light chain of an antibody of the invention (or an antigen binding fragment thereof) under conditions favorable to expression of the polynucleotide; and optionally, recovering the antibody or antigen binding fragment from the host cell and/or culture medium. In one embodiment, the polynucleotide encoding the heavy chain and the polynucleotide encoding the light chain are in a single vector. In another embodiment, the polynucleotide encoding the heavy chain and the polynucleotide encoding the light chain are in different vectors.

In another aspect, the present disclosure provides a method of treating a subject in need thereof comprising administering to the subject a therapeutically effective amount of a hLY6G6D binding protein described herein or the pharmaceutical composition described herein. In some embodiments, the subject in need thereof has been identified or diagnosed as having a cancer.

In another aspect, the present disclosure provides a method of producing a target binding protein of the invention, comprising: culturing the target cell described herein in a culture medium under a condition sufficient to produce the target binding protein; and recovering the target binding protein from the cell or the culture medium. In some embodiments, the method further comprises isolating the target binding protein recovered from the cell or the culture medium. In some embodiments, the method further comprises formulating the target binding protein into a pharmaceutical composition.

An understanding of the features and advantages of the present invention will be obtained by reference to the following detailed description that sets forth illustrative embodiments, in which the principles of the invention may be utilized, and the accompanying drawings.

### BRIEF DESCRIPTION OF THE FIGURES

FIG. 1A: guide tree mapping of sequence-binding relationships for LY6G6D binding antibodies of the invention.

FIG. 1B: Martin tree alignment of heavy chain CDR3 sequences for binding to a target membrane-proximal epitope on LY6G6D and the corresponding binding characteristics using cells displaying LY6G6D surface-exposed epitopes.

FIG. 2A multipoint BLI kinetic analysis of LY6G6D binding by LY6G6D binding antibodies of the invention.

FIG. 2B multipoint BLI kinetic analysis of LY6G6D binding by LY6G6D binding antibodies of the invention.

FIG. 3: parental antibodies CB21-498, CB21-499, CB21-512, CB21-476, and CB21-501 evaluated for polyreactivity using a BVP ELISA. Results for reference antibody enokizumab, gantenerumab, and LY6G6D binder 20A12.QNTv12 are shown for comparison.

FIG. 4: accelerated thermal stability SEC results comparing day zero to day 14 at 40° C. for three selected LY6G6D binding antibodies of the invention in an IgG format

FIG. 5: q ribbon representation of a model anti-LY6G6D immunoglobulin binding domain showing various substitutions examined during engineering of CB21-498. LFR3 and HFR2 indicate light chain framework region 3 and heavy chain framework region 2, respectively.

FIG. 6: A ribbon representation of a model anti-LY6G6D immunoglobulin binding domain showing various substitutions examined during engineering of CB21-498.

FIG. 7: depicts target binding affinity of an LY6G6D binding antibody of the invention comprising a T69Q substitution in HFR3.

FIG. 8: compares target binding affinity of LY6G6D binding antibody CB21-499 to an equivalent antibody CB21-499-177 comprising a D59E substitution in CDRH2.

FIG. 9: an isoaffinity plot for various LY6G6D binding antibody CB21-498 and CB21-499 family antibodies characterized by BLI.

FIG. 10: human/cyno crossreactivity for various LY6G6D binding antibody CB21-498 and CB21-499 family antibodies characterized by BLI.

FIG. 11: an AC-SINS analysis for LY6G6D binding antibody CB21-498-142 and CB21-499-177 as compared to various antibodies providing developability standards.

FIG. 12: a BVP-ELISA analysis for LY6G6D binding antibody CB21-498-142 and CB21-499-177 as compared to various antibodies providing developability standards.

FIG. 13: AbMap epitope mapping of CB21-499-177 and CB21-498-142 IgG vs. LY6G6D binding antibody 20A12.QNTv12.

### DETAILED DESCRIPTION OF THE INVENTION

Provided herein are target binding proteins that specifically bind to human lymphocyte antigen 6 family member G6D (hLY6G6D). In one aspect, the target binding protein may include a heavy chain variable domain and a light chain variable domain, which form an immunoglobulin variable region that specifically binds to HLY6G6D. In some embodiments, the hLY6G6D binding proteins of the invention may be single chain proteins, such as single chain antibodies. For example, the hLY6G6D binding proteins may be single chain fragment variable (scFv) antibodies. In some embodiments, the target binding proteins may be multichain proteins (e.g., intact IgG antibodies), at least one arm of which binds LY6G6D.

In some embodiments, the target binding proteins may be multispecific (e.g., bispecific) binding proteins that bind to one or more additional targets other than hLY6G6D. For example, the multispecific proteins may specifically bind to hLY6G6D and one or more other targets. By way of example, the other target may be a T cell protein such as CD3. In another example, the other target may be an immune checkpoint molecule such as PD-L1.

Also provided herein are related compositions, kits, nucleic acids, vectors, and recombinant cells, as well as related methods, including methods of using and methods of producing any of the target binding proteins described herein.

### Definitions

The term “a” and “an” refers to one or more (i.e., at least one) of the grammatical object of the article. By way of example, “a cell” encompasses one or more cells.

As used herein, the terms “about” and “approximately,” when used to modify an amount specified in a numeric value or range, indicate that the numeric value includes ±10%, and preferably ±5% or ±1% of the stated value.

In understanding the scope of the present disclosure, the terms “including” or “comprising” and their derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of,” as used herein, is intended to specify the presence of the stated features, elements, components, groups, integers, and/or steps as well as those that do not materially affect the basic and novel characteristic(s) of features, elements, components, groups, integers, and/or steps. It is understood that reference to any one of these transition terms (i.e. “comprising,” “consisting,” or “consisting essentially”) provides direct support for replacement to any of the other transition term not specifically used. For example, amending a term from “comprising” to “consisting essentially of” or “consisting of” would find direct support due to this definition for any elements disclosed throughout this disclosure. Based on this definition, any element disclosed herein or incorporated by reference may be included in or excluded from the claimed invention.

As used herein, a plurality of compounds, elements, or steps may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

The term “exemplary” is used herein to mean serving as an example, instance, or illustration. Any aspect or design described herein as “exemplary” is not necessarily to be construed as preferred or advantageous over other aspects or designs. Rather, use of the word exemplary is intended to present concepts in a more concrete fashion.

Certain molecules, constructs, compositions, elements, moieties, excipients, disorders, conditions, properties, steps, or the like may be discussed in the context of one specific embodiment or aspect or in a separate paragraph or section of this disclosure. It is understood that this is merely for convenience and brevity, and any such disclosure is equally applicable to and intended to be combined with any other embodiments or aspects found anywhere in the present disclosure and claims, which all form the application and claimed invention at the filing date. For example, a list of constructs, molecules, method steps, kits, or compositions described with respect to a construct, composition, or method is intended to and does find direct support for embodiments related to constructs, compositions, formulations, and methods described in any other part of this disclosure, even if those method steps, active agents, kits, or compositions are not re-listed in the context or section of that embodiment or aspect.

hLY6G6D Binding Proteins of the Invention

As used herein, a “target binding protein” of the invention comprises a heavy chain variable domain and a light chain variable domain, which together form at least one immunoglobulin variable region that specifically binds to human hLY6G6D. In each case, a target binding protein of the invention that comprises two or more binding domains may comprise only hLY6G6D binding domains, or may comprise at least one hLY6G6D binding domain and a binding domain that specifically binds to another target (e.g., CD3 or an immune checkpoint molecule such as PD-L1). Thus, references to a target binding protein or an anti-hLY6G6D antibody herein refer to both a monospecific (only hLY6G6D binding) or a multispecific (e.g., bispecific) target binding protein or anti-hLY6G6D antibody.

In some embodiments, the heavy chain variable domain and the light chain variable domain are disposed within the same polypeptide. In certain embodiments, the heavy chain variable domain and the light chain variable domain are coupled together by one or more linkers. The linker may be a peptide linker described in the Linkers section below. In some examples, the immunoglobulin variable region in the single chain polypeptide may be an scFv. Alternatively, the heavy chain variable domain and the light chain variable domain are disposed within two different polypeptides.

In some embodiments, the target binding protein may be a protein complex that comprises multiple polypeptides (e.g., two, three, four, five, six, seven, eight, nine, ten, or more polypeptides). In some examples, some or all (e.g., two, three, four, five, six, seven, eight, nine, ten, or more polypeptides) of the multiple polypeptides may be identical. In some examples, each of the multiple polypeptides in the target binding complex is different from the other.

The immunoglobulin variable region may reside within any of a variety of different constructs, including an antibody or a fragment thereof, a VH domain, a VHH domain, a VNAR domain, and a single chain fragment variable (scFv), BiTE or a component thereof, a (scFv)2, a NANOBOD Y®, a nanobody-HSA, VHH-scAb, a VHH-Fab, a Dual scFab, a F(ab′)2, a diabody, a CROSSMAB®, a DAF (two-in-one), a DAE (four-in-one), a DUTAMAB®, a DT-IgG, a knobs-in-holes common light chain, a knobs-in-holes assembly, a charge pair, a Fab-arm exchange, a SEEDbody, a LUZ-Y, a FcAb, a kl-body, an orthogonal Fab, a DVD-IgG, a IgG (H)-scFv, a scFv-(H) IgG, IgG (L)-scFv, scFv-(L) IgG, IgG (L,H)-Fv, IgG (H)-V, V(H)-IgG, IgG (L)-V, V(L)-IgG, KIH IgG-scFab, 2scFv-IgG, IgG-2scFv, scFv4-Ig, ZYBODY™, DVLIgG, Diabody-CH3, a triple body, a miniantibody, a minibody, a TriBi minibody, scFv-CH3 KIH, Fab-scFv, a F(ab′)2-scFv2, a scFv-KIH, a Fab-scFv-Fc, a tetravalent HCAb, a scDiabody-Fc, a Diabody-Fc, a tandem scFv-Fc, a VHH-Fc, a tandem VHH-Fc, a L′HH-Fc KiH, a Fab-VHH-Fc, an Intrabody, a dock and lock, an ImmTAC® (immune-mobilizing monoclonal TCRs (T cell receptors) against cancer), an IgG-IgG conjugate, a Cov-X-Body, a scFv1-PEG-scFv2, an Adnectin, a DARPin®, a fibronectin, an IgG, an IgM, an IgA, an IgE, an IgD, a DEP conjugate, TMEAbody™, SAFEbody®, TRITAC®, a dual affinity retargeting (DART®) bispecific antibody, a simultaneous multiple interaction T-cell engagers (SMITE), or SHIELD antibody.

Whether a target binding protein specifically binds to a polypeptide sequence (e.g., human LY6G6D) can be determined using any assay known in the art. Examples of assays known in the art to determining binding affinity include surface plasmon resonance (e.g., BIACORE® (Cytiva Sweden AB) or a similar technique (e.g. KinExa® (Sapidyne Instruments Inc.) or OCTET® (Sartorius)).

In preferred embodiments, a target binding protein of the invention is an antibody. As used herein, the term “antibody” refers to any form of antibody that exhibits the desired biological activity. The term antibody includes antigen-binding portions, i.e., “antigen binding sites,” (e.g., fragments, subsequences, complementarity determining regions (CDRs)) that retain capacity to bind antigen, including (i) a Fab fragment, a monovalent fragment consisting of the V_{L}, V_{H}, C_{L} and C_{H}l domains; (ii) a F(ab′)2 fragment, a bivalent fragment comprising two Fab fragments linked by a disulfide bridge at the hinge region; (iii) a Fd fragment consisting of the V_{H} and C_{H}l domains; (iv) a Fv fragment consisting of the V_{L} and V_{H} domains of a single arm of an antibody, (v) a dAb fragment (Ward et al., (1989) Nature 341:544-546), which consists of a V_{H} domain; and (vi) an isolated complementarity determining region (CDR). Single chain antibodies are also included by reference in the term “antibody.” Preferred therapeutic antibodies are intact IgG antibodies. The term “intact IgG” as used herein is meant as a polypeptide belonging to the class of antibodies that are substantially encoded by a recognized immunoglobulin gamma gene. In humans this class comprises IgG1, IgG2, IgG3, and IgG4. In mice this class comprises IgG1, IgG2a, IgG2b, IgG3. The known Ig domains in the IgG class of antibodies are V_{H}, Cγ1, Cγ2, Cγ3, V_{L}, and C_{L}.

The present invention includes anti-hLY6G6D antigen-binding fragments and methods of use thereof.

As used herein, a “full length antibody” is, in the case of an IgG, a bivalent molecule comprising two heavy chains and two light chains. Each heavy chain comprises a V_{H} domain followed by a constant domain (C_{H1}), a hinge region, and two more constant (C_{H2} and C_{H3}) domains; while each light chain comprises one V_{L} domain and one constant (C_{L}) domain. A full length antibody in the case of an IgM is a decavalent or dodecavalent molecule comprising 5 or 6 linked immunoglobulins in which each immunoglobulin monomer has two antigen binding sites formed of a heavy and light chain.

As used herein, unless otherwise indicated, “antibody fragment” or “antigen-binding fragment” refers to antigen-binding fragments of antibodies, i.e. antibody fragments that retain the ability to bind specifically to the antigen bound by the full-length antibody, e.g. fragments that retain one or more CDR regions. Examples of antigen-binding fragments include, but are not limited to, Fab, Fab′, F(ab′)₂, and Fv fragments; diabodies; linear antibodies; single-chain antibody molecules, e.g., sc-Fv; nanobodies and multispecific antibodies formed from antibody fragments.

The present invention includes anti-hLY6G6D Fab fragments and methods of use thereof. A “Fab fragment” is comprised of one light chain and the C_{H}1 and variable regions of one heavy chain. The heavy chain of a Fab molecule cannot form a disulfide bond with another heavy chain molecule. A “Fab fragment” can be the product of papain cleavage of an antibody.

The present invention includes anti-hLY6G6D antibodies and antigen-binding fragments thereof which comprise an Fc region and methods of use thereof. An “Fc” region contains two heavy chain fragments comprising the C_{H}3 and C_{H}2 domains of an antibody. The two heavy chain fragments are held together by two or more disulfide bonds and by hydrophobic interactions of the C_{H}3 domains.

The present invention includes anti-hLY6G6D Fab′ fragments and methods of use thereof. A “Fab′ fragment” contains one light chain and a portion or fragment of one heavy chain that contains the V_{H} domain and the C_{H}1 domain and also the region between the C_{H}1 and C_{H}2 domains, such that an interchain disulfide bond can be formed between the two heavy chains of two Fab′ fragments to form a F(ab′)₂ molecule.

The present invention includes anti-hLY6G6D F(ab′)₂ fragments and methods of use thereof. A “F(ab′)₂ fragment” contains two light chains and two heavy chains containing a portion of the constant region between the C_{H1} and C_{H2} domains, such that an interchain disulfide bond is formed between the two heavy chains. A F(ab′)₂ fragment thus is composed of two Fab′ fragments that are held together by a disulfide bond between the two heavy chains. An “F(ab′)₂ fragment” can be the product of pepsin cleavage of an antibody.

The present invention includes anti-hLY6G6D Fv fragments and methods of use thereof. The “Fv region” comprises the variable regions from both the heavy and light chains, but lacks the constant regions.

The present invention includes anti-hLY6G6D scFv fragments and methods of use thereof. The term “single-chain Fv” or “scFv” antibody refers to antibody fragments comprising the V_{H} and V_{L} domains of an antibody, wherein these domains are present in a single polypeptide chain. Generally, the Fv polypeptide further comprises a polypeptide linker between the V_{H} and V_{L} domains which enables the scFv to form the desired structure for antigen-binding. For a review of scFv, see Pluckthun (1994) The Pharmacology of Monoclonal Antibodies, vol. 113, Rosenburg and Moore eds. Springer-Verlag, New York, pp. 269-315. See also, International Patent Application Publication No. WO 88/01649 and U.S. Pat. Nos. 4,946,778 and 5,260,203.

The present invention includes anti-hLY6G6D domain antibodies and methods of use thereof. A “domain antibody” is an immunologically functional immunoglobulin fragment containing only the variable region of a heavy chain or the variable region of a light chain. In some instances, two or more V_{H} regions are covalently joined with a peptide linker to create a bivalent domain antibody. The two V_{H} regions of a bivalent domain antibody may target the same or different antigens.

The present invention includes anti-hLY6G6D bivalent antibodies and methods of use thereof. A “bivalent antibody” comprises two antigen-binding sites. In some instances, the two binding sites have the same antigen specificities. However, bivalent antibodies may be bispecific (see below).

The present invention includes anti-hLY6G6D diabodies and methods of use thereof. As used herein, the term “diabodies” refers to small antibody fragments with two antigen-binding sites, which fragments comprise a heavy chain variable domain (V_{H}) connected to a light chain variable domain (V_{L}) in the same polypeptide chain (V_{H}-V_{L} or V_{L}-V_{H}). By using a linker that is too short to allow pairing between the two domains on the same chain, the domains are forced to pair with the complementary domains of another chain and create two antigen-binding sites. Diabodies are described more fully in, e.g., EP 404,097; WO 93/11161; and Holliger et al. (1993) Proc. Natl. Acad. Sci. USA 90:6444-6448. Duobodies are described in Labrijn et al., 2013, Proc. Natl. Acad. Sci. USA 110 (13): 5145-5150. For a review of engineered antibody variants generally see Holliger and Hudson (2005) Nat. Biotechnol. 23:1126-1136.

Typically, an antibody or antigen-binding fragment of the invention which is modified in some way retains at least 10% of its binding activity (when compared to the parental antibody) when that activity is expressed on a molar basis. Preferably, an antibody or antigen-binding fragment of the invention retains at least 20%, 50%, 70%, 80%, 90%, 95% or 100% or more of the hLY6G6D binding affinity as the parental antibody. It is also intended that an antibody or antigen-binding fragment of the invention can include conservative or non-conservative amino acid substitutions (referred to as “conservative variants” or “function conserved variants” of the antibody) that do not substantially alter its biologic activity.

The present invention includes isolated anti-hLY6G6D antibodies and antigen-binding fragments thereof and methods of use thereof. Herein, the term “isolated” is not intended to refer to a complete absence of such biological molecules or to an absence of water, buffers, or salts or to components of a pharmaceutical formulation that includes the antibodies or fragments. An “isolated” antibody, antigen-binding fragment, nucleic acid, etc., is one which has been identified and separated and/or recovered from one or more components of its natural environment. In preferred embodiments, the antibody, antigen-binding fragment, nucleic acid, etc., is purified to 75% by weight or more, more preferably to 90% by weight or more, still more preferably to 95% by weight or more, and still more preferably to 98% by weight or more. Thus, “isolated” biological molecules are at least partially free of other biological molecules from the cells or cell cultures in which they are produced. Such biological molecules include nucleic acids, proteins, lipids, carbohydrates, or other material such as cellular debris and growth medium. An isolated antibody or antigen-binding fragment may further be at least partially free of expression system components such as biological molecules from a host cell or of the growth medium thereof.

The present invention includes anti-hLY6G6D chimeric antibodies (e.g., human constant domain/mouse variable domain) and methods of use thereof. As used herein, a “chimeric antibody” is an antibody having the variable domain from a first antibody and the constant domain from a second antibody, where the first and second antibodies are from different species. (U.S. Pat. No. 4,816,567; and Morrison et al., (1984) Proc. Natl. Acad. Sci. USA 81:6851-6855). Typically, the variable domains are obtained from an antibody from an experimental animal (the “parental antibody”), such as a rodent, and the constant domain sequences are obtained from human antibodies, so that the resulting chimeric antibody will be less likely to elicit an adverse immune response in a human subject than the parental (e.g., mouse) antibody.

The present invention includes anti-hLY6G6D humanized antibodies and antigen-binding fragments thereof (e.g., rat or mouse antibodies that have been humanized) and methods of use thereof. As used herein, the term “humanized antibody” refers to forms of antibodies that contain sequences from both human and non-human (e.g., mouse or rat) antibodies. In general, the humanized antibody will comprise substantially of at least one, and typically two, variable domains, in which all or substantially all of the hypervariable loops correspond to those of a non-human immunoglobulin, and all or substantially all of the framework (FR) regions are those of a human immunoglobulin sequence. The humanized antibody may optionally comprise at least a portion of a human immunoglobulin constant region (Fc). For more details about humanized antibodies, see, e.g., Jones et al., Nature, 321:522-525 (1986); Reichmann et al., Nature, 332:323-329 (1988); Presta, Curr. Op. Struct. Biol., 2:593-596 (1992); and Clark, Immunol. Today 21:397-402 (2000).

In general, the basic antibody structural unit comprises a tetramer. Each tetramer includes two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of the heavy chain may define a constant region primarily responsible for effector function. Typically, human light chains are classified as kappa and lambda light chains. Furthermore, human heavy chains are typically classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989).

The variable regions of each light/heavy chain pair form the antibody binding site. Thus, in general, an intact antibody has two binding sites. Except in bifunctional or bispecific antibodies, the two binding sites are, in general, the same.

Typically, the variable domains of both the heavy and light chains comprise three hypervariable regions, also called complementarity determining regions (CDRs), located within relatively conserved framework regions (FR). The CDRs are usually aligned by the framework regions, enabling binding to a specific epitope. In general, from N-terminal to C-terminal, both light and heavy chains variable domains comprise FR1, CDR1, FR2, CDR2, FR3, CDR3 and FR4. The assignment of amino acids to each domain is, generally, in accordance with the definitions of Sequences of Proteins of Immunological Interest, Kabat, et al.; National Institutes of Health, Bethesda, MD; 5^{th} ed.; NIH Publ. No. 91-3242 (1991); Kabat (1978) Adv. Prot. Chem. 32:1-75; Kabat, et al., (1977) J. Biol. Chem. 252:6609-6616; Chothia, et al., (1987) J Mol. Biol. 196:901-917 or Chothia, et al., (1989) Nature 342:878-883.

As used herein, the term “hypervariable region” refers to the amino acid residues of an antibody or antigen-binding fragment thereof that are responsible for antigen-binding. The hypervariable region comprises amino acid residues from a “complementarity determining region” or “CDR” (i.e. CDRL1, CDRL2 and CDRL3 in the light chain variable domain and CDRH1, CDRH2 and CDRH3 in the heavy chain variable domain). See Kabat et al. (1991) Sequences of Proteins of Immunological Interest, 5th Ed. Public Health Service, National Institutes of Health, Bethesda, Md. (defining the CDR regions of an antibody by sequence); see also Chothia and Lesk (1987) J. Mol. Biol. 196:901-917 (defining the CDR regions of an antibody by structure). As used herein, the term “framework” or “FR” residues refers to those variable domain residues other than the hypervariable region residues defined herein as CDR residues.

“Isolated nucleic acid molecule” or “isolated polynucleotide” means a DNA or RNA of genomic, mRNA, cDNA, or synthetic origin or some combination thereof which is not associated with all or a portion of a polynucleotide in which the isolated polynucleotide is found in nature, or is linked to a polynucleotide to which it is not linked in nature. For purposes of this disclosure, it should be understood that “a nucleic acid molecule comprising” a particular nucleotide sequence does not encompass intact chromosomes. Isolated nucleic acid molecules “comprising” specified nucleic acid sequences may include, in addition to the specified sequences, coding sequences for up to ten or even up to twenty or more other proteins or portions or fragments thereof, or may include operably linked regulatory sequences that control expression of the coding region of the recited nucleic acid sequences, and/or may include vector sequences.

The phrase “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to use promoters, polyadenylation signals, and enhancers.

A nucleic acid or polynucleotide is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, but not always, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

As used herein, the expressions “cell,” “cell line,” and “cell culture” are used interchangeably and all such designations include progeny. Thus, the words “transformants” and “transformed cells” include the primary subject cell and cultures derived therefrom without regard for the number of transfers. It is also understood that not all progeny will have precisely identical DNA content, due to deliberate or inadvertent mutations. Mutant progeny that have the same function or biological activity as screened for in the originally transformed cell are included. Where distinct designations are intended, it will be clear from the context.

As used herein, “germline sequence” refers to a sequence of unrearranged immunoglobulin DNA sequences. Any suitable source of unrearranged immunoglobulin sequences may be used. Human germline sequences may be obtained, for example, from JOINSOLVER germline databases on the website for the National Institute of Arthritis and Musculoskeletal and Skin Diseases of the United States National Institutes of Health. Mouse germline sequences may be obtained, for example, as described in Giudicelli et al. (2005) Nucleic Acids Res. 33: D256-D261.

### Binding Affinity

By way of example, and not limitation, the antibodies and antigen-binding fragments disclosed herein may bind hLY6G6D bivalently with a KD value of 10×10−9 M or lower as determined by surface plasmon resonance (e.g., BIACORE®) or a similar technique (e.g. KinExa® or bio-layer interferometry (OCTET®)). In one embodiment, the antibodies and antigen-binding fragments disclosed herein may bind human CD3 bivalently with a K_{D} value of about 5-10×10⁻⁹ M as determined by surface plasmon resonance (e.g., BIACORE®) or a similar technique (e.g. KinExa® or OCTET®). Affinity is calculated as K_{D}=K_{off}/kₒₙ (k_{off} is the dissociation rate constant, Kₒₙ is the association rate constant and K_{D} is the equilibrium constant). Affinity can be determined at equilibrium by measuring the fraction bound (r) of labeled ligand at various concentrations (c). The data are graphed using the Scatchard equation: r/c=K(n−r): where r=moles of bound ligand/mole of receptor at equilibrium; c=free ligand concentration at equilibrium; K=equilibrium association constant; and n=number of ligand binding sites per receptor molecule. By graphical analysis, r/c is plotted on the Y-axis versus r on the X-axis, thus producing a Scatchard plot. Antibody affinity measurement by Scatchard analysis is well known in the art. See, e.g., van Erp et al., J. Immunoassay 12:425-43, 1991; Nelson and Griswold, Comput. Methods Programs Biomed. 27:65-8, 198.

Methods of Making Anti-hLY6G6D antibodies and Antigen-binding Fragments Thereof

The present invention includes methods for making an anti-hLY6G6D antibody or antigen-binding fragment thereof of the present invention.

The anti-hLY6G6D antibodies disclosed herein may be produced recombinantly (e.g., in an E. coli/T7 expression system, a mammalian cell expression system or a lower eukaryote expression system). In this embodiment, nucleic acids encoding the antibody immunoglobulin molecules of the invention (e.g., V_{H} or V_{L}) may be inserted into a pET-based plasmid and expressed in the E. coli/T7 system. For example, the present invention includes methods for expressing an antibody or antigen-binding fragment thereof or immunoglobulin chain thereof in a host cell (e.g., bacterial host cell such as E. coli such as BL21 or BL21DE3) comprising expressing T7 RNA polymerase in the cell which also includes a polynucleotide encoding an immunoglobulin chain that is operably linked to a T7 promoter. For example, in an embodiment of the invention, a bacterial host cell, such as a E. coli, includes a polynucleotide encoding the T7 RNA polymerase gene operably linked to a lac promoter and expression of the polymerase and the chain is induced by incubation of the host cell with IPTG (isopropyl-beta-D-thiogalactopyranoside).

There are several methods by which to produce recombinant antibodies which are known in the art. One example of a method for recombinant production of antibodies is disclosed in U.S. Pat. No. 4,816,567.

Transformation can be by any known method for introducing polynucleotides into a host cell. Methods for introduction of heterologous polynucleotides into mammalian cells are well known in the art and include dextran-mediated transfection, calcium phosphate precipitation, polybrene-mediated transfection, protoplast fusion, electroporation, encapsulation of the polynucleotide(s) in liposomes, biolistic injection and direct microinjection of the DNA into nuclei. In addition, nucleic acid molecules may be introduced into mammalian cells by viral vectors. Methods of transforming cells are well known in the art. See, for example, U.S. Pat. Nos. 4,399,216; 4,912,040; 4,740,461 and 4,959,455.

Thus, the present invention includes recombinant methods for making an anti-hLY6G6D antibody or antigen-binding fragment thereof of the present invention, or an immunoglobulin chain thereof, comprising introducing a polynucleotide encoding one or more immunoglobulin chains of the antibody or fragment (e.g., heavy and/or light immunoglobulin chain); culturing the host cell (e.g., CHO or Pichia or Pichia pastoris) under condition favorable to such expression and, optionally, isolating the antibody or fragment or chain from the host cell and/or medium in which the host cell is grown.

Anti-hLY6G6D antibodies can also be synthesized by any of the methods set forth in U.S. Pat. No. 6,331,415.

Eukaryotic and prokaryotic host cells, including mammalian cells as hosts for expression of the antibodies or fragments or immunoglobulin chains disclosed herein are well known in the art and include many immortalized cell lines available from the American Type Culture Collection (ATCC). These include, inter alia, Chinese hamster ovary (CHO) cells, NSO, SP2 cells, HeLa cells, baby hamster kidney (BHK) cells, monkey kidney cells (COS), human hepatocellular carcinoma cells (e.g., Hep G2), A549 cells, 3T3 cells, HEK-293 cells and a number of other cell lines. Mammalian host cells include human, mouse, rat, dog, monkey, pig, goat, bovine, horse and hamster cells. Cell lines of particular preference are selected through determining which cell lines have high expression levels. Other cell lines that may be used are insect cell lines, such as Sf9 cells, amphibian cells, bacterial cells, plant cells and fungal cells. Fungal cells include yeast and filamentous fungus cells including, for example, Pichia pastoris, Pichia finlandica, Pichia trehalophila, Pichia koclamae, Pichia membranaefaciens, Pichia minuta (Ogataea minuta, Pichia lindneri), Pichia opuntiae, Pichia thermotolerans, Pichia salictaria, Pichia guercuum, Pichia pijperi, Pichia stiptis, Pichia methanolica, Pichia sp., Saccharomyces cerevisiae, Saccharomyces sp., Hansenula polymorpha, Kluyveromyces sp., Kluyveromyces lactis, Candida albicans, Aspergillus nidulans, Aspergillus niger, Aspergillus oryzae, Trichoderma reesei, Chrysosporium lucknowense, Fusarium sp., Fusarium gramineum, Fusarium venenatum, Physcomitrella patens and Neurospora crassa. Pichia sp., any Saccharomyces sp., Hansenula polymorpha, any Kluyveromyces sp., Candida albicans, any Aspergillus sp., Trichoderma reesei, Chrysosporium lucknowense, any Fusarium sp., Yarrowia lipolytica, and Neurospora crassa. When recombinant expression vectors encoding the heavy chain or antigen-binding portion or fragment thereof, and/or the light chain or antigen-binding fragment thereof are introduced into mammalian host cells, the antibodies are produced by culturing the host cells for a period of time sufficient to allow for expression of the antibody or fragment or chain in the host cells or secretion into the culture medium in which the host cells are grown.

Antibodies and antigen-binding fragments thereof and immunoglobulin chains can be recovered from the culture medium using standard protein purification methods. Further, expression of antibodies and antigen-binding fragments thereof and immunoglobulin chains of the invention (or other moieties therefrom) from production cell lines can be enhanced using a number of known techniques. For example, the glutamine synthetase gene expression system (the GS system) is a common approach for enhancing expression under certain conditions. The GS system is discussed in whole or part in connection with European Patent Nos. 0216846, 0256055, and 0323997 and 0338841. Thus, in an embodiment of the invention, the mammalian host cells (e.g., CHO) lack a glutamine synthetase gene and are grown in the absence of glutamine in the medium wherein, however, the polynucleotide encoding the immunoglobulin chain comprises a glutamine synthetase gene which complements the lack of the gene in the host cell.

The present invention includes methods for purifying an anti-hLY6G6D antibody or antigen-binding fragment thereof of the present invention comprising introducing a sample comprising the antibody or fragment to a purification medium (e.g., cation exchange medium, anion exchange medium, hydrophobic exchange medium, affinity purification medium (e.g., protein-A, protein-G, protein-A/G, protein-L)) and either collecting purified antibody or fragment from the flow-through fraction of said sample that does not bind to the medium; or, discarding the flow-through fraction and eluting bound antibody or fragment from the medium and collecting the eluate. In an embodiment of the invention, the medium is in a column to which the sample is applied. In an embodiment of the invention, the purification method is conducted following recombinant expression of the antibody or fragment in a host cell, e.g., wherein the host cell is first lysed and, optionally, the lysate is purified of insoluble materials prior to purification on a medium.

In general, glycoproteins produced in a particular cell line or transgenic animal will have a glycosylation pattern that is characteristic for glycoproteins produced in the cell line or transgenic animal. Therefore, the particular glycosylation pattern of an antibody will depend on the particular cell line or transgenic animal used to produce the antibody. However, all antibodies encoded by the nucleic acid molecules provided herein, or comprising the amino acid sequences provided herein, comprise the instant invention, independent of the glycosylation pattern the antibodies may have. Similarly, in particular embodiments, antibodies with a glycosylation pattern comprising only non-fucosylated N-glycans may be advantageous, because these antibodies have been shown to typically exhibit more potent efficacy than their fucosylated counterparts both in vitro and in vivo (See for example, Shinkawa et al., J. Biol. Chem. 278:3466-3473 (2003); U.S. Pat. Nos. 6,946,292 and 7,214,775). These antibodies with non-fucosylated N-glycans are not likely to be immunogenic because their carbohydrate structures are a normal component of the population that exists in human serum IgG.

The present invention further includes anti-hLY6G6D antigen-binding fragments of the anti-hLY6G6D antibodies disclosed herein. The antibody fragments include F (ab) 2 fragments, which may be produced by enzymatic cleavage of an IgG by, for example, pepsin. Fab fragments may be produced by, for example, reduction of F(ab) 2 with dithiothreitol or mercaptoethylamine.

Immunoglobulins may be assigned to different classes depending on the amino acid sequences of the constant domain of their heavy chains. In some embodiments, different constant domains may be appended to humanized VL and VH regions derived from the CDRs provided herein. There are at least five major classes of immunoglobulins: IgA, IgD, IgE, IgG and IgM, and several of these may be further divided into subclasses (isotypes), e.g. IgG1, IgG2, IgG3 and IgG4; IgA1 and IgA2. The invention comprises antibodies and antigen-binding fragments of any of these classes or subclasses of antibodies.

In one embodiment, the antibody or antigen-binding fragment comprises a heavy chain constant region, e.g. a human constant region, such as γ1, γ2, 3, or γ4 human heavy chain constant region or a variant thereof. In another embodiment, the antibody or antigen-binding fragment comprises a light chain constant region, e.g. a human light chain constant region, such as lambda or kappa human light chain region or variant thereof. By way of example, and not limitation the human heavy chain constant region can be γ4 and the human light chain constant region can be kappa. In an alternative embodiment, the Fc region of the antibody is γ4 with a Ser228Pro mutation (Schuurman, J et. al., Mol. Immunol. 38:1-8, 2001).

In one embodiment, the antibody or antigen-binding fragment comprises a heavy chain constant region of the IgG1 subtype. In one embodiment, the antibody or antigen-binding fragment comprises a heavy chain constant region of the IgG2 subtype. In one embodiment, the antibody or antigen-binding fragment comprises a heavy chain constant region of the IgG4 subtype.

### Antibody Engineering

Further included are embodiments in which the anti-hLY6G6D antibodies and antigen-binding fragments thereof are engineered antibodies to include modifications to framework residues within the variable domains the antibody, e.g. to improve the properties of the antibody or fragment. Typically, such framework modifications are made to decrease the immunogenicity of the antibody or fragment. This is usually accomplished by replacing non-CDR residues in the variable domains (i.e. framework residues) in a parental (e.g. rodent) antibody or fragment with analogous residues from the immune repertoire of the species in which the antibody is to be used, e.g. human residues in the case of human therapeutics. Such an antibody or fragment is referred to as a “humanized” antibody or fragment. In some cases, it is desirable to increase the affinity, or alter the specificity of an engineered (e.g. humanized) antibody. One approach is to mutate one or more framework residues to the corresponding germline sequence. More specifically, an antibody or fragment that has undergone somatic mutation can contain framework residues that differ from the germline sequence from which the antibody is derived. Such residues can be identified by comparing the antibody or fragment framework sequences to the germline sequences from which the antibody or fragment is derived. Another approach is to revert to the original parental (e.g., rodent) residue at one or more positions of the engineered (e.g. humanized) antibody, e.g. to restore binding affinity that may have been lost in the process of replacing the framework residues. (See, e.g., U.S. Pat. Nos. 5,693,762, 5,585,089 and 5,530,101).

In certain embodiments, the anti-hLY6G6D antibodies and antigen-binding fragments thereof are engineered (e.g. humanized) to include modifications in the framework and/or CDRs to improve their properties. Such engineered changes can be based on molecular modelling. A molecular model for the variable region for the parental (non-human) antibody sequence can be constructed to understand the structural features of the antibody and used to identify potential regions on the antibody that can interact with the antigen. Conventional CDRs are based on alignment of immunoglobulin sequences and identifying variable regions. Kabat et al., (1991) Sequences of Proteins of Immunological Interest, Kabat, et al.; National Institutes of Health, Bethesda, MD; 5^{th} ed.; NIH Publ. No. 91-3242; Kabat (1978) Adv. Prot. Chem. 32:1-75; Kabat, et al., (1977) J. Biol. Chem. 252:6609-6616. Chothia and coworkers carefully examined conformations of the loops in crystal structures of antibodies and proposed hypervariable loops. Chothia, et al., (1987) J Mol. Biol. 196:901-917 or Chothia, et al., (1989) Nature 342:878-883. There are variations between regions classified as “CDRs” and “hypervariable loops”. Later studies (Raghunathan et al, (2012) J. Mol Recog. 25, 3, 103-113) analyzed several antibody-antigen crystal complexes and observed that the antigen binding regions in antibodies do not necessarily conform strictly to the “CDR” residues or “hypervariable” loops. The molecular model for the variable region of the non-human antibody can be used to guide the selection of regions that can potentially bind to the antigen. In practice the potential antigen binding regions based on the model differ from the conventional “CDR”s or “hypervariable” loops. Commercial scientific software such as Discovery Studio (BIOVIA, Dassault Systems)) can be used for molecular modeling. Human frameworks can be selected based on best matches with the non-human sequence both in the frameworks and in the CDRs. For FR4 (framework 4) in VH, VJ regions for the human germlines are compared with the corresponding non-human region. In the case of FR4 (framework 4) in VL, J-kappa and J-Lambda regions of human germline sequences are compared with the corresponding non-human region. Once suitable human frameworks are identified, the CDRs are grafted into the selected human frameworks. In some cases, certain residues in the VL-VH interface can be retained as in the non-human (parental) sequence. Molecular models can also be used for identifying residues that can potentially alter the CDR conformations and hence binding to antigen. In some cases, these residues are retained as in the non-human (parental) sequence. Molecular models can also be used to identify solvent exposed amino acids that can result in unwanted effects such as glycosylation, deamidation and oxidation. Developability filters can be introduced early on in the design stage to eliminate/minimize these potential problems.

Another type of framework modification involves mutating one or more residues within the framework region, or even within one or more CDR regions, to remove T cell epitopes to thereby reduce the potential immunogenicity of the antibody. This approach is also referred to as “deimmunization” and is described in further detail in U.S. Pat. No. 7,125,689.

In particular embodiments, it will be desirable to change certain amino acids containing exposed side-chains to another amino acid residue in order to provide for greater chemical stability of the final antibody, so as to avoid deamidation or isomerization. The deamidation of asparagine may occur on NG, DG, NG, NS, NA, NT, QG or QS sequences and result in the creation of an isoaspartic acid residue that introduces a kink into the polypeptide chain and decreases its stability (isoaspartic acid effect). Isomerization can occur at DG, DS, DA or DT sequences. In certain embodiments, the antibodies of the present disclosure do not contain deamidation or asparagine isomerism sites.

For example, an asparagine (Asn) residue may be changed to Gln or Ala to reduce the potential for formation of isoaspartate at any Asn-Gly sequences, particularly within a CDR. A similar problem may occur at a Asp-Gly sequence. Reissner and Aswad (2003) Cell. Mol. Life Sci. 60:1281. Isoaspartate formation may debilitate or completely abrogate binding of an antibody to its target antigen. See, Presta (2005) J. Allergy Clin. Immunol. 116:731 at 734. In one embodiment, the asparagine is changed to glutamine (Gln). It may also be desirable to alter an amino acid adjacent to an asparagine (Asn) or glutamine (Gln) residue to reduce the likelihood of deamidation, which occurs at greater rates when small amino acids occur adjacent to asparagine or glutamine. See, Bischoff & Kolbe (1994) J. Chromatog. 662:261. In addition, any methionine residues (typically solvent exposed Met) in CDRs may be changed to Lys, Leu, Ala, or Phe or other amino acids in order to reduce the possibility that the methionine sulfur would oxidize, which could reduce antigen-binding affinity and also contribute to molecular heterogeneity in the final antibody preparation. Id. Additionally, in order to prevent or minimize potential scissile Asn-Pro peptide bonds, it may be desirable to alter any Asn-Pro combinations found in a CDR to Gln-Pro, Ala-Pro, or Asn-Ala. Antibodies with such substitutions are subsequently screened to ensure that the substitutions do not decrease the affinity or specificity of the antibody for hLY6G6D, or other desired biological activity to unacceptable levels.

TABLE 3
Exemplary stabilizing CDR variants

| CDR Residue | Stabilizing Variant Sequence |
|---|---|
| Asn-Gly | Gln-Gly, Ala-Gly, or Asn-Ala |
| (N-G) | (Q-G), (A-G), or (N-A) |
| Asp-Gly | Glu-Gly, Ala-Gly or Asp-Ala |
| (D-G) | (E-G), (A-G), or (D-A) |
| Met | Lys, Leu, Ala, or Phe |
| (M) | (K), (L), (A), or (F) |
| Asn | Gln or Ala |
| (N) | (Q) or (A) |
| Asn-Pro | Gln-Pro, Ala-Pro, or Asn-Ala |
| (N-P) | (Q-P), (A-P), or (N-A) |
| Asn-Ser | Asn-Gly (N-G), Gly-Gly (G-G), |
| (N-S) | Ala-Ser (A-S), Asp-Ser (D-S) |

Another type of framework modification involves mutating one or more residues within the framework regions to prevent aggregation. The risk of an antibody to aggregate can be assessed using the spatial aggregation propensity. See, Chennamsetty, et al. (2010) J. Phys. Chem. 114, 6614-6624. The method requires the calculation of the Solvent Accessible Area (SAA) for each atom. The molecular aggregation score is then calculated as the sum of all atomic scores. For a given radius and size of molecule, this is an approximate indication of its overall tendency to aggregate. Residues with a high aggregation score are replaced by residues with a lower score (e.g. more hydrophilic amino acids).

### Antibody Engineering of the Fc Region

The antibodies (e.g., humanized antibodies) and antigen-binding fragments thereof disclosed herein can also be engineered to include modifications within the Fc region, typically to alter one or more properties of the antibody, such as serum half-life, complement fixation, Fc receptor binding, and/or effector function (e.g., antigen-dependent cellular cytotoxicity). Furthermore, the antibodies and antigen-binding fragments thereof disclosed herein can be chemically modified (e.g., one or more chemical moieties can be attached to the antibody) or be modified to alter its glycosylation, again to alter one or more properties of the antibody or fragment. Each of these embodiments is described in further detail below. The numbering of residues in the Fc region is that of the EU index of Kabat.

The antibodies and antigen-binding fragments thereof disclosed herein also include antibodies and fragments with modified (or blocked) Fc regions to provide altered effector functions. See, e.g., U.S. Pat. No. 5,624,821; WO2003/086310; WO2005/120571; WO2006/0057702. Such modifications can be used to enhance or suppress various reactions of the immune system, with possible beneficial effects in diagnosis and therapy. Alterations of the Fc region include amino acid changes (substitutions, deletions and insertions), glycosylation or deglycosylation, and adding multiple Fc regions. Changes to the Fc can also alter the half-life of antibodies in therapeutic antibodies, enabling less frequent dosing and thus increased convenience and decreased use of material. See Presta (2005) J. Allergy Clin. Immunol. 116:731 at 734-35.

In one embodiment, the antibody or antigen-binding fragment of the invention is an IgG4 isotype antibody or fragment comprising a Serine to Proline mutation at a position corresponding to position 228 (S228P; EU index) in the hinge region of the heavy chain constant region. This mutation has been reported to abolish the heterogeneity of inter-heavy chain disulfide bridges in the hinge region (Angal et al (1993). Mol. Immunol. 30:105-108; position 241 is based on the Kabat numbering system).

In one embodiment of the invention, the hinge region of CH1 is modified such that the number of cysteine residues in the hinge region is increased or decreased. This approach is described further in U.S. Pat. No. 5,677,425. The number of cysteine residues in the hinge region of CH1 is altered, for example, to facilitate assembly of the light and heavy chains or to increase or decrease the stability of the antibody.

In another embodiment, the Fc hinge region of an antibody or antigen-binding fragment of the invention is mutated to decrease the biological half-life of the antibody or fragment. More specifically, one or more amino acid mutations are introduced into the CH2-CH3 domain interface region of the Fc-hinge fragment such that the antibody or fragment has impaired Staphylococcyl protein A (SpA) binding relative to native Fc-hinge domain SpA binding. This approach is described in further detail in U.S. Pat. No. 6,165,745.

In another embodiment, the antibody or antigen-binding fragment of the invention is modified to increase its biological half-life. Various approaches are possible. For example, one or more of the following mutations can be introduced: T252L, T254S, T256F, as described in U.S. Pat. No. 6,277,375. Alternatively, to increase the biological half-life, the antibody can be altered within the CH1 or CL region to contain a salvage receptor binding epitope taken from two loops of a CH2 domain of an Fc region of an IgG, as described in U.S. Pat. Nos. 5,869,046 and 6,121,022.

In yet other embodiments, the Fc region is altered by replacing at least one amino acid residue with a different amino acid residue to alter the effector function(s) of the antibody or antigen-binding fragment. For example, one or more amino acids selected from amino acid residues 234, 235, 236, 237, 297, 318, 320 and 322 can be replaced with a different amino acid residue such that the antibody has an altered affinity for an effector ligand and retains the antigen-binding ability of the parent antibody. The effector ligand to which affinity is altered can be, for example, an Fc receptor or the C1 component of complement. This approach is described in further detail in U.S. Pat. Nos. 5,624,821 and 5,648,260.

In another example, one or more amino acids selected from amino acid residues 329, 331 and 322 can be replaced with a different amino acid residue such that the antibody has altered C1q binding and/or reduced or abolished complement dependent cytotoxicity (CDC). This approach is described in further detail in U.S. Pat. No. 6,194,551.

In another example, one or more amino acid residues within amino acid positions 231 and 239 are altered to thereby alter the ability of the antibody to fix complement. This approach is described further in PCT Publication WO 94/29351.

The proteins of the invention, which are preferably antibodies and most preferably IgG antibodies or fragments thereof, may have altered (e.g., relative to an unmodified antibody) FcγR binding properties (examples of binding properties include but are not limited to, binding specificity, equilibrium dissociation constant (K_{D}), dissociation and association rates (k_{off} and kₒₙ respectively), binding affinity and/or avidity) and that certain alterations are more or less desirable. It is known in the art that the equilibrium dissociation constant (K_{D}) is defined as k_{off}/kₒₙ, and Kₐ is the reciprocal of K_{D}.

The affinities and binding properties of an Fc region for its ligand, may be determined by a variety of in vitro assay methods (biochemical or immunological based assays) known in the art for determining Fc-FcγR interactions, i.e., specific binding of an Fc region to an FcγR including but not limited to, equilibrium methods (e.g., enzyme-linked immuno absorbent assay (ELISA) or radioimmunoassay (RIA)), or kinetics (e.g. BIACORE®, Octet®, or KinExa® analysis), and other methods such as indirect binding assays, competitive inhibition assays, fluorescence resonance energy transfer (FRET), gel electrophoresis and chromatography (e.g., gel filtration). These and other methods may utilize a label on one or more of the components being examined and/or employ a variety of detection methods including but not limited to chromogenic, fluorescent, luminescent, or isotopic labels.

In certain embodiments, the proteins of the present invention bind to one or more human FcγRs selected from the group consisting of FcγRI, FcγRIIB, FcγRIIC, FcγRIIIA-F158, and FcγRIIIA-V158 with an affinity at least 10-fold, preferably at least 30-fold, and more preferably at least 100-fold, less than equivalent protein having a wild-type human IgG1 heavy chain constant domain Fc region or a wild-type human IgG4 heavy chain constant domain Fc region.

In various embodiments, the proteins of the invention comprise an immunoglobulin Fc region comprising an immunoglobulin C2 region and an immunoglobulin C3 region and an immunoglobulin hinge region. By way of example, the immunoglobulin Fc region may be an IgG Fc region, an IgE Fc region, or an IgA Fc region. In certain preferred embodiments, the protein comprises two immunoglobulin Fc regions, each immunoglobulin Fc region comprising an immunoglobulin C2 region and an immunoglobulin C3 region and an immunoglobulin hinge region, wherein the hinge region of one of the immunoglobulin Fc regions is bound to the hinge region of the other immunoglobulin Fc region to form a dimeric Fc structure. Most preferably, such a protein is a human or humanized IgG protein.

In certain embodiments, the proteins of the invention comprise a mutated IgG4 Fc region, and preferably the protein is an IgG comprising two mutated IgG4 Fc regions to form a dimeric Fc structure. By way of example, a mutated IgG4 Fc region may comprise one of the mutations, or mutational combinations, recited in Table 3. The numbering system of the constant region referred to in this table is that of the EU index as set forth in Kabat et al. (1991, NIH Publication 91-3242, National Technical Information Service, Springfield, VA). In the table, the first letter and number represent the unmodified amino acid and its position and the second letter represents the substituted amino acid at said position. For those entries that include combinations of more than one mutation, each mutation in the combination is separated by a “/”. Deletions are indicated by “Δ.

TABLE 4

| | | |
|---|---|---|
| N297Q | L235E | N297Q/L235E |
| F234A | Q268A | F234A/L235A/G237A/ |
| | | P238A |
| F234A/L235A/ΔG236/ | F234A/L235A/G237A/ | F234A/L235A/ΔG236/ |
| G237A/P238A | P238A/Q268A | G237A/P238A/Q268A |
| F234A/L235A | L235E/P329G | L235A/G237A/E318A |
| F234A/L235A/G237A/ | F234A/L235A/ΔG236/ | F234A/L235A/G237A/ |
| P238S | G237A/P238S | P238S/Q268A |
| F234A/L235A/ΔG236/ | | |
| G237A/P238S/Q268A | | |

In certain embodiments, the proteins of the invention comprise a mutated IgG1 Fc region, and preferably the protein is an IgG comprising two mutated IgG1 Fc regions to form a dimeric Fc structure. By way of example, a mutated IgG1 Fc region may comprise one of the mutations recited in Table 4. The numbering system of the constant region referred to in this table is that of the EU index as set forth in Kabat et al. (1991, NIH Publication 91-3242, National Technical Information Service, Springfield, VA). In the table, the first letter and number represent the unmodified amino acid and its position and the second letter represents the substituted amino acid at said position.

TABLE 5

| | | |
|---|---|---|
| K222Y | P232K | A231K |
| E233N | E233Q | E233R |
| E233S | E233T | E233H |
| E233A | E233V | E233L |
| E233F | E233M | E233Y |
| E233W | E233G | L234D |
| L234E | L234N | L234Q |
| L234T | L234H | L234F |
| L234K | L234R | L234S |
| L234A | L234M | L234V |
| L235E | L235T | L235F |
| L235K | L235R | L235A |
| L235M | L235W | L235N |
| L235Q | L235H | L235V |
| G236A | G236N | G236R |
| G236H | G236L | G236F |
| G236P | G237A | G237E |
| G237N | G237Q | G237K |
| G237R | G237S | G237T |
| G237H | G237L | G237I |
| G237F | G237M | G237Y |
| G237P | P238K | P238N |
| P238R | P238S | P238T |
| P238Y | P238G | P238A |
| S239A | S239N | S239F |
| S239K | S239R | S239V |
| S239W | S239P | S239H |
| S239Y | D249H | V240A |
| F241W | F241L | F243W |
| F243L | F243E | P244H |
| P245A | P247V | P247G |
| V253I | V263I | V263T |
| V263M | V264D | V264E |
| V264K | V264F | V264M |
| V264H | V264W | V264G |
| V264Q | V264A | V264L |
| D265A | D265E | D265Q |
| D265S | D265H | D265V |
| D265L | D265F | D265M |
| D265Y | D265N | D265G |
| V266T | V266M | V266A |
| S267G | S267H | S267N |
| S267P | S267R | S267T |
| S267F | S267W | E269A |
| E269K | E269S | E269V |
| E269F | E269I | E269M |
| E269W | E269H | E269T |
| E269L | E269N | E269Y |
| E269R | E269P | E269G |
| D270A | D270N | D270E |
| D270Q | D270T | D270H |
| D270R | D270S | D270L |
| D270I | D270F | D270W |
| D270P | D270G | P271H |
| P271Q | P271K | P271R |
| P271S | P271V | P271F |
| P271W | D280L | D280W |
| D280P | E293F | E294A |
| E293Y | E294K | E294R |
| E294S | E294V | E294L |
| E294F | Q295A | Q295W |
| Q295P | Q295G | Y296E |
| Y296Q | Y296D | Y296N |
| Y296S | Y296T | Y296L |
| Y296I | Y296A | Y296V |
| Y296M | N297S | N297D |
| N297Q | N297A | S298T |
| S298N | S298K | S298R |
| T299A | T299H | T299D |
| T299E | T299N | T299Q |
| T299K | T299R | T299I |
| T299F | T299M | T299Y |
| T299W | T299S | T299V |
| T299P | T299G | Y300E |
| Y300K | Y300R | Y300S |
| Y300P | Y300W | V303A |
| V303D | W313F | E318A |
| E318V | E318Q | E318H |
| E318L | E318Y | K320A |
| K322A | K322E | N325A |
| N325V | N325H | N325K |
| N325Y | N325W | N325P |
| N325G | N325Q | N325D |
| N325E | N325L | N325I |
| A327Q | A327E | A327N |
| A327L | A327I | A327F |
| A327W | L328N | L328F |
| L328H | L328R | L328T |
| L328V | L328I | L328P |
| L328M | L328E | L328A |
| P329A | P329F | P329D |
| P329N | P329Q | P329K |
| P329S | P329T | P329H |
| P329V | P329L | P329M |
| P329Y | P329W | P329G |
| P329R | A330L | A330R |
| A330P | A330T | A330V |
| A330F | A330H | P331A |
| P331S | P331N | P331E |
| I332K | I332N | I332Q |
| I332T | I332H | I332Y |
| I332A | I332R | E333N |
| E333R | I336E | I336Y |
| S337H | | |

In certain embodiments, a mutated IgG1 Fc region may comprise one of the mutational combinations recited in Table 5. The numbering system of the constant region referred to in this table is that of the EU index as set forth in Kabat et al. (1991, NIH Publication 91-3242, National Technical Information Service, Springfield, VA). In the table, the first letter and number represent the unmodified amino acid and its position and the second letter represents the substituted amino acid at said position. For each of the combinations of more than one mutation, each mutation in the combination is separated by a “/” and deletions are indicated by a “Δ”.

TABLE 6

| | | |
|---|---|---|
| C220S/C226S/C229S/P238S | C226S/C229S/E233P/L234V/ | E233P/L234V/L235A |
| | L235A | |
| E233P/L234V/L235A/ΔG236 | E233P/L234V/L235A/ΔG236/ | L234A/L235A |
| | A327G/A330S/P331S | |
| L235A/G237A | L235A/G237A/E318S/K320S/ | L235A/G237A/P331A |
| | K322S | |
| L234F/L235E | L234F/L235E/D265A | L234F/L235E/D265A/N297Q/ |
| | | P331S |
| L234F/L235E/N297Q | L234F/L235E/P329G | L234F/L235A/K322Q/M252Y/ |
| | | S254T/T256E |
| L234F/L235Q/K322Q/M252Y/ | L234F/L235Q/P331G/M252Y/ | G236R/L328R |
| S254T/T256E | S254T/T256E | |
| S239D/D265I/N297D/1332E | S239D/D265L/N297D/I332E | S239D/D265F/N297D/I332E |
| S239D/D265Y/N297D/1332E | S239D/D265T/N297D/I332E | S239D/N297D/A330Y/I332E |
| S239D/F241S/F243H/V262T/ | V264E/N297D/I332E | D265A/P331S |
| V264T/N297D/K326E/I332E | | |
| D265A/N297Q | N297D/D265Y/T299L/I332E | N297D/D265Y/1332E |
| N297D/I332E/Y296D | N297D/I332E | N297D/I332E/Y296E |
| N297D/I332E/Y296N | N297D/I332E/Y296Q | N297D/I332E/Y296H |
| N297D/I332E/Y296T | N297D/I332E/T299V | N297D/I332E/T299I |
| N297D/I332E/T299L | N297D/I332E/T299F | N297D/I332E/T299H |
| N297D/I332E/T299E | N297D/I332E/A330Y | N297D/I332E/S298A/A330Y |
| N297E/D265F/I332E | N297E/I332E | F241E/F243R/V262E/V264R |
| F241E/F243Q/V262T/V264E | F241L/F243L/V262I/V264I | F241W/F243W |
| F241W/F243W/V262A/V264A | F241L/V262I | F243L/V262I/V264W |
| F241Y/F243Y/V262T/V264T | F241E/F243R/V262E/V264R | F241E/F243Q/V262T/V264E |
| F241R/F243Q/V262T/V264R | F241E/F243Y/V262T/V264R | P244H/P245A/P247V |
| F241E/F243R/V262E/V264R/ | F241E/F243Y/V262T/V264R | F241E/F243Y/V262T/V264R/ |
| I332E | | I332E |
| S239E/D265G | S239E/D265N | S239E/D265Q |
| M252Y/S254T/T256E | S267Q/A327S | S267L/A327S |
| N297S/I332E | S239N/I332N | S239N/I332Q |
| S239Q/I332N | S239Q/I332Q | S298N/Y300S |
| S298N/T299A/Y300S | N297Q/S298N/Y300S | E318S/K320S/K322S |
| E318S/K320S/K322S/P311A | L328E/I332E | L328N/I332E |
| L234A/L235A/G237A/P238A/ | L234A/L235A/G237A/P238S/ | L234A/L235A/G237A/P238A/ |
| H268A/A330S/P331S | H268A/A330S/P331S | H268A/A330S/P331S |
| L328Q/I332E | L328H/1332E | |

In certain embodiments, the proteins of the invention comprise a wild type or mutated IgG2 Fc region, and preferably the protein is an IgG comprising two wild type or mutated IgG2 Fc regions to form a dimeric Fc structure. A mutated IgG2 Fc region may comprise one of the mutations, or mutational combinations, recited in Table 6. The numbering system of the constant region referred to in this table is that of the EU index as set forth in Kabat et al. (1991, NIH Publication 91-3242, National Technical Information Service, Springfield, VA). In the table, the first letter and number represent the unmodified amino acid and its position and the second letter represents the substituted amino acid at said position. For those entries that include combinations of more than one mutation, each mutation in the combination is separated by a “/”.

TABLE 7

| | | |
|---|---|---|
| V234A | G237A | A235E/G237A |
| V234A/A235E/G237A | V234A/G237A | V234A/G237A/P238S |
| H268Q/V309L/A330S/P331S | V234A/G237A/H268A/V309L/ | V234A/G237A/H268Q/V309L/ |
| | A330S/P331S | A330S/P331S |
| V234A/G237A/P238S/H268A/ | P233S/V234A/G237A/P238S | P233S/V234A/G237A/H268A/ |
| V309L/A330S/P331S | | V309L/A330S/P331S |
| P233S/V234A/G237A/H268Q/ | P233S/V234A/G237A/P238S/ | |
| V309L/A330S/P331S | H268A/V309L/A330S/P331S | |

Production of Antibodies with Modified Glycosylation

In still another embodiment, the antibodies or antigen-binding fragments of the invention comprise a particular glycosylation pattern. For example, an afucosylated or an aglycosylated antibody or fragment can be made (i.e., the antibody lacks fucose or glycosylation, respectively). The glycosylation pattern of an antibody or fragment may be altered to, for example, increase the affinity or avidity of the antibody or fragment for a hLY6G6D antigen. Such modifications can be accomplished by, for example, altering one or more of the glycosylation sites within the antibody or fragment sequence. For example, one or more amino acid substitutions can be made that result in removal of one or more of the variable region framework glycosylation sites to thereby eliminate glycosylation at that site. Such deglycosylation may increase the affinity or avidity of the antibody or fragment for antigen. See, e.g., U.S. Pat. Nos. 5,714,350 and 6,350,861.

Antibodies and antigen-binding fragments disclosed herein may further include those produced in lower eukaryote host cells, in particular fungal host cells such as yeast and filamentous fungi have been genetically engineered to produce glycoproteins that have mammalian- or human-like glycosylation patterns (See for example, Choi et al, (2003) Proc. Natl. Acad. Sci. 100:5022-5027; Hamilton et al., (2003) Science 301:1244-1246; Hamilton et al., (2006) Science 313:1441-1443; Nett et al., Yeast 28 (3): 237-52 (2011); Hamilton et al., Curr Opin Biotechnol. 18 (5): 387-92 (2007)). A particular advantage of these genetically modified host cells over currently used mammalian cell lines is the ability to control the glycosylation profile of glycoproteins that are produced in the cells such that compositions of glycoproteins can be produced wherein a particular N-glycan structure predominates (see, e.g., U.S. Pat. Nos. 7,029,872 and 7,449,308). These genetically modified host cells have been used to produce antibodies that have predominantly particular N-glycan structures (See for example, Li et al., (2006) Nat. Biotechnol. 24:210-215).

In particular embodiments, the antibodies and antigen-binding fragments thereof disclosed herein further include those produced in lower eukaryotic host cells and which comprise fucosylated and non-fucosylated hybrid and complex N-glycans, including bisected and multiantennary species, including but not limited to N-glycans such as GlcNAc₍₁₋₄₎Man₃GlcNAc₂; Gal₍₁₋₄₎GlcNAc₍₁₋₄₎Man₃GlcNAc₂; NANA₍₁₋₄₎Gal₍₁₋₄₎GlcNAc₍₁₋₄₎Man₃GlcNAc₂.

In particular embodiments, the antibodies and antigen-binding fragments thereof provided herein may comprise antibodies or fragments having at least one hybrid N-glycan selected from the group consisting of GlcNAcMan₅GlcNAc₂; GalGlcNAcMan₅GlcNAc₂; and NANAGalGlcNAcMan₅GlcNAc₂. In particular aspects, the hybrid N-glycan is the predominant N-glycan species in the composition.

In particular embodiments, the antibodies and antigen-binding fragments thereof provided herein comprise antibodies and fragments having at least one complex N-glycan selected from the group consisting of GlcNAcMan₃GlcNAc₂; GalGlcNAcMan₃GlcNAc₂; NANAGalGlcNAcMan₃GlcNAc₂; GlcNAc₂Man₃GlcNAc₂; GalGlcNAc₂Man₃GlcNAc₂; Gal₂GlcNAc₂Man₃GlcNAc₂; NANAGal₂GlcNAc₂Man₃GlcNAc₂; and NANA₂Gal₂GlcNAc₂Man₃GlcNAc₂. In particular aspects, the complex N-glycan are the predominant N-glycan species in the composition. In further aspects, the complex N-glycan is a particular N-glycan species that comprises about 30%, 40%, 50%, 60%, 70%, 80%, 90%, 95%, 97%, 98%, 99%, or 100% of the complex N-glycans in the composition. In one embodiment, the antibody and antigen binding fragments thereof provided herein comprise complex N-glycans, wherein at least 50%, 60%, 70%, 80%, 90%, 95%, 97%, 98%, 99%, or 100% of the complex N-glycans comprise the structure NANA₂Gal₂GlcNAc₂Man₃GlcNAc₂, wherein such structure is afucosylated. Such structures can be produced, e.g., in engineered Pichia pastoris host cells.

In particular embodiments, the N-glycan is fucosylated. In general, the fucose is in an α1,3-linkage with the GlcNAc at the reducing end of the N-glycan, an α1,6-linkage with the GlcNAc at the reducing end of the N-glycan, an α1,2-linkage with the Gal at the non-reducing end of the N-glycan, an α1,3-linkage with the GlcNac at the non-reducing end of the N-glycan, or an α1,4-linkage with a GlcNAc at the non-reducing end of the N-glycan.

Therefore, in particular aspects of the above the glycoprotein compositions, the glycoform is in an α1,3-linkage or α1,6-linkage fucose to produce a glycoform selected from the group consisting of Man₅GlcNAc₂(Fuc), GlcNAcMan₅GlcNAc₂(Fuc), Man₃GlcNAC₂(Fuc), GlcNAcMan₃GlcNAC₂(Fuc), GlcNAc₂Man₃GlcNAc₂(Fuc), GalGlcNAc₂Man₃GlcNAC₂(Fuc), Gal₂GlcNAc₂Man₃GlcNAC₂(Fuc), NANAGal₂GlcNAc₂Man₃GlcNAC₂(Fuc), and NANA₂Gal₂GlcNAc₂Man₃GlcNAc₂(Fuc); in an α1,3-linkage or α1,4-linkage fucose to produce a glycoform selected from the group consisting of GlcNAc(Fuc)MansGlcNAc₂, GlcNAc(Fuc)Man₃GlcNAc₂, GlcNAc₂(Fuc₁₋₂)Man₃GlcNAc₂, GalGlcNAC₂(Fuc₁₋₂)Man₃GlcNAc₂, Gal₂GlcNAc₂(Fuc₁₋₂)Man₃GlcNAc₂, NANAGal₂GlcNAc₂(Fuc₁₋₂)Man₃GlcNAc₂, and NANA₂Gal₂GlcNAC₂(Fuc₁₋₂)Man₃GlcNAc₂; or in an a_{1,2}-linkage fucose to produce a glycoform selected from the group consisting of Gal(Fuc)GlcNAc₂Man₃GlcNAc₂, Gal₂(Fuc₁₋₂) GlcNAc₂Man₃GlcNAc₂, NANAGal₂(Fuc₁₋₂) GlcNAc₂Man₃GlcNAc₂, and NANA₂Gal₂(Fuc₁₋₂) GlcNAc₂Man₃GlcNAC₂.

In further aspects, the antibodies (e.g., humanized antibodies) or antigen-binding fragments thereof comprise high mannose N-glycans, including but not limited to, Man₈GlcNAC₂, Man₇GlcNAc₂, Man₆GlcNAc₂, Man₅GlcNAc₂, Man₄GlcNAc₂, or N-glycans that consist of the Man₃GlcNAc₂ N-glycan structure.

In further aspects of the above, the complex N-glycans further include fucosylated and non-fucosylated bisected and multiantennary species.

As used herein, the terms “N-glycan” and “glycoform” are used interchangeably and refer to an N-linked oligosaccharide, for example, one that is attached by an asparagine-N-acetylglucosamine linkage to an asparagine residue of a polypeptide. N-linked glycoproteins contain an N-acetylglucosamine residue linked to the amide nitrogen of an asparagine residue in the protein. The predominant sugars found on glycoproteins are glucose, galactose, mannose, fucose, N-acetylgalactosamine (GalNAc), N-acetylglucosamine (GlcNAc) and sialic acid (e.g., N-acetyl-neuraminic acid (NANA)). The processing of the sugar groups occurs co-translationally in the lumen of the ER and continues post-translationally in the Golgi apparatus for N-linked glycoproteins.

N-glycans have a common pentasaccharide core of Man₃GlcNAc₂ (“Man” refers to mannose; “Glc” refers to glucose; and “NAc” refers to N-acetyl; GlcNAc refers to N-acetylglucosamine). Usually, N-glycan structures are presented with the non-reducing end to the left and the reducing end to the right. The reducing end of the N-glycan is the end that is attached to the Asn residue comprising the glycosylation site on the protein. N-glycans differ with respect to the number of branches (antennae) comprising peripheral sugars (e.g., GlcNAc, galactose, fucose and sialic acid) that are added to the Man₃GlcNAC₂ (“Man3”) core structure which is also referred to as the “trimannose core”, the “pentasaccharide core” or the “paucimannose core”. N-glycans are classified according to their branched constituents (e.g., high mannose, complex or hybrid). A “high mannose” type N-glycan has five or more mannose residues. A “complex” type N-glycan typically has at least one GlcNAc attached to the 1,3 mannose arm and at least one GlcNAc attached to the 1,6 mannose arm of a “trimannose” core. Complex N-glycans may also have galactose (“Gal”) or N-acetylgalactosamine (“GalNAc”) residues that are optionally modified with sialic acid or derivatives (e.g., “NANA” or “NeuAc”, where “Neu” refers to neuraminic acid and “Ac” refers to acetyl). Complex N-glycans may also have intrachain substitutions comprising “bisecting” GlcNAc and core fucose (“Fuc”). Complex N-glycans may also have multiple antennae on the “trimannose core,” often referred to as “multiple antennary glycans.” A “hybrid” N-glycan has at least one GlcNAc on the terminal of the 1,3 mannose arm of the trimannose core and zero or more mannoses on the 1,6 mannose arm of the trimannose core. The various N-glycans are also referred to as “glycoforms.”

With respect to complex N-glycans, the terms “G-2”, “G-1”, “G0”, “G1”, “G2”, “A1”, and “A2” mean the following. “G-2” refers to an N-glycan structure that can be characterized as Man₃ GlcNAc₂; the term “G-1” refers to an N-glycan structure that can be characterized as GlcNAcMan₃GlcNAc₂; the term “G0” refers to an N-glycan structure that can be characterized as GlcNAc₂Man₃GlcNAc₂; the term “G1” refers to an N-glycan structure that can be characterized as GalGlcNAc₂Man₃GlcNAc₂; the term “G2” refers to an N-glycan structure that can be characterized as Gal₂GlcNAc₂Man₃GlcNAc₂; the term “A1” refers to an N-glycan structure that can be characterized as NANAGal₂GlcNAc₂Man₃GlcNAc₂; and, the term “A2” refers to an N-glycan structure that can be characterized as NANA₂Gal₂GlcNAc₂Man₃GlcNAc₂. Unless otherwise indicated, the terms G-2”, “G-1”, “G0”, “G1”, “G2”, “A1”, and “A2” refer to N-glycan species that lack fucose attached to the GlcNAc residue at the reducing end of the N-glycan. When the term includes an “F”, the “F” indicates that the N-glycan species contains a fucose residue on the GlcNAc residue at the reducing end of the N-glycan. For example, G0F, G1F, G2F, A1F, and A2F all indicate that the N-glycan further includes a fucose residue attached to the GlcNAc residue at the reducing end of the N-glycan. Lower eukaryotes such as yeast and filamentous fungi do not normally produce N-glycans that produce fucose.

With respect to multiantennary N-glycans, the term “multiantennary N-glycan” refers to N-glycans that further comprise a GlcNAc residue on the mannose residue comprising the non-reducing end of the 1,6 arm or the 1,3 arm of the N-glycan or a GlcNAc residue on each of the mannose residues comprising the non-reducing end of the 1,6 arm and the 1,3 arm of the N-glycan. Thus, multiantennary N-glycans can be characterized by the formulas GlcNAc₍₂₋₄₎Man₃GlcNAc₂, Gal₍₁₋₄₎GlcNAc₍₂₋₄)Man₃GlcNAc₂, or NANA₍₁₋₄₎Gal₍₁₋₄₎GlcNAc₍₂₋₄₎Man₃GlcNAc₂. The term “1-4” refers to 1, 2, 3, or 4 residues.

With respect to bisected N-glycans, the term “bisected N-glycan” refers to N-glycans in which a GlcNAc residue is linked to the mannose residue at the reducing end of the N-glycan. A bisected N-glycan can be characterized by the formula GlcNAc₃Man₃GlcNAc₂ wherein each mannose residue is linked at its non-reducing end to a GlcNAc residue. In contrast, when a multiantennary N-glycan is characterized as GlcNAc₃Man₃GlcNAc₂, the formula indicates that two GlcNAc residues are linked to the mannose residue at the non-reducing end of one of the two arms of the N-glycans and one GlcNAc residue is linked to the mannose residue at the non-reducing end of the other arm of the N-glycan.

In certain embodiments, the proteins of the invention comprise an aglycosylated Fc region. By way of example, an IgG1 Fc region may be aglycosylayed by deleting or substituting residue N297.

### Antibody Physical Properties

The antibodies and antigen-binding fragments thereof disclosed herein may further contain one or more glycosylation sites in either the light or heavy chain immunoglobulin variable region. Such glycosylation sites may result in increased immunogenicity of the antibody or fragment or an alteration of the pK of the antibody due to altered antigen-binding (Marshall et al. (1972) Annu Rev Biochem 41:673-702; Gala and Morrison (2004) J Immunol 172:5489-94; Wallick et al (1988) J Exp Med 168:1099-109; Spiro (2002) Glycobiology 12: 43R-56R; Parekh et al (1985) Nature 316:452-7; Mimura et al. (2000) Mol Immunol 37:697-706). Glycosylation has been known to occur at motifs containing an N-X-S/T sequence.

Each antibody or antigen-binding fragment will have a unique isoelectric point (pI), which generally falls in the pH range between 6 and 9.5. The pI for an IgG1 antibody typically falls within the pH range of 7-9.5 and the pI for an IgG4 antibody typically falls within the pH range of 6-8.

Each antibody or antigen-binding fragment will have a characteristic melting temperature, with a higher melting temperature indicating greater overall stability in vivo (Krishnamurthy R and Manning MC (2002) Curr Pharm Biotechnol 3:361-71). In general, the T_{M1} (the temperature of initial unfolding) may be greater than 60° C., greater than 65° C., or greater than 70° C. The melting point of an antibody or fragment can be measured using differential scanning calorimetry (Chen et al (2003) Pharm Res 20:1952-60; Ghirlando et al (1999) Immunol Lett 68:47-52) or circular dichroism (Murray et al. (2002) J. Chromatogr Sci 40:343-9).

In a further embodiment, antibodies and antigen-binding fragments thereof are selected that do not degrade rapidly. Degradation of an antibody or fragment can be measured using capillary electrophoresis (CE) and MALDI-MS (Alexander A J and Hughes D E (1995) Anal Chem 67:3626-32).

In a further embodiment, antibodies and antigen-binding fragments thereof are selected that have minimal aggregation effects, which can lead to the triggering of an unwanted immune response and/or altered or unfavorable pharmacokinetic properties. Generally, antibodies and fragments are acceptable with aggregation of 25% or less, 20% or less, 15% or less, 10% or less, or 5% or less. Aggregation can be measured by several techniques, including size-exclusion column (SEC), high performance liquid chromatography (HPLC), and light scattering.

### Antibody Conjugates

The anti-LY6G6D antibodies and antigen-binding fragments thereof disclosed herein may also be conjugated to a chemical moiety. The chemical moiety may be, inter alia, a polymer, a radionucleotide or a cytotoxic factor. In particular embodiments, the chemical moiety is a polymer which increases the half-life of the antibody or fragment in the body of a subject. Suitable polymers include, but are not limited to, hydrophilic polymers which include but are not limited to polyethylene glycol (PEG) (e.g., PEG with a molecular weight of 2 kDa, 5 kDa, 10 kDa, 12 kDa, 20 kDa, 30 kDa or 40 kDa), dextran and monomethoxypolyethylene glycol (mPEG). Lee, et al., (1999) (Bioconj. Chem. 10:973-981) discloses PEG conjugated single-chain antibodies. Wen, et al., (2001) (Bioconj. Chem. 12:545-553) disclose conjugating antibodies with PEG which is attached to a radiometal chelator (diethylenetriaminpentaacetic acid (DTPA)).

The antibodies and antigen-binding fragments thereof disclosed herein may also be conjugated with labels such as ⁹⁹Tc, ^{99m}Tc, ⁸⁶Y, ⁸⁸Y, ⁹⁰Y, ¹¹¹In, ³²P, ¹⁴C, ¹²³I, ¹²⁴I, ¹²⁵I, ³H, ¹³¹I, ¹¹C, ¹⁵O, ¹³N, ¹⁸F, ¹⁹F, ³⁵S, ⁵¹Cr, ⁵⁷To, ²²⁶Ra, ⁶⁰Co, ⁵⁹Fe, ⁵⁷Se, ¹⁵²Eu, ⁶¹Cu, ⁶²Cu, ⁶⁴Cu, ⁶⁷Cu, ⁶⁶Ga, ⁶⁷Ga, ⁶⁸Ga, ⁷²Ga, ⁴⁵Ti, ⁸⁹Zr, ²¹⁷Ci, ²¹¹ At, ²¹²Pb, ¹⁷⁷Lu, ⁴⁴Sc, ⁴⁷Sc, ¹⁰⁹Pd, ²³⁴Th, and ⁴⁰K, ¹⁵⁷Gd, ⁵⁵Mn, ⁵²Tr, and ⁵⁶Fe.

The antibodies and antigen-binding fragments disclosed herein may also be PEGylated, for example to increase its biological (e.g., serum) half-life. To PEGylate an antibody or fragment, the antibody or fragment, typically is reacted with a reactive form of polyethylene glycol (PEG), such as a reactive ester or aldehyde derivative of PEG, under conditions in which one or more PEG groups become attached to the antibody or antibody fragment. In particular embodiments, the PEGylation is carried out via an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (C1-C10) alkoxy- or aryloxy-polyethylene glycol or polyethylene glycol-maleimide. In certain embodiments, the antibody or fragment to be PEGylated is an aglycosylated antibody or fragment. Methods for PEGylating proteins are known in the art and can be applied to the antibodies of the invention. See, e.g., EP 0 154 316 and EP 0 401 384.

The antibodies and antigen-binding fragments disclosed herein may also be conjugated with fluorescent or chemilluminescent labels, including fluorophores such as rare earth chelates, fluorescein and its derivatives, rhodamine and its derivatives, isothiocyanate, phycoerythrin, phycocyanin, allophycocyanin, o-phthaladehyde, fluorescamine, ¹⁵²Eu, dansyl, umbelliferone, luciferin, luminal label, isoluminal label, an aromatic acridinium ester label, an imidazole label, an acridimium salt label, an oxalate ester label, an aequorin label, 2,3-dihydrophthalazinediones, biotin/avidin, spin labels and stable free radicals.

The antibodies and antigen-binding fragments thereof of the invention may also be conjugated to a cytotoxic factor such as diptheria toxin, Pseudomonas aeruginosa exotoxin A chain, ricin A chain, abrin A chain, modeccin A chain, alpha-sarcin, Aleurites fordii proteins and compounds (e.g., fatty acids), dianthin proteins, Phytoiacca americana proteins PAPI, PAPII, and PAP-S, Momordica charantia inhibitor, curcin, crotin, Saponaria officinalis inhibitor, mitogellin, restrictocin, phenomycin, and enomycin.

The antibodies and antigen-binding fragments herein may be detectably labeled using paramagnetic chelates, microparticles, superparamagnetic particles; incorporated into ultrasound bubbles, microparticles, microspheres, emulsions, etc.

A metal chelator(s) is a molecule having one or more polar groups that act as a ligand for, and complex with, a paramagnetic metal. Suitable chelators are known in the art and include acids with methylene phosphonic acid groups, methylene carbohydroxamine acid groups, carboxyethylidene groups, or carboxymethylene groups. Examples of chelators include, but are not limited to, diethylenetriaminepentaacetic acid (DTPA), 1,4,7,10-tetraazacyclo-tetradecane-1,4,7,10-tetraacetic acid (DOTA), 1-substituted 1,4,7,-tricarboxymethyl-1,4,7,10-teraazacyclododecane (DO3A), ethylenediaminetetraacetic acid (EDTA), and 1,4,8,11-tetra-azacyclotetradecane-1,4,8,11-tetraacetic acid (TETA). Additional chelating ligands are ethylene bis-(2-hydroxy-phenylglycine) (EHPG), and derivatives thereof, including 5-C1-EHPG, 5Br-EHPG, 5-Me-EHPG, 5t-Bu-EHPG, and 5 sec-Bu-EHPG; benzodiethylenetriamine pentaacetic acid (benzo-DTPA) and derivatives thereof, including dibenzo-DTPA, phenyl-DTPA, diphenyl-DTPA, benzyl-DTPA, and dibenzyl DTPA; bis-2 (hydroxybenzyl)-ethylene-diaminediacetic acid (HBED) and derivatives thereof; the class of macrocyclic compounds, which contain at least 3 carbon atoms, more preferably at least 6, and at least two heteroatoms (O and/or N), which macrocyclic compounds can consist of one ring, or two or three rings joined together at the hetero ring elements, e.g., benzo-DOTA, dibenzo-DOTA, and benzo-NOTA, where NOTA is 1,4,7-triazacyclononane N,N′,N″-triacetic acid, benzo-TETA, benzo-DOTMA, where DOTMA is 1,4,7,10-tetraazacyclotetradecane-1,4,7,10-tetra(methyl tetraacetic acid), and benzo-TETMA, where TETMA is 1,4,8,11-tetraazacyclotetradecane-1,4,8,11-(methyl tetraacetic acid); derivatives of 1,3-propylene-diaminetetraacetic acid (PDTA) and triethylenetetraaminehexaacetic acid (TTHA); derivatives of 1,5,10-N,N′,N″-tris(2,3-dihydroxybenzoyl)-tricatecholate (LICAM); and 1,3,5-N,N′,N″-tris(2,3-dihydroxybenzoyl)aminomethylbenzene (MECAM). Examples of representative chelators and chelating groups contemplated by the present invention are described in WO 98/18496, WO 86/06605, WO 91/03200, WO 95/28179, WO 96/23526, WO 97/36619, PCT/US98/01473, PCT/US98/20182, and U.S. Pat. Nos. 4,899,755, 5,474,756, 5,846,519 and 6,143,274, all of which are hereby incorporated by reference.

Any method known in the art for conjugating the antibodies and antigen-binding fragments thereof of the invention to the various moieties may be employed, including those methods described by Hunter, et al., (1962) Nature 144:945; David, et al., (1974) Biochemistry 13:1014; Pain, et al., (1981) J. Immunol. Meth. 40:219; and Nygren, J., (1982) Histochem. and Cytochem. 30:407. Methods for conjugating antibodies and fragments are conventional and very well known in the art.

Chemical cross-linkers may be classified on the basis of the following:
1. Functional groups and chemical specificity;
2. length and composition of the cross-bridge;
3. whether the cross-linking groups are similar (homobifunctional) or different (heterobifunctional);
4. whether the groups react chemically or photochemically;
5. whether the reagent is cleavable; and
6. whether the reagent can be radiolabeled or tagged with another label.

Reactive groups on antibodies and labels that can be targeted using a cross-linker include primary amines, carbonyls, carbohydrates and carboxylic acids. In addition, many reactive groups can be coupled nonselectively using a cross-linker such as photoreactive phenyl azides. For suitable reagents, see Pierce 2003-2004 Applications Handbook and Catalog #1600926, which is hereby incorporated by reference.

Many factors must be considered to determine optimum cross-linker-to-target molar ratios. Depending on the application, the degree of conjugation is an important factor. For example, when preparing immunogen conjugates, a high degree of conjugation is normally desired to increase the immunogenicity of the antigen. However, when conjugating to an antibody or an enzyme, a low-to-moderate degree of conjugation may be optimal to ensure that the biological activity of the protein is retained. It is also important to consider the number of reactive groups on the surface of the protein. If there are numerous target groups, a lower cross-linker-to-protein ratio can be used. For a limited number of potential targets, a higher cross-linker-to-protein ratio may be required. This translates into more cross-linker per gram for a small molecular weight protein.

Conformational changes of proteins associated with a particular interaction may also be analyzed by performing cross-linking studies before and after the interaction. A comparison is made by using different arm-length cross-linkers and analyzing the success of conjugation. The use of cross-linkers with different reactive groups and/or spacer arms may be desirable when the conformation of the protein changes such that hindered amino acids become available for cross-linking.

Cross-linkers are available with varying lengths of spacer arms or bridges connecting the reactive ends. The most apparent attribute of the bridge is its ability to deal with steric considerations of the moieties to be linked. Because steric effects dictate the distance between potential reaction sites for cross-linking, different lengths of bridges may be considered for the interaction. Shorter spacer arms are often used in intramolecular cross-linking studies, while intermolecular cross-linking is favored with a cross-linker containing a longer spacer arm.

The inclusion of polymer portions (e.g., polyethylene glycol (“PEG”) homopolymers, polypropylene glycol homopolymers, other alkyl-polyethylene oxides, bis-polyethylene oxides and co-polymers or block co-polymers of poly(alkylene oxides)) in cross-linkers can, under certain circumstances be advantageous. See, e.g., U.S. Pat. Nos. 5,643,575, 5,672,662, 5,705,153, 5,730,990, 5,902,588, and 5,932,462; and Topchieva et al., Bioconjug. Chem. 6:380-8, 1995). For example, U.S. Pat. No. 5,672,662 discloses bifunctional cross-linkers comprising a PEG polymer portion and a single ester linkage. Such molecules are said to provide a half-life of about 10 to 25 minutes in water.

Designing a cross-linker involves selection of the functional moieties to be employed. The choice of functional moieties is entirely dependent upon the target sites available on the species to be crosslinked. Some species (e.g., proteins) may present a number of available sites for targeting (e.g., lysine ¿-amino groups, cysteine sulfhydryl groups, glutamic acid carboxyl groups, etc.), and selection of a particular functional moiety may be made empirically in order to best preserve a biological property of interest (e.g., binding affinity of an antibody, catalytic activity of an enzyme, etc.)

### Coupling Through Amine Groups

Imidoester and N-hydroxysuccinimidyl (“NHS”) esters are typically employed as amine-specific functional moieties. NHS esters yield stable products upon reaction with primary or secondary amines. Coupling is efficient at physiological pH, and NHS-ester cross-linkers are more stable in solution than their imidate counterparts. Homobifunctional NHS-ester conjugations are commonly used to cross-link amine-containing proteins in either one-step or two-step reactions. Primary amines are the principal targets for NHS-esters. Accessible α-amine groups present on the N-termini of proteins react with NHS-esters to form amides. However, because α-amines on a protein are not always available, the reaction with side chains of amino acids become important. While five amino acids have nitrogen in their side chains, only the ε-amino group of lysine reacts significantly with NHS-esters. A covalent amide bond is formed when the NHS-ester cross-linking agent reacts with primary amines, releasing N-hydroxysuccinimide.

### Coupling Through Sulfhydryl Groups

Maleimides, alkyl and aryl halides, α-haloacyls, and pyridyl disulfides are typically employed as sulfhydryl-specific functional moieties. The maleimide group is specific for sulfhydryl groups when the pH of the reaction mixture is kept between pH 6.5 and 7.5. At pH 7, the reaction of the maleimides with sulfhydryls is 1000-fold faster than with amines. Maleimides do not react with tyrosines, histidines or methionines. When free sulfhydryls are not present in sufficient quantities, they can often be generated by reduction of available disulfide bonds.

### Coupling Through Carboxyl Groups

Carbodiimides couple carboxyls to primary amines or hydrazides, resulting in formation of amide or hydrazone bonds. Carbodiimides are unlike other conjugation reactions in that no cross-bridge is formed between the carbodiimide and the molecules being coupled; rather, a peptide bond is formed between an available carboxyl group and an available amine group. Carboxy termini of proteins can be targeted, as well as glutamic and aspartic acid side chains. In the presence of excess cross-linker, polymerization may occur because proteins contain both carboxyls and amines. No cross-bridge is formed, and the amide bond is the same as a peptide bond, so reversal of the cross-linking is impossible without destruction of the protein.

### Nonselective Labeling

A photoaffinity reagent is a compound that is chemically inert but becomes reactive when exposed to ultraviolet or visible light. Arylazides are photoaffinity reagents that are photolyzed at wavelengths between 250-460 nm, forming a reactive aryl nitrene. The aryl nitrene reacts nonselectively to form a covalent bond. Reducing agents must be used with caution because they can reduce the azido group.

### Carbonyl Specific Cross-Linkers

Carbonyls (aldehydes and ketones) react with amines and hydrazides at pH 5-7. The reaction with hydrazides is faster than with amines, making this useful for site-specific cross-linking. Carbonyls do not readily exist in proteins; however, mild oxidation of sugar moieties using sodium metaperiodate will convert vicinal hydroxyls to aldehydes or ketones.

### Additional Immunoglobulin Variable Regions

The target binding proteins of the invention may comprise one or more immunoglobulin variable regions in addition to the hLY6G6D-binding domain described herein. In some embodiments, the target binding protein may comprise an additional light chain variable domain and an additional heavy chain variable domain. The additional light chain variable domain and the additional heavy chain variable domain may form an additional immunoglobulin variable region. Tn some examples, the hLY6G6D-binding domain and the additional immunoglobulin variable region may be identical. In some examples, the HLY6G6D-binding domain and the additional immunoglobulin variable region may be different from each other (e.g., may specifically bind to the same or different antigens or epitopes).

In some embodiments, the hLY6G6D-binding domain and the additional immunoglobulin variable region may be both Fv fragments, or at least one may be a Fv fragment. In some embodiments, the hLY6G6D-binding domain and the additional immunoglobulin variable region may be both Fab fragments, or at least one may be a Fab fragment. In some embodiment, the hLY6G6D-binding domain and an additional immunoglobulin variable region may be a Fab′ fragment, or at least one can be a Fab′ fragment.

In some embodiments, the target binding protein may be multispecific (e.g., bispecific, trispecific, tetraspecific, and other multispecific target binding proteins), e.g., binding to hLY6G6D and one or more additional targets. In some embodiments, the multispecific target binding protein may be multivalent, e g, comprising multiple target binding sites regardless of whether the binding sites recognize the same or different targets. In some embodiment, the target binding protein may be bispecific. The term “bispecific” means that target binding protein is able to specifically bind to two distinct targets. Typically, a bispecific target binding protein comprises two immunoglobulin variable regions, each of which is capable of specifically binding to a different target. In some embodiments, the bispecific target binding protein may be capable of simultaneously binding two targets, e.g., two target proteins expressed on two distinct cells.

In some embodiments, the target binding protein may comprise the hLY6G6D-binding domain and an additional immunoglobulin variable region capable of binding to a molecule on the surface of a cell associated with a disease (e.g., a tumor cell). Such target binding proteins may simultaneously bind to an immune cell (e.g., T cell) and a cell associated with a disease (e.g., a tumor cell), thus activating the immune cell and crosslinking the activated immune cell to the cell associated with the disease. In some embodiments, the target binding protein may be formulated as part of chimeric antigen receptor (CAR), a T cell engaging antibody (e.g., bispecific T cell engaging antibody or BiTE), a pro-Bispecific T Cell Engager (pro-BiTE) molecule, pro-Chimeric Antigen Receptor (pro-CAR) modified T cell, or other engineered receptor or other immune effector cell, such as a CAR modified NK cell.

In some examples, the target binding protein may be a monovalent bispecific antibody comprising the hLY6G6D-binding immunoglobulin variable region and an additional immunoglobulin variable region described herein. As used herein, the term “monovalent bispecific antibody” refers to a bispecific antibody, in which only one antigen-binding domain is directed against a given target. The inventors have surprisingly discovered that certain hLY6G6D-binding domains described herein display improved stability, manufacturability, and/or hLY6G6D binding affinity in the context of a monovalent hLY6G6D binding protein, including monovalent bispecific antibodies that specifically bind hLY6G6D (e.g., an anti-hLY6G6D scFv).

The target of the additional immunoglobulin variable region may be a protein or other type of molecules, e.g., cell surface receptors and secreted binding proteins (e.g., growth factors), soluble enzymes, structural proteins (e.g. collagen, fibronectin) and the like.

In some examples, the additional immunoglobulin variable region may bind to a target that is a molecule on or inside a cell that is associated with a disease. For example, the additional immunoglobulin variable region may bind to a tumor cell. In such cases, the additional immunoglobulin variable region may bind to a tumor associated antigen. As used herein, the term “tumor associated antigen” refers to any antigen including a protein, glycoprotein, ganglioside, carbohydrate, lipid that is associated with cancer. Such antigen may be expressed on tumor cells (e.g., malignant cells) or in the tumor microenvironment such as on tumor-associated blood vessels, extracellular matrix, mesenchymal stroma, or immune infiltrates. In some embodiments, the tumor associated antigen may be human epidermal growth factor receptor 2 (HER2). For example, the additional target binding domain may be trastuzumab or a fragment thereof. In another example, the additional immunoglobulin variable region may be pertuzumab or a fragment thereof.

Many techniques for making bispecific antibodies are known to those skilled in the art. In some embodiments, a bispecific antibody comprises heavy chain constant regions with modifications in the amino acids that are part of the interface between the two heavy chains. These modifications are made to enhance heterodimer formation and generally reduce or eliminate homodimer formation. In some embodiments, the bispecific antibody is generated using a knobs-into-holes (KIH) strategy. In some embodiments, the bispecific antibody comprises variant hinge regions incapable of forming disulfide linkages between identical heavy chains (e.g., reduce homodimer formation). In some embodiments, the bispecific antibody comprises heavy chains with changes in amino acids that result in altered electrostatic interactions. In some embodiments, the bispecific antibodies comprise heavy chains with changes in amino acids that result in altered hydrophobic/hydrophilic interactions.

### Activatable Target Binding Proteins

In one aspect, the target binding proteins herein include activatable target binding proteins. In general, an activatable target binding protein may comprise a prodomain, which refers to a polypeptide that, when linked to a target binding protein, functions to inhibit target binding by the target binding protein and includes an amino acid sequence that form a protease cleavable substrate. The portion of the prodomain that inhibits target binding is referred to as a masking moiety (MM) and the amino acid sequence that is a protease cleavable substrate is referred to as a cleavable moiety (CM). The prodomain may include a linker (L) between the MM and the CM and/or at the prodomain's terminus (e.g., carboxyl and/or amino terminus to facilitate the linkage of the prodomain to the target binding protein). In certain embodiments, a prodomain may comprise one of the following formulae (representing an amino acid sequence in an N- to C-terminal direction): MM-CM, MM-L-CM, MM-CM-L, MM-L-CM-L, CM-MM, CM-L-MM, L-CM-MM, or L-CM-L-MM, wherein each represents a direct or indirect (e.g., via a linker) linkage.

As used herein, the term “activatable target binding protein” refers to a target binding protein in its inactive (uncleaved or native) state. It will be apparent to the ordinarily skilled artisan that in some embodiments a cleaved activatable target binding protein may be connected to a MM that is not reducing, inhibiting, or interfering with binding between the immunoglobulin variable region and its target. In some embodiments, a cleaved activatable target binding protein may lack a MM due to cleavage of the CM (e.g., by a protease), resulting in release of the MM. As used herein, the term “cleaved state” or “active state” refers to the condition of the activatable target binding proteins following cleavage of the CM by at least one protease. The term “uncleaved state” or “inactive state” refers to the condition of the activatable target binding proteins in the absence of cleavage of the CM by a protease.

By activatable is meant that the activatable target binding protein exhibits a first level of binding to a target when the activatable target binding protein is in an inhibited, masked or uncleaved state (i.e., a first conformation), and a second level of binding to the target in the uninhibited, unmasked and/or cleaved state (i.e., a second conformation), where the second level of target binding is greater than the first level of binding. In general, the access of target to the immunoglobulin variable region of the activatable target binding protein is greater in the presence of a cleaving agent capable of cleaving the CM, i.e., a protease, than in the absence of such a cleaving agent. Thus, when the activatable target binding protein is in the uncleaved state, the target binding domain is inhibited from target binding and can be masked from target binding (i.e., the first conformation is such that the immunoglobulin variable region cannot bind the target or is inhibited in binding the target), and in the cleaved state the immunoglobulin variable region is not inhibited or is unmasked to target binding.

In some embodiments, an activatable target binding protein may be designed by selecting an immunoglobulin variable region of interest and constructing the remainder of the activatable target binding protein so that, when conformationally constrained, the MM provides for masking of the immunoglobulin variable region or reduction of binding of the immunoglobulin variable region to its target. Structural design criteria can be to be taken into account to provide for this functional feature.

Activatable target binding proteins herein may exhibit an activatable phenotype of a desired dynamic range for target binding in an inhibited versus an uninhibited conformation. Dynamic range generally refers to a ratio of (a) a maximum detected level of a parameter under a first set of conditions to (b) a minimum detected value of that parameter under a second set of conditions. For example, in the context of an activatable target binding protein, the dynamic range refers to the ratio of (a) a maximum detected level of target protein binding to an activatable target binding protein in the presence of a protease capable of cleaving a CM in the activatable target binding proteins to (b) a minimum detected level of target protein binding to an activatable target binding protein in the absence of the protease. The dynamic range of an activatable target binding protein can be calculated as the ratio of the dissociation constant of an activatable target binding protein cleaving agent (e.g., enzyme) treatment to the dissociation constant of the activatable target binding proteins cleaving agent treatment. The greater the dynamic range of an activatable target binding protein, the better the activatable phenotype of the activatable target binding protein. Activatable target binding proteins having relatively higher dynamic range values (e.g., greater than 1) exhibit more desirable activatable phenotypes such that target protein binding by the activatable target binding proteins occurs to a greater extent (e.g., predominantly occurs) in the presence of a cleaving agent (e.g., enzyme) capable of cleaving the CM of the activatable target binding proteins than in the absence of a cleaving agent.

The activatable target binding protein herein may comprise a immunoglobulin variable region (TB), one or more masking moieties (MMs) reducing, inhibiting, or interfering with the binding of the immunoglobulin variable region to its target(s), one or more cleavable moieties (CMs) that couple the one or more MMs to the TB, and optionally one or more half-life extending moieties (EMs). In some embodiments, the activatable target binding protein may comprise the TB that specifically binds to hLY6G6D, and a masking moiety (MM) inhibiting the binding of the TB and hLY6G6D, wherein the MM is coupled to the TB via a cleavable moiety (CM) (either directly or indirectly, e.g., via one or more linkers). As used herein and unless otherwise stated, components of the activatable target binding protein that are “coupled” may be coupled either via a direct covalent linkage or indirect covalent linkage, e.g., via one or more linking peptides (also referred to as “linkers”), cleavable moieties, or other components of the activatable protein.

In some embodiments, the activatable target binding protein may comprise more than one immunoglobulin variable regions (TBs). For example, the activatable target binding protein may comprise the first TB that specifically binds to hLY6G6D, a first MM (MM1) inhibiting the binding of the TB1 and hLY6G6D, wherein the MM1 is coupled to the TB1 via a first cleavable moiety (CM1) (either directly or indirectly, e.g., via one or more linkers), a second immunoglobulin variable region (TB2) that specifically binds to a second target, a second masking moiety (MM2) inhibiting the binding of the TB2 and the second target, wherein the MM2 is coupled to the TB2 via a second cleavable moiety (CM2) (either directly or indirectly, e.g., via one or more linkers). The activatable target binding protein may further comprise a half-life extending moiety (EM). In one example, the activatable target binding protein comprise a scFv comprising the TB1.

### Masking Moieties (MMs)

The activatable target binding proteins herein may comprise one or more masking moieties (MMs) capable of interfering with the binding of the TBs to the targets. A masking moiety in an activatable molecule (that is not yet activated) “masks” or reduces or otherwise inhibits the binding of the immunoglobulin variable region to its target. In some embodiments, the coupling or modifying of target binding protein with a MM may inhibit the ability of the protein to specifically bind its target by means of inhibition known in the art (e.g., structural change and competition for antigen-binding domain). In some embodiments, the coupling or modifying of a target binding protein with a MM may effect a structural change that reduces or inhibits the ability of the protein to specifically bind its target. In some embodiments, the coupling or modifying of a target binding protein with a MM sterically blocks, reduces or inhibits the ability of the antigen-binding domain to specifically bind its target.

A MM may be coupled to a TB by a CM and optionally one or more linkers described herein. In some embodiments, when an activatable target binding protein is not activated, the MM prevents the TB from target binding; but when the activatable target binding protein is activated (when the CM is cleaved by a protease), the MMs does not substantially or significantly interfere with the TB's binding to the target.

In the activatable target binding protein, a MM interfering with the target binding of a TB may be coupled to the TB (either directly or indirectly, e.g., via one or more linkers). Alternatively, a MM interfering with the target binding of a TB may be coupled, either directly or indirectly, to a component of the activatable target binding protein that is not the TB. For example, the MM may be coupled, either directly or indirectly, to a different TB. In another example, the MM may be coupled, either directly or indirectly, with an EM. In either case, in the tertiary or quaternary structure of the activatable structure, the MM may be in a position (e.g., proximal to the TB to be masked) that allows the MM to mask the TB.

In some embodiments, a MM may interact with the TB, thus reducing or inhibiting the interaction between the TB and its binding partner. In some embodiments, the MM may comprise at least a partial or complete amino acid sequence of a naturally occurring binding partner of the TB. For example, the MM may be a fragment of a naturally occurring binding partner. The fragment may retain no more than 95%, 90%, 80%, 75%, 70%, 60%, 50%, 40%, 30%, 25%, or 20% nucleic acid or amino acid sequence homology to the naturally occurring binding partner. In some embodiments, the MM may be a cognate peptide of the TB. For example, the MM may comprise a sequence of the TB's epitope or a fragment thereof. The term “naturally occurring” as used herein as applied to an object refers to the fact that an object can be found in nature. For example, a polypeptide or polynucleotide sequence that is present in an organism (including viruses) that can be isolated from a source in nature and that has not been intentionally modified by man in the laboratory or otherwise is naturally occurring.

In some embodiments, the MM may comprise an amino acid sequence that is not naturally occurring or does not contain the amino acid sequence of a naturally occurring binding partner or target protein. In certain embodiments, the MM is not a natural binding partner of the TB. The MM may be a modified binding partner for the TB which contains amino acid changes that decrease affinity and/or avidity of binding to the TB. In some embodiments the MM may contain no or substantially no nucleic acid or amino acid homology to the TB's natural binding partner. In other embodiments the MM is no more than 5%, 10%, 15%, 20%, 25%, 30%, 35%, 40%, 45%, 50%, 55%, 60%, 65%, 70%, 75%, or 80% similar to the natural binding partner of the TB.

In some embodiments, the MM may not specifically bind to the TB, but still interfere with TB's binding to its binding partner through non-specific interactions such as steric hindrance. For example, the MM may be positioned in the activatable target binding protein such that the tertiary or quaternary structure of the activatable target binding protein allows the MM to mask the TB through charge-based interaction, thereby holding the MM in place to interfere with binding partner access to the TB. In some embodiments, the MM may have a dissociation constant for binding to the TB that is no more than the dissociation constant of the TB to the target. In some embodiments, the MM may not interfere or compete with the TB for binding to the target in a cleaved state.

The structural properties of the MMs may be selected according to factors such as the minimum amino acid sequence required for interference with protein binding to target, the target protein-protein binding pair of interest, the size of the TB, the presence or absence of linkers, and the like.

In some embodiments, the MM may be unique for the coupled TB. Examples of MMs include MMs that were specifically screened to bind a binding domain of the TB or fragment thereof (e.g., affinity masks). Methods for screening MMs to obtain MMs unique for the TB and those that specifically and/or selectively bind a binding domain of a binding partner/target are provided herein and can include protein display methods.

As used herein, the term “masking efficiency” refers to the activity (e.g., EC50) of the activatable target binding protein in the inactivated state divided by the activity of a control antibody, wherein the control antibody may be either cleavage product of the activatable target binding protein or the antibody or fragment thereof used as the TB of the activatable target binding protein. An activatable target binding protein having a reduced level of a TB activity may have a masking efficiency that is greater than 10. In some embodiments, the activatable target binding proteins described herein may have a masking efficiency that is greater than 10, 100, 1000, or 5000.

In some embodiments, the MM may be a polypeptide of about 2 to 50 amino acids in length. For example, the MM may be a polypeptide of from 2 to 40, from 2 to 30, from 2 to 20, from 2 to 10, from 5 to 15, from 10 to 20, from 15 to 25, from 20 to 30, from 25 to 35, from 30 to 40, from 35 to 45, from 40 to 50 amino acids in length. For example, the MM may be a polypeptide with 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids in length. In some examples, the MM may be a polypeptide of more than 50 amino acids in length, e.g., 100, 200, 300, 400, 500, 600, 700, 800, or more amino acids.

In some embodiments, in an inactive state of the activatable target binding protein with an TB and an interfering MM, in the presence of the target of an TB, there is no binding or substantially no binding of the TB to the target, or no more than 0 001%, 0.01%, 0.1%, 1%, 2%, 3%, 4%, 5%, 6%, 7%, 8%, 9%, 10%, 15%, 20%, 25%, 30%, 35%, 40%, or 50% binding of the TB to its target, as compared to the binding of an counterpart antibody without the interfering MM, for at least 0.1, 0.5, 1, 2, 4, 6, 8, 12, 28, 24, 30, 36, 48, 60, 72, 84, 96 hours, or 5, 10, 15, 30, 45, 60, 90, 120, 150, 180 days, or 1, 2, 3, 4, 5, 6, 7, 8, 9, 10, 11, or 12 months when measured in vitro immunoabsorbent assay, e.g., as described in US20200308243A1.

The binding affinity of the TB towards the target or binding partner with an interfering MM may be at least 5, 10, 25, 50, 100, 250, 500, 1,000, 2,500, 5,000, 10,000, 50,000, 100,000, 500,000, 1,000,000, 5,000,000, 10,000,000, 50,000,000 times lower than the binding affinity of the TB towards its binding partner without an interfering MM, or between 5-10, 10-100, 10-1,000, 10-10,000, 10-100,000, 10-1,000,000, 10-10,000,000, 100-1,000, 100-10,000, 100-100,000, 100-1,000,000, 100-10,000,000, 1,000-10,000, 1,000-100,000, 1,000-1,000,000, 1000-10,000,000, 10,000-100,000, 10,000-1,000,000, 10,000-10,000,000, 100,000-1,000,000, or 100,000-10,000,000 times lower than the binding affinity of the TB towards its binding partner when there is no interfering MM.

The dissociation constant of the MM towards the TB it masks, may be greater than the dissociation constant of the TB towards the target. The dissociation constant of the MM towards the masked TB may be at least 5, 10, 25, 50, 100, 250, 500, 1,000, 2,500, 5,000, 10,000, 100,000, 1,000,000 or even 10,000,000 times greater than the dissociation constant of the TB towards the target. Conversely, the binding affinity of the MM towards the masked TB may be lower than the binding affinity of the TB towards the target. The binding affinity of MM towards the TB may be at least 5, 10, 25, 50, 100, 250, 500, 1,000, 2,500, 5,000, 10,000, 100,000, 1,000,000 or even 10,000,000 times lower than the binding affinity of the TB towards the target.

In some embodiments, the MMs may contain genetically encoded or genetically nonencoded amino acids. Examples of genetically non-encoded amino acids include but are not limited to D-amino acids, P-amino acids, and y-amino acids. In specific embodiments, the MMs contain no more than 50%, 40%, 30%, 20%, 15%, 10%, 5% or 1% of genetically non-encoded amino acids.

In some embodiments, once released from the activatable target binding protein and in a free state, the MM may have a biological activity or a therapeutic effect, such as binding capability. For example, the free peptide may bind with the same or a different binding partner. In certain embodiments, the free MM may exert a therapeutic effect, providing a secondary function to the compositions disclosed herein. In some embodiments, once uncoupled from the activatable target binding protein and in a free state, the MM may advantageously not exhibit biological activity. For example, in some embodiments the MM in a free state does not elicit an immune response in the subject.

Suitable MMs may be identified and/or further optimized through a screening procedure from a library of candidate activatable target binding proteins having variable MMs. For example, a TB and a CM may be selected to provide for a desired enzyme/target combination, and the amino acid sequence of the MM can be identified by the screening procedure described below to identify a MM that provides for a switchable phenotype. For example, a random peptide library (e.g., of peptides comprising 2 to 40 amino acids or more) may be used in the screening methods disclosed herein to identify a suitable MM.

In some embodiments, MMs with specific binding affinity for a TB may be identified through a screening procedure that includes providing a library of peptide scaffolds comprising candidate MMs wherein each scaffold is made up of a transmembrane protein and the candidate MM. The library may then be contacted with an entire or portion of a protein such as a full length protein, a naturally occurring protein fragment, or a non-naturally occurring fragment containing a protein (also capable of binding the binding partner of interest), and identifying one or more candidate MMs having detectably bound protein. The screening may be performed by one more rounds of magnetic-activated sorting (MACS) or fluorescence-activated sorting (FACS), as well as determination of the binding affinity of MM towards the TB and subsequent determination of the masking efficiency, e.g., as described in WO2009025846 and US20200308243A1, which are incorporated herein by reference in their entireties.

In some embodiments, a MM may be selected for use with a specific antibody or antibody fragment. For example, suitable MM for use with a TB that binds to an epitope may comprise the sequence of the epitope. In some examples, suitable MM for masking the anti-hLY6G6D binding proteins disclosed herein include MMs comprising the sequences of GYLWGCEWNCGGITT (SEQ ID NO: 2658), NAFRCWWDPPCQPMT (SEQ ID NO: 2659), ARGLCWWDPPCTHDL (SEQ ID NO: 2660), or NHSLCYWDPPCEPST (SEQ ID NO: 2661). Additional masking moieties for anti-hLY6G6D binding proteins include the sequences of MMYCGGNEVLCGPRV (SEQ ID NO: 2662), GYRWGCEWNCGGITT (SEQ ID NO: 2663), MMYCGGNEIFCEPRG (SEQ ID NO: 2664), GYGWGCEWNCGGSSP (SEQ ID NO: 2665), and MMYCGGNEIFCGPRG (SEQ ID NO: 2666).

Examples of suitable MMs are disclosed in WO2021207657, WO2021142029, WO2021061867, WO2020252349, WO2020252358, WO2020236679, WO2020176672, WO2020118109, WO2020092881, WO2020086665, WO2019213444, WO2019183218, WO2019173771, WO2019165143, WO2019075405, WO2019046652, WO2019018828, WO2019014586, WO2018222949, WO2018165619, WO2018085555, WO2017011580, WO2016179335, WO2016179285, WO2016179257, WO2016149201, and WO2016014974, which are incorporated herein by reference in their entireties.

### Cleavable Moieties (CMs)

The activatable target binding protein may comprise one or more cleavable moieties (CMs). The terms “cleavable moiety” and “CM” are used interchangeably herein to refer to a peptide, the amino acid sequence of which comprises a substrate for a sequence-specific protease. In some embodiments, the CM may be positioned between a TB and a MM.

The CM and the TB of the activatable target binding proteins may be selected so that the TB represents a binding moiety for a given target, and the CM represents a substrate for one or more proteases, where the protease is co-localized with the target in a tissue (e.g., at a treatment site or diagnostic site in a subject). The protease may cleave the CM in the activatable target binding protein when the activatable target binding protein is exposed to the protease. In some embodiments, the activatable target binding proteins may find particular use where, for example, one or more proteases capable of cleaving a site in the CM, is present at relatively higher levels in target-containing tissue of a treatment site or diagnostic site than in tissue of non-treatment sites (for example in healthy tissue).

In some embodiments, the CMs herein may comprise substrates for proteases that have known substrates have been reported in a number of cancers. See, e.g., La Roca et al., British J. Cancer 90 (7): 1414-1421, 2004. Substrates suitable for use in the CM components employed herein include those which are more prevalently found in cancerous cells and tissue. Thus, in certain embodiments, the CM may comprise a substrate for a protease that is more prevalently found in diseased tissue associated with a cancer. Examples of the cancers include gastric cancer, breast cancer, osteosarcoma, esophageal cancer, breast cancer, a HER2-positive cancer, Kaposi sarcoma, hairy cell leukemia, chronic myeloid leukemia (CML), follicular lymphoma, renal cell cancer (RCC), melanoma, neuroblastoma, basal cell carcinoma, cutaneous T-cell lymphoma, nasopharyngeal adenocarcinoma, ovarian cancer, bladder cancer, BCG-resistant nonmuscle invasive bladder cancer (NMIBC), endometrial cancer, pancreatic cancer, non-small cell lung cancer (NSCLC), colorectal cancer, esophageal cancer, gallbladder cancer, glioma, head and neck carcinoma, uterine cancer, cervical cancer, or testicular cancer, and the like. In some embodiments, the CM components comprise substrates for protease(s) that is/are more prevalent in tumor tissue. For example, the protease(s) may be produced by a tumor in a subject.

In some embodiments, the activatable target binding protein may comprise two CMs (e.g., for coupling MMs to multiple TBs). In some examples, the first and the second CMs may comprise the substrates of the same protease. In some examples, the first and the second CMs may comprise the substrates of different proteases. In some examples, the first and the second CMs may comprise or consist of the same sequence. In some examples, the first and the second CMs may comprise or consist of different sequences.

Suitable CMs for use in the activatable target binding protein herein include any of the protease substrates that are known the art. In some examples, the CM may comprise a substrate of a serine protease (e.g., u-type plasminogen activator (uPA, also referred to as urokinase), matriptase (also referred to herein as MT-SP1 or MTSP1). In some examples, the CM may comprise a substrate of a matrix metalloprotease (MMP). In some examples, the CM may comprise a substrate of cysteine protease (CP) (e.g., legumain).

In some embodiments, the CM may comprise a substrate for a disintegrin and metalloproteinase (ADAM) or disintegrin and metalloproteinase with thrombospondin motifs (ADAMTS) (e.g., ADAM8, ADAM9, ADAM10, ADAM12, ADAM15, ADAM17/TACE, ADEMDEC1, ADAMTS1, ADAMTS4, ADAMTS5), aspartate protease (e.g. BACE, Renin), aspartic cathepsin (e.g., Cathepsin D, Cathepsin E), Caspase (e.g., Caspase 1, Caspase 2, Caspase 3, Caspase 4, Caspase 5, Caspase 6, Caspase 7, Caspase 8, Caspase 9, Caspase 10, Caspase 14), cysteine cathepsin (e.g., Cathepsin A, Cathepsin B, Cathepsin C, Cathepsin G, Cathepsin K, Cathepsin L, Cathepsin S, Cathepsin V/L2, Cathepsin X/Z/P), cysteine proteinase (e.g., Cruzipain, Legumain, Otubain-2), Chymase, DESCI, DPP-4, FAP, Elastase, FVIIa, FIXA, FXa, FXIa, FXIIa, Granzyme B, Guanidinobenzoatase, Hepsin, HtrAl, Human Neutrophil Elastase, KLK (e.g., KLK4, KLK5, KLK6, KLK7, KLK8, KLK10, KLK11, KLK13, KLK14), metallo proteinase (e g., Meprin, Neprilysin, PSMA, BMP-1), Lactoferrin, Marapsin, Matriptase-2, MT-SPl/Matriptase, NS3/4A, PACE4, Plasmin, PSA, a MMP (e.g., MMP1, MMP2, MMP3, MMP7, MMP8, MMP9, MMP10, MMP11, MMP12, MMP13, MMP14, MMP15, MMP16, MMP17, MMP19, MMP20, MMP23, MMP24, MMP26, MMP27), TMPRSS2, TMPRSS3, TMPRSS4, tPA, Thrombin, Tryptase, and uPA. In some embodiments, the protease substrate in the CM may comprise a peptide sequence that is not substantially identical (e.g., no more than 90%, 80%, 70%, 60%, or 50% identical) to any polypeptide sequence that is naturally cleaved by the same protease.

Examples of CMs include those described in WO 2010/081173, WO2021207669, WO2021207657, WO2021142029, WO2021061867, WO2020252349, WO2020252358, WO2020236679, WO2020176672, WO2020118109, WO2020092881, WO2020086665, WO2019213444, WO2019183218, WO2019173771, WO2019165143, WO2019075405, WO2019046652, WO2019018828, WO2019014586, WO2018222949, WO2018165619, WO2018085555, WO2017011580, WO2016179335, WO2016179285, WO2016179257, WO2016149201, WO2016014974, which are incorporated herein by reference in their entireties for all purposes.

In some embodiments, the CM may be or comprise a combination, a C-terminal truncation variant, or an N-terminal truncation variant of the example sequences discussed above. Truncation variants of the aforementioned amino acid sequences that are suitable for use in a CM may be any that retain the recognition site for the corresponding protease. These include C-terminal and/or N-terminal truncation variants comprising at least 3 contiguous amino acids of the above-described amino acid sequences, or at least 4, 5, 6, 7, 8, 9, or 10 amino acids of the foregoing amino acid sequences that retain a recognition site for a protease. In certain embodiments, the truncation variant of the above-described amino acid sequences may be an amino acid sequence corresponding to any of the above, but that is C- and/or N-terminally truncated by 1 to 10 amino acids, 1 to 9 amino acids, 1 to 8 amino acids, 1 to 7 amino acids, 1 to 6 amino acids, 1 to 5 amino acids, 1 to 4 amino acids, or 1 to 3 amino acids, and which: (1) has at least three amino acid residues; and (2) retains a recognition site for a protease. In some of the foregoing embodiments, the truncated CM is an N-terminally truncated CM. In some embodiments, the truncated CM is a C-terminally truncated CM. In some embodiments, the truncated C is a C- and an N-terminally truncated CM. In some embodiments, the CM may comprise a total of 3 amino acids to 25 amino acids. In some embodiments, the CM may comprise a total of 3 to 25, 3 to 20, 3 to 15, 3 to 10, 3 to 5, 5 to 25, 5 to 20, 5 to 15, 5 to 10, 10 to 25, 10 to 20, 10 to 15, 15 to 25, 15 to 20, or 20 to 25 amino acids.

In some embodiments, the CM may be specifically cleaved by at least a protease at a rate of about 0.001-1500×10⁴ M\hat{ }S′¹ or at least 0.001, 0.005, 0.01, 0.05, 0.1, 0.5, I, 2.5, 5, 7.5, 10, 15, 20, 25, 50, 75, 100, 125, 150, 200, 250, 500, 750, 1000, 1250, or 1500×10⁴ M\hat{ }S′¹. The rate may be measured as substrate cleavage kinetics (k_{c}at/Kₘ) as disclosed in WO2016118629. Conjugation agents

In some aspects, the target binding proteins (including the activatable target binding proteins) may further comprise one or more additional agents, e.g., a targeting moiety to facilitate delivery to a cell or tissue of interest, a therapeutic agent (e.g., an antineoplastic agent such as chemotherapeutic or anti-neoplastic agent), a toxin, a radioisotope, a small molecule, a diagnostic agent, a targeting moiety, or a detectable moiety, or a fragment thereof. The additional agents may be conjugated to the target binding proteins. The term “agent” is used herein to denote a chemical compound, a mixture of chemical compounds, a biological macromolecule, or an extract made from biological materials.

### Pharmaceutical Compositions and Administration

To prepare pharmaceutical or sterile compositions of the target binding proteins (e.g., anti-hLY6G6D antibodies and antigen-binding fragments) of the invention, the antibody or antigen-binding fragment thereof is admixed with a pharmaceutically acceptable carrier or excipient. See, e.g., Remington's Pharmaceutical Sciences and U.S. Pharmacopeia: National Formulary, Mack Publishing Company, Easton, PA (1984).

Formulations of therapeutic agents may be prepared by mixing with acceptable carriers, excipients, or stabilizers in the form of, e.g., lyophilized powders, slurries, aqueous solutions or suspensions (see, e.g., Hardman, et al. (2001) Goodman and Gilman's The Pharmacological Basis of Therapeutics, McGraw-Hill, New York, NY; Gennaro (2000) Remington: The Science and Practice of Pharmacy, Lippincott, Williams, and Wilkins, New York, NY; Avis, et al. (eds.) (1993) Pharmaceutical Dosage Forms: Parenteral Medications, Marcel Dekker, NY; Lieberman, et al. (eds.) (1990) Pharmaceutical Dosage Forms: Tablets, Marcel Dekker, NY; Lieberman, et al. (eds.) (1990) Pharmaceutical Dosage Forms: Disperse Systems, Marcel Dekker, NY; Weiner and Kotkoskie (2000) Excipient Toxicity and Safety, Marcel Dekker, Inc., New York, NY).

Toxicity and therapeutic efficacy of the target binding proteins of the invention, administered alone or in combination with another therapeutic agent, can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index (LD₅₀/ED₅₀). The data obtained from these cell culture assays and animal studies can be used in formulating a range of dosage for use in human. The dosage of such compounds lies preferably within a range of circulating concentrations that include the ED₅₀ with little or no toxicity. The dosage may vary within this range depending upon the dosage form employed and the route of administration.

Suitable routes of administration include parenteral administration, such as intramuscular, intravenous, or subcutaneous administration and oral administration. Administration of target binding proteins, used in the pharmaceutical composition or to practice the method of the present invention can be carried out in a variety of conventional ways, such as oral ingestion, inhalation, topical application or cutaneous, subcutaneous, intraperitoneal, parenteral, intraarterial or intravenous injection. In one embodiment, the target binding protein of the invention is administered intravenously. In another embodiment, the target binding proteins of the invention is administered subcutaneously.

Alternatively, one may administer the target binding proteins in a local rather than systemic manner, for example, via injection of the antibody directly into the site of action, often in a depot or sustained release formulation. Furthermore, one may administer the antibody in a targeted drug delivery system.

In a further embodiment, a further therapeutic agent that is administered to a subject in association with the target binding proteins of the invention in accordance with the Physicians' Desk Reference 2003 (Thomson Healthcare; 57th edition (Nov. 1, 2002)). Methods for co-administration or treatment with a second therapeutic agent are well known in the art, see, e.g., Hardman, et al. (eds.), 2001, Goodman and Gilman's The Pharmacological Basis of Therapeutics, 10th ed., McGraw-Hill, New York, NY; Poole and Peterson (eds.), 2001, Pharmacotherapeutics for Advanced Practice: A Practical Approach, Lippincott, Williams & Wilkins, Phila., PA; Chabner and Longo (eds.), 2001, Cancer Chemotherapy and Biotherapy, Lippincott, Williams & Wilkins, Phila., PA. The other agents include, but are not limited to, a cytotoxic, chemotherapeutic, cytostatic, anti-angiogenic or antimetabolite agents, a tumor targeted agent, an immune stimulating or immune modulating agent or an antibody conjugated to a cytotoxic, cytostatic, or otherwise toxic agent. The pharmaceutical composition can also be employed with other therapeutic modalities such as surgery, chemotherapy and radiation.

In particular embodiments, an anti-hLY6G6D antibody or antigen-binding fragment thereof of the invention can be administered by an invasive route such as by injection. In further embodiments of the invention, an anti-hLY6G6D antibody or antigen-binding fragment thereof, or pharmaceutical composition thereof, is administered intravenously, subcutaneously, intramuscularly, intraarterially, intratumorally, or by inhalation, aerosol delivery. Administration by non-invasive routes (e.g., orally; for example, in a pill, capsule or tablet) is also within the scope of the present invention.

The present invention provides a vessel (e.g., a plastic or glass vial, e.g., with a cap or a chromatography column, hollow bore needle or a syringe cylinder) comprising any of the target binding proteins of the invention or a pharmaceutical composition thereof. The present invention also provides an injection device comprising any of the target binding proteins of the invention or a pharmaceutical composition thereof. An injection device is a device that introduces a substance into the body of a patient via a parenteral route, e.g., intramuscular, subcutaneous or intravenous. For example, an injection device may be a syringe (e.g., pre-filled with the pharmaceutical composition, such as an auto-injector) which, for example, includes a cylinder or barrel for holding fluid to be injected (e.g., antibody or fragment or a pharmaceutical composition thereof), a needle for piecing skin and/or blood vessels for injection of the fluid; and a plunger for pushing the fluid out of the cylinder and through the needle bore. In an embodiment of the invention, an injection device that comprises an antibody or antigen-binding fragment thereof of the present invention or a pharmaceutical composition thereof is an intravenous (IV) injection device. Such a device includes the antibody or fragment or a pharmaceutical composition thereof in a cannula or trocar/needle which may be attached to a tube which may be attached to a bag or reservoir for holding fluid (e.g., saline; or lactated ringer solution comprising NaCl, sodium lactate, KCl, CaCl₂) and optionally including glucose) introduced into the body of the patient through the cannula or trocar/needle. The antibody or fragment or a pharmaceutical composition thereof may, in an embodiment of the invention, be introduced into the device once the trocar and cannula are inserted into the vein of a subject and the trocar is removed from the inserted cannula. The IV device may, for example, be inserted into a peripheral vein (e.g., in the hand or arm); the superior vena cava or inferior vena cava, or within the right atrium of the heart (e.g., a central IV); or into a subclavian, internal jugular, or a femoral vein and, for example, advanced toward the heart until it reaches the superior vena cava or right atrium (e.g., a central venous line). In an embodiment of the invention, an injection device is an autoinjector; a jet injector or an external infusion pump. A jet injector uses a high-pressure narrow jet of liquid which penetrate the epidermis to introduce the antibody or fragment or a pharmaceutical composition thereof to a patient's body. External infusion pumps are medical devices that deliver the antibody or fragment or a pharmaceutical composition thereof into a patient's body in controlled amounts. External infusion pumps may be powered electrically or mechanically. Different pumps operate in different ways, for example, a syringe pump holds fluid in the reservoir of a syringe, and a moveable piston controls fluid delivery, an elastomeric pump holds fluid in a stretchable balloon reservoir, and pressure from the elastic walls of the balloon drives fluid delivery. In a peristaltic pump, a set of rollers pinches down on a length of flexible tubing, pushing fluid forward. In a multi-channel pump, fluids can be delivered from multiple reservoirs at multiple rates.

The pharmaceutical compositions disclosed herein may also be administered with a needleless hypodermic injection device; such as the devices disclosed in U.S. Pat. Nos. 6,620,135; 6,096,002; 5,399,163; 5,383,851; 5,312,335; 5,064,413; 4,941,880; 4,790,824 or 4,596,556. Such needleless devices comprising the pharmaceutical composition are also part of the present invention. The pharmaceutical compositions disclosed herein may also be administered by infusion. Examples of well-known implants and modules for administering the pharmaceutical compositions include those disclosed in: U.S. Pat. No. 4,487,603, which discloses an implantable micro-infusion pump for dispensing medication at a controlled rate; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments. Many other such implants, delivery systems, and modules are well known to those skilled in the art and those comprising the pharmaceutical compositions of the present invention are within the scope of the present invention.

Alternately, one may administer the target binding proteins of the invention in a local rather than systemic manner, for example, via injection of the antibody or fragment directly into a tumor. Furthermore, one may administer the antibody or fragment in a targeted drug delivery system, for example, in a liposome coated with a tissue-specific antibody, targeting, for example, a tumor. The liposomes will be targeted to and taken up selectively by the afflicted tissue. Such methods and liposomes are part of the present invention.

The administration regimen depends on several factors, including the serum or tissue turnover rate of the target binding proteins (anti-hLY6G6D antibody or antigen-binding fragment), the level of symptoms, the immunogenicity of the target binding proteins, and the accessibility of the target cells in the biological matrix. Preferably, the administration regimen delivers sufficient therapeutic antibody or fragment to effect improvement in the target disease state, while simultaneously minimizing undesired side effects. Accordingly, the amount of biologic delivered depends in part on the particular therapeutic antibody and the severity of the condition being treated. Guidance in selecting appropriate doses of therapeutic antibodies or fragments is available (see, e.g., Wawrzynczak (1996) Antibody Therapy, Bios Scientific Pub. Ltd, Oxfordshire, UK; Kresina (ed.) (1991) Monoclonal Antibodies, Cytokines and Arthritis, Marcel Dekker, New York, NY; Bach (ed.) (1993) Monoclonal Antibodies and Peptide Therapy in Autoimmune Diseases, Marcel Dekker, New York, NY; Baert, et al. (2003) New Engl. J. Med. 348:601-608; Milgrom et al. (1999) New Engl. J. Med. 341:1966-1973; Slamon et al. (2001) New Engl. J. Med. 344:783-792; Beniaminovitz et al. (2000) New Engl. J. Med. 342:613-619; Ghosh et al. (2003) New Engl. J. Med. 348:24-32; Lipsky et al. (2000) New Engl. J. Med. 343:1594-1602).

Determination of the appropriate dose is made by the clinician, e.g., using parameters or factors known or suspected in the art to affect treatment. Generally, the dose begins with an amount somewhat less than the optimum dose and it is increased by small increments thereafter until the desired or optimum effect is achieved relative to any negative side effects. Important diagnostic measures include those of symptoms of, e.g., the inflammation or level of inflammatory cytokines produced. In general, it is desirable that a biologic that will be used is derived from the same species as the animal targeted for treatment, thereby minimizing any immune response to the reagent. In the case of human subjects, for example, humanized and fully human antibodies may be desirable.

The target binding proteins disclosed herein may be provided by continuous infusion, or by doses administered, e.g., daily, 1-7 times per week, weekly, bi-weekly, monthly, bimonthly, quarterly, semiannually, annually etc. Doses may be provided, e.g., intravenously, subcutaneously, topically, orally, nasally, rectally, intramuscular, intracerebrally, intraspinally, or by inhalation. A total weekly dose is generally at least 0.05 μg/kg body weight, more generally at least 0.2 μg/kg, 0.5 μg/kg, 1 μg/kg, 10 μg/kg, 100 μg/kg, 0.25 mg/kg, 1.0 mg/kg, 2.0 mg/kg, 5.0 mg/mL, 10 mg/kg, 25 mg/kg, 50 mg/kg or more (see, e.g., Yang, et al. (2003) New Engl. J. Med. 349:427-434; Herold, et al. (2002) New Engl. J. Med. 346:1692-1698; Liu, et al. (1999) J. Neurol. Neurosurg. Psych. 67:451-456; Portielji, et al. (20003) Cancer Immunol. Immunother. 52:151-144). Doses may also be provided to achieve a pre-determined target concentration of target binding proteins in the subject's serum, such as 0.1, 0.3, 1, 3, 10, 30, 100, 300 μg/mL or more. In other embodiments, a target binding protein of the present invention is administered, e.g., subcutaneously or intravenously, on a weekly, biweekly, “every 4 weeks,” monthly, bimonthly, or quarterly basis at 10, 20, 50, 80, 100, 200, 500, 1000 or 2500 mg/subject.

The target binding proteins can be administered over a period of at least about 1 week, 2 weeks, 1 month (4 weeks), 6 weeks, 2 months, 3 months, 6 months, 1 year, 2 years, 3 years or longer, or as deemed appropriate by the treating physician. A chronic condition can exist for, e.g., at least about 6 weeks, 2 months, a year, or longer. The target binding proteins can be administered over a period of at least about 6 weeks, 2 months, 3 months or 6 months, a year, or even multiple years as required for medical care of an individual.

The target binding proteins can also be administered in an irregular manner. Early achievement of an effective target antibody concentration (a therapeutic dose level) with a loading dose followed by maintenance dosing with the antibody (frontloading) may be more effective than conventional therapy in terms of requiring a lower total antibody dose and faster time to maximum target engagement. As used herein, such an administration protocol is referred to as a “loading/maintenance administration protocol.” An effective target antibody concentration may be reached in 4 weeks or less, preferably 3 weeks or less, more preferably 2 weeks or less, most preferably 1 week or less, including 1 day or less using a loading dose. The target serum concentration is then maintained by administration of an equal or smaller (or less frequent) maintenance dose during the remainder of the treatment regimen or until suppression of disease symptoms is achieved.

The term “frontloading” when referring to drug administration refers to the initial loading dose, followed by the maintenance dose. The initial loading dose (single or multiple) is intended to more quickly increase the serum drug concentration of an animal or human patient to an effective target serum concentration. In various embodiments, frontloading is accomplished by initial dosing delivered over 3 weeks or less so that the antibody reaches the target serum concentration. Preferably, the loading dose or series of doses is administered for 2 weeks or less, more preferably 1 week or less, e.g. 1 day or less. Most preferably, the loading dosing is a single dosing, with no maintenance dosing thereafter for at least one week, and the loading dosing is administered in 1 day or less. In order to avoid adverse immune reactions to antibody drugs, it may be preferred to deliver the loading dose of antibody is administered by intravenous injection. The present invention includes loading and maintenance doses of frontloading drug delivery by intravenous or subcutaneous administration.

Administration of the loading dose can be, for example, one or more dosings at a time interval of at least about 1, 2, 3, 4, 5, 6, 7 or 8 weeks apart. In some embodiments, the at least one loading dose is administered by one or more intravenous injections and then at least one maintenance dose by one or more intravenous or subcutaneous administrations. In other embodiments, the instructions can be for administering at least one loading dose by, for example, one or more intravenous or subcutaneous administrations and at least one maintenance dose by one or more intravenous or subcutaneous administrations. In certain embodiments, both the at least one loading dose as well as the at least one maintenance dose is administered subcutaneously. In other embodiments, the at least one loading dose is administered by intravenous infusion followed by at least one maintenance dose administered subcutaneously. For example, the method of treatment can comprise administering a loading dose of 150-1350 mg of the anti-hLY6G6D antibody by intravenous infusion or subcutaneous injection. After the loading dose (e.g. 1 week, 2 weeks, 3 weeks or 4 weeks after the loading dose), a maintenance dose of 600 mg or less of the anti-hLY6G6D antibody can be administered every 4 weeks or less, preferably every 3 weeks or less, more preferably every 2 weeks or less, and in embodiments every 1 week or less, by subcutaneous injection. The choice of loading and maintenance dosages and intervals can be made according to the ability of the animal or human patient to tolerate administration of the antibody to the body and according to a desired serum level of therapeutic to achieve.

A loading dose of a drug can be larger (e.g., about 1.5, 2, 3, 4 or 5 times larger) than a subsequent maintenance dose. The one or more therapeutically effective maintenance doses can be any therapeutically effective amount described herein. The loading dose can be about 2 or 3 times larger than the maintenance dose. The anti-hLY6G6D antibody can be administered in two (or more) loading doses prior to the maintenance dose. A first loading dose of the antibody or fragment thereof can be administered on day 1, a second loading dose can be administered, e.g., about 1 or 2 weeks later, and a maintenance dose can be administered, e.g., once weekly or once every 2 weeks thereafter for the duration of treatment. The first loading dose can be about 3 or 4 times larger than the maintenance dose, and the second loading dose can be about 2, 3, 4, 5, or more times larger than the maintenance dose.

As used herein, “inhibit” or “treat” or “treatment” includes a postponement of development of the symptoms associated with disease and/or a reduction in the severity of such symptoms that will or are expected to develop with said disease. The terms further include ameliorating existing symptoms, preventing additional symptoms, and ameliorating or preventing the underlying causes of such symptoms. Thus, the terms denote that a beneficial result has been conferred on a vertebrate subject with a disease.

As used herein, the term “effective amount” refers to an amount of a target binding proteins f of the invention that, when administered alone or in combination with an additional therapeutic agent to a cell, tissue, or subject, is effective to cause a measurable improvement in one or more symptoms of disease, for example cancer or the progression of cancer. An effective dose further refers to that amount of the antibody or fragment sufficient to result in at least partial amelioration of symptoms, e.g., tumor shrinkage or elimination, lack of tumor growth, increased survival time. When applied to an individual active ingredient administered alone, an effective dose refers to that ingredient alone. When applied to a combination, an effective dose refers to combined amounts of the active ingredients that result in the therapeutic effect, whether administered in combination, serially or simultaneously. An effective amount of a therapeutic will result in an improvement of a diagnostic measure or parameter by at least 10%; usually by at least 20%; preferably at least about 30%; more preferably at least 40%, and most preferably by at least 50%. An effective amount can also result in an improvement in a subjective measure in cases where subjective measures are used to assess disease severity.

### Kits

Further provided are kits comprising one or more components that include, but are not limited to, a target binding protein as described herein in association with one or more additional components including, but not limited to a pharmaceutically acceptable carrier and/or a therapeutic agent, as discussed herein. The antibody or fragment and/or the therapeutic agent can be formulated as a pure composition or in combination with a pharmaceutically acceptable carrier, in a pharmaceutical composition.

In one embodiment, the kit includes an anti-hLY6G6D antibody or antigen-binding fragment thereof of the invention or a pharmaceutical composition thereof in one container (e.g., in a sterile glass or plastic vial) and/or a therapeutic agent and a pharmaceutical composition thereof in another container (e.g., in a sterile glass or plastic vial).

In another embodiment, the kit comprises a combination of the invention, including an anti-hLY6G6D antibody or antigen-binding fragment thereof of the invention along with a pharmaceutically acceptable carrier, optionally in combination with one or more therapeutic agents formulated together, optionally, in a pharmaceutical composition, in a single, common container.

If the kit includes a pharmaceutical composition for parenteral administration to a subject, the kit can include a device for performing such administration. For example, the kit can include one or more hypodermic needles or other injection devices as discussed above.

The kit can include a package insert including information concerning the pharmaceutical compositions and dosage forms in the kit. Generally, such information aids patients and physicians in using the enclosed pharmaceutical compositions and dosage forms effectively and safely. For example, the following information regarding a combination of the invention may be supplied in the insert: pharmacokinetics, pharmacodynamics, clinical studies, efficacy parameters, indications and usage, contraindications, warnings, precautions, adverse reactions, overdosage, proper dosage and administration, how supplied, proper storage conditions, references, manufacturer/distributor information and patent information.

### Preferred Embodiments of the Invention

Embodiment 1: An antibody or antigen binding fragment thereof that binds to human LY6G6D, wherein the antibody or antigen binding fragment comprises heavy chain CDRs H1, H2, and H3 and light chain CDRs L1, L2, and L3 amino acid sequences as recited for one of the Identifiers in Table 1.

Embodiment 2: The antibody or antigen binding fragment of embodiment 1, wherein the antigen or binding fragment comprises a heavy chain variable region and a light chain variable region comprising amino acid sequences as recited for one of the Identifiers in Table 2; or a heavy chain variable region and a light chain variable region having at least 90% sequence identity, at least 95% sequence identity, at least 97% sequence identity, at least 98% sequence identity, or at least 99% sequence identity with a heavy chain variable region and a light chain variable region comprising amino acid sequences as recited for one of the Identifiers in Table 2.

Embodiment 3: The antibody or antigen binding fragment of embodiment 1 or 2, wherein the antibody or fragment thereof is an scFv.

Embodiment 4: The antibody or antigen binding fragment of one of embodiments 1 or 2, wherein the antibody or antigen binding fragment is provided as an intact IgG antibody.

Embodiment 5: The antibody or antigen binding fragment of embodiment 4, wherein the IgG antibody comprises a wild-type or mutated IgG2 Fc region.

Embodiment 6: The antibody or antigen binding fragment of embodiment 4, wherein the IgG antibody comprises an IgG1 Fc region.

Embodiment 7: The antibody or antigen binding fragment of embodiment 4, wherein the IgG1 Fc region comprises one or mutations that reduce effector functions of the IgG antibody relative to a wild-type IgG1 Fc region.

Embodiment 8: The antibody or antigen binding fragment of embodiment 4, wherein the IgG antibody comprises an IgG4 Fc region.

Embodiment 9: The antibody or antigen binding fragment of any of embodiments 1-10, wherein the antibody or antigen binding fragment is human or is humanized.

Embodiment 10: The antibody or antigen binding fragment of any one of embodiments 1-9 that comprises a glycosylation pattern characteristic of expression by a mammalian cell.

Embodiment 11: The antibody or antigen binding fragment of any one of embodiments 1-9 that comprises a glycosylation pattern characteristic of expression by a CHO cell.

Embodiment 12: The antibody or antigen binding fragment of any one of embodiments 1-11, wherein the antibody or antigen binding fragment is provided as a bispecific T cell engaging antibody (BiTE), a (SCFV)2, a NANOBODY®, a nanobody-HSA VHH-scAb, a VHH-Fab, a Dual scFab, a F(ab′)2, a diabody, a CROSSMAB®, a DAF (two-in-one), a DAE (four-in-one), a DUTAMAB®, a DT-TgG, a knobs-in-holes common light chain, a knobs-in-holes assembly, a charge pair, a Fab-arm exchange, a SEEDbody, a LUZ-Y, a FcAb, a kl-body, an orthogonal Fab, a DVD-IgG, a IgG (H)-scFv, a scFv-(H) IgG, IgG (L)-scFv, scFv-(L) IgG, IgG (L,H)-Fv, IgG (H)-V, V(H)-IgG, IgG (L)-V, V(L)-IgG, KIH IgG-scFab, 2scFv-IgG, IgG-2scFv, scFv4-Ig, ZYBODY™, DVI-IgG, Diabody-CH3, a triple body, a miniantibody, a minibody, a TriBi minibody, scFv-CH3 KIH, Fab-scFv, a F(ab′)2-scFv2, a scFv-KIH, a Fab-scFv-Fc, a tetravalent HCAb, a scDiabody-Fc, a Diabody-Fc, a tandem scFv-Fc, a VHH-Fc, a tandem VHH-Fc, a LTIH-Fc KiH, a Fab-VHH-Fc, an Intrabody, a dock and lock, an ImmTAC® (immune-mobilizing monoclonal TCRs (T cell receptors) against cancer), an IgG-IgG conjugate, a Cov-X-Body, a scFv1-PEG-scFv2, an Adnectin, a DARPin, or a fibronectin, an IgG, an IgM, an IgA, an IgE, an IgD, or a DEP conjugate, TMEAbody™, SAFEbody®, TRITAC®, a dual affinity retargeting (DART®) bispecific antibody, a simultaneous multiple interaction T-cell engagers (SMITE), or a SHIELD.

Embodiment 13: The antibody or antigen binding fragment of any one of embodiments 1-12, wherein the antibody or antigen binding fragment thereof comprises an immunoglobulin binding motif that binds to an antigen selected from the group consisting CD16, γδ TCR, CTLA-4, PD-1, PD-L1, 4-1BB, IL-13, IL-4, VEGF, and CD3.

Embodiment 14: The antibody according to any one of embodiments 1-13, wherein the antibody comprises heavy chain CDRs H1, H2, and H3 and light chain CDRs L1, L2, and L3 amino acid sequences as recited for an Identifier in Table 1 selected from the group consisting of:
CB21-498, CB21-498-001, CB21-498-002, CB21-498-003, CB21-498-004, CB21-498-005, CB21-498-006, CB21-498-007, CB21-498-008, CB21-498-009, CB21-498-010, CB21-498-011, CB21-498-012, CB21-498-013, CB21-498-014, CB21-498-015, CB21-498-016, CB21-498-017, CB21-498-018, CB21-498-019, CB21-498-020, CB21-498-021, CB21-498-022, CB21-498-023, CB21-498-024, CB21-498-025, CB21-498-026, CB21-498-027, CB21-498-028, CB21-498-029, CB21-498-030, CB21-498-031, CB21-498-032, CB21-498-033, CB21-498-034, CB21-498-035, CB21-498-036, CB21-498-037, CB21-498-038, CB21-498-039, CB21-498-040, CB21-498-041, CB21-498-042, CB21-498-043, CB21-498-044, CB21-498-045, CB21-498-046, CB21-498-047, CB21-498-048, CB21-498-049, CB21-498-050, CB21-498-051, CB21-498-052, CB21-498-053, CB21-498-054, CB21-498-055, CB21-498-056, CB21-498-057, CB21-498-058, CB21-498-059, CB21-498-060, CB21-498-061, CB21-498-062, CB21-498-063, CB21-498-064, CB21-498-065, CB21-498-066, CB21-498-067, CB21-498-068, CB21-498-069, CB21-498-070, CB21-498-071, CB21-498-072, CB21-498-073, CB21-498-074, CB21-498-075, CB21-498-076, CB21-498-077, CB21-498-078, CB21-498-079, CB21-498-080, CB21-498-081, CB21-498-082, CB21-498-083, CB21-498-084, CB21-498-085, CB21-498-086, CB21-498-087, CB21-498-088, CB21-498-089, CB21-498-090, CB21-498-091, CB21-498-092, CB21-498-093, CB21-498-094, CB21-498-095, CB21-498-096, CB21-498-097, CB21-498-098, CB21-498-099, CB21-498-100, CB21-498-101, CB21-498-102, CB21-498-103, CB21-498-104, CB21-498-105, CB21-498-106, CB21-498-107, CB21-498-108, CB21-498-109, CB21-498-110, CB21-498-111, CB21-498-112, CB21-498-113, CB21-498-114, CB21-498-115, CB21-498-116, CB21-498-117, CB21-498-118, CB21-498-119, CB21-498-120, CB21-498-121, CB21-498-122, CB21-498-123, CB21-498-124, CB21-498-125, CB21-498-126, CB21-498-127, CB21-498-128, CB21-498-129, CB21-498-130, CB21-498-131, CB21-498-132, CB21-498-133, CB21-498-134, CB21-498-135, CB21-498-136, CB21-498-137, CB21-498-138, CB21-498-139, CB21-498-140, CB21-498-141, CB21-498-142, CB21-498-143, CB21-499, CB21-499-001, CB21-499-002, CB21-499-003, CB21-499-004, CB21-499-005, CB21-499-006, CB21-499-007, CB21-499-008, CB21-499-009, CB21-499-010, CB21-499-011, CB21-499-012, CB21-499-013, CB21-499-014, CB21-499-015, CB21-499-016, CB21-499-017, CB21-499-018, CB21-499-019, CB21-499-020, CB21-499-021, CB21-499-022, CB21-499-023, CB21-499-024, CB21-499-025, CB21-499-026, CB21-499-027, CB21-499-028, CB21-499-029, CB21-499-030, CB21-499-031, CB21-499-032, CB21-499-033, CB21-499-034, CB21-499-035, CB21-499-036, CB21-499-037, CB21-499-038, CB21-499-039, CB21-499-040, CB21-499-041, CB21-499-042, CB21-499-043, CB21-499-044, CB21-499-045, CB21-499-046, CB21-499-047, CB21-499-048, CB21-499-049, CB21-499-050, CB21-499-051, CB21-499-052, CB21-499-053, CB21-499-054, CB21-499-055, CB21-499-056, CB21-499-057, CB21-499-058, CB21-499-059, CB21-499-060, CB21-499-061, CB21-499-062, CB21-499-063, CB21-499-064, CB21-499-065, CB21-499-066, CB21-499-067, CB21-499-068, CB21-499-069, CB21-499-070, CB21-499-071, CB21-499-072, CB21-499-073, CB21-499-074, CB21-499-075, CB21-499-076, CB21-499-077, CB21-499-078, CB21-499-079, CB21-499-080, CB21-499-081, CB21-499-082, CB21-499-083, CB21-499-084, CB21-499-085, CB21-499-086, CB21-499-087, CB21-499-088, CB21-499-089, CB21-499-090, CB21-499-091, CB21-499-092, CB21-499-093, CB21-499-094, CB21-499-095, CB21-499-096, CB21-499-097, CB21-499-098, CB21-499-099, CB21-499-100, CB21-499-101, CB21-499-102, CB21-499-103, CB21-499-104, CB21-499-105, CB21-499-106, CB21-499-107, CB21-499-108, CB21-499-109, CB21-499-110, CB21-499-111, CB21-499-112, CB21-499-113, CB21-499-114, CB21-499-115, CB21-499-116, CB21-499-117, CB21-499-118, CB21-499-119, CB21-499-120, CB21-499-121, CB21-499-122, CB21-499-123, CB21-499-124, CB21-499-125, CB21-499-126, CB21-499-127, CB21-499-128, CB21-499-129, CB21-499-130, CB21-499-131, CB21-499-132, CB21-499-133, CB21-499-134, CB21-499-135, CB21-499-136, CB21-499-137, CB21-499-138, CB21-499-139, CB21-499-140, CB21-499-141, CB21-499-142, CB21-499-143, CB21-499-144, CB21-499-145, CB21-499-146, CB21-499-147, CB21-499-148, CB21-499-149, CB21-499-150, CB21-499-151, CB21-499-152, CB21-499-153, CB21-499-154, CB21-499-155, CB21-499-156, CB21-499-157, CB21-499-158, CB21-499-159, CB21-499-160, CB21-499-161, CB21-499-162, CB21-499-163, CB21-499-164, CB21-499-165, CB21-499-166, CB21-499-167, CB21-499-168, CB21-499-169, CB21-499-170, CB21-499-171, CB21-499-172, CB21-499-174, CB21-499-175, CB21-499-176, CB21-499-177, CB21-499-178, and CB21-499-179.

Embodiment 15: The antibody according to any one of embodiments 1-13, wherein the antigen or binding fragment comprises a heavy chain variable region and a light chain variable region comprising amino acid sequences as recited for an Identifier in Table 2 selected from the group consisting of: CB21-498, CB21-498-001, CB21-498-002, CB21-498-003, CB21-498-004, CB21-498-005, CB21-498-006, CB21-498-007, CB21-498-008, CB21-498-009, CB21-498-010, CB21-498-011, CB21-498-012, CB21-498-013, CB21-498-014, CB21-498-015, CB21-498-016, CB21-498-017, CB21-498-018, CB21-498-019, CB21-498-020, CB21-498-021, CB21-498-022, CB21-498-023, CB21-498-024, CB21-498-025, CB21-498-026, CB21-498-027, CB21-498-028, CB21-498-029, CB21-498-030, CB21-498-031, CB21-498-032, CB21-498-033, CB21-498-034, CB21-498-035, CB21-498-036, CB21-498-037, CB21-498-038, CB21-498-039, CB21-498-040, CB21-498-041, CB21-498-042, CB21-498-043, CB21-498-044, CB21-498-045, CB21-498-046, CB21-498-047, CB21-498-048, CB21-498-049, CB21-498-050, CB21-498-051, CB21-498-052, CB21-498-053, CB21-498-054, CB21-498-055, CB21-498-056, CB21-498-057, CB21-498-058, CB21-498-059, CB21-498-060, CB21-498-061, CB21-498-062, CB21-498-063, CB21-498-064, CB21-498-065, CB21-498-066, CB21-498-067, CB21-498-068, CB21-498-069, CB21-498-070, CB21-498-071, CB21-498-072, CB21-498-073, CB21-498-074, CB21-498-075, CB21-498-076, CB21-498-077, CB21-498-078, CB21-498-079, CB21-498-080, CB21-498-081, CB21-498-082, CB21-498-083, CB21-498-084, CB21-498-085, CB21-498-086, CB21-498-087, CB21-498-088, CB21-498-089, CB21-498-090, CB21-498-091, CB21-498-092, CB21-498-093, CB21-498-094, CB21-498-095, CB21-498-096, CB21-498-097, CB21-498-098, CB21-498-099, CB21-498-100, CB21-498-101, CB21-498-102, CB21-498-103, CB21-498-104, CB21-498-105, CB21-498-106, CB21-498-107, CB21-498-108, CB21-498-109, CB21-498-110, CB21-498-111, CB21-498-112, CB21-498-113, CB21-498-114, CB21-498-115, CB21-498-116, CB21-498-117, CB21-498-118, CB21-498-119, CB21-498-120, CB21-498-121, CB21-498-122, CB21-498-123, CB21-498-124, CB21-498-125, CB21-498-126, CB21-498-127, CB21-498-128, CB21-498-129, CB21-498-130, CB21-498-131, CB21-498-132, CB21-498-133, CB21-498-134, CB21-498-135, CB21-498-136, CB21-498-137, CB21-498-138, CB21-498-139, CB21-498-140, CB21-498-141, CB21-498-142, CB21-498-143, CB21-499, CB21-499-001, CB21-499-002, CB21-499-003, CB21-499-004, CB21-499-005, CB21-499-006, CB21-499-007, CB21-499-008, CB21-499-009, CB21-499-010, CB21-499-011, CB21-499-012, CB21-499-013, CB21-499-014, CB21-499-015, CB21-499-016, CB21-499-017, CB21-499-018, CB21-499-019, CB21-499-020, CB21-499-021, CB21-499-022, CB21-499-023, CB21-499-024, CB21-499-025, CB21-499-026, CB21-499-027, CB21-499-028, CB21-499-029, CB21-499-030, CB21-499-031, CB21-499-032, CB21-499-033, CB21-499-034, CB21-499-035, CB21-499-036, CB21-499-037, CB21-499-038, CB21-499-039, CB21-499-040, CB21-499-041, CB21-499-042, CB21-499-043, CB21-499-044, CB21-499-045, CB21-499-046, CB21-499-047, CB21-499-048, CB21-499-049, CB21-499-050, CB21-499-051, CB21-499-052, CB21-499-053, CB21-499-054, CB21-499-055, CB21-499-056, CB21-499-057, CB21-499-058, CB21-499-059, CB21-499-060, CB21-499-061, CB21-499-062, CB21-499-063, CB21-499-064, CB21-499-065, CB21-499-066, CB21-499-067, CB21-499-068, CB21-499-069, CB21-499-070, CB21-499-071, CB21-499-072, CB21-499-073, CB21-499-074, CB21-499-075, CB21-499-076, CB21-499-077, CB21-499-078, CB21-499-079, CB21-499-080, CB21-499-081, CB21-499-082, CB21-499-083, CB21-499-084, CB21-499-085, CB21-499-086, CB21-499-087, CB21-499-088, CB21-499-089, CB21-499-090, CB21-499-091, CB21-499-092, CB21-499-093, CB21-499-094, CB21-499-095, CB21-499-096, CB21-499-097, CB21-499-098, CB21-499-099, CB21-499-100, CB21-499-101, CB21-499-102, CB21-499-103, CB21-499-104, CB21-499-105, CB21-499-106, CB21-499-107, CB21-499-108, CB21-499-109, CB21-499-110, CB21-499-111, CB21-499-112, CB21-499-113, CB21-499-114, CB21-499-115, CB21-499-116, CB21-499-117, CB21-499-118, CB21-499-119, CB21-499-120, CB21-499-121, CB21-499-122, CB21-499-123, CB21-499-124, CB21-499-125, CB21-499-126, CB21-499-127, CB21-499-128, CB21-499-129, CB21-499-130, CB21-499-131, CB21-499-132, CB21-499-133, CB21-499-134, CB21-499-135, CB21-499-136, CB21-499-137, CB21-499-138, CB21-499-139, CB21-499-140, CB21-499-141, CB21-499-142, CB21-499-143, CB21-499-144, CB21-499-145, CB21-499-146, CB21-499-147, CB21-499-148, CB21-499-149, CB21-499-150, CB21-499-151, CB21-499-152, CB21-499-153, CB21-499-154, CB21-499-155, CB21-499-156, CB21-499-157, CB21-499-158, CB21-499-159, CB21-499-160, CB21-499-161, CB21-499-162, CB21-499-163, CB21-499-164, CB21-499-165, CB21-499-166, CB21-499-167, CB21-499-168, CB21-499-169, CB21-499-170, CB21-499-171, CB21-499-172, CB21-499-174, CB21-499-175, CB21-499-176, CB21-499-177, CB21-499-178, and CB21-499-179.

Embodiment 16: The antibody according to any one of embodiments 1-13, wherein the antigen or binding fragment comprises a heavy chain variable region and a light chain variable region comprising amino acid sequences as recited for an Identifier in Table 2 selected from the group consisting of CB21-499-177 and CB21-498-142.

Embodiment 17: An isolated nucleic acid encoding any one of the antibodies or antigen binding fragments of embodiments 1-16.

Embodiment 18: An expression vector comprising the isolated nucleic acid of embodiment 17.

Embodiment 19: A host cell comprising expression vector of embodiment 18.

Embodiment 20: The host cell of embodiment 19, which is a bacterial cell, a human cell, a mammalian cell, a Pichia cell, a plant cell, an HEK293 cell, or a CHO cell.

Embodiment 21: A composition comprising the antibody or antigen binding fragment of any one of embodiments 1-16 and a pharmaceutically acceptable carrier or diluent.

Embodiment 22: The composition according to embodiment 21, further comprising one or more agents selected from the group consisting of anti-CD27 antibody, anti-CD47 antibody, anti-APRIL antibody, anti-PD-1 antibody, anti-PD-L1 antibody, anti-TIGIT antibody, anti-CTLA4 antibody, anti-CS1 antibody, anti-KIR2DL1/2/3 antibody, anti-CD137 antibody, anti-GITR antibody, anti-PD-L2 antibody, anti-ILT1 antibody, anti-ILT2 antibody, anti-ILT3 antibody, anti-ILT4 antibody, anti-ILT5 antibody, anti-ILT6 antibody, anti-ILT7 antibody, anti-ILT8 antibody, anti-CD40 antibody, anti-OX40 antibody, anti-ICOS, anti-KIR2DL1 antibody, anti-KIR2DL2/3 antibody, anti-KIR2DL4 antibody, anti-KIR2DL5A antibody, anti-KIR2DL5B antibody, anti-KIR3DL1 antibody, anti-KIR3DL2 antibody, anti-KIR3DL3 antibody, anti-NKG2A antibody, anti-NKG2C antibody, anti-NKG2E antibody, anti-4-1BB antibody, anti-TSLP antibody, anti-IL-10 antibody, IL-10 PEGylated IL-10, an agonist (e.g., an agonistic antibody or antigen-binding fragment thereof, or a soluble fusion) of a TNF receptor protein, an Immunoglobulin-like protein, a cytokine receptor, an integrin, a signaling lymphocytic activation molecules (SLAM proteins), an activating NK cell receptor, a Toll like receptor, OX40, CD2, CD7, CD27, CD28, CD30, CD40, ICAM-1, LFA-1 (CDl 1a/CD18), 4-1BB (CD137), B7-H3, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CDl 1d, ITGAE, CD103, ITGAL, ITGAM, CDl 1b, ITGAX, CDl 1c, ITGB1, CD29, ITGB2, CD18, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), SLAM7, BLAME (SLAMF8), SELPLG (CD162), LTBR, LAT, GADS, PAG/Cbp, CD19a, a ligand that specifically binds with CD83, an inhibitor of CD47, an inhibitor of PD-1, an inhibitor of PD-L1, an inhibitor of PD-L2, an inhibitor of CTLA4, an inhibitor of TIM3, an inhibitor of LAG3, an inhibitor of CEACAM (e.g., CEACAM-1, -3 and/or -5), an inhibitor of VISTA, an inhibitor of BTLA, an inhibitor of TIGIT, an inhibitor of LAIR1, an inhibitor of IDO, an inhibitor of TDO, an inhibitor of CD160, an inhibitor of TGFR beta, and a cyclic dinculeotide or other STING pathway agonist.

Embodiment 23: A method of producing an antibody or antigen binding fragment comprising:
culturing a host cell comprising one or more polynucleotides encoding any one of the antibodies or antigen binding fragments of embodiments 1-16 under conditions favorable to expression of the polynucleotide; and optionally, recovering the antibody or antigen binding fragment from the host cell and/or culture medium.

Embodiment 24: An antibody or antigen binding fragment according to any one of embodiments 1-16 or a composition according to embodiment 18 or 19, for the treatment of cancer.

Embodiment 25. A method of treating cancer in a human subject, comprising administering to the subject an effective amount of an antibody or antigen binding fragment of any one of claims 1-16, or an expression vector according to claim 18, or a composition according one of claim 21 or 22, optionally in association with a further therapeutic agent or therapeutic procedure.

### General Methods

Standard methods in molecular biology are described Sambrook, Fritsch and Maniatis (1982 & 1989 2^{nd} Edition, 2001 3^{rd} Edition) Molecular Cloning, A Laboratory Manual, Cold Spring Harbor Laboratory Press, Cold Spring Harbor, NY; Sambrook and Russell (2001) Molecular Cloning, 3^{rd} ed., Cold Spring Harbor Laboratory Press, Cold Spring Harbor, NY; Wu (1993) Recombinant DNA, Vol. 217, Academic Press, San Diego, CA). Standard methods also appear in Ausbel, et al. (2001) Current Protocols in Molecular Biology, Vols. 1-4, John Wiley and Sons, Inc. New York, NY, which describes cloning in bacterial cells and DNA mutagenesis (Vol. 1), cloning in mammalian cells and yeast (Vol. 2), glycoconjugates and protein expression (Vol. 3), and bioinformatics (Vol. 4).

Methods for protein purification including immunoprecipitation, chromatography, electrophoresis, centrifugation, and crystallization are described (Coligan, et al. (2000) Current Protocols in Protein Science, Vol. 1, John Wiley and Sons, Inc., New York). Chemical analysis, chemical modification, post-translational modification, production of fusion proteins, glycosylation of proteins are described (see, e.g., Coligan, et al. (2000) Current Protocols in Protein Science, Vol. 2, John Wiley and Sons, Inc., New York; Ausubel, et al. (2001) Current Protocols in Molecular Biology, Vol. 3, John Wiley and Sons, Inc., NY, NY, pp. 16.0.5-16.22.17; Sigma-Aldrich, Co. (2001) Products for Life Science Research, St. Louis, MO; pp. 45-89; Amersham Pharmacia Biotech (2001) BioDirectory, Piscataway, N.J., pp. 384-391). Production, purification, and fragmentation of polyclonal and monoclonal antibodies are described (Coligan, et al. (2001) Current Protocols in Immunology, Vol. 1, John Wiley and Sons, Inc., New York; Harlow and Lane (1999) Using Antibodies, Cold Spring Harbor Laboratory Press, Cold Spring Harbor, NY; Harlow and Lane, supra). Standard techniques for characterizing ligand/receptor interactions are available (see, e.g., Coligan, et al. (2001) Current Protocols in Immunology, Vol. 4, John Wiley, Inc., New York).

Monoclonal, polyclonal, and humanized antibodies can be prepared (see, e.g., Sheperd and Dean (eds.) (2000) Monoclonal Antibodies, Oxford Univ. Press, New York, NY; Kontermann and Dubel (eds.) (2001) Antibody Engineering, Springer-Verlag, New York; Harlow and Lane (1988) Antibodies A Laboratory Manual, Cold Spring Harbor Laboratory Press, Cold Spring Harbor, NY, pp. 139-243; Carpenter, et al. (2000) J. Immunol. 165:6205; He, et al. (1998) J. Immunol. 160:1029; Tang et al. (1999) J. Biol. Chem. 274:27371-27378; Baca et al. (1997) J. Biol. Chem. 272:10678-10684; Chothia et al. (1989) Nature 342:877-883; Foote and Winter (1992) J. Mol. Biol. 224:487-499; U.S. Pat. No. 6,329,511).

An alternative to humanization is to use human antibody libraries displayed on phage or human antibody libraries in transgenic mice (Vaughan et al. (1996) Nature Biotechnol. 14:309-314; Barbas (1995) Nature Medicine 1:837-839; Mendez et al. (1997) Nature Genetics 15:146-156; Hoogenboom and Chames (2000) Immunol. Today 21:371-377; Barbas et al. (2001) Phage Display: A Laboratory Manual, Cold Spring Harbor Laboratory Press, Cold Spring Harbor, New York; Kay et al. (1996) Phage Display of Peptides and Proteins: A Laboratory Manual, Academic Press, San Diego, CA; de Bruin et al. (1999) Nature Biotechnol. 17:397-399).

Single chain antibodies and diabodies are described (see, e.g., Malecki et al. (2002) Proc. Natl. Acad. Sci. USA 99:213-218; Conrath et al. (2001) J. Biol. Chem. 276:7346-7350; Desmyter et al. (2001) J. Biol. Chem. 276:26285-26290; Hudson and Kortt (1999) J. Immunol. Methods 231:177-189; and U.S. Pat. No. 4,946,778). Bifunctional antibodies are provided (see, e.g., Mack, et al. (1995) Proc. Natl. Acad. Sci. USA 92:7021-7025; Carter (2001) J. Immunol. Methods 248:7-15; Volkel, et al. (2001) Protein Engineering 14:815-823; Segal, et al. (2001) J. Immunol. Methods 248:1-6; Brennan, et al. (1985) Science 229:81-83; Raso, et al. (1997) J. Biol. Chem. 272:27623; Morrison (1985) Science 229:1202-1207; Traunecker, et al. (1991) EMBO J. 10:3655-3659; and U.S. Pat. Nos. 5,932,448, 5,532,210, and 6,129,914).

Bispecific antibodies are also provided (see, e.g., Azzoni et al. (1998) J. Immunol. 161:3493; Kita et al. (1999) J. Immunol. 162:6901; Merchant et al. (2000) J. Biol. Chem. 74:9115; Pandey et al. (2000) J. Biol. Chem. 275:38633; Zheng et al. (2001) J. Biol Chem. 276:12999; Propst et al. (2000) J. Immunol. 165:2214; Long (1999) Ann. Rev. Immunol. 17:875).

Purification of antigen is not necessary for the generation of antibodies. Animals can be immunized with cells bearing the antigen of interest. Splenocytes can then be isolated from the immunized animals, and the splenocytes can fused with a myeloma cell line to produce a hybridoma (see, e.g., Meyaard et al. (1997) Immunity 7:283-290; Wright et al. (2000) Immunity 13:233-242; Preston et al., supra; Kaithamana et al. (1999) J. Immunol. 163:5157-5164).

Antibodies can be conjugated, e.g., to small drug molecules, enzymes, liposomes, polyethylene glycol (PEG). Antibodies are useful for therapeutic, diagnostic, kit or other purposes, and include antibodies coupled, e.g., to dyes, radioisotopes, enzymes, or metals, e.g., colloidal gold (see, e.g., Le Doussal et al. (1991) J. Immunol. 146:169-175; Gibellini et al. (1998) J. Immunol. 160:3891-3898; Hsing and Bishop (1999) J. Immunol. 162:2804-2811; Everts et al. (2002) J. Immunol. 168:883-889).

Methods for flow cytometry, including fluorescence activated cell sorting (FACS), are available (see, e.g., Owens, et al. (1994) Flow Cytometry Principles for Clinical Laboratory Practice, John Wiley and Sons, Hoboken, NJ; Givan (2001) Flow Cytometry, 2^{nd} ed.; Wiley-Liss, Hoboken, NJ; Shapiro (2003) Practical Flow Cytometry, John Wiley and Sons, Hoboken, NJ). Fluorescent reagents suitable for modifying nucleic acids, including nucleic acid primers and probes, polypeptides, and antibodies, for use, e.g., as diagnostic reagents, are available (Molecular Probes (2003) Catalogue, Molecular Probes, Inc., Eugene, OR; Sigma-Aldrich (2003) Catalogue, St. Louis, MO).

Standard methods of histology of the immune system are described (see, e.g., Muller-Harmelink (ed.) (1986) Human Thymus: Histopathology and Pathology, Springer Verlag, New York, NY; Hiatt, et al. (2000) Color Atlas of Histology, Lippincott, Williams, and Wilkins, Phila, PA; Louis, et al. (2002) Basic Histology: Text and Atlas, McGraw-Hill, New York, NY).

Software packages and databases for determining, e.g., antigenic fragments, leader sequences, protein folding, functional domains, glycosylation sites, and sequence alignments, are available (see, e.g., GenBank, Vector NTI® Suite (Informax, Inc, Bethesda, MD); GCG Wisconsin Package (Accelrys, Inc., San Diego, CA); DeCypher® (TimeLogic Corp., Crystal Bay, Nevada); Menne, et al. (2000) Bioinformatics 16:741-742; Menne, et al. (2000) Bioinformatics Applications Note 16:741-742; Wren, et al. (2002) Comput. Methods Programs Biomed. 68:177-181; von Heijne (1983) Eur. J. Biochem. 133:17-21; von Heijne (1986) Nucleic Acids Res. 14:4683-4690).

### EXAMPLES

The following examples serve to illustrate the present invention. These examples are in no way intended to limit the scope of the invention.

### Example 1

Standard molecular cloning methods were used to construct plasmids for antibody expression. Antibody variable domains of anti-LY6G6D variants were synthesized as linear gene fragments by external provider Integrated DNA Technologies. Custom knob-human Fc, hole-human Fc, and light chain expression vectors were built from a pcDNA3.4 parental vector (Thermo Scientific). These custom antibody expression vectors were prepared by linearation with BamHI (NEB R3136M) digest. Gibson Assembly was subsequently performed with variable domain inserts using NEBuilder HiFi DNA Assembly Mastermix (cat. no. M5520AAVIAL). The resulting plasmid DNA was sequence verified by plasmid sequencing and scaled for transient transfection in Expi293 cells at 3 or 30 ml/antibody. Both IgG and bispecific TCE formats were produced for each clone.

Human (SEQ ID NO: 1) and Cynomolgus (NRMRCYNCGGSPSSSCKEAVTTCGEGRPQPGLEQIKLPGNPPVTLIHQHPACVA ARHCNQVETESVGDVTYPAHRDCYLGDLCNS; SEQ ID NO: 2667) LY6G6D ectodomains, truncating after the signal peptide and before the GPI anchor sequence, were cloned as N-terminal fusions with a GGGGS linker to mouse IgG2A Fc. Specifically, mouse IgG2A Fc utilized knob-in-hole mutations to monovalent protein display. The ectodomain-mouse Fc hole plasmid was co-transfected with empty knob mouse Fc into Expi293F cells with Expifectamine. Supernatant was harvested and NiNTA-purified after five days.

Protein Expression, Titers & Purification: All antibodies were expressed in and purified from Expi293F (Thermo Fisher Scientific) or modified Expi293 BirA-KDEL (PMID: 29359686) cells according to an established protocol from the manufacturer (Thermo Fisher Scientific). Briefly, pcDNA3.4 vector (Thermo Scientific) vector encoding the protein of interest was transiently transfected into Expi293F cells at a fixed density of 3×10⁶/mL using the Expifectamine transfection kit and manufacturer's protocol (Thermo Fisher Scientific). Expression volumes varied, but a fixed vector transfection reagent ratio (1 mg: 1 mL) was used. For multi-chain proteins, a stoichiometric equivalent of each respective vector was used. Culture media for BirA cells was additionally supplemented with 100 pM of biotin prior to transfection for in vivo biotinylation. Enhancing supplements from the Expifectamine kit were added 20 hours post-transfection. Cells were maintained for four days at 37° C. with 8% CO2 on a Celltron shaking platform (Infors) at 118 rpm. Culture supernatants were harvested and clarified via centrifugation at 4000×g for 45 mins. Fc-fusion proteins were purified by Protein A (Cytiva MabSelect PrismA) affinity chromatography and His-tagged proteins were purified by Ni-NTA (Roche cOmplete™ His-Tag Resin) affinity chromatography. For Protein A affinity chromatography, resin was washed 3-5× with at least 30-50 total bed volumes of 1×HBS (50 mM HEPES-NaOH pH 7.4+150 mM NaCl). Purified protein was eluted with 5 bed volumes of Pro A Elution Buffer (20 mM Acetate, pH 3.5 (Ricca Chemical). To neutralize the acidic eluates, Tris-HCl (pH 8.0) was added to the eluted fractions to a final concentration of 100 mM. For Ni-NTA affinity chromatography, resin was washed with a series of 10-20 bed volumes of three sequential wash buffers: Wash buffer 1 (1×HBS with 10 mM imidazole), Wash buffer 2 (0.5 mM EDTA in 1×HBS with 10 mM imidazole), Wash buffer 3 (0.5 M NaCl in 1×HBS with 10 mM imidazole). Purified protein was eluted with 500 mM imidazole in 1×HBS with 10% glycerol. Eluted fractions were buffer exchanged to 1×HBS with 10% glycerol. Purity and integrity were assessed by SDS-PAGE with 4-12% Mini-PROTEAN TGX stain-free precast gels (Bio-Rad). Samples were stored at either 4° C. or −80° C. with storage buffer in 50-uL or 100-uL aliquots.

Supernatant titer quantitation was performed accordingly. Briefly, supernatants from 24-well plates were harvested, centrifuged at 4000 g for 45 minutes to pellet expression cells, and clarified supernatants were transferred to a GatorBio BLI 96-well polypropylene flat plate. GatorBio BLI Protein A probes were used with standard manufactures protocol for sample quantitation to measure antibody concentrations. Sample concentrations were extrapolated from a standard curve generated with an IgG reference molecule diluted in Expi293F media.

### Example 2

The LY6G6D cyclic peptide was designed to interrogate and quantify the specificity of recognition of the protein region most proximal to the GPI anchor. The peptide sequence consisting of Ac-RDC*YLGDLC*NS-PEG2-3-mercaptopropionate, was designed with C* a disulfide bridge stabilized with a non-reducible methylene group (Chemitope Glycopeptide, custom order). The intention behind this conformationally-stabilized peptide was to recapitulate the predicted structure of the most-membrane-proximal peptide stretch as a specific peptide detection reagent. The mercaptoproprionate handle was utilized to conjugate this cyclic peptide into an octomerized multivalent reagent. A 7:1 ratio of Ac-RDC*YLGDLC*NS-PEG2-3-mercaptopropionate to SH-PEG-Biotin (Biopharma PEG HE003041-2K) was used to conjugate to a 8-Arm PEG-MAL, MW 10 k (Creative PEGWorks PSB-861). The NHS ester conjugation was quenched with fresh

2-Mercaptoethanol (Sigma-Aldrich M6250). Finally, the octomer cyclic peptide was diluted to 100 uM with ethylene glycol (Sigma-Aldrich 102466). This peptide was coupled to streptavidin beads, and these peptide-bead complexes were utilized in a flow binding assays to detect peptide-specific binding. Here, beads are incubated with LY6G6D-binding supernatant or serum, and fluorescence signal is detected with species-specific secondary antibody.

Genetic immunization with Alloy ATX-GK BALB/c mice yielded strong reactive titers against LY6G6D-overexpression cells and the “RDCYLGDLCNS” cyclic peptide, representing the most membrane-proximal epitope of LY6G6D. Lymph node (CD138+) and bone marrow-derived (CD138+/CD45R−) plasma cells were enriched and then screened on the Bruker Cellular Analysis Beacon platform. A total of 194 cells were exported, cDNA was amplified, and binding was tested via transient heterologous expression.

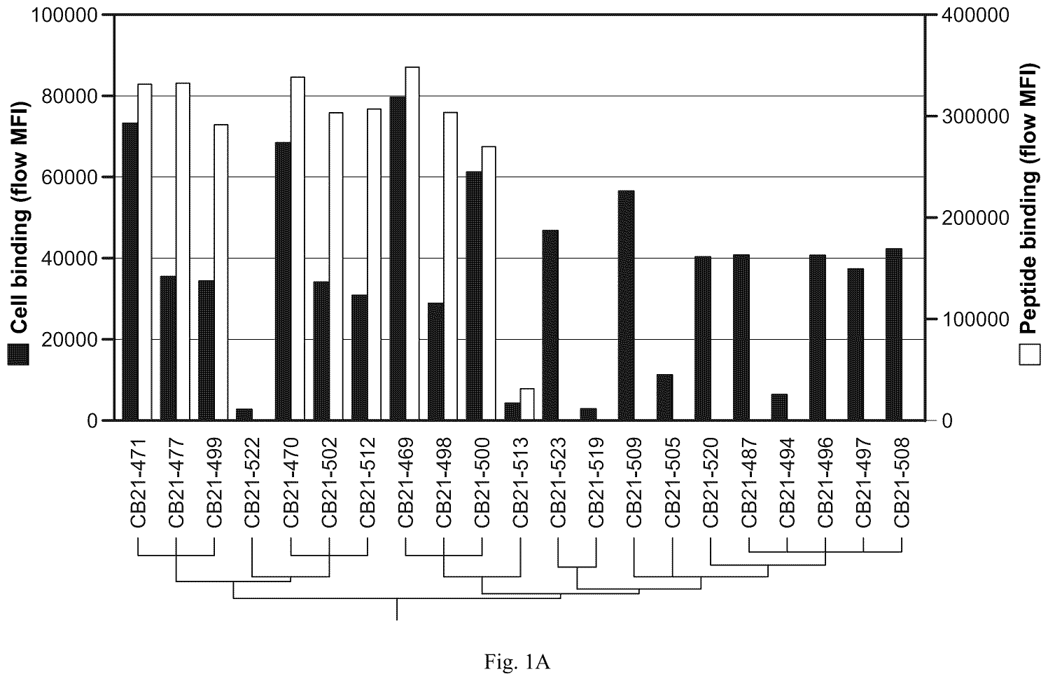

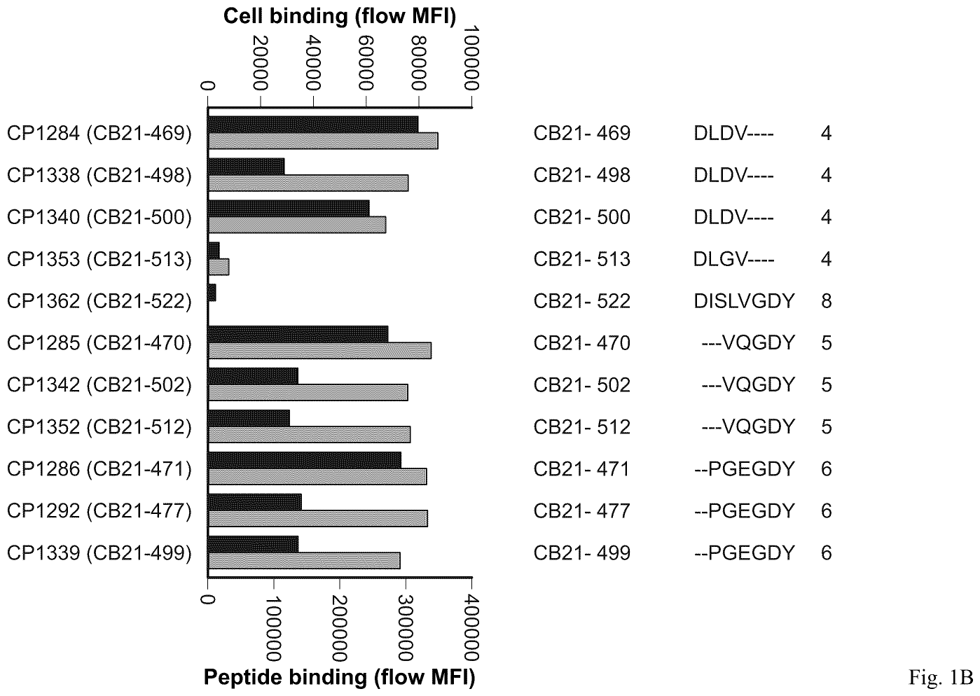

Ultimately, 56 clones were recovered following re-evaluation of binding to LY6G6D-overexpression cells and the “RDCYLGDLCNS” cyclic peptide. CDRH3s were assembled into a guide tree alongside these binding data to begin mapping sequence-binding relationships as shown in FIG. 1A. A branch of eleven related antibody sequences (identified as CB21-469, CB21-470, CB21-471, CB21-477, CB21-498, CB21-499, CB21-500, CB21-502, CB21-512, CB21-513 and CB21-522) demonstrating strong binding to a target membrane-proximal epitope on LY6G6D, were selected for further study. A Martin tree alignment of the heavy chain CDR3 and the corresponding binding characteristics are shown in FIG. 1B.

A related branch that exhibited cell binding but not the desired target peptide binding was identified as including antibodies CB21-519, CB21-509, CB21-505, CB21-520, CB21-487, CB21-494, CB21-496, CB21-497, and CB21-508.

### Example 3

Anti-LY6G6D clones were screened for binding to LY6G6D ectodomain constructs as described above (Human construct referred to in the Figures as “CP203”) by Biolayer Interferometry (BLI) using a Gator Bio Plus instrument. Binding was analyzed using Streptaidin (SA) probes to capture biotinylated LY6G6D ectodomain protein at 20 nM, followed by a 300s association of LY6G6D monovalent antibody in a concentration series from 100 nM diluted 4-fold down to 6.25 nM. A dissociation step was performed in Gator Q buffer and kinetics were determined using a 1:1 Langmuir binding model processed using Gator analysis software.

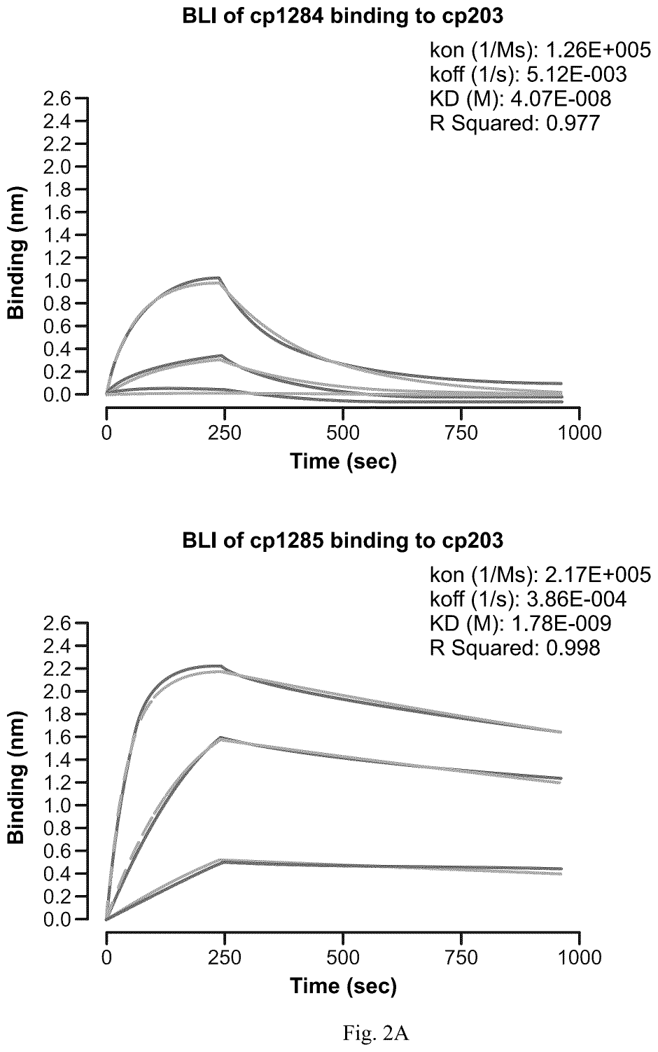

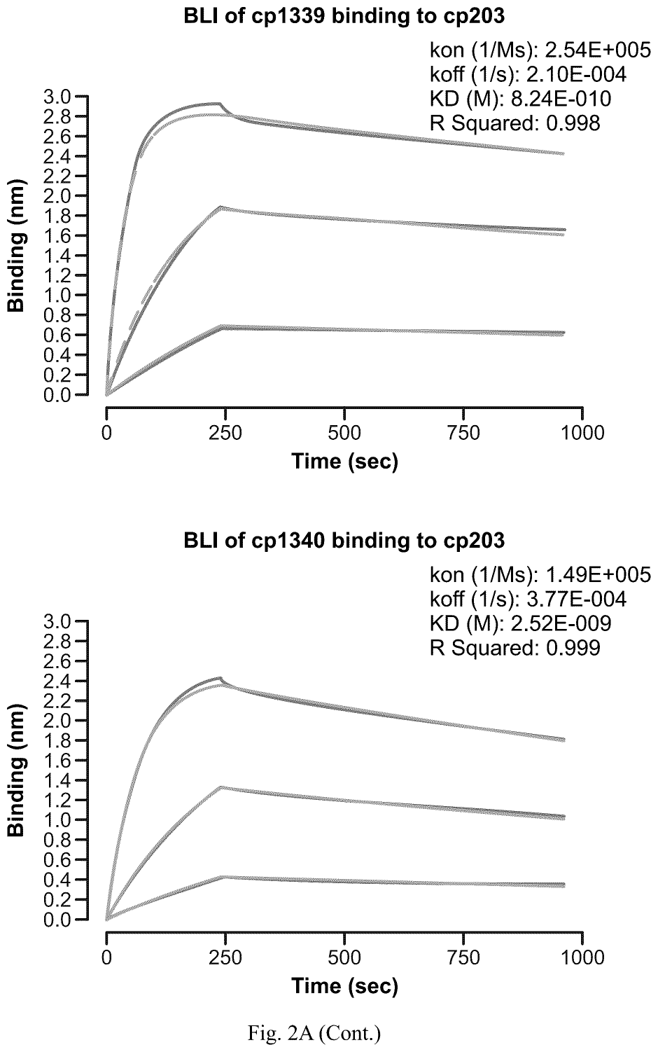

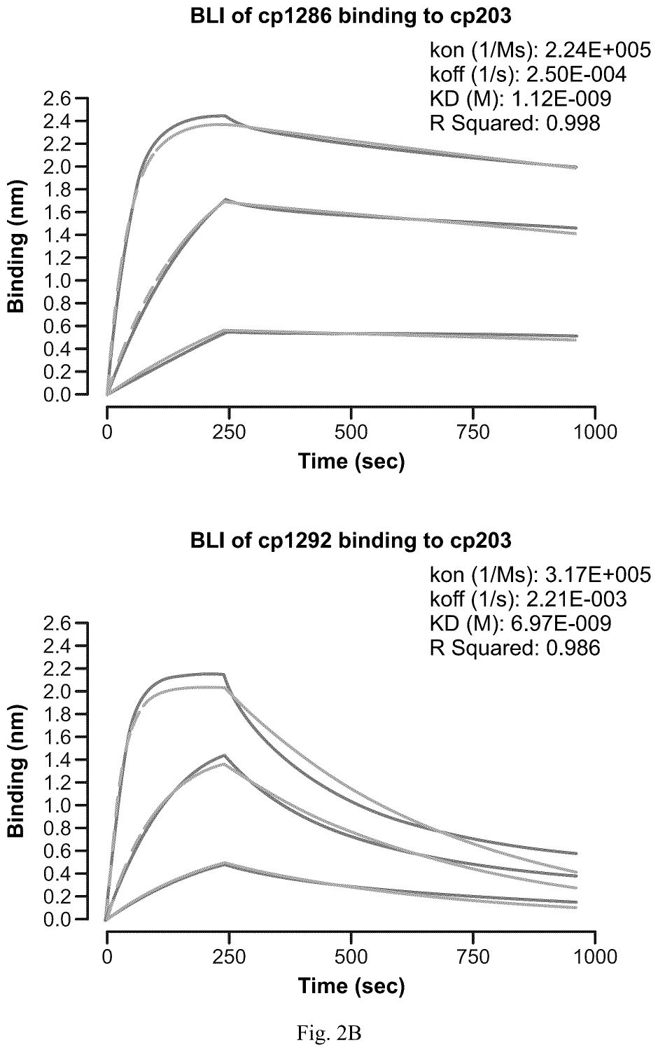

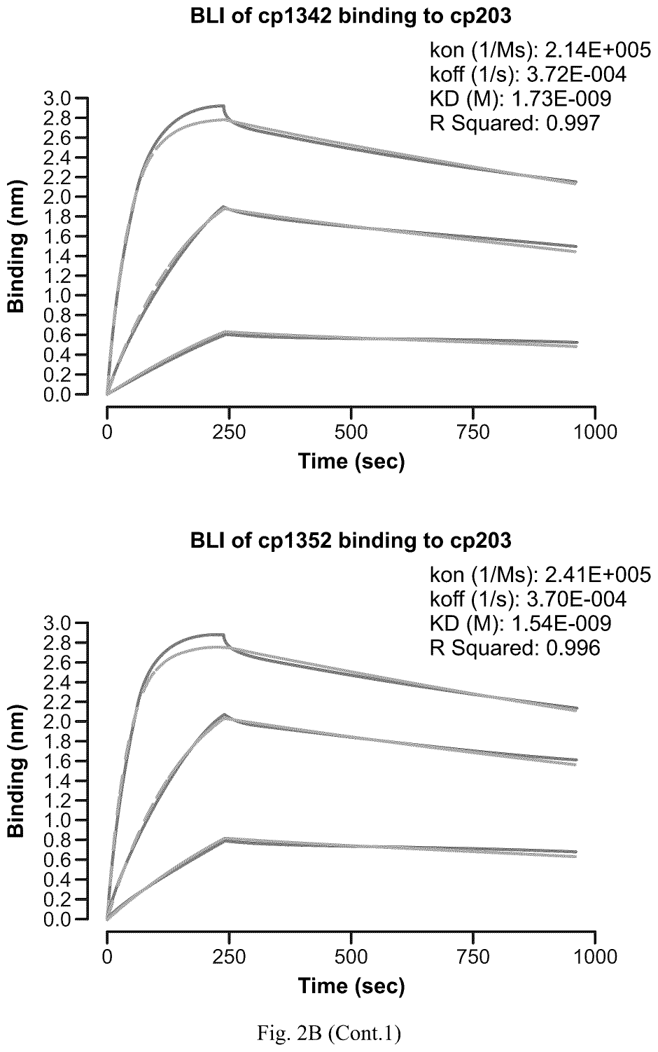

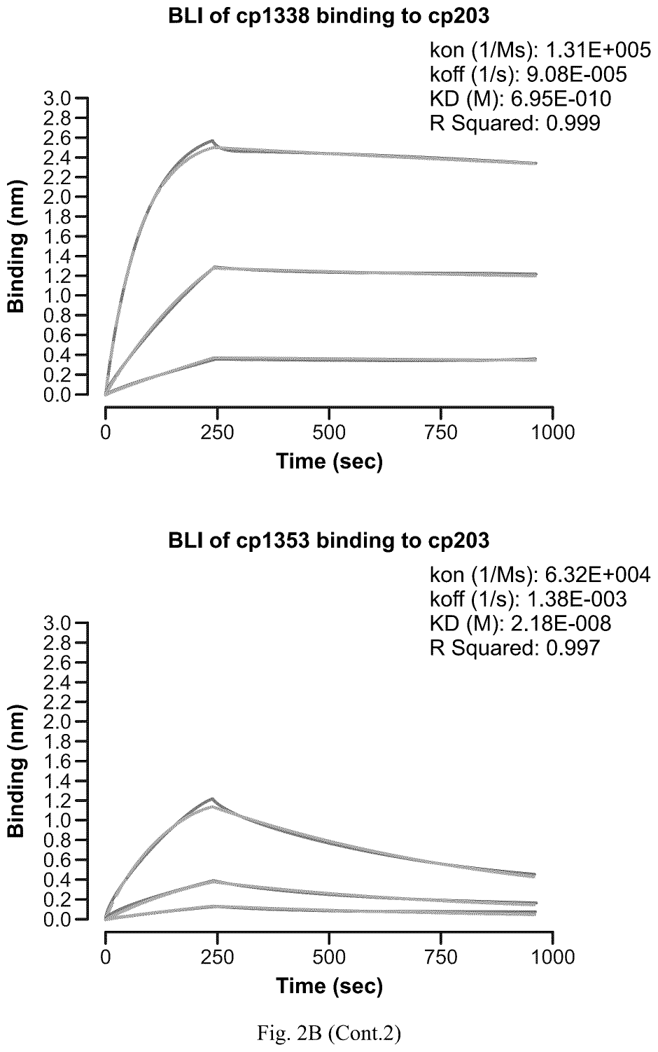

The Multipoint BLI kinetic analysis of LY6G6D binding by the 11 parental antibody clones described in FIGS. 1A and B are depicted in FIGS. 2A and B, showing a range of association and dissociation rates against recombinant monovalent LY6G6D ECD Fc-fusion protein.

### Example 4

### Baculovirus Particle ELISA (BVP-ELISA)

Baculovirus Particle ELISA was used to evaluate the polyspecificity of test antibodies by measuring their binding to baculovirus-derived particles. Baculovirus particles were immobilized onto a 96-well ELISA plate using standard coating procedures. Each test antibody was then added at a concentration of 100 g/mL and incubated to allow binding to the particles. Detection of bound antibody was performed using an anti-human Fc secondary antibody conjugated to horseradish peroxidase (HRP), followed by colorimetric development. Elevated binding to baculovirus particles is indicative of increased nonspecific interactions and may correlate with reduced in vivo stability, including accelerated serum clearance.

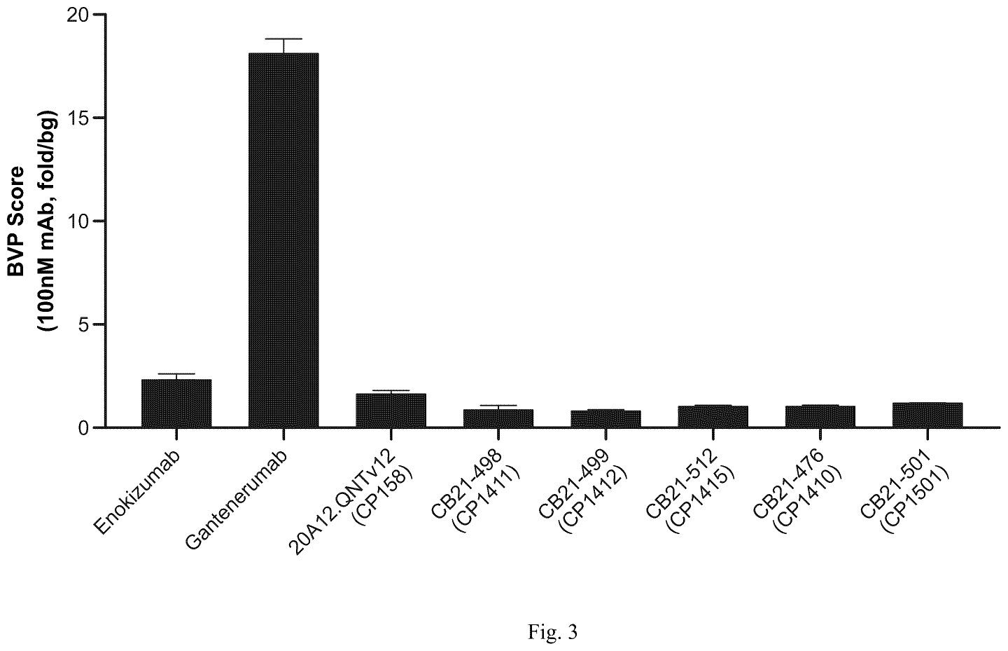

Parental antibodies CB21-498, CB21-499, CB21-512, CB21-476, and CB21-501 were evaluated for polyreactivity using the BVP ELISA. As shown in FIG. 3, all clones exhibited low polyreactivity comparable to or lower than the reference antibody enokizumab, and significantly lower than gantenerumab, a known highly polyreactive antibody. These clones also demonstrated slightly lower polyreactivity than the literature-reported LY6G6D binder 20A12.QNTv12.

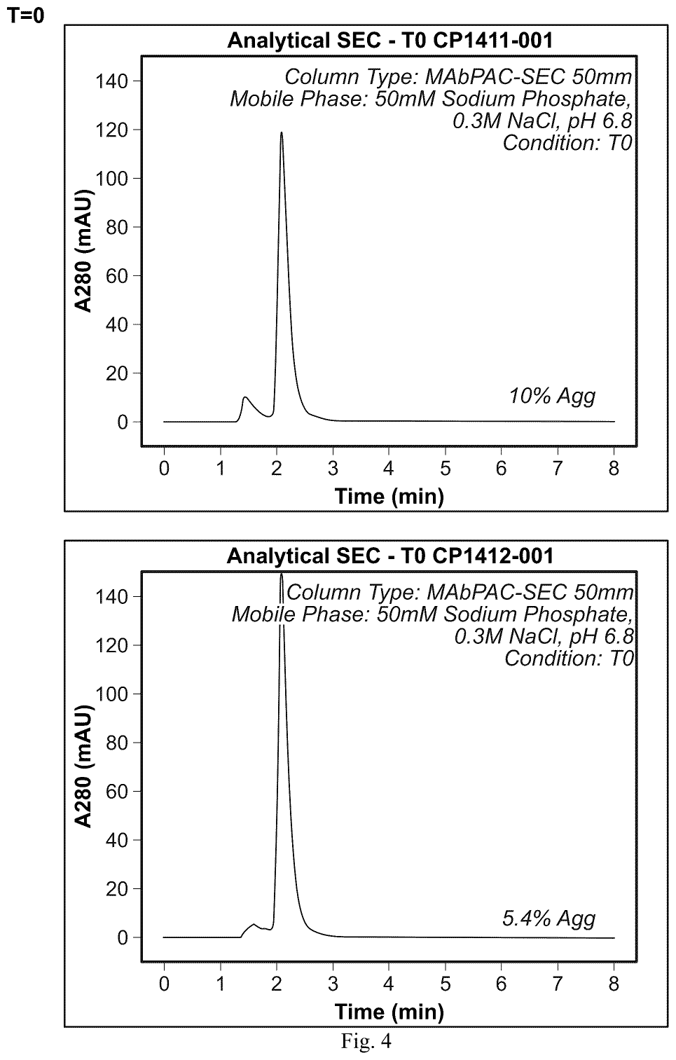

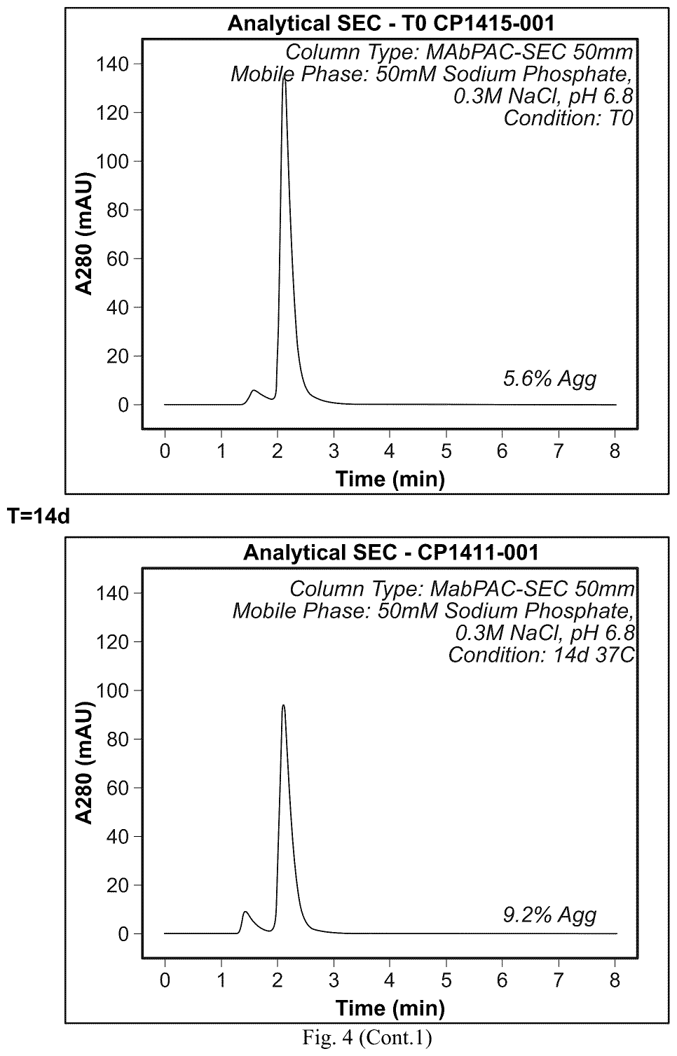

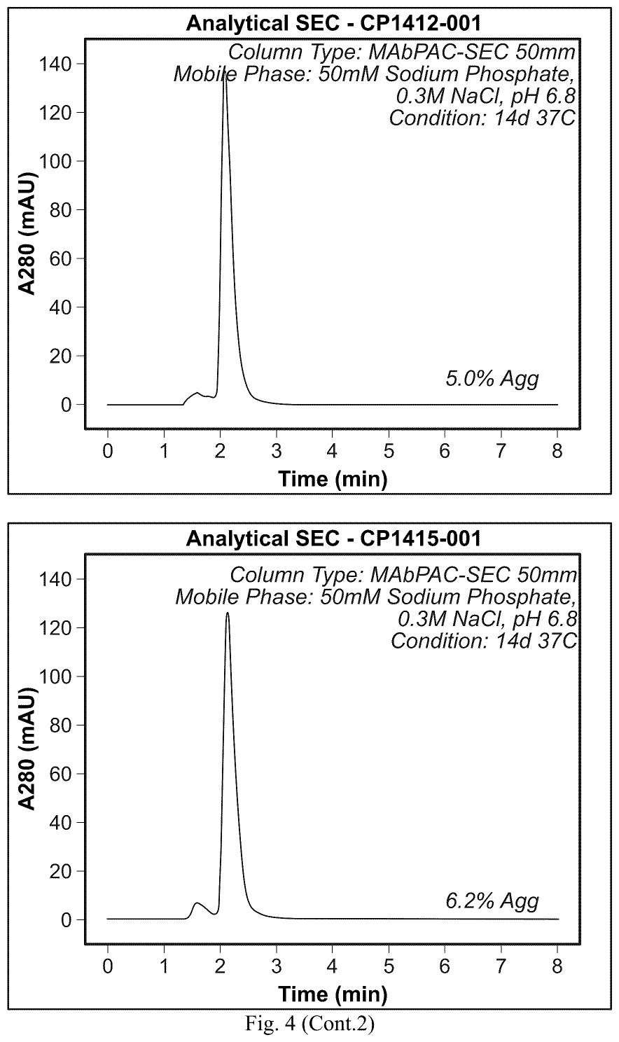

Three parental antibody clones (CB21-498, CB21-499, and CB21-512) were subjected to accelerated thermal stress at 40° C. for 14 days and analyzed by SEC-HPLC at T0 and Day 14. Analytical Size Exclusion Chromatography: SEC-HPLC was performed on a Thermo Scientific Vanquish Flex UHPLC system. Proteins were injected neat, with 2.5 μg loaded onto a MabPAC™ SEC-1 4×50 mm (5 μm) column, using a Mobile Phase of 50 mM Sodium Phosphate and 0.3M Sodium Chloride, at pH 6.8. Acquisition of chromatograms were acquired continuously at 280 nm and analyzed using Thermo Scientific's Chromeleon 7 software. As shown in FIG. 4, all three clones demonstrated minimal change in aggregate levels, indicating resistance to thermal degradation and low formation of high molecular weight (HMW) species.

### Example 5

### AbMap (Antibody Binding Epitope Mapping)

AbMap was carried out using NEB's Ph.D.12 Phage Display Library (NEB E8210S), a combinatorial library of random 12-mer peptides displayed on M13 phage. The phage library was first amplified, purified, and quantified. Approximately 1011 plaque-forming units were incubated overnight with 0.4 μg of the antibody. Antibody-bound phage were captured post-incubation using Dynabeads Protein G (Thermo Scientific). Bound phage were then lysed at 95° C. for 10 minutes, and two rounds of PCR were used to amplify peptide-coding sequences and append Illumina adapters. The resulting libraries were sequenced on an Illumina NextSeq 2000 platform using paired-end 2×100 bp reads. Sequencing data was quality filtered, and peptide abundance was quantified relative to a no-antibody control. De-novo peptide motifs were computationally derived from the set of enriched peptides and subsequently mapped onto the linear sequence of the target protein, enabling high-resolution localization of antibody-binding epitopes.

### Example 6

### In Silico Antibody Engineering

For thermostability and aggregation propensity analysis, Schrödinger BioLuminate was used. Structural models were first generated from primary antibody sequences using ABodyBuilder2 (Abanades et al., Communications Biology, 2023), and these models served as the input for structure-based calculations in BioLuminate. Aggregation risk was predicted using surface descriptor analysis, while thermostability was evaluated through residue scanning with ΔΔG calculations. Additional variant suggestions were obtained from a general protein language model-based tool for antibody optimization (Hie et al., Nature Biotechnology, 2024).

### Example 7

Lead optimization engineering rounds for CB21-498

### Overview:

| | | Key mutations |
|---|---|---|
| Round | CB21-498 goals | identified |
| 1 | Shuffle related heavy and light chains | LC: CB21-469 |
| 2 | Remove liabilities, test LLM | H: M2V, H: N54S, |
| | | L: G34A, L: A72S |
| 3 | Remove liabilities, focused on unpaired | H: C47G |
| | Cys | |
| 4 | Reduce aggregation | |
| 5 | Merge liability-scrubbing mutations | |
| 6 | Bump titers via aggregation/stability | H: K43R, H: T69Q, |
| | | H: S31D |
| 7 | Final merge of aggregation/stability/ | |
| | titer mutations | |

In round 1, heavy and light chains from CB21-469, CB21-498, CB21-500, and CB21-513 were recombinantly shuffled. Surprisingly, the light chain from CB21-469 paired with the heavy chain from CB21-499 was found to improve binding strength, as measured by BLI as shown in the following table.

TABLE 8

| | Heavy | Light | | | |
|---|---|---|---|---|---|
| Protein | chain from: | chain from: | KD (M) | Kon (1/Ms) | Koff (1/s) |
| CP1470 | CB21-469 | CB21-498 | | | |
| CP1471 | CB21-469 | CB21-500 | 1.27 × 10⁻⁸ | 1.34 × 10⁵ | 1.70 × 10−03 |
| CP1472 | CB21-469 | CB21-513 | | | |
| CP1473 | CB21-498 | CB21-469 | 5.54 × 10⁻¹⁰ | 1.52 × 10⁵ | 8.40 × 10⁻⁵ |
| CP1474 | CB21-498 | CB21-500 | 6.99 × 10⁻¹⁰ | 1.41 × 10⁵ | 9.87 × 10⁻⁵ |
| CP1475 | CB21-498 | CB21-513 | 9.10 × 10⁻¹⁰ | 1.16 × 10⁵ | 1.05 × 10⁻⁴ |
| CP1476 | CB21-500 | CB21-469 | 3.15 × 10⁻⁹ | 1.31 × 10⁵ | 4.13 × 10⁻⁴ |
| CP1477 | CB21-500 | CB21-498 | | | |
| CP1478 | CB21-500 | CB21-513 | 5.35 × 10⁻⁹ | 1.32 × 10⁵ | 7.05 × 10⁻⁴ |
| CP1479 | CB21-513 | CB21-469 | 1.65 × 10⁻⁸ | 3.22 × 10⁴ | 5.30 × 10⁻⁴ |
| CP1480 | CB21-513 | CB21-498 | | | |
| CP1481 | CB21-513 | CB21-500 | 1.76 × 10⁻⁸ | 1.50 × 10⁴ | 2.64 × 10⁻⁴ |

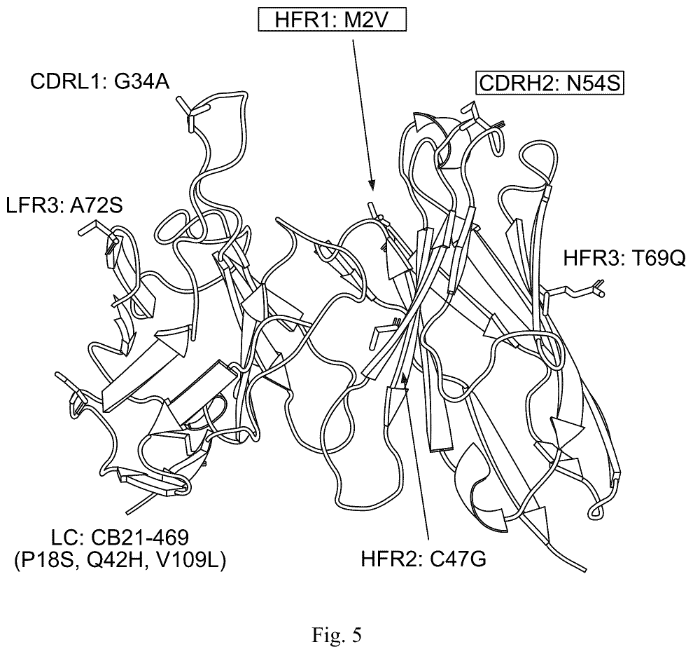

In round 2, putative solvent-accessible chemical liabilities inherent in the parent binder and germline reversion of unusual residues were addressed without impacting affinity or other biophysical properties. Mutations suggested by a recently published large language model (LLM)-based tool for antibody optimization (Hie et al., Nat Biotechnol 2024 PMID: 37095349) were considered, though none of these mutations resulted in significant improvements to binding. A substitution M2V (FIG. 5) addressed an unusual methionine and potential oxidation liability. The mutation N54S mitigated a potential deamidation site in CDRH2. G34A (FIG. 6) was introduced to reduce deamidation risk in CDRL1. A72S was selected to replace an unusual residue. However, an unpaired cystine C47 could not be removed without negatively impacting binding.

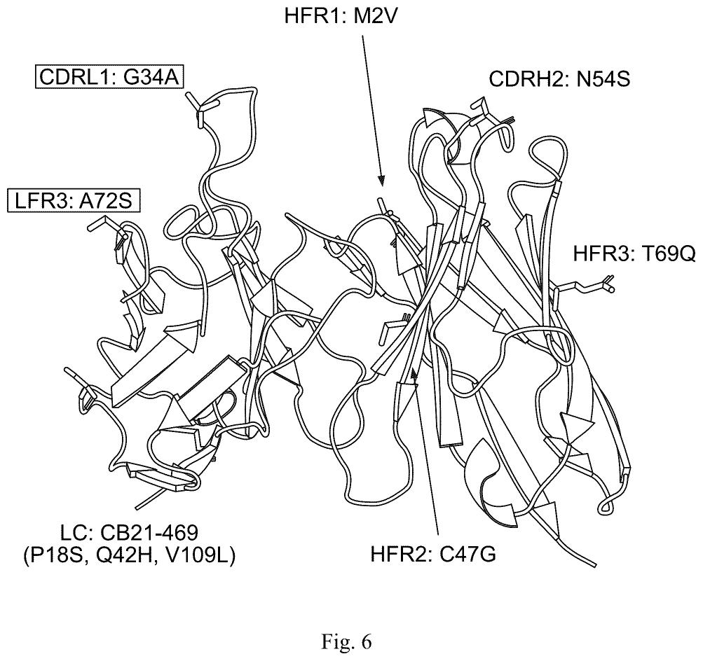

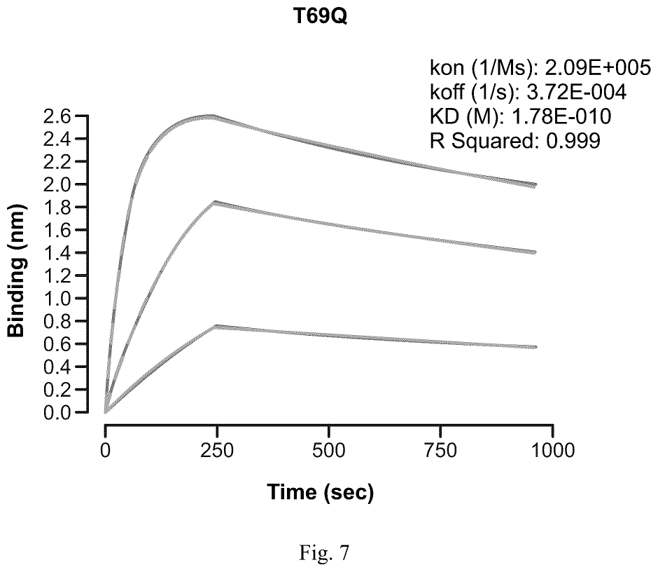

In silico analysis was performed to identify positions where mutation could potentially improve thermostability or reduce aggregation propensity. In particular, residue T69 was identified as a potential contributor to both properties. Through mutagenesis, the T69Q substitution was found to improve physical properties while maintaining potent binding (FIG. 7).

### Example 8

### Lead Optimization Engineering Rounds for CB21-498

### Overview:

| | | Key mutations |
|---|---|---|
| Round | CB21-499 goals | identified |
| 1 | Shuffle related heavy and light chains | N/A |
| 2 | Remove liabilities, test LLM | H: G55A, H: D62P |
| 3 | Merge liability-scrubbing mutations, | H: D59E |
| | improve thermostability | |
| 4 | Final merge of affinity-bumping mutation | |

In round 1 of CB21-499 engineering, closely related sequences containing the same CDRH3 from the original Beacon screen were identified. Heavy and light chains from CB21-471, CB21-477, and CB21-499 were recombinantly shuffled. Shuffling yielded no improvement over the original CB21-499 pairing, which remained the optimal clone in terms of binding affinity.

In round 2, two potential sites of isomerization and proteolysis were targeted for removal. Through mutagenesis, G55A in CDRH2 and D62P in HFR3 were identified as substitutions that mitigated these liabilities without significantly impacting binding affinity. Mutations suggested by a recently published large language model (LLM)-based tool for antibody optimization did not yield meaningful improvements in affinity.

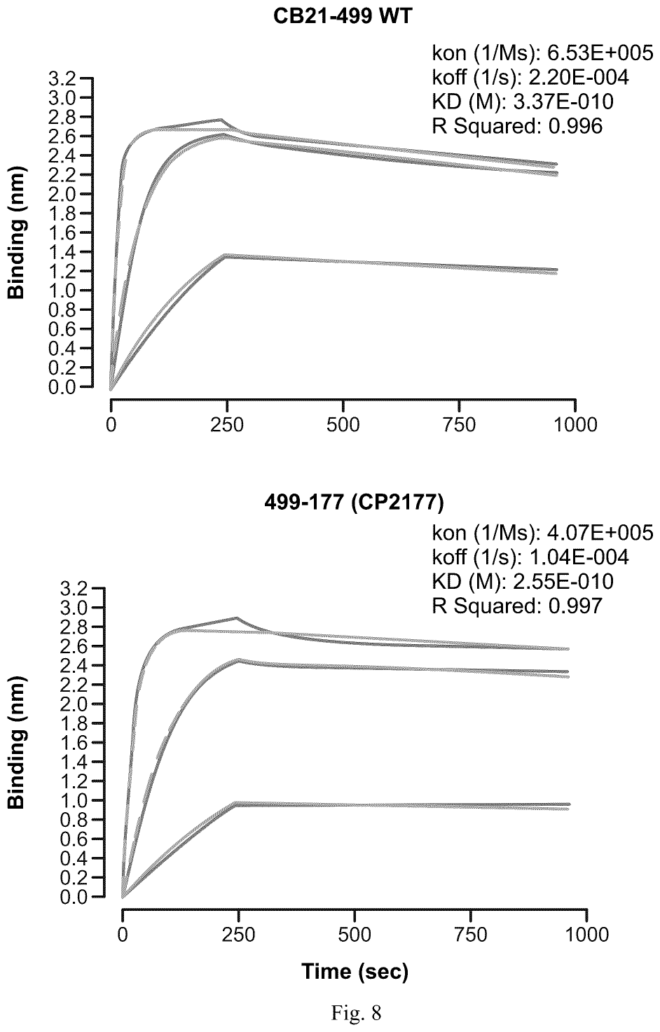

In round 3, CB21-499 physical liabilities were targeted, with a particular focus on improving thermostability. In silico analysis was performed to identify positions where mutation could potentially enhance thermostability. Through mutagenesis across 14 selected positions, we identified a D59E substitution in CDRH2 that, unexpectedly, improved both predicted thermostability and binding affinity as measured by BLI (FIG. 8).

### Example 9

### Characterization of CB21-498 and CB21-499 Antibody Families

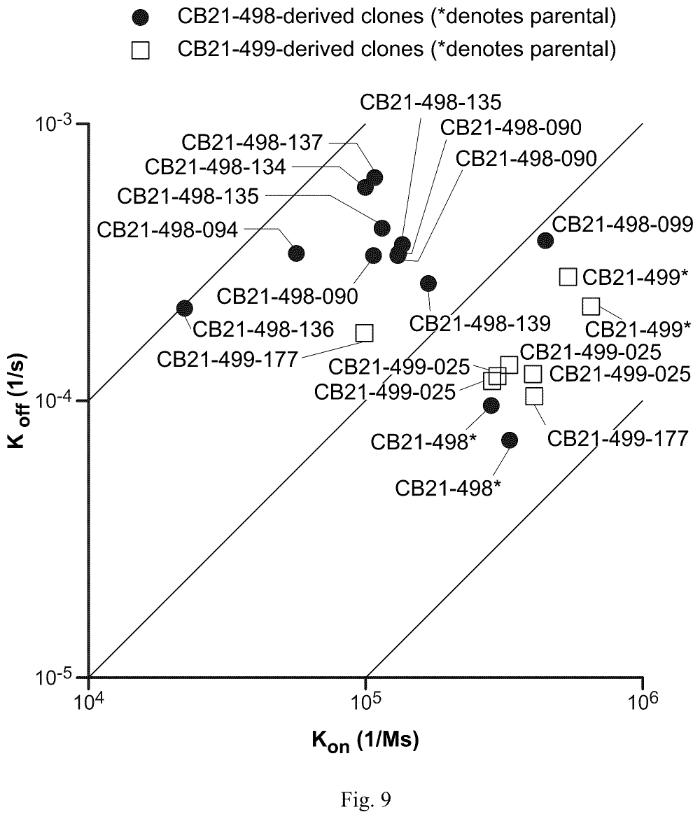

An isoaffinity plot of BLI kinetic analysis shows association (Kon) and dissociation (Koff) rates for CB21-498 and CB21-499 clonal variants binding to LY6G6D is shown in FIG. 9. Clones derived from the CB21-498 lineage are shown as filled circles, and those from CB21-499 are shown as open squares. Diagonal lines represent isoaffinity bands for 10 nM, 1 nM, and 0.1 nM apparent KD.

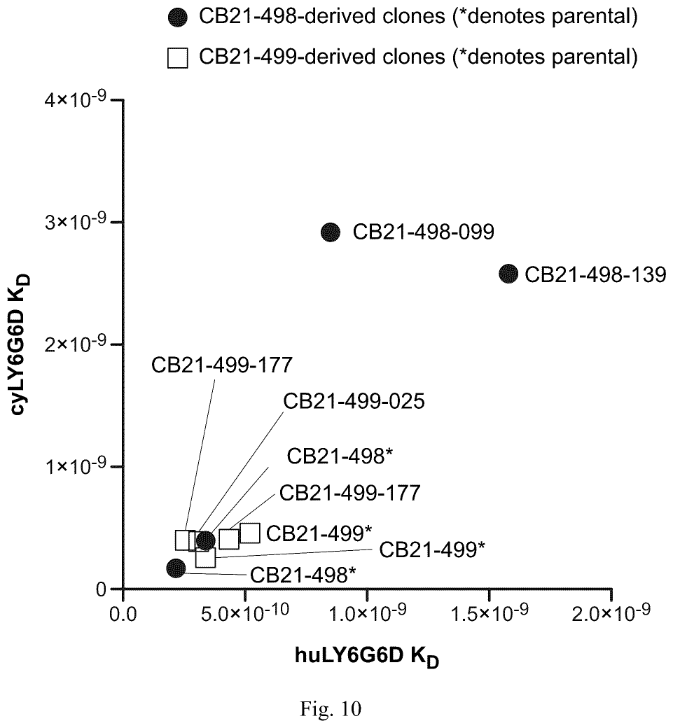

A BLI-derived binding affinity plot showing cross-reactivity of CB21-498 (gray) and CB21-499 (black) clonal variants to human and cynomolgus monkey LY6G6D proteins is shown in FIG. 10. Comparable KD values were observed for both species, indicating similar binding affinities across species.

Affinity-Capture Self-Interaction Nanoparticle Spectroscopy (AC-SINS) analysis was performed to assess the colloidal stability and self-association propensity of optimized clones, a common approach for evaluating antibody developability. A plate-based method utilized gold nanoparticles to capture test proteins of interest via affinity capture. Gold nanoparticles were coated with an anti-human Fc antibody and incubated with either control monoclonal antibodies or samples of interest. Absorbance was measured, and the maximum absorption wavelength (λmax) was determined. The shift in λmax (Δλmax, in nanometers) was calculated by subtracting the λmax obtained in the absence of antibody from the λmax measured for each sample. Test proteins that possess a propensity to self-associate will aggregate the particles, resulting in a change in the wavelength of maximum absorbance. A greater shift signifies a greater degree of self-association, which may imply that such proteins are less suitable for drug development as they may exhibit issues with high concentration formulation.

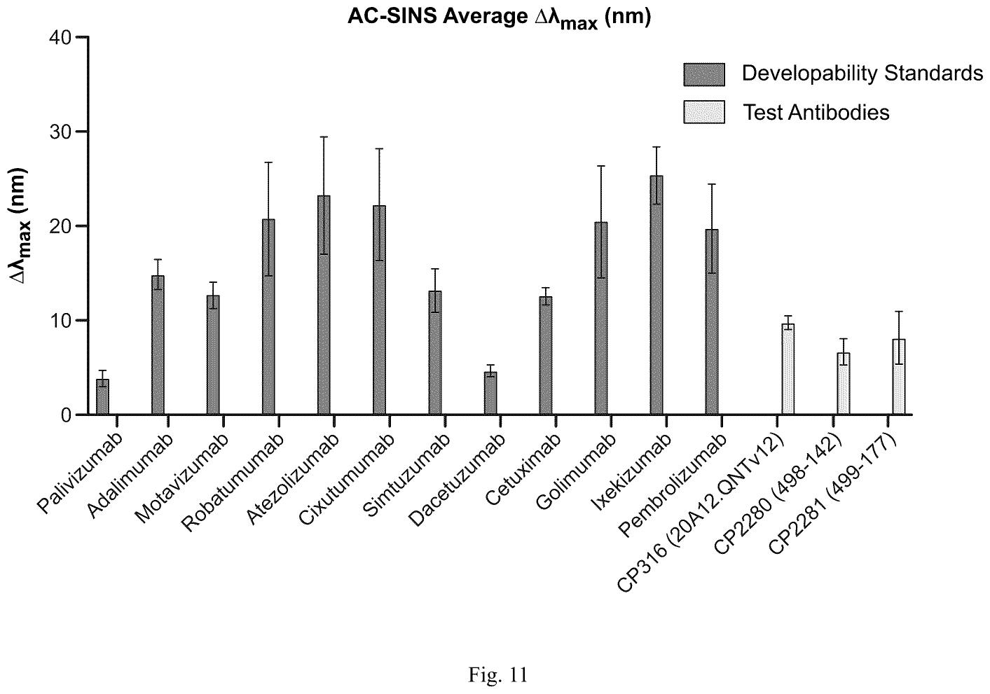

Test antibodies were benchmarked against a broad panel of clinical-stage antibodies used as developability standards. As shown in FIG. 11, clones derived from CB21-498-142 and CB21-499-177 exhibited favorable AC-SINS profiles, comparable to each other and to the literature-reported LY6G6D binder 20A12.QNTv12.

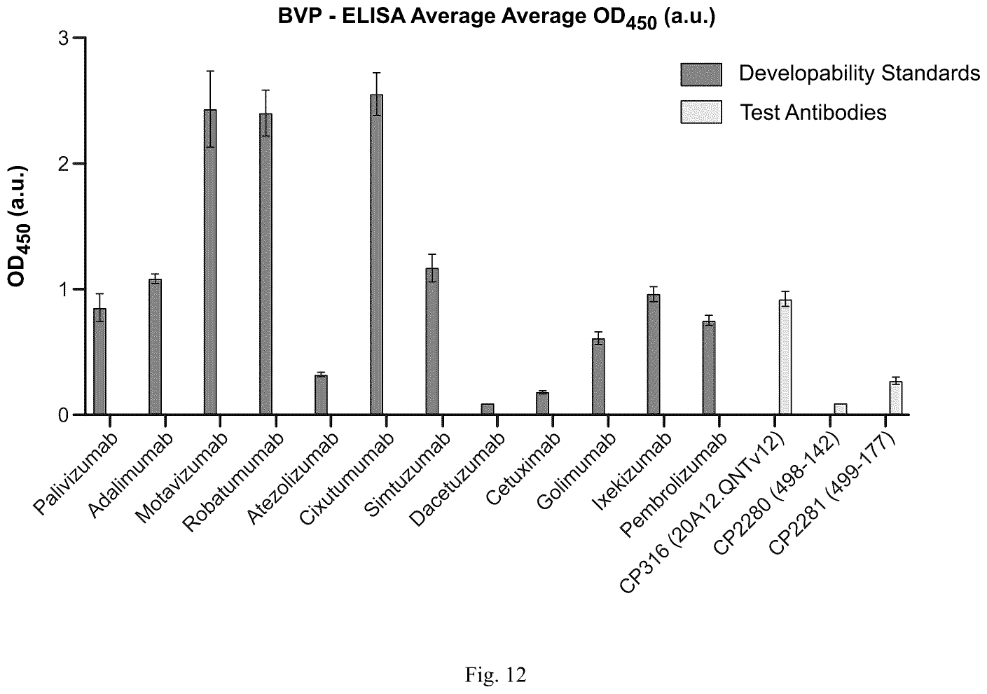

BVP-ELISA analysis was conducted to assess the polyreactivity of optimized clones, a commonly evaluated parameter in antibody developability characterization. Test clones were benchmarked against a panel of clinical-stage antibodies and the literature-reported LY6G6D binder 20A12.QNTv12. As shown in FIG. 12, optimized clones derived from CB21-498-142 and CB21-499-177 exhibited low polyreactivity, with OD450 values falling in the favorable range (<0.7) and well below those of several clinical-stage antibodies known to exhibit elevated polyreactivity. These clones also demonstrated slightly more favorable scores than 20A12.QNTv12, which showed a moderately elevated polyreactivity profile.

SPR multipoint kinetic analyses against human and cynomolgus monkey LY6G6D was also performed. LY6G6D binding kinetics were performed via surface plasmon resonance (SPR) on a Biacore 8K+. A Mouse Antibody Capture Kit, type 2 (Cytiva 29215281) was used for amine coupling to a CM5 sensor chip (BR100530) and the immobilization was performed exactly according to the manufacturer's standard settings. To begin an SPR run, 7 startup cycles were run following chip immobilization to establish sensor stability. In these, LY6G6D-mouse Fc fusion antigen was first flowed into just flow cell 2 at 10 uL/min at a 5 nM concentration with a 120 sec contact time. 1×HBS-EP+ buffer, pH 7.6 was then flowed at 30 μL/min with a 420 sec contact time into the chip. Finally regeneration solution (10 mM Glycine-HCl pH 1.7) was flowed at 30 uL/min with a 120 sec contact time.

After this point, 16 analysis cycles were run, 8 first with human LY6G6D-mouse Fc fusion antigen and 8 next for cyno LY6G6D-mouse Fc fusion antigen. In both antigen formats, the antigen was captured at 10 uL/min at a 5 nM concentration diluted into 1×HBS-EP+ buffer, pH 7.6 with a 120 sec contact time. Next, selected antibodies diluted into 1×HBS-EP+ buffer, pH 7.6 were run as the analytes across a 7-point dilution series, 50 nM to 0.7813 nM with two-fold dilutions. A contact time of 300 sec followed by dissociation time of 1200 sec at a flow rate of 30 μL/min was used. Regeneration solution (10 mM Glycine-HCl pH 1.7) was flowed at 30 μL/min with a 120 sec contact time. Finally, a wait step of 60 sec with flow rate of 30 μL/min 1×HBS-EP+ buffer, pH 7.6 was used to re-establish sensor stability. These SPR binding studies were performed at 25° C.

In both species, CB21-499-177 demonstrated a slower off-rate and improved binding affinity relative to the benchmark antibody 20A12.QNTv12, highlighting its enhanced target engagement. All clones tested showed comparable binding to human and cyno LY6G6D.

TABLE 9
SPR multipoint human LY6G6D binding
kinetics for optimized clones

| | 20A12.QNTv12 | CB21-498-142 | CB21-499-177 |
|---|---|---|---|
| ka (1/Ms) | 1.09 × 10⁵ | 9.67 × 10⁴ | 1.85 × 10⁵ |
| Kd (1/s) | 3.00 × 10⁻⁴ | 3.10 × 10⁻⁴ | 7.12 × 10⁻⁵ |
| Rmax (RU) | 69.3 | 75.7 | 89.7 |
| KD (M) | 3.67 × 10⁻⁹ | 3.20 × 10⁻⁹ | 3.85 × 10⁻¹⁰ |

TABLE 10
SPR multipoint cyno LY6G6D binding
kinetics for optimized clones

| | 20A12.QNTv12 | CB21-498-142 | CB21-499-177 |
|---|---|---|---|
| ka (1/Ms) | 1.12 × 10⁵ | 8.05 × 10⁴ | 1.66 × 10⁵ |
| Kd (1/s) | 3.80 × 10⁻⁴ | 4.48 × 10⁻⁴ | 1.49 × 10⁻⁴ |
| Rmax (RU) | 56.5 | 67.3 | 74.9 |
| KD (M) | 3.40 × 10⁻⁹ | 5.57 × 10⁻⁹ | 9.00 × 10⁻¹⁰ |

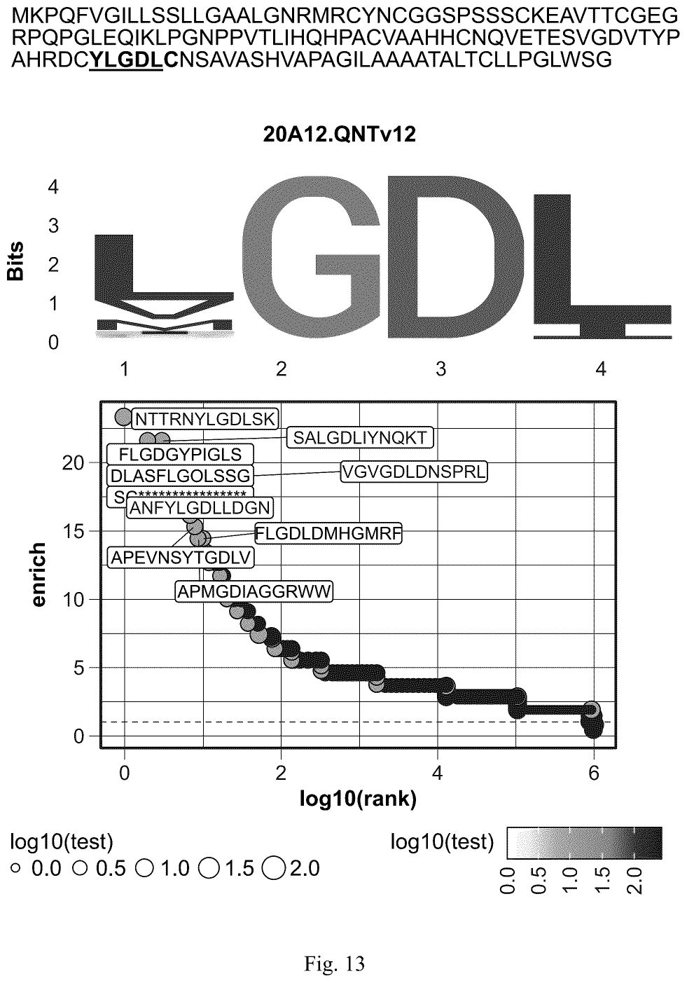

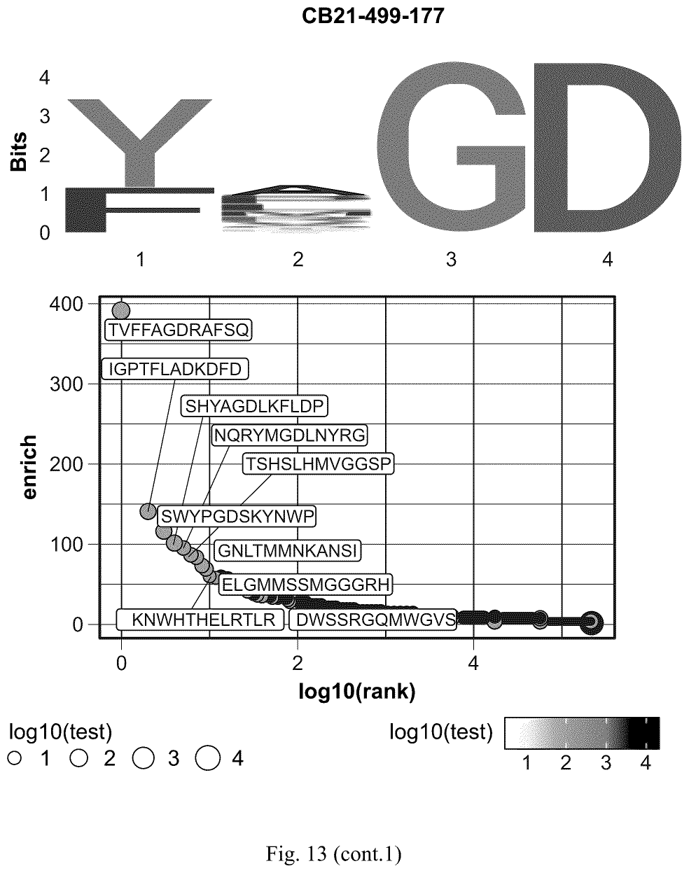

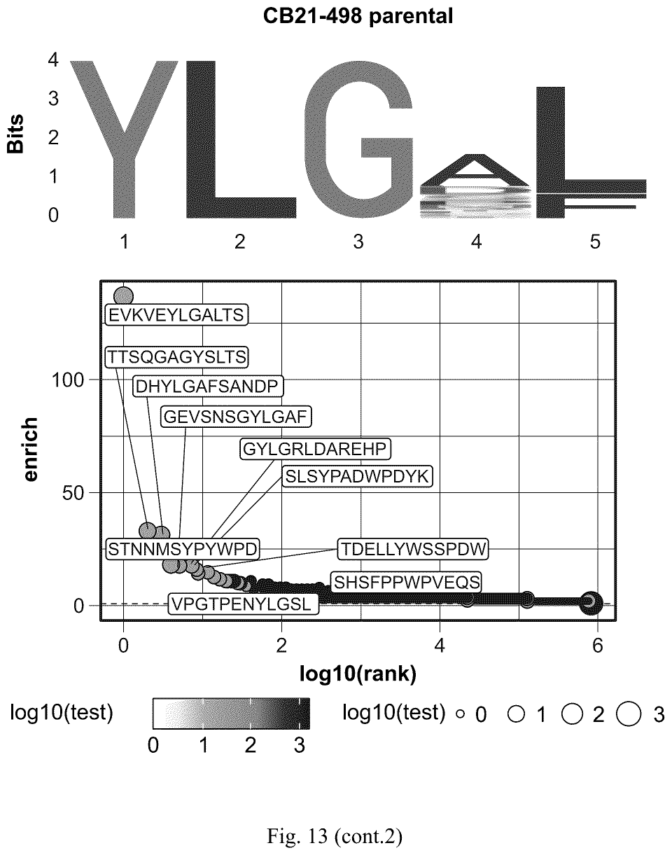

Epitope mapping was performed using AbMap to compare CB21-499-177, CB21-498, and the reference antibody 20A12.QNTv12. All three antibodies mapped to a similar region on the LY6G6D ECD, centered around the YLGDL motif, with subtle differences in fine specificity as shown in FIG. 13. AbMap phage enrichments were performed in triplicate and a representative sequence logo plot is shown for each antibody clone. These results suggest that while the antibodies share a common membrane-proximal epitope, the core binding region is distinct to each clone.

All references cited herein are incorporated by reference to the same extent as if each individual publication, database entry (e.g. Genbank sequences or GeneID entries), patent application, or patent, was specifically and individually indicated to be incorporated by reference. This statement of incorporation by reference is intended by Applicants, pursuant to 37 C.F.R. § 1.57(b)(1), to relate to each and every individual publication, database entry (e.g. Genbank sequences or GeneID entries), patent application, or patent, each of which is clearly identified in compliance with 37 C.F.R. § 1.57(b)(2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents. To the extent that the references provide a definition for a claimed term that conflicts with the definitions provided in the instant specification, the definitions provided in the instant specification shall be used to interpret the claimed invention.

While the invention has been described and exemplified in sufficient detail for those skilled in this art to make and use it, various alternatives, modifications, and improvements should be apparent without departing from the spirit and scope of the invention. The examples provided herein are representative of preferred embodiments, are exemplary, and are not intended as limitations on the scope of the invention. Modifications therein and other uses will occur to those skilled in the art. These modifications are encompassed within the spirit of the invention and are defined by the scope of the claims.

It will be readily apparent to a person skilled in the art that varying substitutions and modifications may be made to the invention disclosed herein without departing from the scope and spirit of the invention.

All patent applications, patents, publications and other references mentioned in the specification are indicative of the levels of those of ordinary skill in the art to which the invention pertains and are each incorporated herein by reference. The references cited herein are not admitted to be prior art to the claimed invention.

Unless otherwise defined, all technical and scientific terms used herein have the same meaning as commonly understood by one of ordinary skill in the art to which this invention belongs. In the case of conflict, the present specification, including definitions, will control.

The use of the articles “a”, “an”, and “the” in both the description and claims are to be construed to cover both the singular and the plural, unless otherwise indicated herein or clearly contradicted by context. The terms “comprising”, “having”, “being of” as in “being of a chemical formula”, “including”, and “containing” are to be construed as open terms (i.e., meaning “including but not limited to”) unless otherwise noted. Additionally, whenever “comprising” or another open-ended term is used in an embodiment, it is to be understood that the same embodiment can be more narrowly claimed using the intermediate term “consisting essentially of” or the closed term “consisting of”.

The term “about”, “approximately”, or “approximate”, when used in connection with a numerical value, means that a collection or range of values is included. For example, “about X” includes a range of values that are ±20%, ±10%, ±5%, ±2%, ±1%, ±0.5%, ±0.2%, or ±0.1% of X, where X is a numerical value. In one embodiment, the term “about” refers to a range of values which are 10% more or less than the specified value. In another embodiment, the term “about” refers to a range of values which are 5% more or less than the specified value. In another embodiment, the term “about” refers to a range of values which are 1% more or less than the specified value.

Recitation of ranges of values are merely intended to serve as a shorthand method of referring individually to each separate value falling within the range, unless otherwise indicated herein, and each separate value is incorporated into the specification as if it were individually recited herein. A range used herein, unless otherwise specified, includes the two limits of the range. For example, the terms “between X and Y” and “range from X to Y, are inclusive of X and Y and the integers there between. On the other hand, when a series of individual values are referred to in the disclosure, any range including any of the two individual values as the two end points is also conceived in this disclosure. For example, the expression “a dose of about 100 mg, 200 mg, or 400 mg” can also mean “a dose ranging from 100 to 200 mg”, “a dose ranging from 200 to 400 mg”, or “a dose ranging from 100 to 400 mg”.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

1. An antibody or antigen binding fragment thereof that binds to human LY6G6D, wherein the antibody or antigen binding fragment comprises heavy chain CDRs H1, H2, and H3 and light chain CDRs L1, L2, and L3 amino acid sequences as recited for one of the Identifiers in Table 1.

2. The antibody or antigen binding fragment of claim 1, wherein the antigen or binding fragment comprises a heavy chain variable region and a light chain variable region comprising amino acid sequences as recited for one of the Identifiers in Table 2; or a heavy chain variable region and a light chain variable region having at least 90% sequence identity, at least 95% sequence identity, at least 97% sequence identity, at least 98% sequence identity, or at least 99% sequence identity with a heavy chain variable region and a light chain variable region comprising amino acid sequences as recited for one of the Identifiers in Table 2.

3. The antibody or antigen binding fragment of claim 1, wherein the antibody or fragment thereof is an scFv.

4. The antibody or antigen binding fragment of claim 1, wherein the antibody or antigen binding fragment is provided as an intact IgG antibody.

5. The antibody or antigen binding fragment of claim 4, wherein the IgG antibody comprises a wild-type or mutated IgG2 Fc region.

6. The antibody or antigen binding fragment of claim 4, wherein the IgG antibody comprises an IgG1 Fc region.

7. The antibody or antigen binding fragment of claim 4, wherein the IgG1 Fc region comprises one or mutations that reduce effector functions of the IgG antibody relative to a wild-type IgG1 Fc region.

8. The antibody or antigen binding fragment of claim 4, wherein the IgG antibody comprises an IgG4 Fc region.

9. The antibody or antigen binding fragment of claim 1, wherein the antibody or antigen binding fragment is human or is humanized.

10. The antibody or antigen binding fragment of claim 1 that comprises a glycosylation pattern characteristic of expression by a mammalian cell.

11. The antibody or antigen binding fragment of claim 9 that comprises a glycosylation pattern characteristic of expression by a CHO cell.

12. The antibody or antigen binding fragment of claim 1, wherein the antibody or antigen binding fragment is provided as a bispecific T cell engaging antibody (BiTE), a (SCFV)2, a NANOBODY®, a nanobody-HSA VHH-scAb, a VHH-Fab, a Dual scFab, a F(ab′)₂, a diabody, a CROSSMAB®, a DAF (two-in-one), a DAE (four-in-one), a DUTAMAB®, a DT-TgG, a knobs-in-holes common light chain, a knobs-in-holes assembly, a charge pair, a Fab-arm exchange, a SEEDbody, a LUZ-Y, a FcAb, a kl-body, an orthogonal Fab, a DVD-IgG, a IgG (H)-scFv, a scFv-(H) IgG, IgG (L)-scFv, scFv-(L) IgG, IgG (L,H)-Fv, IgG (H)-V, V(H)-IgG, IgG (L)-V, V(L)-IgG, KIH IgG-scFab, 2scFv-IgG, IgG-2scFv, scFv4-Ig, ZYBODY™, DVI-IgG, Diabody-C_{H}3, a triple body, a miniantibody, a minibody, a TriBi minibody, scFv-C_{H}3 KIH, Fab-scFv, a F(ab′)₂-scFv2, a scFv-KIH, a Fab-scFv-Fc, a tetravalent HCAb, a scDiabody-Fc, a Diabody-Fc, a tandem scFv-Fc, a VHH-Fc, a tandem VHH-Fc, a LTIH-Fc KiH, a Fab-VHH-Fc, an Intrabody, a dock and lock, an ImmTAC® (immune-mobilizing monoclonal TCRs (T cell receptors) against cancer), an IgG-IgG conjugate, a Cov-X-Body, a scFv1-PEG-scFv2, an Adnectin, a DARPin, or a fibronectin, an IgG, an IgM, an IgA, an IgE, an IgD, or a DEP conjugate, TMEAbody™, SAFEbody®, TRITAC®, a dual affinity retargeting (DART®) bispecific antibody, a simultaneous multiple interaction T-cell engagers (SMITE), or a SHIELD.

13. The antibody or antigen binding fragment of claim 1, wherein the antibody or antigen binding fragment thereof comprises an immunoglobulin binding motif that binds to an antigen selected from the group consisting CD16, γδ TCR, CTLA-4, PD-1, PD-L1, 4-1BB, IL-13, IL-4, VEGF, and CD3.

14. The antibody according to claim 1, wherein the antibody comprises heavy chain CDRs H1, H2, and H3 and light chain CDRs L1, L2, and L3 amino acid sequences as recited for an Identifier in Table 1 selected from the group consisting of CB21-498, CB21-498-001, CB21-498-002, CB21-498-003, CB21-498-004, CB21-498-005, CB21-498-006, CB21-498-007, CB21-498-008, CB21-498-009, CB21-498-010, CB21-498-011, CB21-498-012, CB21-498-013, CB21-498-014, CB21-498-015, CB21-498-016, CB21-498-017, CB21-498-018, CB21-498-019, CB21-498-020, CB21-498-021, CB21-498-022, CB21-498-023, CB21-498-024, CB21-498-025, CB21-498-026, CB21-498-027, CB21-498-028, CB21-498-029, CB21-498-030, CB21-498-031, CB21-498-032, CB21-498-033, CB21-498-034, CB21-498-035, CB21-498-036, CB21-498-037, CB21-498-038, CB21-498-039, CB21-498-040, CB21-498-041, CB21-498-042, CB21-498-043, CB21-498-044, CB21-498-045, CB21-498-046, CB21-498-047, CB21-498-048, CB21-498-049, CB21-498-050, CB21-498-051, CB21-498-052, CB21-498-053, CB21-498-054, CB21-498-055, CB21-498-056, CB21-498-057, CB21-498-058, CB21-498-059, CB21-498-060, CB21-498-061, CB21-498-062, CB21-498-063, CB21-498-064, CB21-498-065, CB21-498-066, CB21-498-067, CB21-498-068, CB21-498-069, CB21-498-070, CB21-498-071, CB21-498-072, CB21-498-073, CB21-498-074, CB21-498-075, CB21-498-076, CB21-498-077, CB21-498-078, CB21-498-079, CB21-498-080, CB21-498-081, CB21-498-082, CB21-498-083, CB21-498-084, CB21-498-085, CB21-498-086, CB21-498-087, CB21-498-088, CB21-498-089, CB21-498-090, CB21-498-091, CB21-498-092, CB21-498-093, CB21-498-094, CB21-498-095, CB21-498-096, CB21-498-097, CB21-498-098, CB21-498-099, CB21-498-100, CB21-498-101, CB21-498-102, CB21-498-103, CB21-498-104, CB21-498-105, CB21-498-106, CB21-498-107, CB21-498-108, CB21-498-109, CB21-498-110, CB21-498-111, CB21-498-112, CB21-498-113, CB21-498-114, CB21-498-115, CB21-498-116, CB21-498-117, CB21-498-118, CB21-498-119, CB21-498-120, CB21-498-121, CB21-498-122, CB21-498-123, CB21-498-124, CB21-498-125, CB21-498-126, CB21-498-127, CB21-498-128, CB21-498-129, CB21-498-130, CB21-498-131, CB21-498-132, CB21-498-133, CB21-498-134, CB21-498-135, CB21-498-136, CB21-498-137, CB21-498-138, CB21-498-139, CB21-498-140, CB21-498-141, CB21-498-142, CB21-498-143, CB21-499, CB21-499-001, CB21-499-002, CB21-499-003, CB21-499-004, CB21-499-005, CB21-499-006, CB21-499-007, CB21-499-008, CB21-499-009, CB21-499-010, CB21-499-011, CB21-499-012, CB21-499-013, CB21-499-014, CB21-499-015, CB21-499-016, CB21-499-017, CB21-499-018, CB21-499-019, CB21-499-020, CB21-499-021, CB21-499-022, CB21-499-023, CB21-499-024, CB21-499-025, CB21-499-026, CB21-499-027, CB21-499-028, CB21-499-029, CB21-499-030, CB21-499-031, CB21-499-032, CB21-499-033, CB21-499-034, CB21-499-035, CB21-499-036, CB21-499-037, CB21-499-038, CB21-499-039, CB21-499-040, CB21-499-041, CB21-499-042, CB21-499-043, CB21-499-044, CB21-499-045, CB21-499-046, CB21-499-047, CB21-499-048, CB21-499-049, CB21-499-050, CB21-499-051, CB21-499-052, CB21-499-053, CB21-499-054, CB21-499-055, CB21-499-056, CB21-499-057, CB21-499-058, CB21-499-059, CB21-499-060, CB21-499-061, CB21-499-062, CB21-499-063, CB21-499-064, CB21-499-065, CB21-499-066, CB21-499-067, CB21-499-068, CB21-499-069, CB21-499-070, CB21-499-071, CB21-499-072, CB21-499-073, CB21-499-074, CB21-499-075, CB21-499-076, CB21-499-077, CB21-499-078, CB21-499-079, CB21-499-080, CB21-499-081, CB21-499-082, CB21-499-083, CB21-499-084, CB21-499-085, CB21-499-086, CB21-499-087, CB21-499-088, CB21-499-089, CB21-499-090, CB21-499-091, CB21-499-092, CB21-499-093, CB21-499-094, CB21-499-095, CB21-499-096, CB21-499-097, CB21-499-098, CB21-499-099, CB21-499-100, CB21-499-101, CB21-499-102, CB21-499-103, CB21-499-104, CB21-499-105, CB21-499-106, CB21-499-107, CB21-499-108, CB21-499-109, CB21-499-110, CB21-499-111, CB21-499-112, CB21-499-113, CB21-499-114, CB21-499-115, CB21-499-116, CB21-499-117, CB21-499-118, CB21-499-119, CB21-499-120, CB21-499-121, CB21-499-122, CB21-499-123, CB21-499-124, CB21-499-125, CB21-499-126, CB21-499-127, CB21-499-128, CB21-499-129, CB21-499-130, CB21-499-131, CB21-499-132, CB21-499-133, CB21-499-134, CB21-499-135, CB21-499-136, CB21-499-137, CB21-499-138, CB21-499-139, CB21-499-140, CB21-499-141, CB21-499-142, CB21-499-143, CB21-499-144, CB21-499-145, CB21-499-146, CB21-499-147, CB21-499-148, CB21-499-149, CB21-499-150, CB21-499-151, CB21-499-152, CB21-499-153, CB21-499-154, CB21-499-155, CB21-499-156, CB21-499-157, CB21-499-158, CB21-499-159, CB21-499-160, CB21-499-161, CB21-499-162, CB21-499-163, CB21-499-164, CB21-499-165, CB21-499-166, CB21-499-167, CB21-499-168, CB21-499-169, CB21-499-170, CB21-499-171, CB21-499-172, CB21-499-174, CB21-499-175, CB21-499-176, CB21-499-177, CB21-499-178, and CB21-499-179.

15. The antibody according to claim 1, wherein the antigen or binding fragment comprises a heavy chain variable region and a light chain variable region comprising amino acid sequences as recited for an Identifier in Table 2 selected from the group consisting of CB21-498, CB21-498-001, CB21-498-002, CB21-498-003, CB21-498-004, CB21-498-005, CB21-498-006, CB21-498-007, CB21-498-008, CB21-498-009, CB21-498-010, CB21-498-011, CB21-498-012, CB21-498-013, CB21-498-014, CB21-498-015, CB21-498-016, CB21-498-017, CB21-498-018, CB21-498-019, CB21-498-020, CB21-498-021, CB21-498-022, CB21-498-023, CB21-498-024, CB21-498-025, CB21-498-026, CB21-498-027, CB21-498-028, CB21-498-029, CB21-498-030, CB21-498-031, CB21-498-032, CB21-498-033, CB21-498-034, CB21-498-035, CB21-498-036, CB21-498-037, CB21-498-038, CB21-498-039, CB21-498-040, CB21-498-041, CB21-498-042, CB21-498-043, CB21-498-044, CB21-498-045, CB21-498-046, CB21-498-047, CB21-498-048, CB21-498-049, CB21-498-050, CB21-498-051, CB21-498-052, CB21-498-053, CB21-498-054, CB21-498-055, CB21-498-056, CB21-498-057, CB21-498-058, CB21-498-059, CB21-498-060, CB21-498-061, CB21-498-062, CB21-498-063, CB21-498-064, CB21-498-065, CB21-498-066, CB21-498-067, CB21-498-068, CB21-498-069, CB21-498-070, CB21-498-071, CB21-498-072, CB21-498-073, CB21-498-074, CB21-498-075, CB21-498-076, CB21-498-077, CB21-498-078, CB21-498-079, CB21-498-080, CB21-498-081, CB21-498-082, CB21-498-083, CB21-498-084, CB21-498-085, CB21-498-086, CB21-498-087, CB21-498-088, CB21-498-089, CB21-498-090, CB21-498-091, CB21-498-092, CB21-498-093, CB21-498-094, CB21-498-095, CB21-498-096, CB21-498-097, CB21-498-098, CB21-498-099, CB21-498-100, CB21-498-101, CB21-498-102, CB21-498-103, CB21-498-104, CB21-498-105, CB21-498-106, CB21-498-107, CB21-498-108, CB21-498-109, CB21-498-110, CB21-498-111, CB21-498-112, CB21-498-113, CB21-498-114, CB21-498-115, CB21-498-116, CB21-498-117, CB21-498-118, CB21-498-119, CB21-498-120, CB21-498-121, CB21-498-122, CB21-498-123, CB21-498-124, CB21-498-125, CB21-498-126, CB21-498-127, CB21-498-128, CB21-498-129, CB21-498-130, CB21-498-131, CB21-498-132, CB21-498-133, CB21-498-134, CB21-498-135, CB21-498-136, CB21-498-137, CB21-498-138, CB21-498-139, CB21-498-140, CB21-498-141, CB21-498-142, CB21-498-143, CB21-499, CB21-499-001, CB21-499-002, CB21-499-003, CB21-499-004, CB21-499-005, CB21-499-006, CB21-499-007, CB21-499-008, CB21-499-009, CB21-499-010, CB21-499-011, CB21-499-012, CB21-499-013, CB21-499-014, CB21-499-015, CB21-499-016, CB21-499-017, CB21-499-018, CB21-499-019, CB21-499-020, CB21-499-021, CB21-499-022, CB21-499-023, CB21-499-024, CB21-499-025, CB21-499-026, CB21-499-027, CB21-499-028, CB21-499-029, CB21-499-030, CB21-499-031, CB21-499-032, CB21-499-033, CB21-499-034, CB21-499-035, CB21-499-036, CB21-499-037, CB21-499-038, CB21-499-039, CB21-499-040, CB21-499-041, CB21-499-042, CB21-499-043, CB21-499-044, CB21-499-045, CB21-499-046, CB21-499-047, CB21-499-048, CB21-499-049, CB21-499-050, CB21-499-051, CB21-499-052, CB21-499-053, CB21-499-054, CB21-499-055, CB21-499-056, CB21-499-057, CB21-499-058, CB21-499-059, CB21-499-060, CB21-499-061, CB21-499-062, CB21-499-063, CB21-499-064, CB21-499-065, CB21-499-066, CB21-499-067, CB21-499-068, CB21-499-069, CB21-499-070, CB21-499-071, CB21-499-072, CB21-499-073, CB21-499-074, CB21-499-075, CB21-499-076, CB21-499-077, CB21-499-078, CB21-499-079, CB21-499-080, CB21-499-081, CB21-499-082, CB21-499-083, CB21-499-084, CB21-499-085, CB21-499-086, CB21-499-087, CB21-499-088, CB21-499-089, CB21-499-090, CB21-499-091, CB21-499-092, CB21-499-093, CB21-499-094, CB21-499-095, CB21-499-096, CB21-499-097, CB21-499-098, CB21-499-099, CB21-499-100, CB21-499-101, CB21-499-102, CB21-499-103, CB21-499-104, CB21-499-105, CB21-499-106, CB21-499-107, CB21-499-108, CB21-499-109, CB21-499-110, CB21-499-111, CB21-499-112, CB21-499-113, CB21-499-114, CB21-499-115, CB21-499-116, CB21-499-117, CB21-499-118, CB21-499-119, CB21-499-120, CB21-499-121, CB21-499-122, CB21-499-123, CB21-499-124, CB21-499-125, CB21-499-126, CB21-499-127, CB21-499-128, CB21-499-129, CB21-499-130, CB21-499-131, CB21-499-132, CB21-499-133, CB21-499-134, CB21-499-135, CB21-499-136, CB21-499-137, CB21-499-138, CB21-499-139, CB21-499-140, CB21-499-141, CB21-499-142, CB21-499-143, CB21-499-144, CB21-499-145, CB21-499-146, CB21-499-147, CB21-499-148, CB21-499-149, CB21-499-150, CB21-499-151, CB21-499-152, CB21-499-153, CB21-499-154, CB21-499-155, CB21-499-156, CB21-499-157, CB21-499-158, CB21-499-159, CB21-499-160, CB21-499-161, CB21-499-162, CB21-499-163, CB21-499-164, CB21-499-165, CB21-499-166, CB21-499-167, CB21-499-168, CB21-499-169, CB21-499-170, CB21-499-171, CB21-499-172, CB21-499-174, CB21-499-175, CB21-499-176, CB21-499-177, CB21-499-178, and CB21-499-179.

16. The antibody according to claim 1, wherein the antigen or binding fragment comprises a heavy chain variable region and a light chain variable region comprising amino acid sequences as recited for an Identifier in Table 2 selected from the group consisting of CB21-499-177 and CB21-498-142.

17. An isolated nucleic acid encoding any one of the antibodies or antigen binding fragments of claim 1.

18. An expression vector comprising the isolated nucleic acid of claim 17.

19. A host cell comprising expression vector of claim 18.

20. The host cell of claim 19, which is a bacterial cell, a human cell, a mammalian cell, a Pichia cell, a plant cell, an HEK293 cell, or a CHO cell.

21. A composition comprising the antibody or antigen binding fragment of claim 1 and a pharmaceutically acceptable carrier or diluent.

22. The composition according to claim 21, further comprising one or more agents selected from the group consisting of anti-CD27 antibody, anti-CD47 antibody, anti-APRIL antibody, anti-PD-1 antibody, anti-PD-L1 antibody, anti-TIGIT antibody, anti-CTLA4 antibody, anti-CS1 antibody, anti-KIR2DL1/2/3 antibody, anti-CD137 antibody, anti-GITR antibody, anti-PD-L2 antibody, anti-ILT1 antibody, anti-ILT2 antibody, anti-ILT3 antibody, anti-ILT4 antibody, anti-ILT5 antibody, anti-ILT6 antibody, anti-ILT7 antibody, anti-ILT8 antibody, anti-CD40 antibody, anti-OX40 antibody, anti-ICOS, anti-KIR2DL1 antibody, anti-KIR2DL2/3 antibody, anti-KIR2DL4 antibody, anti-KIR2DL5A antibody, anti-KIR2DL5B antibody, anti-KIR3DL1 antibody, anti-KIR3DL2 antibody, anti-KIR3DL3 antibody, anti-NKG2A antibody, anti-NKG2C antibody, anti-NKG2E antibody, anti-4-1BB antibody, anti-TSLP antibody, anti-IL-10 antibody, IL-10 PEGylated IL-10, an agonist (e.g., an agonistic antibody or antigen-binding fragment thereof, or a soluble fusion) of a TNF receptor protein, an Immunoglobulin-like protein, a cytokine receptor, an integrin, a signaling lymphocytic activation molecules (SLAM proteins), an activating NK cell receptor, a Toll like receptor, OX40, CD2, CD7, CD27, CD28, CD30, CD40, ICAM-1, LFA-1 (CDl 1a/CD18), 4-1BB (CD137), B7-H3, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CDl 1d, ITGAE, CD103, ITGAL, ITGAM, CDl 1b, ITGAX, CDl 1c, ITGB1, CD29, ITGB2, CD18, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), SLAM7, BLAME (SLAMF8), SELPLG (CD162), LTBR, LAT, GADS, PAG/Cbp, CD19a, a ligand that specifically binds with CD83, an inhibitor of CD47, an inhibitor of PD-1, an inhibitor of PD-L1, an inhibitor of PD-L2, an inhibitor of CTLA4, an inhibitor of TIM3, an inhibitor of LAG3, an inhibitor of CEACAM (e.g., CEACAM-1, -3 and/or -5), an inhibitor of VISTA, an inhibitor of BTLA, an inhibitor of TIGIT, an inhibitor of LAIR1, an inhibitor of IDO, an inhibitor of TDO, an inhibitor of CD160, an inhibitor of TGFR beta, and a cyclic dinculeotide or other STING pathway agonist.

23. A method of producing an antibody or antigen binding fragment comprising:
culturing a host cell comprising one or more polynucleotides encoding any one of the antibodies or antigen binding fragments of claim 1 under conditions favorable to expression of the polynucleotide; and optionally, recovering the antibody or antigen binding fragment from the host cell and/or culture medium.

24. An antibody or antigen binding fragment according to claim 1, for the treatment of cancer.

25. A method of treating cancer in a human subject, comprising administering to the subject an effective amount of an antibody or antigen binding fragment of claim 1, optionally in association with a further therapeutic agent or therapeutic procedure.
